(19) United States
(12) Patent Application Publication
(10) Pub. No.: US 2011/0020300 A1
(43) Pub. Date: Jan. 27, 2011

(54) COMPOSITIONS AND METHODS FOR INHIBITING EXPRESSION OF GLUCOCORTICOID RECEPTOR (GCR) GENES

(75) Inventors: JACQUES BAILLY, ZIMMERSHEIM (FR); AGNÈS BÉNARDEAU, SAINT LOUIS (FR); BIRGIT BRAMLAGE, KULMBACH (DE); RAINER CONSTIEN, KULMBACH (DE); ANDREA FORST, BAYREUTH (DE); MARKUS HOSSBACH, KULMBACH (DE); BRIGITTE SCHOTT, LANDSER (FR)

Correspondence Address: GENENTECH, INC., 1 DNA WAY, SOUTH SAN FRANCISCO, CA, 94080, US

(73) Assignee: GENENTECH, INC., SOUTH SAN FRANCISCO, CA (US)

(21) Appl. No.: 12/780,813

(22) Filed: May 14, 2010

(30) Foreign Application Priority Data
May 15, 2009 (EP) EP 09160411.6

(51) Int. Cl. A61K 35/12; C07H 21/02; C12N 15/63; C12N 5/00; A01K 67/027; A61K 31/7052; A61P 3/10; A61P 3/00; A61P 9/00; A61P 25/24

(52) U.S. Cl. 424/93.21; 536/24.5; 435/320.1; 435/325; 800/13; 514/44 A; 435/375

(57) ABSTRACT

This invention relates to a double-stranded ribonucleic acid (dsRNA) for inhibiting the expression of a GCR gene. The invention also relates to a pharmaceutical composition comprising the dsRNA or nucleic acid molecules or vectors encoding the same together with a pharmaceutically acceptable carrier; methods for treating diseases caused by the expression of a GCR gene using said pharmaceutical composition; and methods for inhibiting the expression of GCR in a cell.

### CROSS-REFERENCE TO RELATED APPLICATIONS

This application claims benefit of priority under 35 USC §119(a) to European patent application number 09160411.6, filed 15 May 2009, the contents of which are incorporated herein by reference.

### FIELD OF THE INVENTION

This invention relates to double-stranded ribonucleic acids (dsRNAs), and their use in mediating RNA interference to inhibit the expression of the GCR gene. Furthermore, the use of said dsRNAs to treat/prevent a wide range of diseases/disorders which are associated with the expression of the GCR gene, like diabetes, dyslipidemia, obesity, hypertension, cardiovascular diseases or depression is part of the invention.

### BACKGROUND OF THE INVENTION

Glucocorticoids are responsible for several physiological functions including response to stress, immune and inflammatory responses as well as stimulation of hepatic gluconeogenesis and glucose utilization at the periphery. Glucocorticoids act via an intracellular glucocorticoid receptor (GCR) belonging to the family of the nuclear steroidal receptors. The non-activated GCR is located in the cellular cytoplasm and is associated with several chaperone proteins. When a ligand activates the receptor, the complex is translocated in the cell nucleus and interacts with the glucocorticoid response element which is located in several gene promoters. The receptor could act in the cell nucleus as an homodimer or an heterodimer. Moreover several associated co-activators or co-repressors could also interact with the complex. This large range of possible combinations leads to several GCR conformations and several possible physiological responses making it difficult to identify a small chemical entity which can act as a full and specific GCR inhibitor.

Pathologies like diabetes, Cushing's syndrome or depression have been associated with moderate to severe hypercortisolism (Chiodini et al, Eur. J. Endocrinol. 2005, Vol. 153, pp 837-844; Young, Stress 2004, Vol. 7 (4), pp 205-208). GCR antagonist administration has been proven to be clinically active in depression (Flores et al, Neuropsychopharmacology 2006, Vol. 31, pp 628-636) or in Cushing's syndrome (Chu et al, J. Clin. Endocrinol. Metab. 2001, Vol. 86, pp 3568-3573). These clinical evidences illustrate the potential clinical value of a potent and selective GCR antagonist in many indications like diabetes, dyslipidemia, obesity, hypertension, cardiovascular diseases or depression (Von Geldern et al, J. Med. Chem. 2004, Vol 47 (17), pp 4213-4230; Hu et al, Drug Develop. Res. 2006, Vol. 67, pp 871-883; Andrews, Handbook of the stress and the brain 2005, Vol. 15, pp 437-450). This approach might also improve peripheral insulin sensitivity (Zinker et al, Meta. Clin. Exp. 2007, Vol. 57, pp 380-387) and protect pancreatic beta cells (Delauney et al, J. Clin. Invest. 1997, Vol. (100, pp 2094-2098).

Diabetic patients have an increased level of fasting blood glucose which has been correlated in clinic with an impaired control of gluconeogenesis (DeFronzo, Med. Clin. N. Am. 2004, Vol. 88 pp 787-835). The hepatic gluconeogenesis process is under the control of glucocorticoids. Clinical administration of a non-specific GCR antagonist (RU486/mifepristone) leads acutely to a decrease of fasting plasma glucose in normal volunteers (Garrel et al, J. Clin. Endocrinol. Metab. 1995, Vol. 80 (2), pp 379-385) and chronically to a decrease of plasmatic HbAlc in Cushing's patients (Nieman et al, J. Clin. Endocrinol. Metab. 1985, Vol. 61 (3), pp 536-540). Moreover, this drug given to leptin deficient animals normalizes fasting plasma glucose (ob/ob mice, Gettys et al, Int. J. Obes. 1997, Vol. 21, pp 865-873) as well as the activity of gluconeogenic enzymes (db/db mice, Friedman et al, J. Biol. Chem. 1997, Vol. 272 (50) pp 31475-31481). Liver-specific knockout mice have been produced and these animals display a moderate hypoglycemia when they are fasted for 48 h minimizing the risk of severe hypoglycemia (Opherk et al, Mol. Endocrinol. 2004, Vol. 18 (6), pp 1346-1353). Moreover, hepatic and adipose tissue GCR silencing in diabetic mice (db/db mice) with an antisense approach leads to significant reduction of blood glucose (Watts et al, Diabetes, 2005, Vol 54, pp 1846-1853).

Endogenous corticosteroid secretion at the level of the adrenal gland can be modulated by the Hypothalamus-Pituitary gland-Adrenal gland axis (HPA). Low plasma level of endogenous corticosteroids can activate this axis via a feed-back mechanism which leads to an increase of endogenous corticosteroids circulating in the blood. Mifepristone which crosses the blood brain barrier is known to stimulate the HPA axis which ultimately leads to an increase of endogenous corticosteroids circulating in the blood (Gaillard et al, Pro. Natl. Acad. Sci. 1984, Vol. 81, pp 3879-3882). Mifepristone also induces some adrenal insufficiency symptoms after long term treatment (up to 1 year, for review see: Sitruk-Ware et al, 2003, Contraception, Vol. 68, pp 409-420). Moreover because of its lack of tissue selectivity Mifepristone inhibits the effect of glucocorticoids at the periphery in preclinical models as well as in human (Jacobson et al, 2005 J. Pharm. Exp. Ther. Vol 314 (1) pp 191-200; Gaillard et al, 1985 J. Clin. Endo. Met., Vol. 61 (6), pp 1009-1011)

For GCR modulator to be used in indications such as diabetes, dyslipidemia, obesity, hypertension and cardiovascular diseases it is necessary to limit the risk to activate or inhibit the HPA axis and to inhibit GCR at the periphery in other organs than liver. Silencing directly GCR in hepatocytes can be an approach to modulate/normalize hepatic gluconeogenesis as demonstrated recently. However this effect has been seen only at rather high concentrations (in vitro IC50 in the range of 25 nM/Watts et al, Diabetes, 2005, Vol 54, pp 1846-1853). To minimize the risk of off target effect as well as to limit pharmacological activity at the periphery in other organs than liver it would be necessary to get more potent GCR silencing agent.

All references cited herein, including patent applications and publications, are incorporated by reference in their entirety.

### SUMMARY OF THE INVENTION

Double-stranded ribonucleic acid (dsRNA) molecules have been shown to block gene expression in a highly conserved regulatory mechanism known as RNA interference (RNAi). The invention provides double-stranded ribonucleic acid (dsRNA) molecules able to selectively and efficiently decrease the expression of GCR. The use of GCR RNAi provides a method for the therapeutic and/or prophylactic treatment of diseases/disorders which are associated with any dysregulation of the glucocorticoid pathway. These diseases/disorders can occur due to systemic or local overproduction of endogenous glucocorticoids or due to treatment with synthetic glucocorticoids (e.g. diabetic-like syndrome in patients treated with high doses of glucocorticoids).

Particular disease/disorder states include the therapeutic and/or prophylactic treatment of type 2 diabetes, obesity, dislipidemia, diabetic atherosclerosis, hypertension and depression, which method comprises administration of dsRNA targeting GCR to a human being or animal. Further, the invention provides a method for the therapeutic and/or prophylactic treatment of Metabolic Syndrome X, Cushing's Syndrome, Addison's disease, inflammatory diseases such as asthma, rhinitis, and arthritis, allergy, autoimmune disease, immunodeficiency, anorexia, cachexia, bone loss or bone frailty, and wound healing. Metabolic Syndrome X refers to a cluster of risk factors that include obesity, dyslipidemia, particularly high triglycerides, glucose intolerance, high blood sugar and high blood pressure.

In one preferred embodiment the described dsRNA molecule is capable of inhibiting the expression of a GCR gene by at least 70%, preferably by at least 80%, most preferably by at least 90%. The invention also provides compositions and methods for specifically targeting the liver with GCR dsRNA, for treating pathological conditions and diseases caused by the expression of the GCR gene including those described above. In other embodiments the invention provides compositions and methods for specifically targeting other tissues or organs affected, including, but not limited to adipose tissue, the hypothalamus, kidneys or the pancreas.

In one embodiment, the invention provides double-stranded ribonucleic acid (dsRNA) molecules for inhibiting the expression of a GCR gene, in particular the expression of the mammalian or human GCR gene. The dsRNA comprises at least two sequences that are complementary to each other. The dsRNA comprises a sense strand comprising a first sequence and an antisense strand may comprise a second sequence, see sequences provided in the sequence listing and also provision of specific dsRNA pairs in the appended tables 1 and 4. In one embodiment the sense strand comprises a sequence which has an identity of at least 90% to at least a portion of an mRNA encoding GCR. Said sequence is located in a region of complementarity of the sense strand to the antisense strand, preferably within nucleotides 2-7 of the 5′ terminus of the antisense strand. In one preferred embodiment the dsRNA targets particularly the human GCR gene, in yet another preferred embodiment the dsRNA targets the mouse (Mus musculus) and rat (Rattus norvegicus) GCR gene.

In one embodiment, the antisense strand comprises a nucleotide sequence which is substantially complementary to at least part of an mRNA encoding said GCR gene, and the region of complementarity is most preferably less than 30 nucleotides in length. Furthermore, it is preferred that the length of the herein described inventive dsRNA molecules (duplex length) is in the range of about 16 to 30 nucleotides, in particular in the range of about 18 to 28 nucleotides. Particularly useful in context of this invention are duplex lengths of about 19, 20, 21, 22, 23 or 24 nucleotides. Most preferred are duplex stretches of 19, 21 or 23 nucleotides. The dsRNA, upon contacting with a cell expressing a GCR gene, inhibits the expression of a GCR gene in vitro by at least 70%, preferably by at least 80%, most preferred by 90%.

Appended Table 13 relates to preferred molecules to be used as dsRNA in accordance with this invention. Also modified dsRNA molecules are provided herein and are in particular disclosed in appended tables 1 and 4, providing illustrative examples of modified dsRNA molecules of the present invention. As pointed out herein above, Table 1 provides for illustrative examples of modified dsRNAs of this invention (whereby the corresponding sense strand and antisense strand is provided in this table). The relation of the unmodified preferred molecules shown in Table 13 to the modified dsRNAs of Table 1 is illustrated in Table 14. Yet, the illustrative modifications of these constituents of the inventive dsRNAs are provided herein as examples of modifications.

Tables 2 and 3 provide for selective biological, clinically and pharmaceutical relevant parameters of certain dsRNA molecules of this invention.

Most preferred dsRNA molecules are provided in the appended table 13 and, inter alia and preferably, wherein the sense strand is selected from the group consisting of the nucleic acid sequences depicted in SEQ ID Nos 873, 929, 1021, 1023, 967 and 905 and the antisense strand is selected from the group consisting of the nucleic acid sequences depicted in SEQ ID Nos 874, 930, 1022, 1024, 968 and 906. Accordingly, the inventive dsRNA molecule may, inter alia, comprise the sequence pairs selected from the group consisting of SEQ ID NOs: 873/874, 929/930, 1021/1022, 1023/1024, 967/968 and 905/906. In context of specific dsRNA molecules provided herein, pairs of SEQ ID NOs relate to corresponding sense and antisense strands sequences (5′ to 3′) as also shown in appended and included tables.

In one embodiment said dsRNA molecules comprise an antisense strand with a 3′ overhang of 1-5 nucleotides length, preferably of 1-2 nucleotides length. Preferably said overhang of the antisense strand comprises uracil or nucleotides which are complementary to the mRNA encoding GCR.

In another preferred embodiment, said dsRNA molecules comprise a sense strand with a 3′ overhang of 1-5 nucleotides length, preferably of 1-2 nucleotides length. Preferably said overhang of the sense strand comprises uracil or nucleotides which are identical to the mRNA encoding GCR.

In another preferred embodiment, said dsRNA molecules comprise a sense strand with a 3′ overhang of 1-5 nucleotides length, preferably of 1-2 nucleotides length, and an antisense strand with a 3′ overhang of 1-5 nucleotides length, preferably of 1-2 nucleotides length. Preferably said overhang of the sense strand comprises uracil or nucleotides which are at least 90% identical to the mRNA encoding GCR and said overhang of the antisense strand comprises uracil or nucleotides which are at least 90% complementary to the mRNA encoding GCR

Most preferred dsRNA molecules are provided in the tables 1 and 4 below and, inter alia and preferably, wherein the sense strand is selected from the group consisting of the nucleic acid sequences depicted in SEQ ID NOs: 7, 31, 3, 25, 33, 55, 83, 747 and 764 the antisense strand is selected from the group consisting of the nucleic acid sequences depicted in SEQ ID NOs: 8, 32, 4, 26, 34, 56, 84, 753 and 772. Accordingly, the inventive dsRNA molecule may, inter alia, comprise the sequence pairs selected from the group consisting of SEQ ID NOs: 7/8, 31/32, 3/4, 25/26, 33/34, 55/56, 83/84, 747/753 and 764/772. In context of specific dsRNA molecules provided herein, pairs of SEQ ID NOs relate to corresponding sense and antisense strands sequences (5′ to 3′) as also shown in appended and included tables.

The dsRNA molecules of the invention may be comprised of naturally occurring nucleotides or may be comprised of at least one modified nucleotide, such as a 2′-O-methyl modified nucleotide, a nucleotide comprising a 5′-phosphorothioate group, inverted deoxythymidine and a terminal nucleotide linked to a cholesteryl derivative or dodecanoic acid bisdecylamide group. 2′ modified nucleotides may have the additional advantage that certain immunostimulatory factors or cytokines are suppressed when the inventive dsRNA molecules are employed in vivo, for example in a medical setting. Alternatively and non-limiting, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In one preferred embodiment the dsRNA molecules comprises at least one of the following modified nucleotides: a 2′-O-methyl modified nucleotide, a nucleotide comprising a 5′-phosphorothioate group and a deoxythymidine. In another preferred embodiment all pyrimidines of the sense strand are 2′-O-methyl modified nucleotides, and all pyrimidines of the antisense strand are 2′-deoxy-2′-fluoro modified nucleotides. In one preferred embodiment two deoxythymidine nucleotides are found at the 3′ of both strands of the dsRNA molecule. In another embodiment at least one of these deoxythymidine nucleotides at the 3′ end of both strands of the dsRNA molecule comprises a 5′-phosphorothioate group. In another embodiment all cytosines followed by adenine, and all uracils followed by either adenine, guanine or uracil in the sense strand are 2′-O-methyl modified nucleotides, and all cytosines and uracils followed by adenine of the antisense strand are 2′-β-methyl modified nucleotides. Preferred dsRNA molecules comprising modified nucleotides are given in tables 1 and 4.

In a preferred embodiment the inventive dsRNA molecules comprise modified nucleotides as detailed in the sequences given in tables 1 and 4. In one preferred embodiment the inventive dsRNA molecule comprises sequence pairs selected from the group consisting of SEQ ID NOs: 7/8, 31/32, 3/4, 25/26, 33/34, 55/56 and 83/84, and comprise modifications as detailed in table 1.

In another embodiment the inventive dsRNAs comprise modified nucleotides on positions different from those disclosed in tables 1 and 4. In one preferred embodiment two deoxythymidine nucleotides are found at the 3′ of both strands of the dsRNA molecule. In another preferred embodiment one of those deoxythymidine nucleotides at the 3′ of both strand is a inverted deoxythymidine.

In one embodiment the dsRNA molecules of the invention comprise a sense and an antisense strand wherein both strands have a half-life of at least 9 hours. In one preferred embodiment the dsRNA molecules of the invention comprise a sense and an antisense strand wherein both strands have a half-life of at least 9 hours in human serum. In another embodiment the dsRNA molecules of the invention comprise a sense and an antisense strand wherein both strands have a half-life of at least 24 hours in human serum.

In another embodiment the dsRNA molecules of the invention are non-immunostimulatory, e.g. do not stimulate INF-alpha and TNF-alpha in vitro.

The invention also provides for cells comprising at least one of the dsRNAs of the invention. The cell is preferably a mammalian cell, such as a human cell. Furthermore, also tissues and/or non-human organisms comprising the herein defined dsRNA molecules are comprised in this invention, whereby said non-human organism is particularly useful for research purposes or as research tool, for example also in drug testing.

Furthermore, the invention relates to a method for inhibiting the expression of a GCR gene, in particular a mammalian or human GCR gene, in a cell, tissue or organism comprising the following steps:
(a) introducing into the cell, tissue or organism a double-stranded ribonucleic acid (dsRNA) as defined herein;
(b) maintaining said cell, tissue or organism produced in step (a) for a time sufficient to obtain degradation of the mRNA transcript of a GCR gene, thereby inhibiting expression of a GCR gene in a given cell.

The invention also relates to pharmaceutical compositions comprising the inventive dsRNAs of this invention. These pharmaceutical compositions are particularly useful in the inhibition of the expression of a GCR gene in a cell, a tissue or an organism. The pharmaceutical composition comprising one or more of the dsRNA of the invention may also comprise (a) pharmaceutically acceptable carrier(s), diluent(s) and/or excipient(s).

In another embodiment, the invention provides methods for treating, preventing or managing disorders which are associated type 2 diabetes, obesity, dislipidemia, diabetic atherosclerosis, hypertension and depression, said method comprising administering to a subject in need of such treatment, prevention or management a therapeutically or prophylactically effective amount of one or more of the dsRNAs of the invention. Preferably, said subject is a mammal, most preferably a human patient.

In one embodiment, the invention provides a method for treating a subject having a pathological condition mediated by the expression of a GCR gene. Such conditions comprise disorders associated with diabetes and obesity, as described above. In this embodiment, the dsRNA acts as a therapeutic agent for controlling the expression of a GCR gene. The method comprises administering a pharmaceutical composition of the invention to the patient (e.g., human), such that expression of a GCR gene is silenced. Because of their high specificity, the dsRNAs of the invention specifically target mRNAs of a GCR gene. In one preferred embodiment the described dsRNAs specifically decrease GCR mRNA levels and do not directly affect the expression and/or mRNA levels of off-target genes in the cell. In another preferred embodiment the described dsRNAs specifically decrease GCR mRNA levels as well as mRNA levels of genes that are normally activated by GCR. In another embodiment the inventive dsRNAs decrease glucose levels in vivo.

In one preferred embodiment the described dsRNA decrease GCR mRNA levels in the liver by at least 70%, preferably by at least 80%, most preferably by at least 90% in vivo. Preferably the dsRNAs of the invention decrease glycemia without change in liver transaminases. In another embodiment the described dsRNAs decrease GCR mRNA levels in vivo for at least 4 days. In another embodiment the described dsRNAs decrease GCR mRNA levels in vivo by at least 60% for at least 4 days.

Particularly useful with respect to therapeutic dsRNAs is the set of dsRNAs targeting mouse and rat GCR which can be used to estimate toxicity, therapeutic efficacy and effective dosages and in vivo half-lives for the individual dsRNAs in an animal or cell culture model.

In another embodiment, the invention provides vectors for inhibiting the expression of a GCR gene in a cell, in particular GCR gene comprising a regulatory sequence operable linked to a nucleotide sequence that encodes at least one strand of one of the dsRNA of the invention.

In another embodiment, the invention provides a cell comprising a vector for inhibiting the expression of a GCR gene in a cell. Said vector comprises a regulatory sequence operable linked to a nucleotide sequence that encodes at least one strand of one of the dsRNA of the invention. Yet, it is preferred that said vector comprises, besides said regulatory sequence a sequence that encodes at least one “sense strand” of the inventive dsRNA and at least one “anti sense strand” of said dsRNA. It is also envisaged that the claimed cell comprises two or more vectors comprising, besides said regulatory sequences, the herein defined sequence(s) that encode(s) at least one strand of one of the dsRNA of the invention.

In one embodiment, the method comprises administering a composition comprising a dsRNA, wherein the dsRNA comprises a nucleotide sequence which is complementary to at least a part of an RNA transcript of a GCR gene of the mammal to be treated. As pointed out above, also vectors and cells comprising nucleic acid molecules that encode for at least one strand of the herein defined dsRNA molecules can be used as pharmaceutical compositions and may, therefore, also be employed in the herein disclosed methods of treating a subject in need of medical intervention. It is also of note that these embodiments relating to pharmaceutical compositions and to corresponding methods of treating a (human) subject also relate to approaches like gene therapy approaches. GCR specific dsRNA molecules as provided herein or nucleic acid molecules encoding individual strands of these inventive dsRNA molecules may also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In another aspect of the invention, GCR specific dsRNA molecules that modulate GCR gene expression activity are expressed from transcription units inserted into DNA or RNA vectors (see, e.g., Skillern, A., et al., International PCT Publication No. WO 00/22113). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are preferably DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Danos and Mulligan, Proc. Natl. Acad. Sci. USA (1998) 85:6460-6464). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D 1-thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single GCR gene or multiple GCR genes over a period of a week or more are also contemplated by the invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The following detailed description discloses how to make and use the dsRNA and compositions containing dsRNA to inhibit the expression of a target GCR gene, as well as compositions and methods for treating diseases and disorders caused by the expression of said GCR gene.

### BRIEF DESCRIPTION OF THE DRAWINGS

FIG. 1—Effect of GCR dsRNA comprising SEQ ID pair 55/56 on silencing off-target sequences. Expression of renilla luciferase protein after transfection of COS7 cells expressing dual-luciferase constructs, representative for either 19 mer target site of GCR mRNA (“on”) or in silico predicted off-target sequences (“off 1” to “off 15”; with “off 1”-“off 12” being antisense strand off-targets and “off 13” to “off 15” being sense strand off-targets), with 50 nM GCR dsRNA. Perfect matching off-target dsRNAs are controls.

FIG. 2—Effect of GCR dsRNA comprising SEQ ID pair 83/84 on silencing off-target sequences. Expression of renilla luciferase protein after transfection of COS7 cells expressing dual-luciferase constructs, representative for either 19 mer target site of GCR mRNA (“on”) or in silico predicted off-target sequences (“off 1” to “off 14”; with “off 1”-“off 11” being antisense strand off-targets and “off 12” and “off 14” being sense strand off-targets), with 50 nM GCR dsRNA. Perfect matching off-target dsRNAs are controls.

FIG. 3—Effect of GCR dsRNA comprising SEQ ID pair 7/8 on silencing off-target sequences. Expression of renilla luciferase protein after transfection of COST cells expressing dual-luciferase constructs, representative for either 19 mer target site of GCR mRNA (“on”) or in silico predicted off-target sequences (“off 1” to “off 14”; with “off 1”-“off 11” being antisense strand off-targets and “off 12” to “off 14” being sense strand off-targets), with 50 nM GCR dsRNA. Perfect matching off-target dsRNAs are controls.

FIG. 4—mRNA levels, expressed in Quantigene 2.0 units/cell, for GCR (NR3C1) gene, or for housekeeping gene GUSB, in human primary hepatocytes 96 h post-transfection with GCR dsRNAs or Luciferase dsRNA control, in comparison to control cells exposed to DharmaFECT-1 transfection reagent alone.

FIG. 5—mRNA levels, expressed in Quantigene 2.0 units/cell, for GCR(NR3C1) gene (a), GUSB housekeeping gene (b) and GCR-target genes PCK1 (c), G6Pc (d) and TAT (e), in human primary hepatocytes exposed for 48 h to LNP01-formulated dsRNAs

FIG. 6—Glucose output measured in primary human hepatocytes exposed for 48 h to LNP01-dsRNAs (a) Luciferase dsRNA control b) GCR dsRNA comprising SEQ ID pair 55/56 c) GCR dsRNA comprising SEQ ID pair 83/84, and starved for 96 h before incubation for 5 h in the presence of gluconeogenic precursors (lactate and pyruvate).

FIG. 7—Cell ATP content measured in primary human hepatocytes exposed for 48 h to LNP01-dsRNAs (a) Luciferase dsRNA control b) GCR dsRNA comprising SEQ ID pair 55/56 c) GCR dsRNA comprising SEQ ID pair 83/84, and starved for 96 h before incubation for 5 h in the presence of gluconeogenic precursors (lactate and pyruvate).

FIG. 8—Liver mRNA levels, relative to GUSB housekeeping mRNA level, obtained for GCR(NR3C1 gene, FIG. 8 a) and GCR-upregulated genes TAT (FIG. 8a), PCK1 (FIG. 8b), G6Pc (FIG. 8b), and HES1 (down-regulated by GCR, FIG. 8c), 103 h after single iv administration of LPNO1-formulated dsRNAs for GCR comprising SEQ ID pair 517/518 or Luciferase control SEQ ID pair 681/682 in hyperglycemic and diabetic 14 wks-old male db/db mice.

FIG. 9—Time-course efficacy on blood glucose levels after single iv administration of LPNO1-dsRNAs in hyperglycemic and diabetic 14 wks-old male db/db mice. (*: p<0.05 versus vehicle). Efficacy of LPNO1-dsRNA for GCR comprising SEQ ID pair 517/518 in decreasing glucose level observed at +55-, +79-, +103 h was of −13%, at −31% and −29%, respectively, when compared to the placebo (LNP01-Luciferase dsRNA SEQ ID pair 681/682). n=4, mean values+/−SEM, t-test assuming equal variance for each day.

FIG. 10—Time-course plasma levels in ALT and AST in hyperglycemic and diabetic 14 wks-old male db/db mice, 55, 79 and 103 h after single iv administration of LPNO1-dsRNAs for GCR comprising SEQ ID pair 517/518 or Luciferase control dsRNA (SEQ ID pair 681/682).

FIG. 11—GCR mRNA levels in liver biopsy of cynomolgus monkeys measured by bDNA assay 3 days post single i.v. bolus injection of Luciferase dsRNA (Seq. ID pair 681/682) or GCR dsRNAs (Seq. ID pair 747/753 or Seq. ID pair 764/772). Dose with respect to dsRNA given for each group as mg/kg. N=2 female and male cynomolgus monkeys. Values are normalized to mean of GAPDH values of each individual monkey (a), or relative to Luciferase dsRNA (Seq. ID pair 681/682) with error bars indicating variations between monkeys (b).

### DETAILED DESCRIPTION OF THE INVENTION

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A”, “U” and “T” or “dT” respectively, each generally stand for a nucleotide that contains guanine, cytosine, adenine, uracil and deoxythymidine as a base, respectively. However, the term “ribonucleotide” or “nucleotide” can also refer to a modified nucleotide, as further detailed below, or a surrogate replacement moiety. Sequences comprising such replacement moieties are embodiments of the invention. As detailed below, the herein described dsRNA molecules may also comprise “overhangs”, i.e. unpaired, overhanging nucleotides which are not directly involved in the RNA double helical structure normally formed by the herein defined pair of “sense strand” and “anti sense strand”. Often, such an overhanging stretch comprises the deoxythymidine nucleotide, in most embodiments, 2 deoxythymidines in the 3′ end. Such overhangs will be described and illustrated below.

The term “GCR” as used herein relates in particular to the intracellular glucocorticoid receptor (GCR) and said term relates to the corresponding gene, also known as NR3C1 gene, encoded mRNA, encoded protein/polypeptide as well as functional fragments of the same. Preferred is the human GCR gene. In other preferred embodiments the dsRNAs of the invention target the GCR gene of rat (Rattus norvegicus) and mouse (Mus musculus), in yet another preferred embodiment the dsRNAs of the invention target the human (H. sapiens) and cynomolgous monkey (Macaca fascicularis) GCR gene. The term “GCR gene/sequence” does not only relate to (the) wild-type sequence(s) but also to mutations and alterations which may be comprised in said gene/sequence. Accordingly, the present invention is not limited to the specific dsRNA molecules provided herein. The invention also relates to dsRNA molecules that comprise an antisense strand that is at least 85% complementary to the corresponding nucleotide stretch of an RNA transcript of a GCR gene that comprises such mutations/alterations.

As used herein, “target sequence” refers to a contiguous portion of the nucleotide sequence of an mRNA molecule formed during the transcription of a GCR gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature. However, as detailed herein, such a “strand comprising a sequence” may also comprise modifications, like modified nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

Sequences referred to as “fully complementary” comprise base-pairing of the oligonucleotide or polynucleotide comprising the first nucleotide sequence to the oligonucleotide or polynucleotide comprising the second nucleotide sequence over the entire length of the first and second nucleotide sequence.

However, where a first sequence is referred to as “substantially complementary” with respect to a second sequence herein, the two sequences can be fully complementary, or they may form one or more, but preferably not more than 13 mismatched base pairs upon hybridization.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

The term “double-stranded RNA”, “dsRNA molecule”, or “dsRNA”, as used herein, refers to a ribonucleic acid molecule, or complex of ribonucleic acid molecules, having a duplex structure comprising two anti-parallel and substantially complementary nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. In addition to the duplex structure, a dsRNA may comprise one or more nucleotide overhangs. The nucleotides in said “overhangs” may comprise between 0 and 5 nucleotides, whereby “0” means no additional nucleotide(s) that form(s) an “overhang” and whereas “5” means five additional nucleotides on the individual strands of the dsRNA duplex. These optional “overhangs” are located in the 3′ end of the individual strands. As will be detailed below, also dsRNA molecules which comprise only an “overhang” in one the two strands may be useful and even advantageous in context of this invention. The “overhang” comprises preferably between 0 and 2 nucleotides. Most preferably 2 “dT” (deoxythymidine) nucleotides are found at the 3′ end of both strands of the dsRNA. Also 2 “U” (uracil) nucleotides can be used as overhangs at the 3′ end of both strands of the dsRNA. Accordingly, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. For example the antisense strand comprises 23 nucleotides and the sense strand comprises 21 nucleotides, forming a 2 nucleotide overhang at the 3′ end of the antisense strand. Preferably, the 2 nucleotide overhang is fully complementary to the mRNA of the target gene. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence. Where the region of complementarity is not fully complementary to the target sequence, the mismatches are most tolerated outside nucleotides 2-7 of the 5′ terminus of the antisense strand

The term “sense strand,” as used herein, refers to the strand of a dsRNA that includes a region that is substantially complementary to a region of the antisense strand. “Substantially complementary” means preferably at least 85% of the overlapping nucleotides in sense and antisense strand are complementary.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. It is, for example envisaged that the dsRNA molecules of this invention be administered to a subject in need of medical intervention. Such an administration may comprise the injection of the dsRNA, the vector or a cell of this invention into a diseased side in said subject, for example into liver tissue/cells or into cancerous tissues/cells, like liver cancer tissue. However, also the injection in close proximity of the diseased tissue is envisaged. In vitro introduction into a cell includes methods known in the art such as electroporation and lipofection.

The terms “silence”, “inhibit the expression of” and “knock down”, in as far as they refer to a GCR gene, herein refer to the at least partial suppression of the expression of a GCR gene, as manifested by a reduction of the amount of mRNA transcribed from a GCR gene which may be isolated from a first cell or group of cells in which a GCR gene is transcribed and which has or have been treated such that the expression of a GCR gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the GCR gene transcription, e.g. the amount of protein encoded by a GCR gene which is secreted by a cell, or the number of cells displaying a certain phenotype.

As illustrated in the appended examples and in the appended tables provided herein, the inventive dsRNA molecules are capable of inhibiting the expression of a human GCR by at least about 70%, preferably by at least 80%, most preferably by at least 90% in vitro assays, i.e. in vitro. The term “in vitro” as used herein includes but is not limited to cell culture assays. In another embodiment the inventive dsRNA molecules are capable of inhibiting the expression of a mouse or rat GCR by at least 70%. preferably by at least 80%, most preferably by at least 90%. The person skilled in the art can readily determine such an inhibition rate and related effects, in particular in light of the assays provided herein.

The term “off target” as used herein refers to all non-target mRNAs of the transcriptome that are predicted by in silico methods to hybridize to the described dsRNAs based on sequence complementarity. The dsRNAs of the present invention preferably do specifically inhibit the expression of GCR, i.e. do not inhibit the expression of any off-target.

Particular preferred dsRNAs are provided, for example in appended Table 1 and 2 (sense strand and antisense strand sequences provided therein in 5′ to 3′ orientation), with the most preferred dsRNAs in table 2.

The term “half-life” as used herein is a measure of stability of a compound or molecule and can be assessed by methods known to a person skilled in the art, especially in light of the assays provided herein.

The term “non-immunostimulatory” as used herein refers to the absence of any induction of a immune response by the invented dsRNA molecules. Methods to determine immune responses are well know to a person skilled in the art, for example by assessing the release of cytokines, as described in the examples section.

The terms “treat”, “treatment”, and the like, mean in context of this invention to relief from or alleviation of a disorder related to GCR expression, like diabetes, dyslipidemia, obesity, hypertension, cardiovascular diseases or depression.

As used herein, a “pharmaceutical composition” comprises a pharmacologically effective amount of a dsRNA and a pharmaceutically acceptable carrier. However, such a “pharmaceutical composition” may also comprise individual strands of such a dsRNA molecule or the herein described vector(s) comprising a regulatory sequence operably linked to a nucleotide sequence that encodes at least one strand of a sense or an antisense strand comprised in the dsRNAs of this invention. It is also envisaged that cells, tissues or isolated organs that express or comprise the herein defined dsRNAs may be used as “pharmaceutical compositions”. As used herein, “pharmacologically effective amount,” “therapeutically effective amount” or simply “effective amount” refers to that amount of an RNA effective to produce the intended pharmacological, therapeutic or preventive result.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives as known to persons skilled in the art.

It is in particular envisaged that the pharmaceutically acceptable carrier allows for the systemic administration of the dsRNAs, vectors or cells of this invention. Whereas also the enteric administration is envisaged the parenteral administration and also transdermal or transmucosal (e.g. insufflation, buccal, vaginal, anal) administration as well was inhalation of the drug are feasible ways of administering to a patient in need of medical intervention the compounds of this invention. When parenteral administration is employed, this can comprise the direct injection of the compounds of this invention into the diseased tissue or at least in close proximity. However, also intravenous, intraarterial, subcutaneous, intramuscular, intraperitoneal, intradermal, intrathecal and other administrations of the compounds of this invention are within the skill of the artisan, for example the attending physician.

For intramuscular, subcutaneous and intravenous use, the pharmaceutical compositions of the invention will generally be provided in sterile aqueous solutions or suspensions, buffered to an appropriate pH and isotonicity. In a preferred embodiment, the carrier consists exclusively of an aqueous buffer. In this context, “exclusively” means no auxiliary agents or encapsulating substances are present which might affect or mediate uptake of dsRNA in the cells that express a GCR gene. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in PCT publication WO 91/06309 which is incorporated by reference herein.

As used herein, a “transformed cell” is a cell into which at least one vector has been introduced from which a dsRNA molecule or at least one strand of such a dsRNA molecule may be expressed. Such a vector is preferably a vector comprising a regulatory sequence operably linked to nucleotide sequence that encodes at least one of a sense strand or an antisense strand comprised in the dsRNAs of this invention.

It can be reasonably expected that shorter dsRNAs comprising one of the sequences of Table 1 and 4 minus only a few nucleotides on one or both ends may be similarly effective as compared to the dsRNAs described above. As pointed out above, in most embodiments of this invention, the dsRNA molecules provided herein comprise a duplex length (i.e. without “overhangs”) of about 16 to about 30 nucleotides. Particular useful dsRNA duplex lengths are about 19 to about 25 nucleotides. Most preferred are duplex structures with a length of 19 nucleotides. In the inventive dsRNA molecules, the antisense strand is at least partially complementary to the sense strand.

In one preferred embodiment the inventive dsRNA molecules comprise nucleotides 1-19 of the sequences given in Table 13.

The dsRNA of the invention can contain one or more mismatches to the target sequence. In a preferred embodiment, the dsRNA of the invention contains no more than 13 mismatches. If the antisense strand of the dsRNA contains mismatches to a target sequence, it is preferable that the area of mismatch not be located within nucleotides 2-7 of the 5′ terminus of the antisense strand. In another embodiment it is preferable that the area of mismatch not to be located within nucleotides 2-9 of the 5′ terminus of the antisense strand.

As mentioned above, at least one end/strand of the dsRNA may have a single-stranded nucleotide overhang of 1 to 5, preferably 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Preferably, the single-stranded overhang is located at the 3′-terminal end of the antisense strand or, alternatively, at the 3′-terminal end of the sense strand. The dsRNA may also have a blunt end, preferably located at the 5′-end of the antisense strand. Preferably, the antisense strand of the dsRNA has a nucleotide overhang at the 3′-end, and the 5′-end is blunt. In another embodiment, one or more of the nucleotides in the overhang is replaced with a nucleoside thiophosphate.

The dsRNA of the present invention may also be chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the dsRNA include, without limitation, methylene blue; bifunctional groups, preferably bis-(2-chloroethyl)amine; N-acetyl-N′-(p-glyoxylbenzoyl)cystamine; 4-thiouracil; and psoralen. In one preferred embodiment, the linker is a hexa-ethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is preferably formed by triple-helix bonds.

In certain embodiments, a chemical bond may be formed by means of one or several bonding groups, wherein such bonding groups are preferably poly-(oxyphosphinicooxy-1,3-propandiol)- and/or polyethylene glycol chains. In other embodiments, a chemical bond may also be formed by means of purine analogs introduced into the double-stranded structure instead of purines. In further embodiments, a chemical bond may be formed by azabenzene units introduced into the double-stranded structure. In still further embodiments, a chemical bond may be formed by branched nucleotide analogs instead of nucleotides introduced into the double-stranded structure. In certain embodiments, a chemical bond may be induced by ultraviolet light.

In yet another embodiment, the nucleotides at one or both of the two single strands may be modified to prevent or inhibit the activation of cellular enzymes, for example certain nucleases. Techniques for inhibiting the activation of cellular enzymes are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, inverted thymidine and phosphoramidate (see, e.g., Wagner, Nat. Med. (1995) 1:1116-8). Thus, at least one 2′-hydroxyl group of the nucleotides on a dsRNA is replaced by a chemical group, preferably by a 2′-amino or a 2′-methyl group. Also, at least one nucleotide may be modified to form a locked nucleotide. Such locked nucleotide contains a methylene bridge that connects the 2′-oxygen of ribose with the 4′-carbon of ribose. Introduction of a locked nucleotide into an oligonucleotide improves the affinity for complementary sequences and increases the melting temperature by several degrees.

The compounds of the present invention can be synthesized using one or more inverted nucleotides, for example inverted thymidine or inverted adenine (see, for example, Takei, et al., 2002, JBC 277(26):23800-06).

Modifications of dsRNA molecules provided herein may positively influence their stability in vivo as well as in vitro and also improve their delivery to the (diseased) target side. Furthermore, such structural and chemical modifications may positively influence physiological reactions towards the dsRNA molecules upon administration, e.g. the cytokine release which is preferably suppressed. Such chemical and structural modifications are known in the art and are, inter alia, illustrated in Nawrot (2006) Current Topics in Med Chem, 6, 913-925.

Conjugating a ligand to a dsRNA can enhance its cellular absorption as well as targeting to a particular tissue. In certain instances, a hydrophobic ligand is conjugated to the dsRNA to facilitate direct permeation of the cellular membrane. Alternatively, the ligand conjugated to the dsRNA is a substrate for receptor-mediated endocytosis. These approaches have been used to facilitate cell permeation of antisense oligonucleotides. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Attachment of folic acid to the 3′-terminus of an oligonucleotide results in increased cellular uptake of the oligonucleotide (Li, S.; Deshmukh, H. M.; Huang, L. Pharm. Res. 1998, 15, 1540). Other ligands that have been conjugated to oligonucleotides include polyethylene glycols, carbohydrate clusters, cross-linking agents, porphyrin conjugates, and delivery peptides.

In certain instances, conjugation of a cationic ligand to oligonucleotides often results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See M. Manoharan Antisense & Nucleic Acid Drug Development 2002, 12, 103 and references therein.

The ligand-conjugated dsRNA of the invention may be synthesized by the use of a dsRNA that bears a pendant reactive functionality, such as that derived from the attachment of a linking molecule onto the dsRNA. This reactive oligonucleotide may be reacted directly with commercially-available ligands, ligands that are synthesized bearing any of a variety of protecting groups, or ligands that have a linking moiety attached thereto. The methods of the invention facilitate the synthesis of ligand-conjugated dsRNA by the use of, in some preferred embodiments, nucleoside monomers that have been appropriately conjugated with ligands and that may further be attached to a solid-support material. Such ligand-nucleoside conjugates, optionally attached to a solid-support material, are prepared according to some preferred embodiments of the methods of the invention via reaction of a selected serum-binding ligand with a linking moiety located on the 5′ position of a nucleoside or oligonucleotide. In certain instances, an dsRNA bearing an aralkyl ligand attached to the 3′-terminus of the dsRNA is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The dsRNA used in the conjugates of the invention may be conveniently and routinely made through the well-known technique of solid-phase synthesis. It is also known to use similar techniques to prepare other oligonucleotides, such as the phosphorothioates and alkylated derivatives.

Teachings regarding the synthesis of particular modified oligonucleotides may be found in the following U.S. patents: U.S. Pat. No. 5,218,105, drawn to polyamine conjugated oligonucleotides; U.S. Pat. No. 5,541,307, drawn to oligonucleotides having modified backbones; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. No. 5,587,361 drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N−2 substituted purines; U.S. Pat. No. 5,587,470, drawn to oligonucleotides having 3-deazapurines; U.S. Pat. No. 5,608,046, both drawn to conjugated 4′-desmethyl nucleoside analogs; U.S. Pat. No. 5,610,289, drawn to backbone-modified oligonucleotide analogs; U.S. Pat. No. 6,262,241 drawn to, inter alia, methods of synthesizing 2′-fluoro-oligonucleotides.

In the ligand-conjugated dsRNA and ligand-molecule bearing sequence-specific linked nucleosides of the invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a preferred embodiment, the oligonucleotides or linked nucleosides of the invention are synthesized by an automated synthesizer using phosphoramidites derived from ligand-nucleoside conjugates in addition to commercially available phosphoramidites.

The incorporation of a 2′-O-methyl, 2′-O-ethyl, 2′-O-propyl, 2′-O-allyl, 2′-O-aminoalkyl or 2′-deoxy-2′-fluoro group in nucleosides of an oligonucleotide confers enhanced hybridization properties to the oligonucleotide. Further, oligonucleotides containing phosphorothioate backbones have enhanced nuclease stability. Thus, functionalized, linked nucleosides of the invention can be augmented to include either or both a phosphorothioate backbone or a 2′-O-methyl, 2′-O-ethyl, 2′-O-propyl, 2′-O-aminoalkyl, 2′-O-allyl or 2′-deoxy-2′-fluoro group.

In some preferred embodiments, functionalized nucleoside sequences of the invention possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In a preferred embodiment, ligand molecules may be conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

In one preferred embodiment of the methods of the invention, the preparation of ligand conjugated oligonucleotides commences with the selection of appropriate precursor molecules upon which to construct the ligand molecule. Typically, the precursor is an appropriately-protected derivative of the commonly-used nucleosides. For example, the synthetic precursors for the synthesis of the ligand-conjugated oligonucleotides of the invention include, but are not limited to, 2′-aminoalkoxy-5′-ODMT-nucleosides, 2′-6-aminoalkylamino-5′-ODMT-nucleosides, 5′-6-aminoalkoxy-2′-deoxy-nucleosides, 5′-6-aminoalkoxy-2-protected-nucleosides, 3′-6-aminoalkoxy-5′-ODMT-nucleosides, and 3′-aminoalkylamino-5′-ODMT-nucleosides that may be protected in the nucleobase portion of the molecule. Methods for the synthesis of such amino-linked protected nucleoside precursors are known to those of ordinary skill in the art.

In many cases, protecting groups are used during the preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y., 1991.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821.

Additional amino-protecting groups include, but are not limited to, carbamate protecting groups, such as 2-trimethylsilylethoxycarbonyl (Teoc), 1-methyl-1-(4-biphenyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the invention.

Many solid supports are commercially available and one of ordinary skill in the art can readily select a solid support to be used in the solid-phase synthesis steps. In certain embodiments, a universal support is used. A universal support allows for preparation of oligonucleotides having unusual or modified nucleotides located at the 3′-terminus of the oligonucleotide. For further details about universal supports see Scott et al., Innovations and Perspectives in solid-phase Synthesis, 3rd International Symposium, 1994, Ed. Roger Epton, Mayflower Worldwide, 115-124]. In addition, it has been reported that the oligonucleotide can be cleaved from the universal support under milder reaction conditions when oligonucleotide is bonded to the solid support via a syn-1,2-acetoxyphosphate group which more readily undergoes basic hydrolysis. See Guzaev, A. I.; Manoharan, M. J. Am. Chem. Soc. 2003, 125, 2380.

The nucleosides are linked by phosphorus-containing or non-phosphorus-containing covalent internucleoside linkages. For the purposes of identification, such conjugated nucleosides can be characterized as ligand-bearing nucleosides or ligand-nucleoside conjugates. The linked nucleosides having an aralkyl ligand conjugated to a nucleoside within their sequence will demonstrate enhanced dsRNA activity when compared to like dsRNA compounds that are not conjugated.

The aralkyl-ligand-conjugated oligonucleotides of the invention also include conjugates of oligonucleotides and linked nucleosides wherein the ligand is attached directly to the nucleoside or nucleotide without the intermediacy of a linker group. The ligand may preferably be attached, via linking groups, at a carboxyl, amino or oxo group of the ligand. Typical linking groups may be ester, amide or carbamate groups.

Specific examples of preferred modified oligonucleotides envisioned for use in the ligand-conjugated oligonucleotides of the invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes of the invention, modified oligonucleotides that do not have a phosphorus atom in their intersugar backbone can also be considered to be oligonucleosides.

Specific oligonucleotide chemical modifications are described below. It is not necessary for all positions in a given compound to be uniformly modified. Conversely, more than one modifications may be incorporated in a single dsRNA compound or even in a single nucleotide thereof.

Preferred modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included.

Representative United States patents relating to the preparation of the above phosphorus-atom-containing linkages include, but are not limited to, U.S. Pat. Nos. 4,469,863; 5,023,243; 5,264,423; 5,321,131; 5,399,676; 5,405,939; 5,453,496; 5,455,233 and 5,466,677, each of which is herein incorporated by reference.

Preferred modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents relating to the preparation of the above oligonucleosides include, but are not limited to, U.S. Pat. Nos. 5,034,506; 5,214,134; 5,216,141; 5,264,562; 5,466,677; 5,470,967; 5,489,677; 5,602,240 and 5,663,312, each of which is herein incorporated by reference.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Teaching of PNA compounds can be found for example in U.S. Pat. No. 5,539,082.

Some preferred embodiments of the invention employ oligonucleotides with phosphorothioate linkages and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂—, and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

The oligonucleotides employed in the ligand-conjugated oligonucleotides of the invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents relating to the preparation of certain of the above-noted modified nucleobases as well as other modified nucleobases include, but are not limited to, the above noted U.S. Pat. No. 3,687,808, as well as U.S. Pat. Nos. 5,134,066; 5,459,255; 5,552,540; 5,594,121 and 5,596,091 all of which are hereby incorporated by reference.

In certain embodiments, the oligonucleotides employed in the ligand-conjugated oligonucleotides of the invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. a preferred modification includes 2′-methoxyethoxy [2′-O—CH₂CH₂OCH₃, also known as 2′-O-(2-methoxyethyl) or 2′-MOE], i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, filed on Jan. 30, 1998, the contents of which are incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and, inter alia, those which are disclosed by Delgardo et. al. (Critical Reviews in Therapeutic Drug Carrier Systems 1992, 9:249), which is hereby incorporated by reference in its entirety. Further sugar modifications are disclosed by Cook (Anti-fibrosis Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230. Further representative 2′-substituent groups amenable to the invention include those having one of formula I or II:

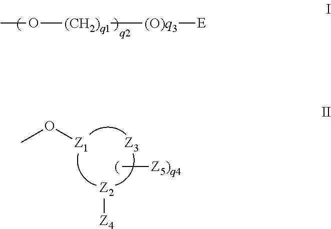

wherein,

E is C₁-C₁₀ alkyl, N(Q₃)(Q₄) or N═C(Q₃)(Q₄); each Q₃ and Q₄ is, independently, H, C₁-C₁₀ alkyl, dialkylaminoalkyl, a nitrogen protecting group, a tethered or untethered conjugate group, a linker to a solid support; or Q₃ and Q₄, together, form a nitrogen protecting group or a ring structure optionally including at least one additional heteroatom selected from N and O;

q₁ is an integer from 1 to 10;

q₂ is an integer from 1 to 10;

q₃ is 0 or 1;

q₄ is 0, 1 or 2;

each Z₁, Z₂ and Z₃ is, independently, C₄-C₇ cycloalkyl, C₅-C₁₄ aryl or C₃-C₁₅ heterocyclyl, wherein the heteroatom in said heterocyclyl group is selected from oxygen, nitrogen and sulfur;

Z₄ is OM₁, SM₁, or N(M₁)₂; each M₁ is, independently, H, C₁-C₈ alkyl, C₁-C₈ haloalkyl, C(═NH)N(H)M₂, C(═O)N(H)M₂ or OC(═O)N(H)M₂; M₂ is H or C₁-C₈ alkyl; and

Z₅ is C₁-C₁₀ alkyl, C₁-C₁₀ haloalkyl, C₂-C₁₀ alkenyl, C₂-C₁₀ alkynyl, C₆-C₁₄ aryl, N(Q₃)(Q₄), OQ₃, halo, SQ₃ or CN.

Representative 2′-O-sugar substituent groups of formula I are disclosed in U.S. Pat. No. 6,172,209, entitled “Capped 2′-Oxyethoxy Oligonucleotides,” hereby incorporated by reference in its entirety. Representative cyclic 2′-O-sugar substituent groups of formula II are disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the invention. Representative substitutions for ring O include, but are not limited to, S, CH₂, CHF, and CF₂.

Oligonucleotides may also have sugar mimetics, such as cyclobutyl moieties, in place of the pentofuranosyl sugar. Representative United States patents relating to the preparation of such modified sugars include, but are not limited to, U.S. Pat. Nos. 5,359,044; 5,466,786; 5,519,134; 5,591,722; 5,597,909; 5,646,265 and 5,700,920, all of which are hereby incorporated by reference.

Additional modifications may also be made at other positions on the oligonucleotide, particularly the 3′ position of the sugar on the 3′ terminal nucleotide. For example, one additional modification of the ligand-conjugated oligonucleotides of the invention involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

The invention also includes compositions employing oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

In certain instances, the oligonucleotide may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate. The use of a cholesterol conjugate is particularly preferred since such a moiety can increase targeting to tissues in the liver, a site of GCR protein production.

Alternatively, the molecule being conjugated may be converted into a building block, such as a phosphoramidite, via an alcohol group present in the molecule or by attachment of a linker bearing an alcohol group that may be phosphorylated.

Importantly, each of these approaches may be used for the synthesis of ligand conjugated oligonucleotides. Amino linked oligonucleotides may be coupled directly with ligand via the use of coupling reagents or following activation of the ligand as an NHS or pentfluorophenolate ester. Ligand phosphoramidites may be synthesized via the attachment of an aminohexanol linker to one of the carboxyl groups followed by phosphitylation of the terminal alcohol functionality. Other linkers, such as cysteamine, may also be utilized for conjugation to a chloroacetyl linker present on a synthesized oligonucleotide.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items of the present invention are now illustrated with the following, non-limiting examples.

Description of Appended Tables:

Table 1—dsRNA targeting human GCR gene. Letters in capitals represent RNA nucleotides, lower case letters “c”, “g”, “a” and “u” represent 2′ O-methyl-modified nucleotides, “s” represents phosphorothioate and “dT” deoxythymidine, “invdT” inverted deoxythymidine, “f” represents 2′ fluoro modification of the preceding nucleotide.

Table 2—Characterization of dsRNAs targeting human GCR: Activity testing for dose response in HepG2 and HeLaS3 cells. IC 50: 50% inhibitory concentration.

Table 3—Characterization of dsRNAs targeting human GCR: Stability and Cytokine Induction. t ½: half-life of a strand as defined in examples, PBMC: Human peripheral blood mononuclear cells.

Table 4—dsRNAs targeting mouse and rat GCR genes. Letters in capitals represent RNA nucleotides, lower case letters “c”, “g”, “a” and “u” represent 2′ O-methyl-modified nucleotides, “s” represents phosphorothioate and “dT” deoxythymidine. “f” represents 2′ fluoro modification of the preceding nucleotide.

Table 5—Characterization of dsRNA targeting mouse and rat GCR genes: Stability and Cytokine Induction. t ½: half-life of a strand as defined in examples, PBMC: Human peripheral blood mononuclear cells.

Table 6—Selected off-targets of dsRNAs targeting human GCR comprising sequence ID pair 55/56.

Table 7—Selected off-targets of dsRNAs targeting human GCR comprising sequence ID pair 83/84.

Table 8—Selected off-targets of dsRNAs targeting human GCR comprising sequence ID pair 7/8.

Table 9—Sequences of bDNA probes for determination of human GAPDH; LE=label extender, CE=capture extender, BL=blocking probe.

Table 10—Sequences of bDNA probes for determination of human GCR; LE=label extender, CE=capture extender, BL=blocking probe.

Table 11—Sequences of bDNA probes for determination of mouse GCR; LE=label extender, CE=capture extender, BL=blocking probe.

Table 12—Sequences of bDNA probes for determination of mouse GAPDH; LE=label extender, CE=capture extender, BL=blocking probe.

Table 13—dsRNA targeting human GCR gene. Letters in capitals represent RNA nucleotides.

Table 14—dsRNA targeting human GCR gene without modifications and their modified counterparts. Letters in capitals represent RNA nucleotides, lower case letters “c”, “g”, “a” and “u” represent 2′ O-methyl-modified nucleotides, “s” represents phosphorothioate and “dT” deoxythymidine, “invdT” inverted deoxythymidine.

### EXAMPLES

### Identification of dsRNAs for Therapeutic Use

dsRNA design was carried out to identify dsRNAs specifically targeting human GCR for therapeutic use. First, the known mRNA sequences of human (Homo sapiens) GCR (NM_{—}000176.2, NM_{—}001018074.1, NM_{—}001018075.1, NM_{—}001018076.1, NM_{—}001018077.1, NM_{—}001020825.1, NM_{—}001024094.1 listed as SEQ ID NO. 659, SEQ ID NO. 660, SEQ ID NO. 661, SEQ ID NO. 662, SEQ ID NO. 663, SEQ ID NO. 664, and SEQ ID NO. 665) were downloaded from NCBI Genbank®.

mRNAs of rhesus monkey (Macaca mulatta) GCR (XM_{—}001097015.1, XM_{—}001097126.1, XM_{—}001097238.1, XM_{—}001097341.1, XM_{—}001097444.1, XM_{—}001097542.1, XM_{—}001097640.1, XM_{—}001097749.1, XM_{—}001097846.1 and XM_{—}001097942.1) were downloaded from NCBI Genbank® (SEQ ID NO. 666, SEQ ID NO. 667, SEQ ID NO. 668, SEQ ID NO. 669, SEQ ID NO. 670, SEQ ID NO. 671, SEQ ID NO. 672, SEQ ID NO. 673, SEQ ID NO. 674, and SEQ ID NO. 675).

An EST of cynomolgus monkey (Macaca fascicularis) GCR (BB878843.1) was downloaded from NCBI Genbank® (SEQ ID NO. 676).

The monkey sequences were examined together with the human GCR mRNA sequences (SEQ ID NO. 677) by computer analysis to identify homologous sequences of 19 nucleotides that yield RNA interference (RNAi) agents cross-reactive to human and rhesus monkey or human and cynomolgus monkey sequences.

In identifying RNAi agents, the selection was limited to 19 mer sequences having at least 2 mismatches in the antisense strand to any other sequence in the human RefSeq database (release 27), which we assumed to represent the comprehensive human transcriptome, by using a proprietary algorithm.

The cynomolgous monkey GCR gene was sequenced (see SEQ ID NO. 678) and examined for target regions of RNAi agents.

dsRNAs cross-reactive to human as well as cynomolgous monkey GCR were defined as most preferable for therapeutic use. All sequences containing 4 or more consecutive G's (poly-G sequences) were excluded from the synthesis.

The sequences thus identified formed the basis for the synthesis of the RNAi agents in appended Tables 1, and 14.

Identification of dsRNAs for In Vivo Proof of Concept Studies

dsRNA design was carried out to identify dsRNAs targeting mouse (Mus musculus) and rat (Rattus norvegicus) for in vivo proof-of-concept experiments. First, the transcripts for mouse GCR (NM_{—}008173.3, SEQ ID NO. 679) and rat GCR (NM_{—}012576.2, SEQ ID NO. 680) were examined by computer analysis to identify homologous sequences of 19 nucleotides that yield RNAi agents cross-reactive between these sequences.

In identifying RNAi agents, the selection was limited to 19 mer sequences having at least 2 mismatches in the antisense strand to any other sequence in the mouse and rat RefSeq database (release 27), which we assumed to represent the comprehensive mouse and rat transcriptome, by using a proprietary algorithm.

All sequences containing 4 or more consecutive G's (poly-G sequences) were excluded from the synthesis. The sequences thus identified formed the basis for the synthesis of the RNAi agents in appended Table 4.

dsRNA Synthesis

Where the source of a reagent is not specifically given herein, such reagent may be obtained from any supplier of reagents for molecular biology at a quality/purity standard for application in molecular biology.

Single-stranded RNAs were produced by solid phase synthesis on a scale of 1 μmole using an Expedite™ 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

Activity Testing

Activity of dsRNAs Targeting Human GCR

The activity of the GCR-dsRNAs for therapeutic use described above was tested in HeLaS3 cells. Cells in culture were used for quantitation of GCR mRNA by branched DNA in total mRNA derived from cells incubated with GCR-specific dsRNAs.

HeLaS3 cells were obtained from American Type Culture Collection (Rockville, Md., cat. No. CCL-2.2) and cultured in Ham's F12 (Biochrom AG, Berlin, Germany, cat. No. FG 0815) supplemented to contain 10% fetal calf serum (FCS) (Biochrom AG, Berlin, Germany, cat. No. S0115), Penicillin 100 U/ml, Streptomycin 100 mg/ml (Biochrom AG, Berlin, Germany, cat. No. A2213) at 37° C. in an atmosphere with 5% CO₂ in a humidified incubator (Heraeus HERAAcell, Kendro Laboratory Products, Langenselbold, Germany).

Cell seeding and transfection of dsRNA were performed at the same time. For transfection with dsRNA, HeLaS3 cells were seeded at a density of 2.0×10⁴ cells/well in 96-well plates. Transfection of dsRNA was carried out with Lipofectamine™ 2000 (Invitrogen GmbH, Karlsruhe, Germany, cat. No. 11668-019) as described by the manufacturer. In a first single dose experiment dsRNAs were transfected at a concentration of 30 nM. Two independent experiments were performed. Most effective dsRNAs showing a mRNA knockdown of more than 80% from the first single dose screen at 30 nM were further characterized by dose response curves. For dose response curves, transfections were performed in HeLaS3 cells as described for the single dose screen above, but with the following concentrations of dsRNA (nM): 24, 6, 1.5, 0.375, 0.0938, 0.0234, 0.0059, 0.0015, 0.0004 and 0.0001 nM. After transfection cells were incubated for 24 h at 37° C. and 5% CO₂ in a humidified incubator (Heraeus GmbH, Hanau, Germany). For measurement of GCR mRNA cells were harvested and lysed at 53° C. following procedures recommended by the manufacturer of the QuantiGene™ 1.0 Assay Kit (Panomics, Fremont, Calif., USA, cat. No. QG-0004) for bDNA quantitation of mRNA. Afterwards, 50 μl of the lysates were incubated with probesets specific to human GCR and human GAPDH (sequence of probesets see table 9 and 10) and processed according to the manufacturer's protocol for QuantiGene™. Chemoluminescence was measured in a Victor2-Light (Perkin Elmer, Wiesbaden, Germany) as RLUs (relative light units) and values obtained with the human GCR probeset were normalized to the respective human GAPDH values for each well. Unrelated control dsRNAs were used as a negative control.

Inhibition data are given in appended tables 1 and 2.

Activity of dsRNAs Targeting Rodent GCR

The activity of the GCR-siRNAs for use in rodent models was tested in Hepa1-6 cells. Hepa1-6 cells in culture were used for quantitation of GCR mRNA by branched DNA assay from whole cell lysates derived from cells transfected with GCR-specific siRNAs.

Hepa1-6 cells were obtained from Deutsche Sammlung von Mikroorganismen and Zellkulturen GmbH (Braunschweig Germany, cat. No. ACC 175) and cultured in DMEM (Biochrom AG, Berlin, Germany, cat. No. FG 0815) supplemented to contain 10% fetal calf serum (FCS) (Biochrom AG, Berlin, Germany, cat. No. S0115), Penicillin 100 U/ml, Streptomycin 100 mg/ml (Biochrom AG, Berlin, Germany, cat. No. A2213), L-Glutamine 4 mM (Biochrom AG, Berlin, Germany, cat. No. K0283) at 37° C. in an atmosphere with 5% CO2 in a humidified incubator (Heraeus HERAcell®, Kendro Laboratory Products, Langenselbold, Germany).

Cell seeding and transfection of siRNA were performed at the same time. For transfection with siRNA, Hepa1-6 cells were seeded at a density of 15000 cells/well in 96-well plates. Transfection of siRNA was carried out with Lipofectamine™ 2000 (Invitrogen GmbH, Karlsruhe, Germany, cat. No. 11668-019) as described by the manufacturer. The two chemically different screening sets of siRNAs were transfected at a concentration of 50 nM. For measurement of GCR mRNA cells were harvested 24 h after transfection and lysed at 53° C. following procedures recommended by the manufacturer of the QuantiGene™ 1.0 Assay Kit (Panomics, Fremont, Calif., USA, cat. No. QG-0004) for bDNA quantitation of mRNA. Afterwards, 50 μl of the lysates were incubated with probesets specific to mouse GCR and mouse GAPDH (sequence of probesets see below) and processed according to the manufacturer's protocol for QuantiGene™. Chemiluminescence was measured in a Victor2-Light (Perkin Elmer, Wiesbaden, Germany) as RLUs (relative light units) and values obtained with the mouse GCR probeset were normalized to the respective mouse GAPDH values for each well. Unrelated control siRNAs were used as a negative control.

Most efficacious three siRNAs were used for pharmacological prove of concept studies in rodent in vivo experiments.

Inhibition data are given in appended table 4.

Stability of dsRNAs

Stability of dsRNAs was determined in in vitro assays with either human serum or plasma from cynomolgous monkey for dsRNAs targeting human GCR and with mouse serum for dsRNAs targeting mouse/rat PTB1B by measuring the half-life of each single strand.

Measurements were carried out in triplicates for each time point, using 3 μl 50 μM dsRNA sample mixed with 30 μl human serum or cynomolgous plasma (Sigma Aldrich). Mixtures were incubated for either 0 min, 30 min, 1 h, 3 h, 6 h, 24 h, or 48 h at 37° C. As control for unspecific degradation dsRNA was incubated with 30 μl 1×PBS pH 6.8 for 48 h. Reactions were stopped by the addition of 40 proteinase K (20 mg/ml), 25 μl of “Tissue and Cell Lysis Solution” (Epicentre) and 38 μl Millipore water for 30 min at 65° C. Samples were afterwards spin filtered through a 0.2 μm 96 well filter plate at 1400 rpm for 8 min, washed with 55 μl Millipore water twice and spin filtered again.

For separation of single strands and analysis of remaining full length product (FLP), samples were run through an ion exchange Dionex Summit HPLC under denaturing conditions using as eluent A 20 mM Na₃PO₄ in 10% ACN pH=11 and for eluent B 1 M NaBr in eluent A.

The following gradient was applied:

| Time | % A | % B |
|---|---|---|
| −1.0 min | 75 | 25 |
| 1.00 min | 75 | 25 |
| 19.0 min | 38 | 62 |
| 19.5 min | 0 | 100 |
| 21.5 min | 0 | 100 |
| 22.0 min | 75 | 25 |
| 24.0 min | 75 | 25 |

For every injection, the chromatograms were integrated automatically by the Dionex Chromeleon® 6.60 HPLC software, and were adjusted manually if necessary. All peak areas were corrected to the internal standard (1S) peak and normalized to the incubation at t=0 min. The area under the peak and resulting remaining FLP was calculated for each single strand and triplicate separately. Half-life (t½) of a strand was defined by the average time point [h] for triplicates at which half of the FLP was degraded.

Results are given in appended tables 3 and 5.

Cytokine Induction

Potential cytokine induction of dsRNAs was determined by measuring the release of INF-a and TNF-a in an in vitro PBMC assay.

Human peripheral blood mononuclear cells (PBMC) were isolated from buffy coat blood of two donors by Ficoll centrifugation at the day of transfection. Cells were transfected in quadruplicates with dsRNA and cultured for 24 h at 37° C. at a final concentration of 130 nM in Opti-MEM®, using either Gene Porter 2 (GP2) or DOTAP. dsRNA sequences that were known to induce INF-a and TNF-a in this assay, as well as a CpG oligo, were used as positive controls. Chemical conjugated dsRNA or CpG oligonucleotides that did not need a transfection reagent for cytokine induction, were incubated at a concentration of 500 nM in culture medium. At the end of incubation, the quadruplicate culture supernatant were pooled.

INF-a and TNF-a was then measured in these pooled supernatants by standard sandwich ELISA with two data points per pool. The degree of cytokine induction was expressed relative to positive controls using a score from 0 to 5, with 5 indicating maximum induction.

Results are given in appended tables 3 and 5.

In Vitro Off-Target Analysis of dsRNA Targeting Human GCR

The psiCHECK™-vector (Promega) contains two reporter genes for monitoring RNAi activity: a synthetic version of the Renilla luciferase (hRluc) gene and a synthetic firefly luciferase gene (hluc+). The firefly luciferase gene permits normalization of changes in Renilla luciferase expression to firefly luciferase expression. Renilla and firefly luciferase activities were measured using the Dual-Glo® Luciferase Assay System (Promega). To use the psiCHECK™ vectors for analyzing off-target effects of the inventive dsRNAs, the predicted off-target sequence was cloned into the multiple cloning region located 3′ to the synthetic Renilla luciferase gene and its translational stop codon. After cloning, the vector is transfected into a mammalian cell line, and subsequently cotransfected with dsRNAs targeting GCR. If the dsRNA effectively initiates the RNAi process on the target RNA of the predicted off-target, the fused Renilla target gene mRNA sequence will be degraded, resulting in reduced Renilla luciferase activity.

In Silico Off-Target Prediction

The human genome was searched by computer analysis for sequences homologous to the inventive dsRNAs. Homologous sequences that displayed less than 6 mismatches with the inventive dsRNAs were defined as a possible off-targets. Off-targets selected for in vitro off-target analysis are given in appended tables 6, 7 and 8.

Generation of psiCHECK Vectors Containing Predicted Off-Target Sequences

The strategy for analyzing off target effects for an dsRNA lead candidate includes the cloning of the predicted off target sites into the psiCHECK™-2 Vector system (Dual Glo®-system, Promega, Braunschweig, Germany cat. No C8021) via XhoI and NotI restriction sites. Therefore, the off target site is extended with 10 nucleotides upstream and downstream of the dsRNA target site. Additionally, a NheI restriction site is integrated to prove insertion of the fragment by restriction analysis. The single-stranded oligonucleotides were annealed according to a standard protocol (e.g. protocol by Metabion) in a Mastercycler® (Eppendorf) and then cloned into psiCHECK™ (Promega) previously digested with XhoI and NotI. Successful insertion was verified by restriction analysis with NheI and subsequent sequencing of the positive clones. The selected primer (Seq ID No. 677) for sequencing binds at position 1401 of vector psiCHECK. After clonal production the plasmids were analyzed by sequencing and than used in cell culture experiments.

Analysis of dsRNA Off-Target Effects

### Cell Culture:

Cos7 cells were obtained from Deutsche Sammlung für Mikroorganismen and Zellkulturen (DSMZ, Braunschweig, Germany, cat. No. ACC-60) and cultured in DMEM (Biochrom AG, Berlin, Germany, cat. No. F0435) supplemented to contain 10% fetal calf serum (FCS) (Biochrom AG, Berlin, Germany, cat. No. S0115), Penicillin 100 U/ml, and Streptomycin 100 μg/ml (Biochrom AG, Berlin, Germany, cat. No. A2213) and 2 mM L-Glutamine (Biochrom AG, Berlin, Germany, cat. No. K0283) as well as 12 μg/ml Natrium-bicarbonate at 37° C. in an atmosphere with 5% CO2 in a humidified incubator (Heraeus HERAcell®, Kendro Laboratory Products, Langenselbold, Germany).

### Transfection and Luciferase Quantification:

For transfection with plasmids, Cos-7 cells were seeded at a density of 2.25×10⁴ cells/well in 96-well plates and transfected directly. Transfection of plasmids was carried out with Lipofectamine™ 2000 (Invitrogen GmbH, Karlsruhe, Germany, cat. No. 11668-019) as described by the manufacturer at a concentration of 50 ng/well. 4 hours after transfection, the medium was discarded and fresh medium was added. Now the dsRNAs were transfected in a concentration at 50 nM using Lipofectamine™ 2000 as described above. 24 h after dsRNA transfection the cells were lysed using Luciferase reagent described by the manufacturer (Dual-Glo™ Luciferase Assay system, Promega, Mannheim, Germany, cat. No. E2980) and Firefly and Renilla Luciferase were quantified according to the manufacturer's protocol. Renilla Luciferase protein levels were normalized to Firefly Luciferase levels. For each dsRNA eight individual data points were collected in two independent experiments. A dsRNA unrelated to all target sites was used as a control to determine the relative Renilla Luciferase protein levels in dsRNA treated cells.

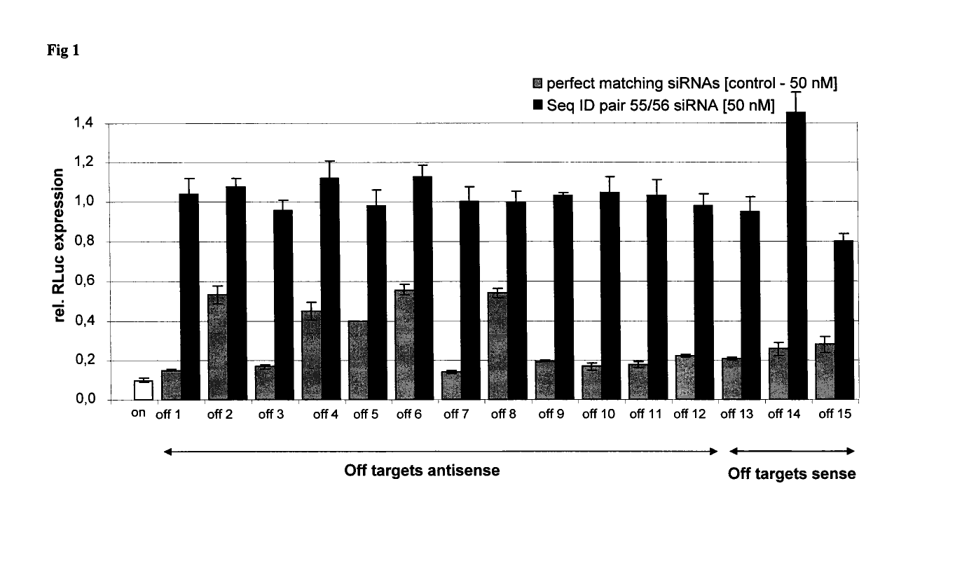

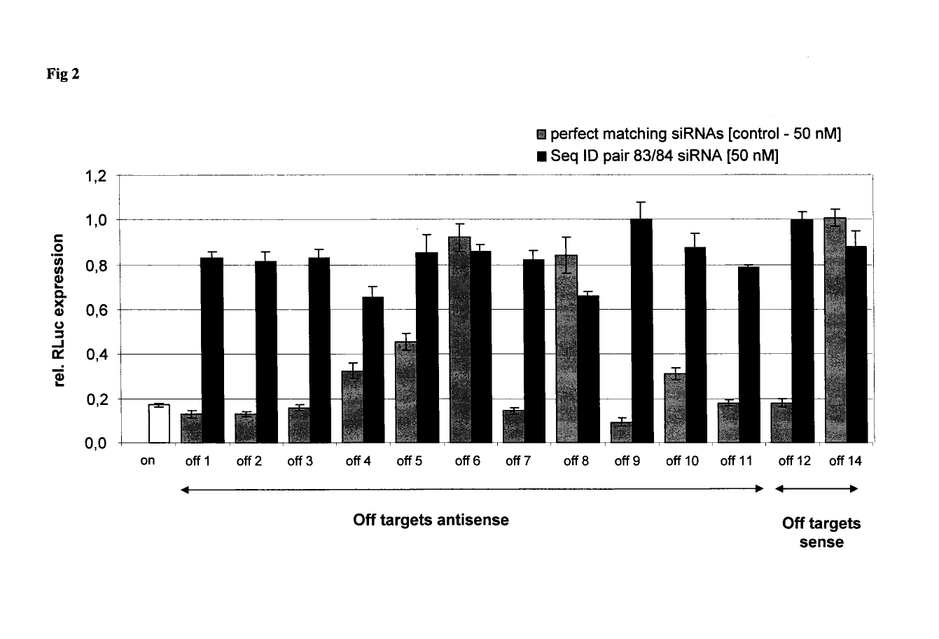

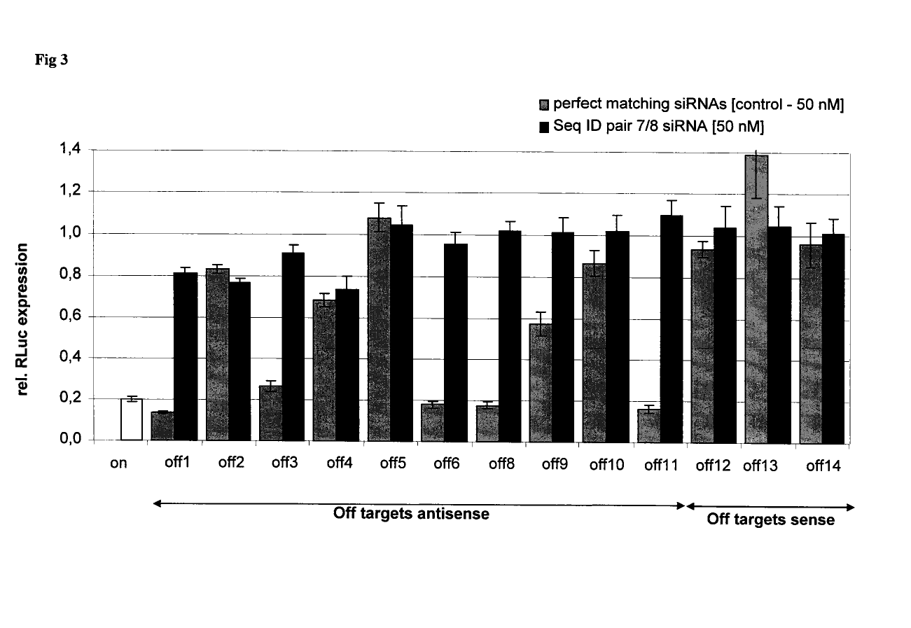

Results are given in FIGS. 1, 2 and 3.

Efficacy of dsRNAs Targeting GCR in Human Primary Hepatocytes

GCR Target Gene Knockdown after Transfection of dsRNAs

Fresh suspensions of human primary hepatocytes, isolated from surgery resections, were purchased from HepaCult GmbH and were plated in 12 well collagen coated plates, at a density of 325 000 cells/well in William's E media (Sigma-Aldrich Inc, cat. No W1878.) supplemented with 10% Fetal Calf Serum (FCS), 1% GlutaMAX™ 200 mM (Invitrogen GmbH, cat. No 35050-038.) and antibiotics (penicillin, streptomycin and gentamycin). After overnight culture (at 37° C. in an atmosphere with 5% CO₂ in a humidified incubator), medium was replaced with DMEM medium (Invitrogen GmbH, cat. No 21885) similarly supplemented, and dsRNAs transfections were performed at a final concentration of 15 nM, using DharmaFECT®-1 transfection reagent (ThermoFisher Scientific Inc, cat. No T2001). 72 h later, medium was replaced with fresh medium supplemented with 2 μM cAMP (Sigma-Aldrich Inc, cat. No S3912) and cells were further cultured overnight to allow for induction of gene expression. Cells were then exposed to Dexamethasone 500 nM (Sigma-Aldrich Inc, cat. No D4902) for 6 h to trigger activation and translocation of GCR to the nuclei and were recovered for gene expression analysis by branched-DNA technology, according to Panomics/Affymetrix Inc protocols for QuantiGene™ 2.0 technology (http://www.panomics.com/index.php?id=product_{—}1). In these conditions, exposure of human primary hepatocytes to dsRNA for GCR led to up to 90% KD of GCR gene expression

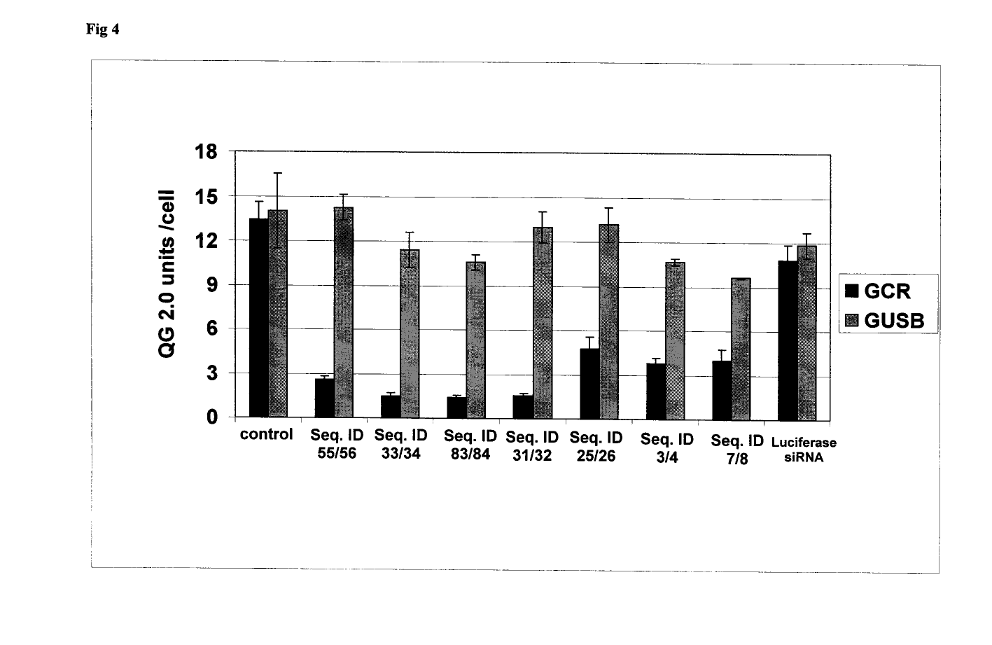

Results are shown in FIG. 4.

Effect of LNP01-Formulated dsRNAs for GCR on GCR and GCR-Regulated Genes Expression

Human primary hepatocytes were plated and cultivated as described above, except that 450 000 cells were seeded per well. After overnight culture, cells were exposed for 48 h to dsRNAs packaged into cationic liposomal formulation LNP01 at doses ranging from 1 to 100 nM. After 32 h exposure to dsRNAs, cAMP was added at 2 uM final concentration. Medium was further supplemented with Dexamethasone at 500 nM final concentration 6 h before cell recovery for gene expression analysis. In these conditions, cell exposure to LNP01-formulated dsRNA for GCR led to dose response inhibition of GCR gene expression, with 80% KD of GCR gene expression reached at 100 nM exposure without change in the expression of GUSB housekeeping gene. GCR KD translated into strong inhibition of expression of TAT and PCK1 genes, and to a lesser extend, to G6Pc gene inhibition, which expressions are induced by GCR receptor upon activation.

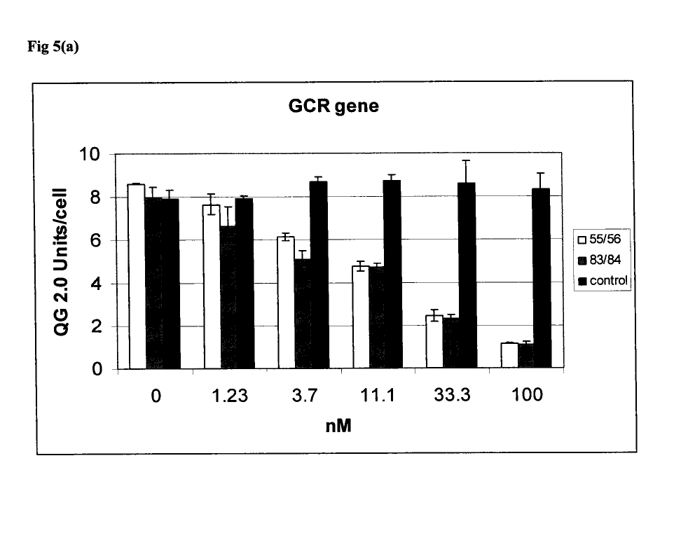

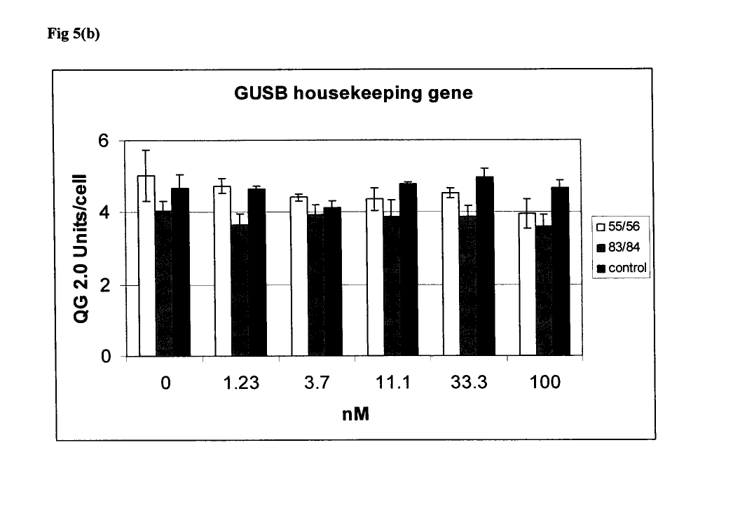

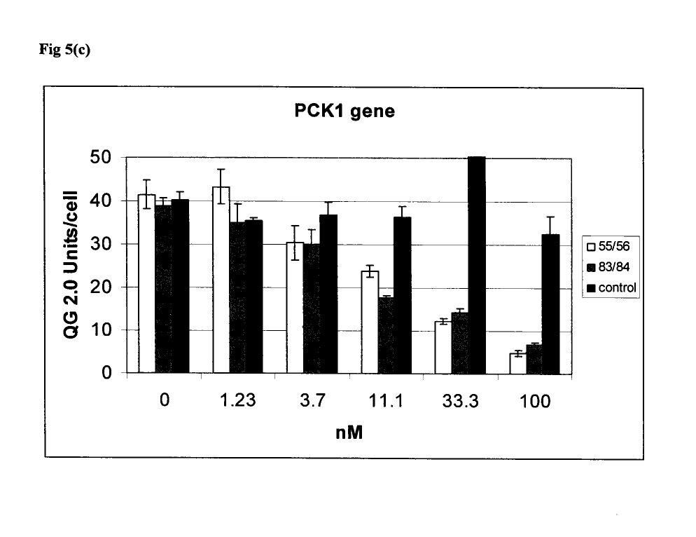

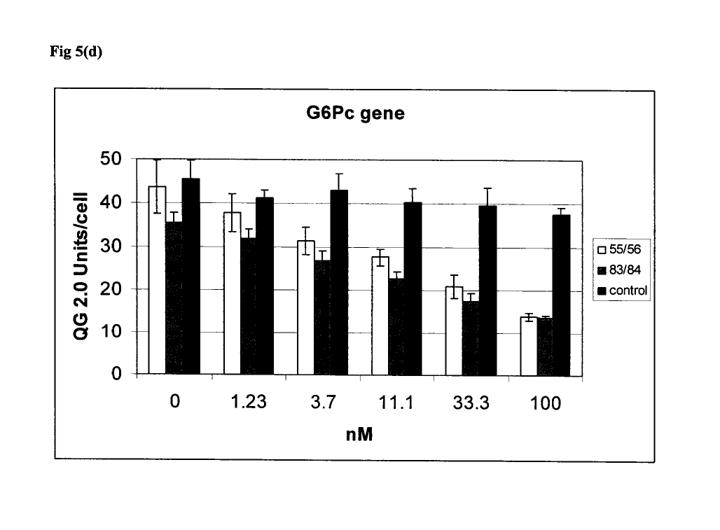

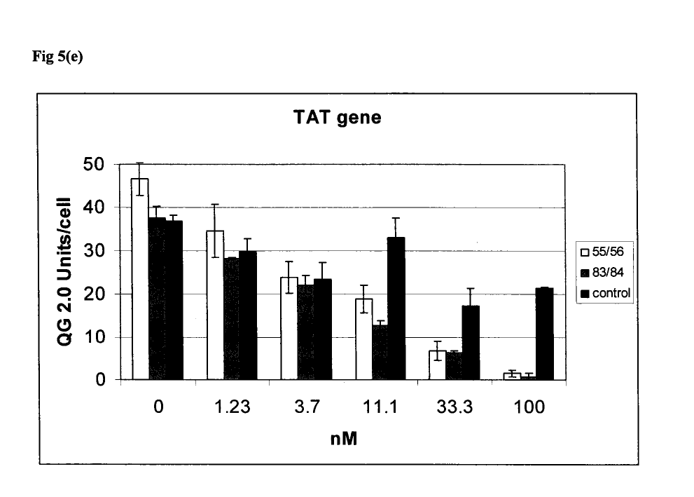

Results are shown in FIG. 5.

Effect of LNP01-Formulated dsRNAs for GCR on Glucose Output

Glucose output assays were performed on primary human hepatocytes seeded and exposed to LNP01-formulated dsRNAs as described above, except that 96 well plates format were used with 35 000 cells seeded/well, and that after 48 h exposure to LNP01-formulated dsRNAs, cells were cultivated in starvation conditions for 72 h in glucose-free RPMI 1640 media (Invitrogen GmbH, cat. No 11879) supplemented with 1% FCS and antibiotics, before medium was refreshed and supplemented with 2 uM cAMP and with 30 nM Dexamethasone for overnight incubation. Control cells treated with cAMP alone, or with cAMP, Dexamethasone and Mifepristone 1 uM (a GCR antagonist), were also performed. Cells were then further incubated in the presence of gluconeogenic precursors (lactate and pyruvate) to induce glucose production for 5 h in DPBS (Invitrogen GmbH, cat. No 1404) containing 0.1% free-fatty acid BSA, 20 mM sodium pyruvate and 2 mM lactate. Glucose produced was evaluated with Amplex® Red Glucose/Glucose oxidase assay kit (Invitrogen GmbH, cat. No A22189) in culture supernatants. As an indicator of cell viability, cellular ATP content was also measured using CellTiter-Glo® luminescent cell viability assay (Promega Corporation, cat. No G7571). Cell exposure to LNP01-formulated dsRNA for GCR led to dose-response inhibition of glucose production up to the maximum level expected from full antagonism of GCR activity achieved by Mifepristone.

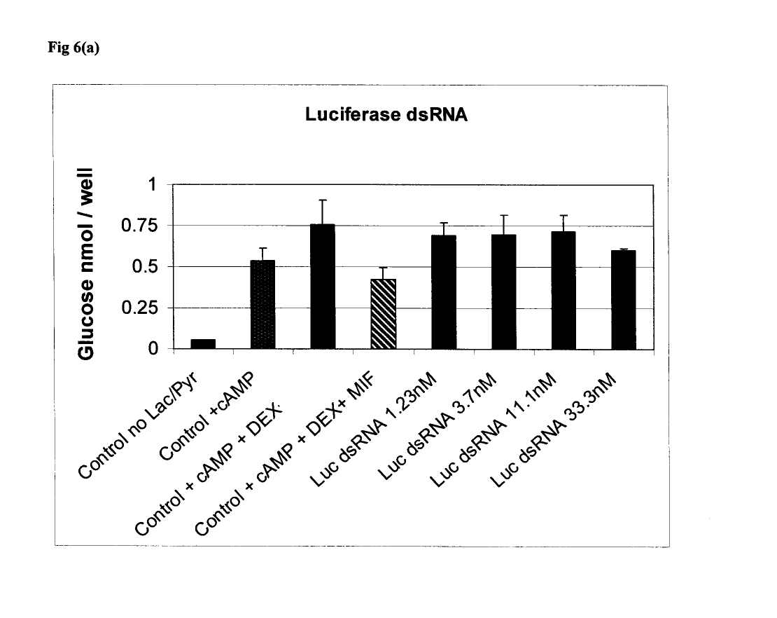

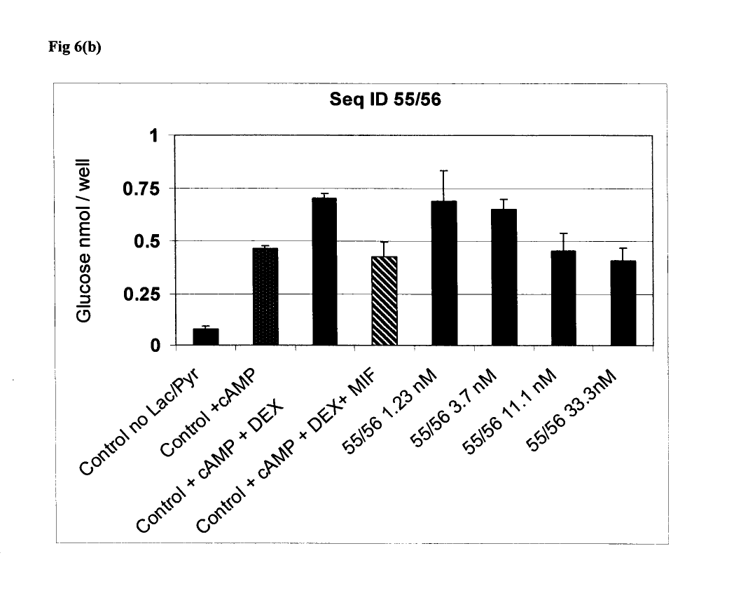

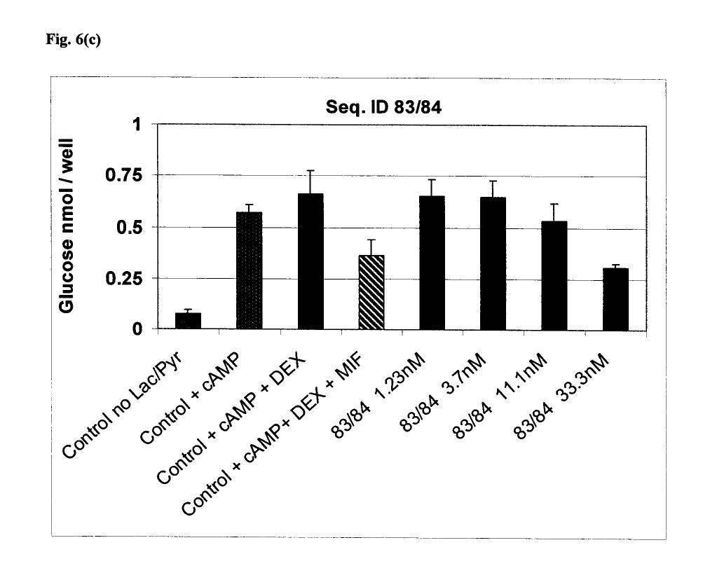

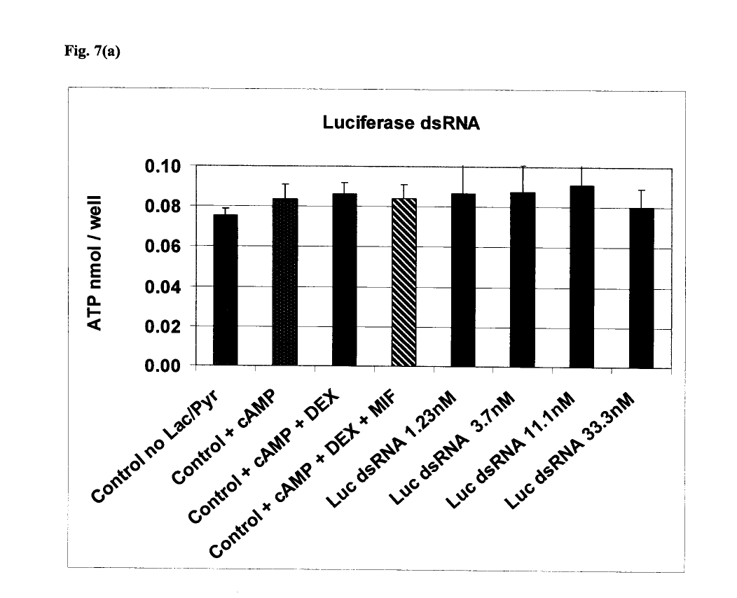

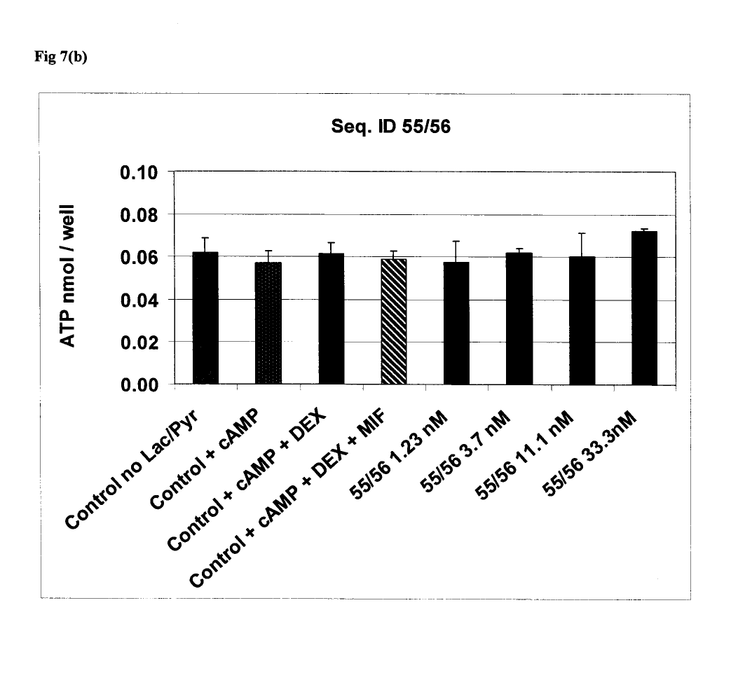

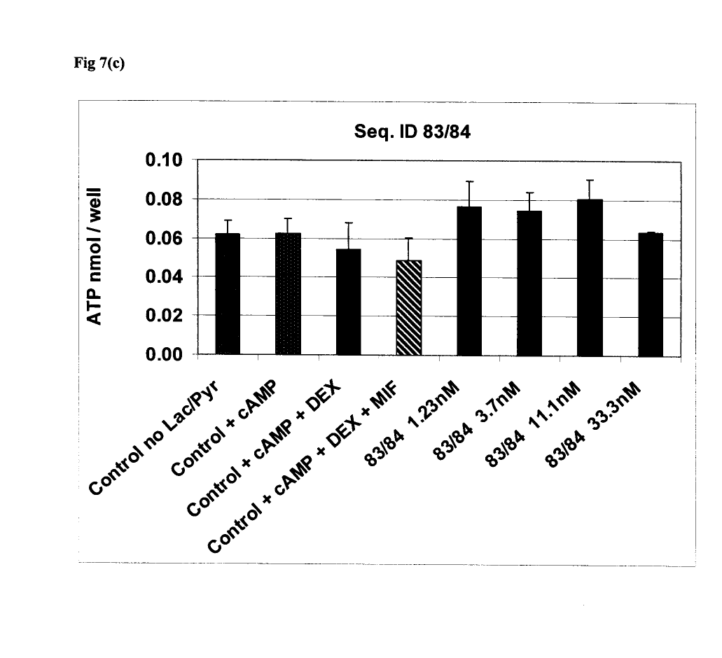

Results are shown in FIGS. 6 and 7.

In Vivo Effects of dsRNA Targeting Mice and Rat GCR

RNAi-Mediated GCR KD in Liver, and Efficacy on Blood Glucose in db/db Mice after Single i.v. Injection.

A group of 30 males db/db mice (Jackson laboratories) were fed a regular chow diet (Kliba 3436). Homogenous groups of 4 mice each were organized according to their BW and blood glucose measured under fed conditions the day of the experiment and 2 h after was food removed.

Mice were treated with single iv injection of either LNP01-formulated ds RNA for Luciferase control (SEQ ID pair 681/682) or LNP01-formulated dsRNA for GCR (SEQ ID pair 517/518) at 5.76 mg/kg for up to 103 h.

Blood glucose levels were measured with Accu-Chek® (Aviva) 2 days, 3 days and 4 days after iv injection (+55 h, +79 h and +103 h post treatment) in the afternoon corresponding to 10 h after food was removed. Mice were then sacrificed. Plasma ALT and AST were analyzed by Hitachi. Liver was harvested and snap frozen in liquid nitrogen for mRNA expression analysis of GCR and GCR-regulated genes (TAT, PCK1, G6Pc and HES1 genes) by branched-DNA, processing the largest lobe (left lateral lobe) according to Panomics/QuantiGene™ 2.0 sample processing protocol for animal tissues (Panomics-Affymetrix Inc, cat. No QS0106). Db/db mice treatment with GCR dsRNA. resulted in significant KD of GCR gene expression in mice liver and in decreased glycemia without change in liver transaminases.

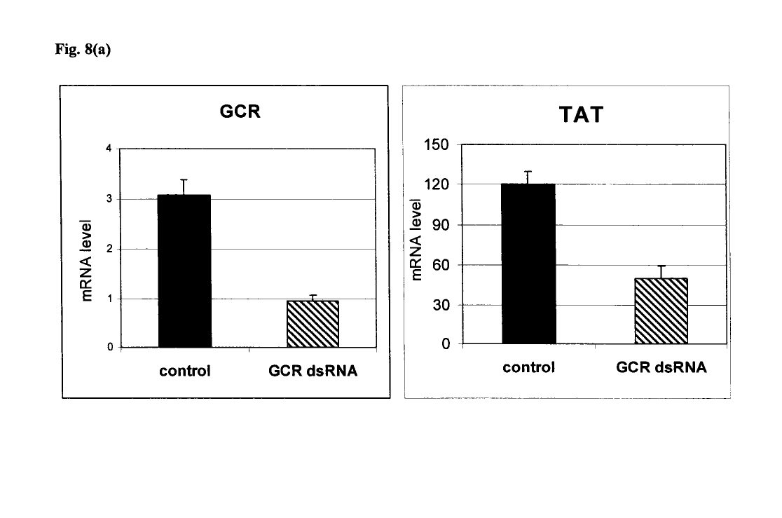

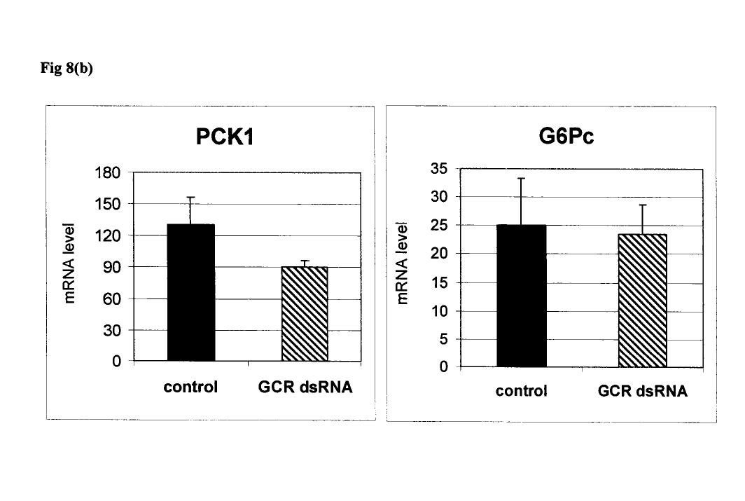

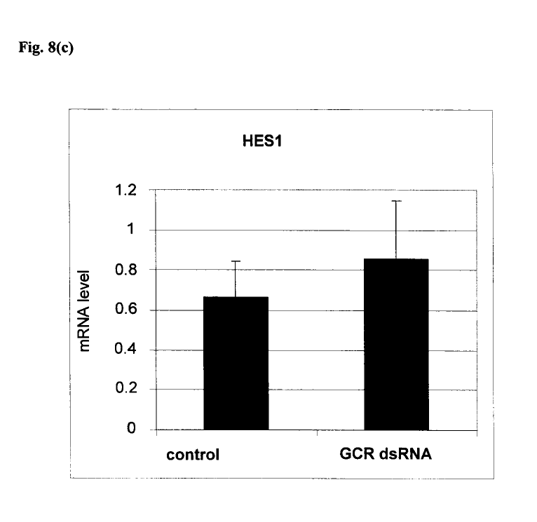

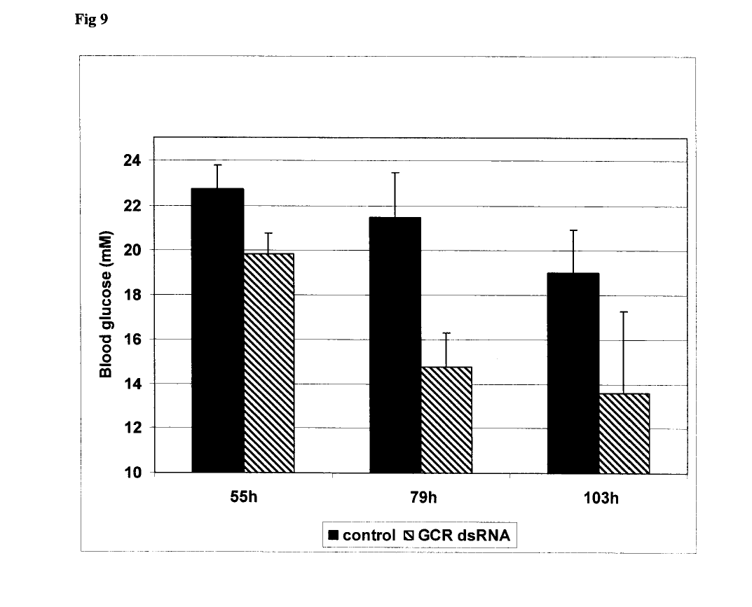

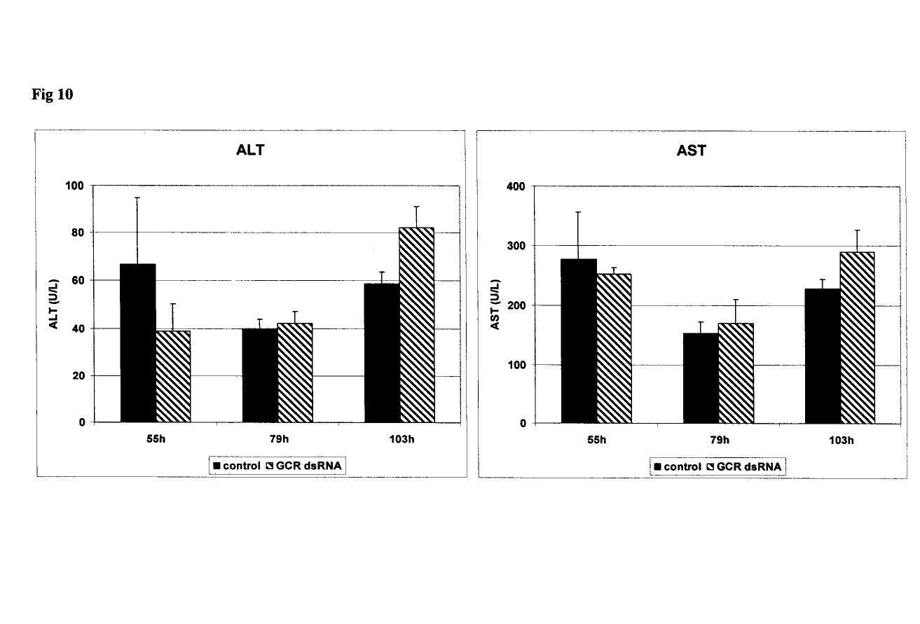

Results are shown in FIGS. 8, 9 and 10.

In Vivo Effects of dsRNA Targeting GCR (Macaca fascicularis)

For the following studies a sterile formulation of dsRNA lipid particles in isotonic buffer (e.g. Semple S C et al., Nat. Biotechnol. 2010 February; 28(2):172-6. Epub 2010 Jan. 17. Rational design of cationic lipids for siRNA delivery.) were used.

Single Dose Titration Study in Monkeys (Macaca fascicularis)

Monkeys received single i.v. bolus injections of GCR dsRNA (Seq. ID pair 747/753) of either 0.5, 1.5 or 3 mg/kg, or dsRNA (Seq. ID pair 764/772) in a dose of 1.5 mg/kg. Control groups received a 1.5 mg/kg of Luciferase dsRNA (Seq. ID pair 681/682) in order to discriminate between effects caused by the lipid particle and RNAi-mediated effects. All treatment groups were run with one male and one female monkey. Liver biopsy samples were taken on day 3 after injection.

GCR mRNA levels were measured from liver biopsy samples by bDNA assay as described above.

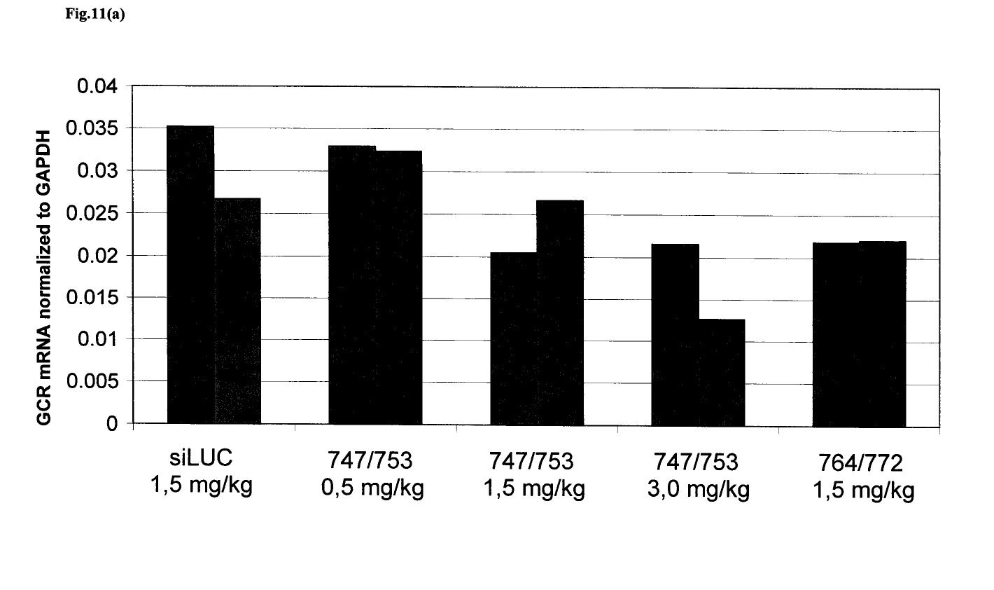

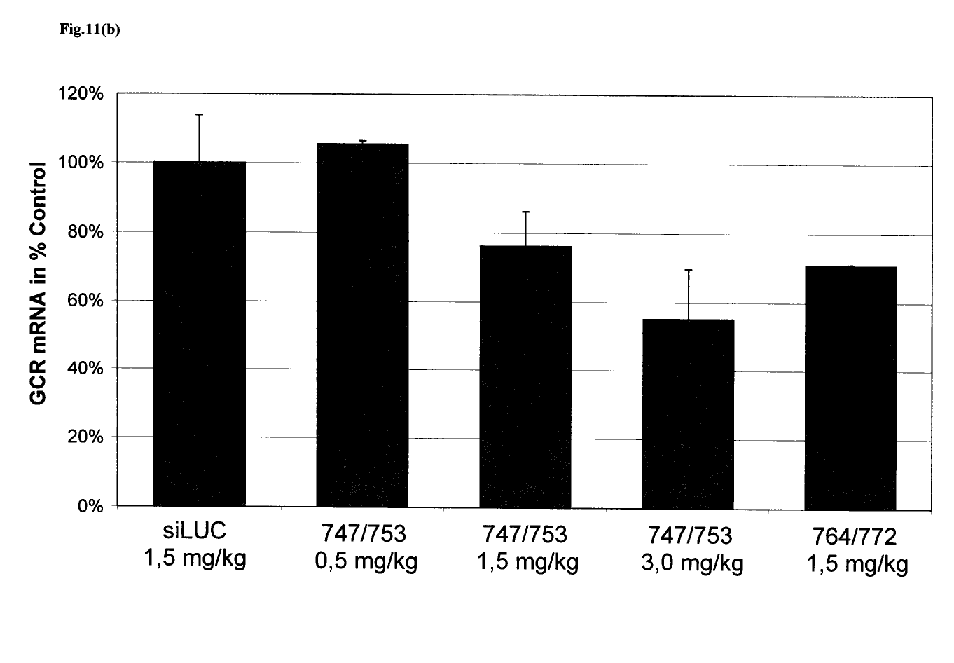

GCR dsRNA treated groups showed a dose-dependent decrease in GCR mRNA levels starting with 1.5 mg/kg of GCR dsRNA resulting in a decrease of about 24% by GCR dsRNA (Seq. ID pair 747/753) and 29% decrease by GCR dsRNA (Seq. ID pair 764/772), and reaching a 45% decrease in GCR mRNA with 3 mg/kg of GCR dsRNA (Seq. ID pair 747/753) (FIG. 11).

TABLE 1

Activity testing with
30 nM dsRNA in
HeLaS3 cells

| | | | | | |
|---|---|---|---|---|---|
| SEQ ID | | SEQ | | mean % | standard |
| NO | Sense strand sequence (5′-3′) | ID NO | Antisense strand sequence (5′-3′) | knock-down | deviation |

| | | | | | |
|---|---|---|---|---|---|
| 757 | ugGucGAAcAGuuuuuuccdT(abasic) | 761 | AGAAAAAACUGUUCGACcAdT(abasic) | 93 | 0 |
| 756 | uGGucGAAcAGuuuuuuccdT(abasic) | 760 | pAGAAAAAACUGUUCGACcAdT(abasic) | 93 | 1 |
| 756 | uGGucGAAcAGuuuuuuccdT(abasic) | 761 | AGAAAAAACUGUUCGACcAdT(abasic) | 93 | 1 |
| 755 | ugGucGAAcAGuuuuuucudT(abasic) | 761 | AGAAAAAACUGUUCGACcAdT(abasic) | 93 | 1 |
| 748 | uGGucGAAcAGuuuuuuccdT(invdT) | 753 | AGAAAAAACUGUUCGACcAdT(invdT) | 93 | 2 |
| 749 | ugGucGAAcAGuuuuuuccdT(invdT) | 753 | AGAAAAAACUGUUCGACcAdT(invdT) | 93 | 2 |
| 749 | ugGucGAAcAGuuuuuuccdT(invdT) | 752 | pAGAAAAAACUGUUCGACcAdT(invdT) | 93 | 0 |
| 758 | ugGucGAAcAGuuuuuucGdT(abasic) | 761 | AGAAAAAACUGUUCGACcAdT(abasic) | 92 | 2 |
| 754 | uGGucGAAcAGuuuuuucudT(abasic) | 760 | pAGAAAAAACUGUUCGACcAdT(abasic) | 92 | 1 |
| 755 | ugGucGAAcAGuuuuuucudT(abasic) | 760 | pAGAAAAAACUGUUCGACcAdT(abasic) | 92 | 0 |
| 754 | uGGucGAAcAGuuuuuucudT(abasic) | 761 | AGAAAAAACUGUUCGACcAdT(abasic) | 92 | 0 |
| 758 | ugGucGAAcAGuuuuuucGdT(abasic) | 760 | pAGAAAAAACUGUUCGACcAdT(abasic) | 92 | 1 |
| 748 | uGGucGAAcAGuuuuuuccdT(invdT) | 752 | pAGAAAAAACUGUUCGACcAdT(invdT) | 92 | 0 |
| 757 | ugGucGAAcAGuuuuuuccdT(abasic) | 760 | pAGAAAAAACUGUUCGACcAdT(abasic) | 92 | 2 |
| 750 | uGGucGAAcAGuuuuuucGdT(invdT) | 753 | AGAAAAAACUGUUCGACcAdT(invdT) | 92 | 0 |
| 750 | uGGucGAAcAGuuuuuucGdT(invdT) | 752 | pAGAAAAAACUGUUCGACcAdT(invdT) | 92 | 1 |
| 751 | ugGucGAAcAGuuuuuucGdT(invdT) | 752 | pAGAAAAAACUGUUCGACcAdT(invdT) | 92 | 1 |
| 759 | uGGucGAAcAGuuuuuucGdT(abasic) | 761 | AGAAAAAACUGUUCGACcAdT(abasic) | 92 | 1 |
| 751 | ugGucGAAcAGuuuuuucGdT(invdT) | 753 | AGAAAAAACUGUUCGACcAdT(invdT) | 92 | 1 |
| 746 | uGGucGAAcAGuuuuuucudT(invdT) | 753 | AGAAAAAACUGUUCGACcAdT(invdT) | 92 | 0 |
| 747 | ugGucGAAcAGuuuuuucudT(invdT) | 753 | AGAAAAAACUGUUCGACcAdT(invdT) | 92 | 1 |
| 740 | uGGucGAAcAGuuuuuuccdTsdT | 744 | pAGAAAAAACUGUUCGACcAdTsdT | 91 | 1 |
| 742 | uGGucGAAcAGuuuuuucGdTsdT | 745 | pAGAAAAAACUGUUCGACcAdTsdT | 91 | 1 |
| 740 | uGGucGAAcAGuuuuuuccdTsdT | 56 | AGAAAAAACUGUUCGACcAdTsdT | 91 | 1 |
| 743 | ugGucGAAcAGuuuuuucGdTsdT | 745 | pAGAAAAAACUGUUCGACcAdTsdT | 91 | 2 |
| 743 | ugGucGAAcAGuuuuuucGdTsdT | 56 | AGAAAAAACUGUUCGACcAdTsdT | 91 | 1 |
| 741 | ugGucGAAcAGuuuuuuccdTsdT | 56 | AGAAAAAACUGUUCGACcAdTsdT | 91 | 1 |
| 742 | uGGucGAAcAGuuuuuucGdTsdT | 56 | AGAAAAAACUGUUCGACcAdTsdT | 91 | 2 |
| 1 | cAuGuAcGAccAAuGuAAAdTsdT | 2 | UfUfUfACfAUfUfGGUfCfGUfACfAUfGdTsdT | 91 | 2 |
| 3 | uuGcuuAAcuAcAuAuAGAdTsdT | 4 | UfCfUfAUfAUfGUfAGUfUfAAGCfAAdTsdT | 90 | 1 |
| 5 | AAAuAAcuuGcuuAAcuAcdTsdT | 6 | GUfAGUfUfAAGCfAAGUfUfAUfUfUfdTsdT | 90 | 2 |
| 741 | ugGucGAAcAGuuuuuuccdTsdT | 744 | pAGAAAAAACUGUUCGACcAdTsdT | 90 | 2 |
| 739 | ugGucGAAcAGuuuuuucudTsdT | 744 | pAGAAAAAACUGUUCGACcAdTsdT | 90 | 2 |
| 739 | ugGucGAAcAGuuuuuucudTsdT | 56 | AGAAAAAACUGUUCGACcAdTsdT | 90 | 1 |
| 747 | ugGucGAAcAGuuuuuucudT(invdT) | 752 | pAGAAAAAACUGUUCGACcAdT(invdT) | 90 | 1 |
| 746 | uGGucGAAcAGuuuuuucudT(invdT) | 752 | pAGAAAAAACUGUUCGACcAdT(invdT) | 90 | 2 |
| 7 | uGcuuAAcuAcAuAuAGAudTsdT | 8 | AUfCfUfAUfAUfGUfAGUfUfAAGCfAdTsdT | 89 | 2 |
| 9 | GuAuGAAAAccuuAcuGcudTsdT | 10 | AGCfAGUfAAGGUfUfUfUfCfAUfACfdTsdT | 89 | 1 |
| 11 | cAGuGAGAGuuGGuuAcucdTsdT | 12 | GAGUfAACfCfAACfUfCfUfCfACfUfGdTsdT | 89 | 2 |
| 13 | GGGuGGAGAucAuAuAGAcdTsdT | 14 | GUCuAuAUGAUCUCcACCCdTsdT | 89 | 2 |
| 762 | GuuccAGAcucAAcuuGGcdTsdT | 770 | pUCcAAGUUGAGUCUGGAACdTsdT | 89 | 2 |
| 762 | GuuccAGAcucAAcuuGGcdTsdT | 84 | UCcAAGUUGAGUCUGGAACdTsdT | 89 | 2 |
| 55 | uGGucGAAcAGuuuuuucudTsdT | 744 | pAGAAAAAACUGUUCGACcAdTsdT | 89 | 3 |
| 763 | GuuccAGAcucAAcuuGGudTsdT | 84 | UCcAAGUUGAGUCUGGAACdTsdT | 89 | 1 |
| 763 | GuuccAGAcucAAcuuGGudTsdT | 770 | pUCcAAGUUGAGUCUGGAACdTsdT | 88 | 0 |
| 765 | GuuccAGAcucAAcuuGGcdT(invdT) | 771 | pUCcAAGUUGAGUCUGGAACdT(invdT) | 88 | 1 |
| 768 | GuuccAGAcucAAcuuGGcdT(abasic) | 774 | UCcAAGUUGAGUCUGGAACdT(abasic) | 88 | 1 |
| 769 | GuuccAGAcucAAcuuGGudT(abasic) | 774 | UCcAAGUUGAGUCUGGAACdT(abasic) | 88 | 1 |
| 769 | GuuccAGAcucAAcuuGGudT(abasic) | 773 | pUCcAAGUUGAGUCUGGAACdT(abasic) | 87 | 1 |
| 765 | GuuccAGAcucAAcuuGGcdT(invdT) | 772 | UCcAAGUUGAGUCUGGAACdT(invdT) | 87 | 1 |
| 766 | GuuccAGAcucAAcuuGGudT(invdT) | 771 | pUCcAAGUUGAGUCUGGAACdT(invdT) | 87 | 1 |
| 766 | GuuccAGAcucAAcuuGGudT(invdT) | 772 | UCcAAGUUGAGUCUGGAACdT(invdT) | 87 | 1 |
| 764 | GuuccAGAcucAAcuuGGAdT(invdT) | 772 | UCcAAGUUGAGUCUGGAACdT(invdT) | 87 | 2 |
| 767 | GuuccAGAcucAAcuuGGAdT(abasic) | 774 | UCcAAGUUGAGUCUGGAACdT(abasic) | 87 | 2 |
| 15 | GGGuGGAGAucAuAuAGAcdTsdT | 16 | GUfCfUfAUfAUfGAUfCfUfCfCfACfCfCfdTsdT | 87 | 2 |
| 17 | cAGuGAGAGuuGGuuAcucdTsdT | 18 | GAGuAACcAACUCUcACUGdTsdT | 87 | 2 |
| 19 | cAuAuAGAcAAucAAGuGcdTsdT | 20 | GCfACfUfUfGAUfUfGUfCfUfAUfAUfGdTsdT | 87 | 2 |
| 21 | ccuAuGuAuGuGuuAucuGdTsdT | 22 | CfAGAUfAACfACfAUfACfAUfAGGdTsdT | 87 | 1 |
| 23 | uuAAuGucAuuccAccAAudTsdT | 24 | AUfUfGGUfGGAAUfGACfAUfUfAAdTsdT | 87 | 2 |
| 25 | uuGcuuAAcuAcAuAuAGAdTsdT | 26 | UCuAuAUGuAGUuAAGcAAdTsdT | 86 | 3 |
| 27 | uGGucGAAcAGuuuuuucudTsdT | 28 | AGAAAAAACfUfGUfUfCfGACfCfAdTsdT | 86 | 1 |
| 29 | cAcAcAuuAAucuGAuuuudTsdT | 30 | AAAAUfCfAGAUfUfAAUfGUfGUfGdTsdT | 86 | 2 |
| 83 | GuuccAGAcucAAcuuGGAdTsdT | 770 | pUCcAAGUUGAGUCUGGAACdTsdT | 86 | 5 |
| 768 | GuuccAGAcucAAcuuGGcdT(abasic) | 773 | pUCcAAGUUGAGUCUGGAACdT(abasic) | 86 | 2 |
| 764 | GuuccAGAcucAAcuuGGAdT(invdT) | 771 | pUCcAAGUUGAGUCUGGAACdT(invdT) | 86 | 3 |
| 767 | GuuccAGAcucAAcuuGGAdT(abasic) | 773 | pUCcAAGUUGAGUCUGGAACdT(abasic) | 83 | 8 |
| 83 | GuuccAGAcucAAcuuGGAdTsdT | 770 | pUCcAAGUUGAGUCUGGAACdTsdT | 86 | 5 |
| 31 | GuAuGAAAAccuuAcuGcudTsdT | 32 | AGcAGuAAGGUUUUcAuACdTsdT | 85 | 3 |
| 33 | cuAcAGGAGucucAcAAGAdTsdT | 34 | UCUUGUGAGACUCCUGuAGdTsdT | 84 | 2 |
| 35 | cuGuAuGAAAAuAcccuccdTsdT | 36 | GGAGGGuAUUUUcAuAcAGdTsdT | 85 | 3 |
| 37 | uccuAuGuAuGuGiniAucudTsdT | 38 | AGAuAAcAcAuAcAuAGGAdTsdT | 85 | 5 |
| 39 | GGuGGAGAucAuAuAGAcAdTsdT | 40 | UfGUfCfUfAUfAUfGAUfCfUfCfCfACfCfdTsdT | 84 | 1 |
| 41 | AuGuAcGAccAAuGuAAAcdTsdT | 42 | GUfUfUfACfAUfUfGGUfCfGUfACfAUfdTsdT | 84 | 2 |
| 43 | AcuGGcAGcGGuuuuAucAdTsdT | 44 | UfGAUfAAAACfCfGCfUfGCfCfAGUfdTsdT | 84 | 2 |
| 45 | AGuGAGAGuuGGuuAcucAdTsdT | 46 | UfGAGUfAACfCfAACfUfCfUfCfACfUfdTsdT | 84 | 2 |
| 47 | AAuAAcuuGcuuAAcuAcAdTsdT | 48 | UfGUfAGUfUfAAGCfAAGUfUfAUfUfdTsdT | 84 | 1 |
| 49 | GuGAGAGuuGGuuAcucAcdTsdT | 50 | GUfGAGUfAACfCfAACfUfCfUfCfACfdTsdT | 83 | 3 |
| 51 | cAucAucGAuAAAAuucGAdTsdT | 52 | UfCfGAAUfUfUfUfAUfCfGAUfGAUfGdTsdT | 83 | 2 |
| 53 | cuGuAuGAAAAuAcccuccdTsdT | 54 | GGAGGGUfAUfUfUfUfCfAUfACfAGdTsdT | 83 | 4 |
| 55 | uGGucGAAcAGuuuuuucudTsdT | 56 | AGAAAAAACUGUUCGACcAdTsdT | 82 | 4 |
| 57 | AcGAuucAuuccuuuuGGAdTsdT | 58 | UfCfCfAAAAGGAAUfGAAUfCfGUfdTsdT | 82 | 2 |
| 59 | cuGuAuGAAAAccuuAcuGdTsdT | 60 | cAGuAAGGUUUUcAuAcAGdTsdT | 82 | 3 |
| 61 | GuGAGAGuuGGuuAcucAcdTsdT | 62 | GUGAGuAACcAACUCUcACdTsdT | 82 | 4 |
| 63 | uGuAcGAccAAuGuAAAcAdTsdT | 64 | UfGUfUfUfACfAUfUfGGUfCfGUfACfAdTsdT | 82 | 3 |
| 65 | uAccGGAcAcuAAAcccAAdTsdT | 66 | UfUfGGGUfUfUfAGUfGUfCfCfGGUfAdTsdT | 82 | 2 |
| 67 | ccGcuAucGAAAAuGucuudTsdT | 68 | AAGACfAUfUfUfUfCfGAUfAGCfGGdTsdT | 81 | 1 |
| 69 | AGAucAGAccuGuuGAuAGdTsdT | 70 | CfUfAUfCfAACfAGGUfCfUfGAUfCfUfdTsdT | 81 | 4 |
| 71 | uccuAuGuAuGuGuuAucudTsdT | 72 | AGAUfAACfACfAUfACfAUfAGGAdTsdT | 81 | 2 |
| 73 | ucuGuAuGAAAAccuuAcudTsdT | 74 | AGUfAAGGUMfUfUfCfAUfACfAGAdTsdT | 81 | 1 |
| 75 | AAAAcAAuAGuuccuGcAAdTsdT | 76 | UfUfGCfAGGAACfUfAUfUfGUfUfUfUfdTsdT | 80 | 3 |
| 77 | GucuuAAcuuGuGGAAGcudTsdT | 78 | AGCfUfUfCfCfACfAAGUfUfAAGACfdTsdT | 80 | 1 |
| 79 | AcAAuAGuuccuGcAAcGudTsdT | 80 | ACfGUfUfGCfAGGAACfUfAUfUfGUfdTsdT | 80 | 3 |
| 81 | AGGcuuuucAuuAAAuGGGdTsdT | 82 | CfCfCfAUfUfUfAAUfGAAAAGCfCfUfdTsdT | 80 | 3 |
| 83 | GuuccAGAcucAAcuuGGAdTsdT | 84 | UCcAAGUUGAGUCUGGAACdTsdT | 80 | 7 |
| 85 | AuGuAcGAccAAuGuAAAcdTsdT | 86 | GUUuAcAUUGGUCGuAcAUdTsdT | 80 | 4 |
| 87 | cuAcAGGAGucucAcAAGAdTsdT | 88 | UfCfUfUfGUfGAGACfUfCfCfUfGUfAGdTsdT | 80 | 2 |
| 89 | uGuAcGAccAAuGuAAAcAdTsdT | 90 | UGUUuAcAUUGGUCGuAcAdTsdT | 79 | 3 |
| 91 | AGGAucAGAAGccuAuuuudTsdT | 92 | AAAAUfAGGCfUfUfCfUfGAUfCfCfUfdTsdT | 79 | 3 |
| 93 | GAAAuuAGAAuGAccuAcAdTsdT | 94 | UGuAGGUcAUUCuAAUUUCdTsdT | 79 | 2 |
| 95 | uucuGuucAuGGuGuGAGudTsdT | 96 | ACfUfCfACfACfCfAUfGAACfAGAAdTsdT | 79 | 2 |
| 97 | GuuccAGAcucAAcuuGGAdTsdT | 98 | UfCfCfAAGUfUfGAGUfCfUfGGAACfdTsdT | 79 | 2 |
| 99 | ccAGAuGuAAGcucuccucdTsdT | 100 | GAGGAGAGCUuAcAUCUGGdTsdT | 79 | 4 |
| 101 | uuucuAAuGGcuAuucAAGdTsdT | 102 | CfUfUfGAAUfAGCfCfAUfUfAGAAAdTsdT | 79 | 2 |
| 103 | AuGccGcuAucGAAAAuGudTsdT | 104 | ACfAUfUfUfUfCfGAUfAGCfGGCfAUfdTsdT | 79 | 2 |
| 105 | ccAGcAuGccGcuAucGAAdTsdT | 106 | UfUfCfGAUfAGCfGGCfAUfGCfUfGGdTsdT | 79 | 2 |
| 107 | uuGGcGcucAAAAAAuAGAdTsdT | 108 | UCuAUUUUUUGAGCGCcAAdTsdT | 78 | 4 |
| 109 | uccAccAAuucccGuuGGudTsdT | 110 | ACfCfAACfGGGAAUfUfGGUfGGAdTsdT | 78 | 2 |
| 111 | AAAcAAuAGuuccuGcAAcdTsdT | 112 | GUfUfGCfAGGAACfUfAUfUfGUfUfUfdTsdT | 78 | 2 |
| 113 | uucuGuucAuGGuGuGAGudTsdT | 114 | ACUcAcACcAUGAAcAGAAdTsdT | 78 | 5 |
| 115 | AGcAuuGcAAAccucAAuAdTsdT | 116 | uAUUGAGGUUUGcAAUGCUdTsdT | 78 | 5 |
| 117 | GccucucAuuuuAccGGAcdTsdT | 118 | GUfCfCfGGUfAAAAUfGAGAGGCfdTsdT | 78 | 2 |
| 119 | cAGcAucccuuucucAAcAdTsdT | 120 | UGUUGAGAAAGGGAUGCUGdTsdT | 77 | 5 |
| 121 | GAGAucAuAuAGAcAAucAdTsdT | 122 | UGAUUGUCuAuAUGAUCUCdTsdT | 77 | 2 |
| 123 | GGcuGuAuGAAAAuAcccudTsdT | 124 | AGGGuAUUUUcAuAcAGCCdTsdT | 77 | 2 |
| 125 | AcGAuucAuuccuuuuGGAdTsdT | 126 | UCcAAAAGGAAUGAAUCGUdTsdT | 77 | 3 |
| 127 | uGGGAAAuGAccuGGGAuudTsdT | 128 | AAUCCcAGGUcAUUUCCcAdTsdT | 77 | 4 |
| 129 | cccAGGuAAAGAGAcGAAudTsdT | 130 | AUfUfCfGUfCfUfCfUfUfUfACfCfUfGGGdTsdT | 77 | 5 |
| 131 | cAGcAucccuuucucAAcAdTsdT | 132 | UfGUfUfGAGAAAGGGAUfGCfUfGdTsdT | 77 | 3 |
| 133 | cAGGuAAAGAGAcGAAuGAdTsdT | 134 | UfCfAUfUfCfGUfCfUfCfUfUfUfACfCfUfGdTsdT | 77 | 4 |
| 135 | AAuAAcuuGcuuAAcuAcAdTsdT | 136 | UGuAGUuAAGcAAGUuAUUdTsdT | 77 | 4 |
| 137 | cuGuAuGAAAAccuuAcuGdTsdT | 138 | CfAGUfAAGGUfUfUfUfCfAUfACfAGdTsdT | 76 | 4 |
| 139 | GcucuGuuccAGAcucAAcdTsdT | 140 | GUfUfGAGUfCfUfGGAACfAGAGCfdTsdT | 76 | 3 |
| 141 | GGcucAGuAAGcAAuGcGcdTsdT | 142 | GCfGCfAUfUfGCfUfUfACfUfGAGCfCfdTsdT | 76 | 5 |
| 143 | GAGAucAuAuAGAcAAucAdTsdT | 144 | UfGAUfUfGUfCfUfAUfAUfGAUfCfUfCfdTsdT | 76 | 2 |
| 145 | AGGAucAGAAGccuAuuuudTsdT | 146 | AAAAuAGGCUUCUGAUCCUdTsdT | 76 | 4 |
| 147 | cAGcAuGccGcuAucGAAAdTsdT | 148 | UUUCGAuAGCGGcAUGCUGdTsdT | 76 | 4 |
| 149 | uGuuAuAuGcAGGAuAuGAdTsdT | 150 | UfCfAUfAUfCfCfUfGCfAUfAUfAACfAdTsdT | 76 | 1 |
| 151 | cGcuAucGAAAAuGucuucdTsdT | 152 | GAAGACfAUfUfUfUfCfGAUfAGCfGdTsdT | 76 | 1 |
| 153 | GGuGGAGAucAuAuAGAcAdTsdT | 154 | UGUCuAuAUGAUCUCcACCdTsdT | 76 | 2 |
| 155 | uuGGcGcucAAAAAAuAGAdTsdT | 156 | UfCfUfAUfUfUfUfUfUfGAGCfGCfCfAAdTsdT | 76 | 2 |
| 157 | ucAuuuuAccGGAcAcuAAdTsdT | 158 | UuAGUGUCCGGuAAAAUGAdTsdT | 75 | 3 |
| 159 | cAucAucGAuAAAAuucGAdTsdT | 160 | UCGAAUUUuAUCGAUGAUGdTsdT | 75 | 8 |
| 161 | ccAGGuAAAGAGAcGAAuGdTsdT | 162 | cAUUCGUCUCUUuACCUGGdTsdT | 75 | 5 |
| 163 | cAGGcuucAGGuAucuuAudTsdT | 164 | AuAAGAuACCUGAAGCCUGdTsdT | 75 | 3 |
| 165 | uuuccAAAAGGcucAGuAAdTsdT | 166 | UuACUGAGCCUUUUGGAAAdTsdT | 75 | 2 |
| 167 | cAcAcAuuAAucuGAuuuudTsdT | 168 | AAAAUcAGAUuAAUGUGUGdTsdT | 75 | 6 |
| 169 | GGcuGuAuGAAAAuAcccudTsdT | 170 | AGGGUfAUfUfUfUfCfAUfACfAGCfCfdTsdT | 75 | 3 |
| 171 | cAGGuuucAGGAAcuuAcAdTsdT | 172 | UGuAAGUUCCUGAAACCUGdTsdT | 75 | 3 |
| 173 | GAAAuuAGAAuGAccuAcAdTsdT | 174 | UfGUfAGGUfCfAUfUfCfUfAAUfUfUfCfdTsdT | 75 | 2 |
| 175 | ccAAGcAGcGAAGAcuuuudTsdT | 176 | AAAAGUfCfUfUfCfGCfUfGCfUfUfGGdTsdT | 74 | 4 |
| 177 | uccAccAAuucccGuuGGudTsdT | 178 | ACcAACGGGAAUUGGUGGAdTsdT | 74 | 8 |
| 179 | ccAAcAAucuuGGcGcucAdTsdT | 180 | UGAGCGCcAAGAUUGUUGGdTsdT | 74 | 7 |
| 181 | cucAGuAAGcAAuGcGcAGdTsdT | 182 | CUGCGcAUUGCUuACUGAGdTsdT | 74 | 4 |
| 183 | ucucAAuGGGAcuGuAuAudTsdT | 184 | AUfAUfACfAGUfCfCfCfAUfUfGAGAdTsdT | 74 | 3 |
| 185 | AAAAAGAAGAuuucAucGAdTsdT | 186 | UfCfGAUfGAAAUfCfUfUfCfUfUfUfUfUfdTsdT | 73 | 3 |
| 187 | GAAcuGGcAGcGGuuuuAudTsdT | 188 | AUfAAAACfCfGCfUfGCfCfAGUfUfCfdTsdT | 73 | 2 |
| 189 | GcucuGuuccAGAcucAAcdTsdT | 190 | GUUGAGUCUGGAAcAGAGCdTsdT | 73 | 1 |
| 191 | cAccAAuucccGuuGGuucdTsdT | 192 | GAACfCfAACfGGGAAUfUfGGUfGdTsdT | 73 | 3 |
| 193 | cGcuAucGAAAAuGucuucdTsdT | 194 | GAAGAcAUUUUCGAuAGCGdTsdT | 73 | 6 |
| 195 | AGcAuGccGcuAucGAAAAdTsdT | 196 | UfUfUfUfCfGAUfAGCfGGCfAUfGCfUfdTsdT | 73 | 2 |
| 197 | cucAAcuuGGAGGAucAuGdTsdT | 198 | cAUGAUCCUCcAAGUUGAGdTsdT | 73 | 7 |
| 199 | ccAGAuGuAAGcucuccucdTsdT | 200 | GAGGAGAGCfUfUfACfAUfCfUfGGdTsdT | 73 | 2 |
| 201 | AGuGAGAGuuGGuuAcucAdTsdT | 202 | UGAGuAACcAACUCUcACUdTsdT | 73 | 5 |
| 203 | GGGcGGcAAGuGAuuGcAGdTsdT | 204 | CUGcAAUcACUUGCCGCCCdTsdT | 72 | 4 |
| 205 | uGuGAuGGAcuucuAuAAAdTsdT | 206 | UfUfUfAUfAGAAGUfCfCfAUfCfACfAdTsdT | 72 | 5 |
| 207 | ccAAGcAGcGAAGAcuuuudTsdT | 208 | AAAAGUCUUCGCUGCUUGGdTsdT | 72 | 4 |
| 209 | AAAAcAAuAGuuccuGcAAdTsdT | 210 | UUGcAGGAACuAUUGUUUUdTsdT | 72 | 3 |
| 211 | ccGcuAucGAAAAuGucuudTsdT | 212 | AAGAcAUUUUCGAuAGCGGdTsdT | 71 | 5 |
| 213 | cAGcAuGccGcuAucGAAAdTsdT | 214 | UfUfUfCfGAUfAGCfGGCfAUfGCfUfGdTsdT | 71 | 3 |
| 215 | cuGGuGuGcucuGAuGAAGdTsdT | 216 | CfUfUfCfAUfCfAGAGCfACfACfCfAGdTsdT | 71 | 3 |
| 217 | AcGcucAAcAuGuuAGGAGdTsdT | 218 | CUCCuAAcAUGUUGAGCGUdTsdT | 71 | 4 |
| 219 | ucccAAcAAucuuGGcGcudTsdT | 220 | AGCfGCfCfAAGAUfUfGUfUfGGGAdTsdT | 71 | 4 |
| 221 | AGAcGAAuGAGAGuccuuGdTsdT | 222 | CfAAGGACfUfCfUfCfAUfUfCfGUfCfUfdTsdT | 71 | 6 |
| 223 | uAccGGAcAcuAAAcccAAdTsdT | 224 | UUGGGUUuAGUGUCCGGuAdTsdT | 70 | 9 |
| 225 | cuGcAAcGuuAccAcAAcudTsdT | 226 | AGUUGUGGuAACGUUGcAGdTsdT | 70 | 4 |
| 227 | ccAGcAuGccGcuAucGAAdTsdT | 228 | UUCGAuAGCGGcAUGCUGGdTsdT | 70 | 4 |
| 229 | AGcAuuGcAAAccucAAuAdTsdT | 230 | UfAUfUfGAGGUfUfUfGCfAAUfGCfUfdTsdT | 70 | 5 |
| 231 | ucccAAcAAucuuGGcGcudTsdT | 232 | AGCGCcAAGAUUGUUGGGAdTsdT | 70 | 6 |
| 233 | ccAccAAuucccGuuGGuudTsdT | 234 | AACcAACGGGAAUUGGUGGdTsdT | 70 | 5 |
| 235 | ucAGAccuGuuGAuAGAuGdTsdT | 236 | CfAUfCfUfAUfCfAACfAGGUfCfUfGAdTsdT | 70 | 4 |
| 237 | uuAccGGAcAcuAAAcccAdTsdT | 238 | UGGGUUuAGUGUCCGGuAAdTsdT | 70 | 8 |
| 239 | cccAAcAAucuuGGcGcucdTsdT | 240 | GAGCfGCfCfAAGAUfUfGUfUfGGGdTsdT | 70 | 4 |
| 241 | uuucuAAuGGcuAuucAAGdTsdT | 242 | CUUGAAuAGCcAUuAGAAAdTsdT | 70 | 8 |
| 243 | uuAAuGucAuuccAccAAudTsdT | 244 | AUUGGUGGAAUGAcAUuAAdTsdT | 70 | 5 |
| 245 | GGcucAGuAAGcAAuGcGcdTsdT | 246 | GCGcAUUGCUuACUGAGCCdTsdT | 69 | 6 |
| 247 | GucuuAAcuuGuGGAAGcudTsdT | 248 | AGCUUCcAcAAGUuAAGACdTsdT | 69 | 5 |
| 249 | ucAuuuuAccGGAcAcuAAdTsdT | 250 | UfUfAGUfGUfCfCfGGUfAAAAUfGAdTsdT | 69 | 5 |
| 251 | AGAcGAAuGAGAGuccuuGdTsdT | 252 | cAAGGACUCUcAUUCGUCUdTsdT | 69 | 6 |
| 253 | AcuGuAAAAccuuGuGuGGdTsdT | 254 | CfCfACfACfAAGGUfUfUfUfACfAGUfdTsdT | 68 | 3 |
| 255 | AAccucAAuAGGucGAccAdTsdT | 256 | UGGUCGACCuAUUGAGGUUdTsdT | 68 | 4 |
| 257 | cAuGcuGAAuAAuAAucuGdTsdT | 258 | CfAGAUfUfAUfUfAUfUfCfAGCfAUfGdTsdT | 68 | 3 |
| 259 | uGcAAAccucAAuAGGucGdTsdT | 260 | CGACCuAUUGAGGUUUGcAdTsdT | 68 | 4 |
| 261 | ccAAcAAucuuGGcGcucAdTsdT | 262 | UfGAGCfGCfCfAAGAUfUfGUfUfGGdTsdT | 68 | 4 |
| 263 | GGuuucAGGAAcuuAcAccdTsdT | 264 | GGUGuAAGUUCCUGAAACCdTsdT | 68 | 2 |
| 265 | GGuuucAGGAAcuuAcAccdTsdT | 266 | GGUfGUfAAGUfUfCfCfUfGAAACfCfdTsdT | 68 | 2 |
| 267 | uAGuGAccAGGuuuucAGGdTsdT | 268 | CCUGAAAACCUGGUcACuAdTsdT | 68 | 3 |
| 269 | cuGcAAcGuuAccAcAAcudTsdT | 270 | AGUfUfGUfGGUfAACfGUfUfGCfAGdTsdT | 68 | 3 |
| 271 | AGcAuGccGcuAucGAAAAdTsdT | 272 | UUUUCGAuAGCGGcAUGCUdTsdT | 67 | 4 |
| 273 | uGcAAcGuuAccAcAAcucdTsdT | 274 | GAGUUGUGGuAACGUUGcAdTsdT | 67 | 3 |
| 275 | uGAAccuGAAGuGuuAuAudTsdT | 276 | AUfAUfAACfACfUfUfCfAGGUfUfCfAdTsdT | 66 | 3 |
| 277 | cAccAAuucccGuuGGuucdTsdT | 278 | GAACcAACGGGAAUUGGUGdTsdT | 66 | 7 |
| 279 | ccAGGuAAAGAGAcGAAuGdTsdT | 280 | CfAUfUfCfGUfCfUfCfUfUfUfACfCfUfGGdTsdT | 66 | 4 |
| 281 | cucucAAuGGGAcuGuAuAdTsdT | 282 | UfAUfACfAGUfCfCfCfAUfUfGAGAGdTsdT | 66 | 6 |
| 283 | uGGcGcucAAAAAAuAGAAdTsdT | 284 | UfUfCfUfAUfUfUfUfUfUfGAGCfGCfCfAdTsdT | 66 | 3 |
| 285 | AuAcccuccucAAAuAAcudTsdT | 286 | AGUfUfAUfUfUfGAGGAGGGUfAUfdTsdT | 65 | 1 |
| 287 | GGGcGGcAAGuGAuuGcAGdTsdT | 288 | CfUfGCfAAUfCfACfUfUfGCfCfGCfCfCfdTsdT | 65 | 2 |
| 289 | uGcuuAAcuAcAuAuAGAudTsdT | 290 | AUCuAuAUGuAGUuAAGcAdTsdT | 65 | 4 |
| 291 | AuuccAccAAuucccGuuGdTsdT | 292 | CfAACfGGGAAUfUfGGUfGGAAUfdTsdT | 64 | 5 |
| 293 | AccucAAuAGGucGAccAGdTsdT | 294 | CUGGUCGACCuAUUGAGGUdTsdT | 64 | 4 |
| 295 | GuucAuGGuGuGAGuAccudTsdT | 296 | AGGUfACfUfCfACfACfCfAUfGAACfdTsdT | 63 | 4 |
| 297 | ccucucAuuuuAccGGAcAdTsdT | 298 | UGUCCGGuAAAAUGAGAGGdTsdT | 63 | 5 |
| 299 | AGccucucAuuuuAccGGAdTsdT | 300 | UfCfCfGGUfAAAAUfGAGAGGCfUfdTsdT | 63 | 5 |
| 301 | ucAAuGGGAcuGuAuAuGGdTsdT | 302 | CfCfAUfAUfACfAGUfCfCfCfAUfUfGAdTsdT | 63 | 6 |
| 303 | cAGGcuucAGGuAucuuAudTsdT | 304 | AUfAAGAUfACfCfUfGAAGCfCfUfGdTsdT | 63 | 4 |
| 305 | AuucAGcAGGccAcuAcAGdTsdT | 306 | CfUfGUfAGUfGGCfCfUfGCfUfGAAUfdTsdT | 63 | 2 |
| 307 | cccAAcAAucuuGGcGcucdTsdT | 308 | GAGCGCcAAGAUUGUUGGGdTsdT | 62 | 4 |
| 309 | AuGAGAccAGAuGuAAGcudTsdT | 310 | AGCUuAcAUCUGGUCUcAUdTsdT | 62 | 5 |
| 311 | cAuGcuGAAuAAuAAucuGdTsdT | 312 | cAGAUuAUuAUUcAGcAUGdTsdT | 62 | 5 |
| 313 | AcuGGcAGcGGuuuuAucAdTsdT | 314 | UGAuAAAACCGCUGCcAGUdTsdT | 62 | 5 |
| 315 | AucuGGuuuuGucAAGcccdTsdT | 316 | GGGCfUfUfGACfAAAACfCfAGAUfdTsdT | 62 | 6 |
| 317 | uGAGAGuuGGuuAcucAcAdTsdT | 318 | UGUGAGuAACcAACUCUcAdTsdT | 61 | 4 |
| 319 | ccAccAAuucccGuuGGuudTsdT | 320 | AACfCfAACfGGGAAUfUfGGUfGGdTsdT | 61 | 4 |
| 321 | AAAcuGGGcAcAGuuuAcudTsdT | 322 | AGUfAAACfUfGUfGCfCfCfAGUfUfUfdTsdT | 61 | 6 |
| 323 | GuucAuGGuGuGAGuAccudTsdT | 324 | AGGuACUcAcACcAUGAACdTsdT | 60 | 8 |
| 325 | AuAcccuccucAAAuAAcudTsdT | 326 | AGUuAUUUGAGGAGGGuAUdTsdT | 59 | 5 |
| 327 | uuAccGGAcAcuAAAcccAdTsdT | 328 | UfGGGUfUfUfAGUfGUfCfCfGGUfAAdTsdT | 59 | 5 |
| 329 | AcuuAcAccuGGAuGAccAdTsdT | 330 | UfGGUfCfAUfCfCfAGGUfGUfAAGUfdTsdT | 59 | 3 |
| 331 | cucAGuAAGcAAuGcGcAGdTsdT | 332 | CfUfGCfGCfAUfUfGCfUfUfACfUfGAGdTsdT | 59 | 7 |
| 333 | uuuGAcAuuuuGcAGGAuudTsdT | 334 | AAUfCfCfUfGCfAAAAUfGUfCfAAAdTsdT | 59 | 9 |
| 335 | ucAGAccuGuuGAuAGAuGdTsdT | 336 | cAUCuAUcAAcAGGUCUGAdTsdT | 58 | 6 |
| 337 | AuucAGcAGGccAcuAcAGdTsdT | 338 | CUGuAGUGGCCUGCUGAAUdTsdT | 57 | 5 |
| 339 | AuAGuuccuGcAAcGuuAcdTsdT | 340 | GUfAACfGUfUfGCfAGGAACfUfAUfdTsdT | 56 | 3 |
| 341 | uGcAAcGuuAccAcAAcucdTsdT | 342 | GAGUfUfGUfGGUfAACfGUfUfGCfAdTsdT | 56 | 5 |
| 343 | uAGuuuuuuAuucAuGcuGdTsdT | 344 | CfAGCfAUfGAAUfAAAAAACfUfAdTsdT | 56 | 6 |
| 345 | uGGGAAAuGAccuGGGAuudTsdT | 346 | AAUfCfCfCfAGGUfCfAUfUfUfCfCfCfAdTsdT | 56 | 7 |
| 347 | uuuGAcAuuuuGcAGGAuudTsdT | 348 | AAUCCUGcAAAAUGUcAAAdTsdT | 56 | 5 |
| 349 | AcGcucAAcAuGuuAGGAGdTsdT | 350 | CfUfCfCfUfAACfAUfGUfUfGAGCfGUfdTsdT | 54 | 6 |
| 351 | uGcuGuucuGGuAuuAccAdTsdT | 352 | UfGGUfAAUfACfCfAGAACfAGCfAdTsdT | 54 | 3 |
| 353 | cccAGGuAAAGAGAcGAAudTsdT | 354 | AUUCGUCUCUUuACCUGGGdTsdT | 53 | 11 |
| 355 | uGcAAAccucAAuAGGucGdTsdT | 356 | CfGACfCfUfAUfUfGAGGUfUfUfGCfAdTsdT | 53 | 5 |
| 357 | GccucucAuuuuAccGGAcdTsdT | 358 | GUCCGGuAAAAUGAGAGGCdTsdT | 52 | 6 |
| 359 | uGcuGuucuGGuAuuAccAdTsdT | 360 | UGGuAAuACcAGAAcAGcAdTsdT | 52 | 4 |
| 361 | GAAcuGGcAGcGGuuuuAudTsdT | 362 | AuAAAACCGCUGCcAGUUCdTsdT | 52 | 4 |
| 363 | ccuAuGuAuGuGuuAucuGdTsdT | 364 | cAGAuAAcAcAuAcAuAGGdTsdT | 51 | 5 |
| 365 | AGAAGAuuucAucGAAcucdTsdT | 366 | GAGUfUfCfGAUfGAAAUfCfUfUfCfUfdTsdT | 51 | 6 |
| 367 | cucuGAAcuucccuGGucGdTsdT | 368 | CfGACfCfAGGGAAGUfUfCfAGAGdTsdT | 51 | 3 |
| 369 | cuGGuGuGcucuGAuGAAGdTsdT | 370 | CUUcAUcAGAGcAcACcAGdTsdT | 51 | 6 |
| 371 | cucAAcuuGGAGGAucAuGdTsdT | 372 | CfAUfGAUfCfCfUfCfCfAAGUfUfGAGdTsdT | 50 | 5 |
| 373 | AGccucucAuuuuAccGGAdTsdT | 374 | UCCGGuAAAAUGAGAGGCUdTsdT | 50 | 7 |
| 375 | AuAGuuccuGcAAcGuuAcdTsdT | 376 | GuAACGUUGcAGGAACuAUdTsdT | 50 | 6 |
| 377 | AAcAAuAGuuccuGcAAcGdTsdT | 378 | CfGUfUfGCfAGGAACfUfAUfUfGUfUfdTsdT | 50 | 3 |
| 379 | AucuGGuuuuGucAAGcccdTsdT | 380 | GGGCUUGAcAAAACcAGAUdTsdT | 49 | 6 |
| 381 | AcuGuAAAAccuuGuGuGGdTsdT | 382 | CcAcAcAAGGUUUuAcAGUdTsdT | 49 | 6 |
| 383 | AAcucuuGGAuucuAuGcAdTsdT | 384 | UGcAuAGAAUCcAAGAGUUdTsdT | 49 | 7 |
| 385 | uAGuGAccAGGuuuucAGGdTsdT | 386 | CfCfUfGAAAACfCfUfGGUfCfACfUfAdTsdT | 49 | 6 |
| 387 | AAccucAAuAGGucGAccAdTsdT | 388 | UfGGUfCfGACfCfUfAUfUfGAGGUfUfdTsdT | 49 | 5 |
| 389 | ccucucAuuuuAccGGAcAdTsdT | 390 | UfGUfCfCfGGUfAAAAUfGAGAGGdTsdT | 48 | 4 |
| 391 | uGAccAAAuGAcccuAcuGdTsdT | 392 | CfAGUfAGGGUfCfAUfUfUfGGUfCfAdTsdT | 48 | 6 |
| 393 | AGAucAGAccuGuuGAuAGdTsdT | 394 | CuAUcAAcAGGUCUGAUCUdTsdT | 48 | 10 |
| 395 | cAGGuuucAGGAAcuuAcAdTsdT | 396 | UfGUfAAGUfUfCfCfUfGAAACfCfUfGdTsdT | 47 | 5 |
| 397 | uAGuuuuuuAuucAuGcuGdTsdT | 398 | cAGcAUGAAuAAAAAACuAdTsdT | 47 | 7 |
| 399 | uGuGAuGGAcuucuAuAAAdTsdT | 400 | UUuAuAGAAGUCcAUcAcAdTsdT | 45 | 4 |
| 401 | uGGcGcucAAAAAAuAGAAdTsdT | 402 | UUCuAUUUUUUGAGCGCcAdTsdT | 45 | 10 |
| 403 | AAcAAuAGuuccuGcAAcGdTsdT | 404 | CGUUGcAGGAACuAUUGUUdTsdT | 44 | 6 |
| 405 | uGAAccuGAAGuGuuAuAudTsdT | 406 | AuAuAAcACUUcAGGUUcAdTsdT | 44 | 5 |
| 407 | cucucAAuGGGAcuGuAuAdTsdT | 408 | uAuAcAGUCCcAUUGAGAGdTsdT | 42 | 6 |
| 409 | ucuGuAuGAAAAccuuAcudTsdT | 410 | AGuAAGGUUUUcAuAcAGAdTsdT | 41 | 2 |
| 411 | AuGccGcuAucGAAAAuGudTsdT | 412 | AcAUUUUCGAuAGCGGcAUdTsdT | 41 | 7 |
| 413 | uAGuuccuGcAAcGuuAccdTsdT | 414 | GGuAACGUUGcAGGAACuAdTsdT | 40 | 7 |
| 415 | AAcAAucuuGGcGcucAAAdTsdT | 416 | UfUfUfGAGCfGCfCfAAGAUfUfGUfUfdTsdT | 40 | 8 |
| 417 | AAAccucAAuAGGucGAccdTsdT | 418 | GGUfCfGACfCfUfAUfUfGAGGUfUfUfdTsdT | 40 | 4 |
| 419 | uuuccAAAAGGcucAGuAAdTsdT | 420 | UfUfACfUfGAGCfCfUfUfUfUfGGAAAdTsdT | 38 | 7 |
| 421 | ucucAAuGGGAcuGuAuAudTsdT | 422 | AuAuAcAGUCCcAUUGAGAdTsdT | 38 | 8 |
| 423 | AAcAAucuuGGcGcucAAAdTsdT | 424 | UUUGAGCGCcAAGAUUGUUdTsdT | 38 | 7 |
| 425 | AAcucuuGGAuucuAuGcAdTsdT | 426 | UfGCfAUfAGAAUfCfCfAAGAGUfUfdTsdT | 38 | 8 |
| 427 | AAAAAGAAGAuuucAucGAdTsdT | 428 | UCGAUGAAAUCUUCUUUUUdTsdT | 37 | 9 |
| 429 | cAuAuAGAcAAucAAGuGcdTsdT | 430 | GcACUUGAUUGUCuAuAUGdTsdT | 37 | 3 |
| 431 | AcuuAcAccuGGAuGAccAdTsdT | 432 | UGGUcAUCcAGGUGuAAGUdTsdT | 34 | 14 |
| 433 | uuuAccGGAcAcuAAAcccdTsdT | 434 | GGGUfUfUfAGUfGUfCfCfGGUfAAAdTsdT | 33 | 8 |
| 435 | cAGGuAAAGAGAcGAAuGAdTsdT | 436 | UcAUUCGUCUCUUuACCUGdTsdT | 32 | 11 |
| 437 | cccAGcAuGccGcuAucGAdTsdT | 438 | UfCfGAUfAGCfGGCfAUfGCfUfGGGdTsdT | 31 | 8 |
| 439 | cccAGcAuGccGcuAucGAdTsdT | 440 | UCGAuAGCGGcAUGCUGGGdTsdT | 31 | 8 |
| 441 | GGAGGAcAGAuGuAccAcudTsdT | 442 | AGUfGGUfACfAUfCfUfGUfCfCfUfCfCfdTsdT | 30 | 5 |
| 443 | cAuGuAcGAccAAuGuAAAdTsdT | 444 | UUuAcAUUGGUCGuAcAUGdTsdT | 30 | 4 |
| 445 | uAGuuccuGcAAcGuuAccdTsdT | 446 | GGUfAACfGUfUfGCfAGGAACfUfAdTsdT | 30 | 7 |
| 447 | AAcuuAcAccuGGAuGAccdTsdT | 448 | GGUfCfAUfCfCfAGGUfGUfAAGUfUfdTsdT | 29 | 5 |
| 449 | AAAcAAuAGuuccuGcAAcdTsdT | 450 | GUUGcAGGAACuAUUGUUUdTsdT | 29 | 11 |
| 451 | uuuuAccGGAcAcuAAAccdTsdT | 452 | GGUfUfUfAGUfGUfCfCfGGUfAAAAdTsdT | 28 | 7 |
| 453 | uGAGAGuuGGuuAcucAcAdTsdT | 454 | UfGUfGAGUfAACfCfAACfUfCfUfCfAdTsdT | 28 | 7 |
| 455 | AcAAuAGuuccuGcAAcGudTsdT | 456 | ACGUUGcAGGAACuAUUGUdTsdT | 27 | 8 |
| 457 | GGuccAcccAGGAuuAGuGdTsdT | 458 | CfACfUfAAUfCfCfUfGGGUfGGACfCfdTsdT | 27 | 7 |
| 459 | uGuuAuAuGcAGGAuAuGAdTsdT | 460 | UcAuAUCCUGcAuAuAAcAdTsdT | 27 | 6 |
| 461 | AuGAGAccAGAuGuAAGcudTsdT | 462 | AGCfUfUfACfAUfCfUfGGUfCfUfCfAUfdTsdT | 26 | 5 |
| 463 | AccucAAuAGGucGAccAGdTsdT | 464 | CfUfGGUfCfGACfCfUfAUfUfGAGGUfdTsdT | 26 | 2 |
| 465 | AGAAGAuuucAucGAAcucdTsdT | 466 | GAGUUCGAUGAAAUCUUCUdTsdT | 26 | 7 |
| 467 | AAAccucAAuAGGucGAccdTsdT | 468 | GGUCGACCuAUUGAGGUUUdTsdT | 25 | 7 |
| 469 | uuccAccAAuucccGuuGGdTsdT | 470 | CfCfAACfGGGAAUfUfGGUfGGAAdTsdT | 24 | 10 |
| 471 | uGAccAAAuGAcccuAcuGdTsdT | 472 | cAGuAGGGUcAUUUGGUcAdTsdT | 23 | 6 |
| 473 | AuuccAccAAuucccGuuGdTsdT | 474 | cAACGGGAAUUGGUGGAAUdTsdT | 23 | 12 |
| 475 | uGGuccAcccAGGAuuAGudTsdT | 476 | ACfUfAAUfCfCfUfGGGUfGGACfCfAdTsdT | 22 | 6 |
| 477 | AGGAAuucAGcAGGccAcudTsdT | 478 | AGUGGCCUGCUGAAUUCCUdTsdT | 22 | 8 |
| 479 | AcuucccuGGucGAAcAGudTsdT | 480 | ACfUfGUfUfCfGACfCfAGGGAAGUfdTsdT | 22 | 8 |
| 481 | uuuuAccGGAcAcuAAAccdTsdT | 482 | GGUUuAGUGUCCGGuAAAAdTsdT | 20 | 12 |
| 483 | AAAuAAcuuGcuuAAcuAcdTsdT | 484 | GuAGUuAAGcAAGUuAUUUdTsdT | 20 | 10 |
| 485 | AAGGcucAGuAAGcAAuGcdTsdT | 486 | GCfAUfUfGCfUfUfACfUfGAGCfCfUfUfdTsdT | 16 | 9 |
| 487 | AGGAAuucAGcAGGccAcudTsdT | 488 | AGUfGGCfCfUfGCfUfGAAUfUfCfCfUfdTsdT | 16 | 8 |
| 489 | cucuGAAcuucccuGGucGdTsdT | 490 | CGACcAGGGAAGUUcAGAGdTsdT | 15 | 9 |
| 491 | ucAAuGGGAcuGuAuAuGGdTsdT | 492 | CcAuAuAcAGUCCcAUUGAdTsdT | 14 | 8 |
| 493 | AAAcuGGGcAcAGuuuAcudTsdT | 494 | AGuAAACUGUGCCcAGUUUdTsdT | 13 | 12 |
| 495 | AAGccucucAuuuuAccGGdTsdT | 496 | CfCfGGUfAAAAUfGAGAGGCfUfUfdTsdT | 9 | 6 |
| 497 | AcuucccuGGucGAAcAGudTsdT | 498 | ACUGUUCGACcAGGGAAGUdTsdT | 8 | 13 |
| 499 | AAcuuAcAccuGGAuGAccdTsdT | 500 | GGUcAUCcAGGUGuAAGUUdTsdT | 8 | 6 |
| 501 | GGAGGAcAGAuGuAccAcudTsdT | 502 | AGUGGuAcAUCUGUCCUCCdTsdT | 8 | 8 |
| 503 | GGuccAcccAGGAuuAGuGdTsdT | 504 | cACuAAUCCUGGGUGGACCdTsdT | 8 | 7 |
| 505 | AAGGcucAGuAAGcAAuGcdTsdT | 506 | GcAUUGCUuACUGAGCCUUdTsdT | 7 | 7 |
| 507 | uuccAccAAuucccGuuGGdTsdT | 508 | CcAACGGGAAUUGGUGGAAdTsdT | 7 | 8 |
| 509 | uuuAccGGAcAcuAAAcccdTsdT | 510 | GGGUUuAGUGUCCGGuAAAdTsdT | 1 | 13 |
| 511 | uGGuccAcccAGGAuuAGudTsdT | 512 | ACuAAUCCUGGGUGGACcAdTsdT | 0 | 15 |
| 513 | AAGccucucAuuuuAccGGdTsdT | 514 | CCGGuAAAAUGAGAGGCUUdTsdT | −1 | 12 |
| 515 | AGGcuuuucAuuAAAuGGGdTsdT | 516 | CCcAUUuAAUGAAAAGCCUdTsdT | −14 | 16 |

TABLE 2

| | Activity testing for dose response in | | | | Activity testing for dose response | | | |
|---|---|---|---|---|---|---|---|---|
| | HeLaS3 cells - transfection 1 | | | | in HeLaS3 cells - transfection 2 | | | |
| | mean | mean | mean | | mean | mean | mean | |
| SEQ ID | IC50 | IC80 | IC20 | mean maximal | IC50 | IC80 | IC20 | mean maximal |
| NO pair | [nM] | [nM] | [nM] | inhibition [%] | [nM] | [nM] | [nM] | inhibition [%] |
| 7/8 | 0.003 | 0.047 | 0 | 87 | n.d. | n.d. | n.d. | n.d. |
| 31/32 | 0.004 | 0.09 | 0 | 89 | n.d. | n.d. | n.d. | n.d. |
| 3/4 | 0.005 | 0.072 | 0.001 | 88 | n.d. | n.d. | n.d. | n.d. |
| 25/26 | 0.006 | 0.139 | 0.001 | 91 | n.d. | n.d. | n.d. | n.d. |
| 33/34 | 0.008 | 0.114 | 0.001 | 86 | n.d. | n.d. | n.d. | n.d. |
| 83/84 | 0.009 | 0.201 | 0.002 | 84 | 0.0033 | 0.0739 | 0.0005 | 84 |
| 55/56 | 0.009 | 0.105 | 0.002 | 84 | 0.0055 | 0.0844 | 0.001 | 81 |
| 27/28 | 0.011 | 0.221 | 0.001 | 83 | n.d. | n.d. | n.d. | n.d. |
| 9/10 | 0.012 | 0.238 | 0.001 | 87 | n.d. | n.d. | n.d. | n.d. |
| 15/16 | 0.015 | 0.131 | 0.003 | 86 | n.d. | n.d. | n.d. | n.d. |
| 35/36 | 0.016 | 0.358 | 0.002 | 89 | n.d. | n.d. | n.d. | n.d. |
| 17/18 | 0.025 | 0.179 | 0.005 | 92 | n.d. | n.d. | n.d. | n.d. |
| 37/38 | 0.025 | 0.563 | 0.003 | 82 | n.d. | n.d. | n.d. | n.d. |
| 11/12 | 0.031 | 0.35 | 0.005 | 88 | n.d. | n.d. | n.d. | n.d. |
| 13/14 | 0.036 | 0.304 | 0.007 | 87 | n.d. | n.d. | n.d. | n.d. |
| 19/20 | 0.04 | 0.446 | 0.009 | 86 | n.d. | n.d. | n.d. | n.d. |
| 57/58 | 0.041 | 1′717 | 0.006 | 83 | n.d. | n.d. | n.d. | n.d. |
| 59/60 | 0.044 | 0.488 | 0.008 | 87 | n.d. | n.d. | n.d. | n.d. |
| 1/2 | 0.052 | 0.397 | 0.011 | 90 | n.d. | n.d. | n.d. | n.d. |
| 21/22 | 0.055 | 0.627 | 0.009 | 86 | n.d. | n.d. | n.d. | n.d. |
| 5/6 | 0.056 | 0.565 | 0.01 | 89 | n.d. | n.d. | n.d. | n.d. |
| 29/30 | 0.058 | 0.824 | 0.011 | 85 | n.d. | n.d. | n.d. | n.d. |
| 23/24 | 0.06 | 0.798 | 0.011 | 85 | n.d. | n.d. | n.d. | n.d. |
| 61/62 | 0.082 | 0.827 | 0.016 | 87 | n.d. | n.d. | n.d. | n.d. |
| 85/86 | 0.083 | 2′072 | 0.017 | 84 | n.d. | n.d. | n.d. | n.d. |
| 83/770 | n.d. | n.d. | n.d. | n.d. | 0.0041 | 0.0889 | 0.0006 | 84 |
| 739/744 | n.d. | n.d. | n.d. | n.d. | 0.0047 | 0.0549 | 0.0008 | 85 |
| 755/760 | n.d. | n.d. | n.d. | n.d. | 0.0051 | 0.0864 | 0.0006 | 87 |
| 55/744 | n.d. | n.d. | n.d. | n.d. | 0.0064 | 0.1011 | 0.0009 | 86 |
| 747/753 | n.d. | n.d. | n.d. | n.d. | 0.0083 | 0.0895 | 0.0013 | 89 |
| 764/771 | n.d. | n.d. | n.d. | n.d. | 0.0087 | 0.2156 | 0.0014 | 83 |
| 747/752 | n.d. | n.d. | n.d. | n.d. | 0.0095 | 0.1057 | 0.0016 | 88 |
| 764/772 | n.d. | n.d. | n.d. | n.d. | 0.0096 | 0.2988 | 0.0015 | 83 |
| 767/773 | n.d. | n.d. | n.d. | n.d. | 0.0105 | 0.2057 | 0.0017 | 85 |
| 755/761 | n.d. | n.d. | n.d. | n.d. | 0.015 | 0.1494 | 0.0024 | 90 |
| 767/774 | n.d. | n.d. | n.d. | n.d. | 0.0268 | 17′741 | 0.0033 | 82 |

TABLE 3

| | Stability Human | | | | Stability | | | | | |
|---|---|---|---|---|---|---|---|---|---|---|
| | Serum | | | | Cynomolgous Serum | | | | | |
| | Sense | | Antisense | | Sense | | Antisense | | Human PBMC | |
| SEQ ID NO | strand | | strand | | strand | | strand | | assay | |
| pair | t½ [hr] | | t½ [hr] | | t½ [hr] | | t½ [hr] | | IFN-a | TNF-a |
| 747/753 | >48 | hrs | >48 | hrs | >48 | hrs | >48 | hrs | 0 | 0 |
| 764/772 | >48 | hrs | 27.3 | | >48 | hrs | 24.1 | | 0 | 0 |
| 3/4 | >24 | | >24 | | 5.5 | | 5.3 | | 0 | 0 |
| 7/8 | >24 | | >24 | | 9.3 | | 6.0 | | 0 | 0 |
| 55/56 | >24 | | >24 | | 21.9 | | 8.2 | | 0 | 0 |
| 25/26 | >24 | | 13.2 | | 5.3 | | 4.4 | | 0 | 0 |
| 83/84 | >24 | | 11.0 | | 4.5 | | 6.4 | | 0 | 0 |
| 31/32 | >24 | | 10.7 | | 15.0 | | 10.0 | | 0 | 0 |
| 33/34 | >24 | | 9.1 | | 6.7 | | 3.9 | | 0 | 0 |

TABLE 4

Activity testing
with 50 nM
dsRNA in Hepa1-
6 cells

| | | | | | | |
|---|---|---|---|---|---|---|
| | | | | | mean % | |
| | SEQ | | SEQ | | knock- | standard |
| Rank | ID NO | Sense strand sequence (5′-3′) | ID NO | Antisense strand sequence (5′-3′) | down | deviation |

| | | | | | | |
|---|---|---|---|---|---|---|
| 1 | 517 | uGAAcuAuGcuuGcucGuudTsdT | 518 | AACGAGcAAGcAuAGUUcAdTsdT | 59 | 7 |
| 2 | 519 | AuGAAuAcAGcAucccuuudTsdT | 520 | AAAGGGAUGCUGuAUUcAUdTsdT | 58 | 6 |
| 3 | 521 | uucucAGGcAGAuuccAAGdTsdT | 522 | CUUGGAAUCUGCCUGAGAAdTsdT | 52 | 6 |
| 4 | 523 | AAcAuuAAuuuccGuGuGAdTsdT | 524 | UcAcACGGAAAUuAAUGUUdTsdT | 52 | 9 |
| 5 | 525 | GAAcuAuGcuuGcucGuuudTsdT | 526 | AAACGAGcAAGcAuAGUUCdTsdT | 51 | 9 |
| 6 | 527 | uccuAGAcGcuAAcAuuAAdTsdT | 528 | UuAAUGUuAGCGUCuAGGAdTsdT | 51 | 6 |
| 7 | 529 | uAAuGucAuuccAccAAuudTsdT | 530 | AAUUGGUGGAAUGAcAUuAdTsdT | 50 | 6 |
| 8 | 531 | uuAuuuuAccGGAcAcuAAdTsdT | 532 | UuAGUGUCCGGuAAAAuAAdTsdT | 50 | 9 |
| 9 | 533 | AAcAuuAAuuuccGuGuGAdTsdT | 534 | UfCfACfACfGGAAAUfUfAAUfGUfUfdTsdT | 50 | 5 |
| 10 | 535 | AuAucAAAGAGcuAGGAAAdTsdT | 536 | UUUCCuAGCUCUUUGAuAUdTsdT | 50 | 14 |
| 11 | 537 | GAcGcuAAcAuuAAuuuccdTsdT | 538 | GGAAAUuAAUGUuAGCGUCdTsdT | 50 | 3 |
| 12 | 539 | uuccGuGuGAAAAuGGGucdTsdT | 540 | GACCcAUUUUcAcACGGAAdTsdT | 49 | 7 |
| 13 | 541 | GuGAAcuAuGcuuGcucGudTsdT | 542 | ACGAGcAAGcAuAGUUcACdTsdT | 47 | 17 |
| 14 | 543 | AuAucAAAGAGcuAGGAAAdTsdT | 544 | UfUfUfCfCfUfAGCfUfCfUfUfUfGAUfAUfdTsdT | 46 | 10 |
| 15 | 545 | uccuAGAcGcuAAcAuuAAdTsdT | 546 | UfUfAAUfGUfUfAGCfGUfCfUfAGGAdTsdT | 46 | 11 |
| 16 | 547 | uGcAuGuAuGAccAAuGuAdTsdT | 548 | UfACfAUfUfGGUfCfAUfACfAUfGCfAdTsdT | 45 | 1 |
| 17 | 549 | cccccuGGuAGAGAcGAAGdTsdT | 550 | CfUfUfGGAAUfCfUfGCfCfUfGAGAAdTsdT | 45 | 5 |
| 18 | 551 | uuuAucAuGAcAuGuuAuAdTsdT | 552 | uAuAAcAUGUcAUGAuAAAdTsdT | 45 | 19 |
| 19 | 553 | AAccucAAuAGGucGAccAdTsdT | 554 | UGGUCGACCuAUUGAGGUUdTsdT | 44 | 6 |
| 20 | 555 | uuAuccAAAGccGuuucAcdTsdT | 556 | GUGAAACGGCUUUGGAuAAdTsdT | 43 | 21 |
| 21 | 557 | uuccGuGuGAAAAuGGGucdTsdT | 558 | GACfCfCfAUfUfUfUfCfACfACfGGAAdTsdT | 43 | 9 |
| 22 | 559 | AccucAAuAGGucGAccAGdTsdT | 560 | CUGGUCGACCuAUUGAGGUdTsdT | 43 | 9 |
| 23 | 561 | GAAcuAuGcuuGcucGuuudTsdT | 562 | AAACfGAGCfAAGCfAUfAGUfUfCfdTsdT | 43 | 6 |
| 24 | 563 | AGAcGcuAAcAuuAAuuucdTsdT | 564 | GAAAUfUfAAUfGUfUfAGCfGUfCfUfdTsdT | 43 | 8 |
| 25 | 565 | uuuAucAuGAcAuGuuAuAdTsdT | 566 | UfAUfAACfAUfGUfCfAUfGAUfAAAdTsdT | 42 | 18 |
| 26 | 567 | GuGAAcuAuGcuuGcucGudTsdT | 568 | ACfGAGCfAAGCfAUfAGUfUfCfACfdTsdT | 42 | 19 |
| 27 | 569 | AGAcGcuAAcAuuAAuuucdTsdT | 570 | GAAAUuAAUGUuAGCGUCUdTsdT | 42 | 11 |
| 28 | 571 | ccGGAcAcuAAAccuAAAAdTsdT | 572 | UfUfUfUfAGGUfUfUfAGUfGUfCfCfGGdTsdT | 41 | 8 |
| 29 | 573 | uGcAAAccucAAuAGGucGdTsdT | 574 | CGACCuAUUGAGGUUUGcAdTsdT | 41 | 16 |
| 30 | 575 | cuGAAAAcuGGAAuAGGuGdTsdT | 576 | CfACfCfUfAUfUfCfCfAGUfUfUfUfCfAGdTsdT | 40 | 3 |
| 31 | 577 | uGuuAuAuGGuuAAAcccAdTsdT | 578 | UGGGUUuAACcAuAuAAcAdTsdT | 38 | 13 |
| 32 | 579 | uGuuAuAuGGuuAAAcccAdTsdT | 580 | UfGGGUfUfUfAACfCfAUfAUfAACfAdTsdT | 36 | 2 |
| 33 | 581 | uGGuuuAAAuuGGucucAAdTsdT | 582 | UfUfGAGACfCfAAUfUfUfAAACfCfAdTsdT | 35 | 6 |
| 34 | 583 | ccGGAcAcuAAAccuAAAAdTsdT | 584 | UUUuAGGUUuAGUGUCCGGdTsdT | 35 | 6 |
| 35 | 585 | uuAAuGucAuuccAccAAudTsdT | 586 | AUfUfGGUfGGAAUfGACfAUfUfAAdTsdT | 34 | 12 |
| 36 | 587 | uGuAAuGGuuuAAAuuGGudTsdT | 588 | ACfCfAAUfUfUfAAACfCfAUfUfACfAdTsdT | 33 | 1 |
| 37 | 589 | uGGuuuAAAuuGGucucAAdTsdT | 590 | UUGAGACcAAUUuAAACcAdTsdT | 33 | 7 |
| 38 | 591 | uuuAAuuAcuGGuAGGAcAdTsdT | 592 | UGUCCuACcAGuAAUuAAAdTsdT | 33 | 6 |
| 39 | 593 | GAcGcuAAcAuuAAuuuccdTsdT | 594 | GGAAAUfUfAAUfGUfUfAGCfGUfCfdTsdT | 32 | 6 |
| 40 | 595 | uuAuuuuAccGGAcAcuAAdTsdT | 596 | UfUfAGUfGUfCfCfGGUfAAAAUfAAdTsdT | 32 | 5 |
| 41 | 597 | uuAuccAAAGccGuuucAcdTsdT | 598 | GUfGAAACfGGCfUfUfUfGGAUfAAdTsdT | 32 | 24 |
| 42 | 599 | uuuAccGGAcAcuAAAccudTsdT | 600 | AGGUUuAGUGUCCGGuAAAdTsdT | 31 | 4 |
| 43 | 601 | uuuAAuuAcuGGuAGGAcAdTsdT | 602 | UfGUfCfCfUfACfCfAGUfAAUfUfAAAdTsdT | 30 | 5 |
| 44 | 603 | uGAAcuAuGcuuGcucGuudTsdT | 604 | AACfGAGCfAAGCfAUfAGUfUfCfAdTsdT | 29 | 10 |
| 45 | 605 | GGuuuAAAuuGGucucAAAdTsdT | 606 | UUUGAGACcAAUUuAAACCdTsdT | 27 | 4 |
| 46 | 607 | GGuuuAAAuuGGucucAAAdTsdT | 608 | UfUfUfGAGACfCfAAUfUfUfAAACfCfdTsdT | 26 | 1 |
| 47 | 609 | uGcuGAAuAAccuGuAGuudTsdT | 610 | AACuAcAGGUuAUUcAGcAdTsdT | 26 | 10 |
| 48 | 611 | AAAuGGGcAAAGGcGAuAcdTsdT | 612 | GUfAUfCfGCfCfUfUfUfGCfCfCfAUfUfUfdTsdT | 26 | 8 |
| 49 | 613 | uGuAAuGGuuuAAAuuGGudTsdT | 614 | ACcAAUUuAAACcAUuAcAdTsdT | 25 | 6 |
| 50 | 615 | AuGAAuAcAGcAucccuuudTsdT | 616 | AAAGGGAUfGCfUfGUfAUfUfCfAUfdTsdT | 23 | 4 |
| 51 | 617 | uGuuAGucAGccAuuuAcAdTsdT | 618 | UfGUfAAAUfGGCfUfGACfUfAACfAdTsdT | 21 | 8 |
| 52 | 619 | uuAAuGucAuuccAccAAudTsdT | 620 | AUUGGUGGAAUGAcAUuAAdTsdT | 21 | 26 |
| 53 | 621 | GuGuGGcuucAuAccGuucdTsdT | 622 | GAACfGGUfAUfGAAGCfCfACfACfdTsdT | 20 | 4 |
| 54 | 623 | GuGuGGcuucAuAccGuucdTsdT | 624 | GAACGGuAUGAAGCcAcACdTsdT | 18 | 5 |
| 55 | 625 | uGuuAGucAGccAuuuAcAdTsdT | 626 | UGuAAAUGGCUGACuAAcAdTsdT | 17 | 10 |
| 56 | 627 | uGuGGcuucAuAccGuuccdTsdT | 628 | GGAACGGuAUGAAGCcAcAdTsdT | 16 | 5 |
| 57 | 629 | uGcuGAAuAAccuGuAGuudTsdT | 630 | AACfUfACfAGGUfUfAUfUfCfAGCfAdTsdT | 14 | 20 |
| 58 | 631 | uuuAccGGAcAcuAAAccudTsdT | 632 | AGGUfUfUfAGUfGUfCfCfGGUfAAAdTsdT | 14 | 13 |
| 59 | 633 | cuGAAAAcuGGAAuAGGuGdTsdT | 634 | cACCuAUUCcAGUUUUcAGdTsdT | 13 | 21 |
| 60 | 635 | AAAccucAAuAGGucGAccdTsdT | 636 | GGUfCfGACfCfUfAUfUfGAGGUfUfUfdTsdT | 12 | 8 |
| 61 | 637 | AAccucAAuAGGucGAccAdTsdT | 638 | UfGGUfCfGACfCfUfAUfUfGAGGUfUfdTsdT | 10 | 1 |
| 62 | 639 | AGuAAAuGuuAGucAGccAdTsdT | 640 | UfGGCfUfGACfUfAACfAUfUfUfACfUfdTsdT | 10 | 3 |
| 63 | 641 | uGcAuGuAuGAccAAuGuAdTsdT | 642 | uAcAUUGGUcAuAcAUGcAdTsdT | 10 | 26 |
| 64 | 643 | uGcAAAccucAAuAGGucGdTsdT | 644 | CfGACfCfUfAUfUfGAGGUfUfUfGCfAdTsdT | 2 | 8 |
| 65 | 645 | AAAccucAAuAGGucGAccdTsdT | 646 | GGUCGACCuAUUGAGGUUUdTsdT | 1 | 4 |
| 66 | 647 | AGuAAAuGuuAGucAGccAdTsdT | 648 | UGGCUGACuAAcAUUuACUdTsdT | −2 | 11 |
| 67 | 649 | ucuuAuuuuAccGGAcAcudTsdT | 650 | AGUGUCCGGuAAAAuAAGAdTsdT | −5 | 5 |
| 68 | 651 | AccucAAuAGGucGAccAGdTsdT | 652 | CfUfGGUfCfGACfCfUfAUfUfGAGGUfdTsdT | −6 | 12 |
| 69 | 653 | AAAuGGGcAAAGGcGAuAcdTsdT | 654 | GuAUCGCCUUUGCCcAUUUdTsdT | −7 | 11 |
| 70 | 655 | uGuGGcuucAuAccGuuccdTsdT | 656 | GGAACfGGUfAUfGAAGCfCfACfAdTsdT | −14 | 3 |
| 71 | 657 | ucuuAuuuuAccGGAcAcudTsdT | 658 | AGUfGUfCfCfGGUfAAAAUfAAGAdTsdT | −19 | 2 |

TABLE 5

| | | Stability Mouse | | Stability Rat | | | |
|---|---|---|---|---|---|---|---|
| | | Serum | | Serum | | Human | |
| | | Sense | Antisense | Sense | Antisense | PBMC assay | |
| | SEQ ID | strand | strand | strand | strand | | TNF- |
| Rank | NO pair | t½ [hr] | t½ [hr] | t½ [hr] | t½ [hr] | IFN-a | a |
| 1 | 517/518 | >24 | 6.3 | >24 | 15.5 | 0 | 0 |
| 9 | 533/534 | 5.1 | 6 | 16.4 | 16.7 | 0 | 0 |
| 2 | 519/520 | 17.5 | 1.7 | 23.7 | 8 | 0 | 0 |

TABLE 6

| | | | | | | mismatch | |
|---|---|---|---|---|---|---|---|
| | | | | | | pos. | |
| | | | | spec. | num. | from 5′ end | |
| | | accession | description | Score | mm | of as | region |
| antisense | ON | NM_001018077.1 | Homo sapiens nuclear receptor subfamily 3, group C, member 1 | 0.00 | 0 | | CDS |
| | | | (glucocorticoid receptor) (NR3C1), transcript variant 1, mRNA | | | | |
| | OFF-1 | NM_002649.2 | Homo sapiens phosphoinositide-3-kinase, catalytic, gamma | 3.00 | 4 | 15 16 17 19 | 3UTR |
| | | | polypeptide (PIK3CG), mRNA | | | | |
| | OFF-2 | NM_017506.1 | Homo sapiens olfactory receptor, family 7, subfamily A, | 3.00 | 4 | 14 17 18 19 | 3UTR |
| | | | member 5 (OR7A5), mRNA | | | | |
| | OFF-3 | NM_003343.4 | Homo sapiens ubiquitin-conjugating enzyme E2G 2 (UBC7 | 3.00 | 5 | 1 13 14 16 | 3UTR |
| | | | homolog, yeast) (UBE2G2), transcript variant 1, mRNA | | | 19 | |
| | OFF-4 | NM_014872.1 | Homo sapiens zinc finger and BTB domain containing 5 | 3.00 | 3 | 14 15 17 | 3UTR |
| | | | (ZBTB5), mRNA | | | | |
| | OFF-5 | NM_003112.3 | Homo sapiens Sp4 transcription factor (SP4), mRNA | 3.20 | 3 | 11 15 17 | 3UTR |
| | OFF-6 | NM_001125.2 | Homo sapiens ADP-ribosylarginine hydrolase (ADPRH), mRNA | 3.20 | 3 | 11 14 17 | 3UTR |
| | OFF-7 | NM_024770.3 | Homo sapiens methyltransferase like 8 (METTL8), mRNA | 3.20 | 4 | 10 14 17 19 | 3UTR |
| | OFF-8 | NM_018424.2 | Homo sapiens erythrocyte membrane protein band 4.1 like 4B | 3.25 | 4 | 9 14 18 19 | 3UTR |
| | | | (EPB41L4B), transcript variant 1, mRNA | | | | |
| | OFF-9 | NM_207303.2 | Homo sapiens attractin-like 1 (ATRNL1), mRNA | 3.50 | 5 | 1 8 12 14 19 | 3UTR |
| | OFF-10 | NM_032811.2 | Homo sapiens transforming growth factor beta regulator 1 | 3.70 | 5 | 1 8 10 15 19 | 3UTR |
| | | | (TBRG1), transcript variant 1, mRNA | | | | |
| | OFF-11 | NM_032714.1 | Homo sapiens chromosome 14 open reading frame 151 | 11.00 | 4 | 1 4 15 19 | 3UTR |
| | | | (C14orf151), mRNA | | | | |
| | OFF-12 | NM_018230.2 | Homo sapiens nucleoporin 133 kDa (NUP133), mRNA | 11.20 | 2 | 5 11 | CDS |
| sense | OFF-13 | NM_001013579.1 | Homo sapiens diacylglycerol O-acyltransferase 2-like 3 | 2 | 3 | 15 17 19 | CDS |
| | | | (DGAT2L3), mRNA | | | | |
| | OFF-14 | NM_032973.1 | Homo sapiens protocadherin 11 Y-linked (PCDH11Y), transcript | 11 | 2 | 5 14 | CDS |
| | | | variant c, mRNA | | | | |
| | OFF-15 | NM_130797.2 | Homo sapiens dipeptidyl-peptidase 6 (DPP6), transcript variant | 11.2 | 3 | 1 6 11 | CDS |
| | | | 1, mRNA | | | | |

TABLE 7

| | | | | | mismatch pos. | |
|---|---|---|---|---|---|---|
| | | | spec. | num. | from 5′ end | |
| | accession | description | Score | mm | of as | region |
| antisense | | | | | | |
| ON | NM_001018077.1 | Homo sapiens nuclear receptor subfamily 3, group C, member 1 | 0.00 | 0 | | CDS |
| | | (glucocorticoid receptor) (NR3C1), transcript variant 1, mRNA | | | | |
| OFF-1 | NM_213607.1 | Homo sapiens coiled-coil domain containing 103 (CCDC103), | 3.00 | 3 | 12 13 14 | 3UTR |
| | | mRNA | | | | |
| OFF-2 | NM_001080485.1 | Homo sapiens zinc finger protein 275 (ZNF275), mRNA | 3.20 | 4 | 10 16 17 19 | 3UTR |
| OFF-3 | NM_002205.2 | Homo sapiens integrin, alpha 5 (fibronectin receptor, alpha | 3.40 | 4 | 10 11 14 19 | 3UTR |
| | | polypeptide) (ITGA5), mRNA | | | | |
| OFF-4 | XM_001716748.1 | PREDICTED: Homo sapiens hypothetical LOC731508 | 3.45 | 4 | 9 10 12 19 | 3UTR |
| | | (LOC731508), mRNA | | | | |
| OFF-5 | NM_020476.2 | Homo sapiens ankyrin 1, erythrocytic (ANK1), transcript variant 1, | 3.45 | 5 | 1 9 11 17 19 | 3UTR |
| | | mRNA | | | | |
| OFF-6 | NM_001025247.1 | Homo sapiens TAF5-like RNA polymerase II, p300/CBP-associated | 3.70 | 4 | 8 10 17 19 | 3UTR |
| | | factor (PCAF)-associated factor, 65 kDa (TAF5L), transcript variant | | | | |
| | | 2, mRNA | | | | |
| OFF-7 | NM_001101396.1 | Homo sapiens similar to cAMP-regulated phosphoprotein | 11.00 | 4 | 1 3 13 19 | 3UTR |
| | | (LOC646227), mRNA | | | | |
| OFF-8 | NM_018667.2 | Homo sapiens sphingomyelin phosphodiesterase 3, neutral membrane | 11.00 | 3 | 1 2 15 | 3UTR |
| | | (neutral sphingomyelinase II) (SMPD3), mRNA | | | | |
| OFF-9 | NM_001080449.1 | Homo sapiens DNA replication helicase 2 homolog (yeast) (DNA2), | 11.20 | 4 | 1 3 11 19 | CDS |
| | | mRNA | | | | |
| OFF-10 | NM_015039.2 | Homo sapiens nicotinamide nucleotide adenylyltransferase 2 | 12.00 | 5 | 1 7 12 14 19 | 3UTR |
| | | (NMNAT2), transcript variant 1, mRNA | | | | |
| OFF-11 | NM_000520.4 | Homo sapiens hexosaminidase A (alpha polypeptide) (HEXA), | 12.20 | 4 | 1 5 10 18 | 3UTR |
| | | mRNA | | | | |
| sense | | | | | | |
| OFF-12 | NM_133432.2 | Homo sapiens titin (TTN), transcript variant novex-1, mRNA | 2 | 4 | 1 13 14 19 | CDS |
| OFF-13 | NM_033210.3 | Homo sapiens zinc finger protein 502 (ZNF502), mRNA | 2.25 | 4 | 1 9 17 19 | 3UTR |
| OFF-14 | NM_005076.2 | Homo sapiens contactin 2 (axonal) (CNTN2), mRNA | 2.5 | 3 | 1 8 13 | CDS |

TABLE 8

| | | | | | mismatch | |
|---|---|---|---|---|---|---|
| | | | | | pos. | |
| | | | spec. | num. | from 5′ end | |
| | accession | description | Score | mm | of as | region |
| antisense | | | | | | |
| ON | NM_001018077.1 | Homo sapiens nuclear receptor subfamily 3, group C, member 1 | 0.00 | 0 | | 3UTR |
| | | (glucocorticoid receptor) (NR3C1), transcript variant 1, mRNA | | | | |
| OFF-1 | NM_006710.4 | Homo sapiens COP9 constitutive photomorphogenic homolog | 3.00 | 4 | 1 12 15 17 | 3UTR |
| | | subunit 8 (Arabidopsis) (COPS8), transcript variant 1, mRNA | | | | |
| OFF-2 | NM_194285.2 | Homo sapiens SPT2, Suppressor of Ty, domain containing 1 (S. cerevisiae) | 3.00 | 4 | 1 16 17 18 | 3UTR |
| | | (SPTY2D1), mRNA | | | | |
| OFF-3 | NM_004929.2 | Homo sapiens calbindin 1, 28 kDa (CALB1), mRNA | 3.20 | 4 | 10 17 18 19 | 3UTR |
| OFF-4 | NM_021101.3 | Homo sapiens claudin 1 (CLDN1), mRNA | 3.25 | 3 | 9 12 18 | 3UTR |
| OFF-5 | NM_058191.3 | Homo sapiens chromosome 21 open reading frame 66 (C21orf66), | 3.50 | 4 | 1 8 13 18 | 3UTR |
| | | transcript variant 4, mRNA | | | | |
| OFF-6 | NM_130446.2 | Homo sapiens kelch-like 6 (Drosophila) (KLHL6), mRNA | 3.75 | 5 | 1 8 9 13 19 | 3UTR |
| OFF-7 | NM_015525.2 | Homo sapiens inhibitor of Bruton agammaglobulinemia tyrosine | 11.00 | 3 | 1 3 12 | 3UTR |
| | | kinase (IBTK), mRNA | | | | |
| OFF-8 | NM_001080.3 | Homo sapiens aldehyde dehydrogenase 5 family, member A1 | 12.00 | 5 | 1 3 13 18 19 | 3UTR |
| | | (succinate-semialdehyde dehydrogenase) (ALDH5A1), nuclear gene | | | | |
| | | encoding mitochondrial protein, transcript variant 2, mRNA | | | | |
| OFF-9 | NM_018003.2 | Homo sapiens uveal autoantigen with coiled-coil domains and | 12.00 | 3 | 6 12 15 | 3UTR |
| | | ankyrin repeats (UACA), transcript variant 1, mRNA | | | | |
| OFF-10 | NM_020346.1 | Homo sapiens solute carrier family 17 (sodium-dependent inorganic | 12.20 | 4 | 5 10 17 19 | 3UTR |
| | | phosphate cotransporter), member 6 (SLC17A6), mRNA | | | | |
| OFF-11 | NM_004969.2 | Homo sapiens insulin-degrading enzyme (IDE), mRNA | 12.20 | 5 | 1 6 11 14 19 | 3UTR |
| sense | | | | | | |
| OFF-12 | NM_024422.3 | Homo sapiens desmocollin 2 (DSC2), transcript variant Dsc2a, | 2 | 4 | 1 13 15 19 | CDS |
| | | mRNA | | | | |
| OFF-13 | NM_003211.3 | Homo sapiens thymine-DNA glycosylase (TDG), mRNA | 2.2 | 4 | 1 10 17 19 | 3UTR |
| OFF-14 | NM_002645.2 | Homo sapiens phosphoinositide-3-kinase, class 2, alpha polypeptide | 11 | 4 | 1 3 16 19 | 3UTR |
| | | (PIK3C2A), mRNA | | | | |

TABLE 9

| | | | SEQ ID |
|---|---|---|---|
| FPL Name | Function | Sequence | No. |
| hGAP001 | CE | GAATTTGCCATGGGTGGAATTTTTTCTCTTGGAAAGAAAGT | 683 |
| hGAP002 | CE | GGAGGGATCTCGCTCCTGGATTTTTCTCTTGGAAAGAAAGT | 684 |
| hGAP003 | CE | CCCCAGCCTTCTCCATGGTTTTTTCTCTTGGAAAGAAAGT | 685 |
| hGAP004 | CE | GCTCCCCCCTGCAAATGAGTTTTTCTCTTGGAAAGAAAGT | 686 |
| hGAP005 | LE | AGCCTTGACGGTGCCATGTTTTTAGGCATAGGACCCGTGTCT | 687 |
| hGAP006 | LE | GATGACAAGCTTCCCGTTCTCTTTTTAGGCATAGGACCCGTGTCT | 688 |
| hGAP007 | LE | AGATGGTGATGGGATTTCCATTTTTTTAGGCATAGGACCCGTGTCT | 689 |
| hGAP008 | LE | GCATCGCCCCACTTGATTTTTTTTTAGGCATAGGACCCGTGTCT | 690 |
| hGAP009 | LE | CACGACGTACTCAGCGCCATTTTTAGGCATAGGACCCGTGTCT | 691 |
| hGAP010 | LE | GGCAGAGATGATGACCCTTTTGTTTTTAGGCATAGGACCCGTGTCT | 692 |
| hGAP011 | BL | GGTGAAGACGCCAGTGGACTC | 693 |

TABLE 10

| | | | SEQ ID |
|---|---|---|---|
| FPL Name | Function | Sequence | No. |
| hGcR3001 | CE | TCCCATGCTAATTATCCAGCACTTTTTCTCTTGGAAAGAAAGT | 694 |
| hGcR3002 | CE | TGGCATGCCCAGAGCTCATTTTTCTCTTGGAAAGAAAGT | 695 |
| hGcR3003 | CE | GGAGCGTGGCTTTCCTTCATTTTTCTCTTGGAAAGAAAGT | 696 |
| hGcR3004 | CE | CCCTGCCTCTGAATTCTGAAGTTTTTCTCTTGGAAAGAAAGT | 697 |
| hGcR3005 | CE | CCTCCTTACACTTTTATTTCCCTTCTTTTTCTCTTGGAAAGAAAGT | 698 |
| hGcR3006 | CE | TTTTCTAGAGAGAAGCAAATCCTTTTTTTTCTCTTGGAAAGAAAGT | 699 |
| hGcR3007 | CE | GAGGGTATTTTCATACAGCCTTTCTTTTTCTCTTGGAAAGAAAGT | 700 |
| hGcR3008 | LE | TTCATAGACACAAATCATGTTAGTTTTCTTTTTAGGCATAGGACCCGTGTCT | 701 |
| hGcR3009 | LE | TCCATGGTGATGTAGTTTTCAGGTTTTTAGGCATAGGACCCGTGTCT | 702 |
| hGcR3010 | LE | ACAAAAACACATTCACCTACAGCTACTTTTTAGGCATAGGACCCGTGTCT | 703 |
| hGcR3011 | LE | TGACACTAAAACCAGACACACACACTTTTTAGGCATAGGACCCGTGTCT | 704 |
| hGcR3012 | LE | AATCTATATGTAGTTAAGCAAGTTATTTGAGTTTTTAGGCATAGGACCCGTGTCT | 705 |
| hGcR3013 | BL | GACTTAGGTGAAACTGGAATTGCT | 706 |
| hGcR3014 | BL | GTTTTTAAAAGGGAACTAAAATTATGA | 707 |
| hGcR3015 | BL | GATCAATGTATTGTATAACAATATTTTTCAT | 708 |

TABLE 11

| | | | SEQ ID |
|---|---|---|---|
| FPL Name | Function | Sequence | No. |
| mmNR3C1 001 | CE | ATCTGGTCTCATTCCAGGGCTTTTTTCTCTTGGAAAGAAAGT | 709 |
| mmNR3C1 002 | CE | CAGGCAGAGTTTGGGAGGTGGTTTTTCTCTTGGAAAGAAAGT | 710 |
| mmNR3C1 003 | CE | TTCCAGGTTCATTCCAGCTTGTTTTTCTCTTGGAAAGAAAGT | 711 |
| mmNR3C1 004 | CE | TTTTTTTCTTCGTTTTTCGAGCTTTTTCTCTTGGAAAGAAAGT | 712 |
| mmNR3C1 005 | CE | AGTGGCTTGCTGAATTCCTTTAATTTTTCTCTTGGAAAGAAAGT | 713 |
| mmNR3C1 006 | CE | GGAACTATTGTTTTGTTAGCGTTTTCTTTTTCTCTTGGAAAGAAAGT | 714 |
| mmNR3C1 007 | LE | TCCCGTTGCTGTGGAGGATTTTTAGGCATAGGACCCGTGTCT | 715 |
| mmNR3C1 008 | LE | CCGAAGCTTCATCGGAGCACACTTTTTAGGCATAGGACCCGTGTCT | 716 |
| mmNR3C1 009 | LE | CAGCACCCCATAATGGCATCTTTTTAGGCATAGGACCCGTGTCT | 717 |
| mmNR3C1 010 | LE | TCCAGCACAAAGGTAATTGTGCTTTTTAGGCATAGGACCCGTGTCT | 718 |
| mmNR3C1 011 | LE | TTTTATCAATGATGCAATCATTTCTTTTTTAGGCATAGGACCCGTGTCT | 719 |
| mmNR3C1 012 | LE | AAGACATTTTCGATAGCGGCATTTTTAGGCATAGGACCCGTGTCT | 720 |
| mmNR3C1 013 | BL | GCTGGACGGAGGAGAACTCAC | 721 |
| mmNR3C1 014 | BL | GAAGACTTTACAGCTTCCACACGT | 722 |
| mmNR3C1 015 | BL | TGTCCTTCCACTGCTCTTTTAAA | 723 |
| mmNR3C1 016 | BL | TGCTGGACAGTTTTTTCTTCGAA | 724 |
| mmNR3C1 017 | BL | AGAAGTGTCTTGTGAGACTCCTGC | 725 |

TABLE 12

| | | | SEQ ID |
|---|---|---|---|
| FPL Name | Function | Sequence | No. |
| mGAP001 | CE | CAAATGGCAGCCCTGGTGATTTTTCTCTTGGAAAGAAAGT | 726 |
| mGAP002 | CE | CCTTGACTGTGCCGTTGAATTTTTTTTCTCTTGGAAAGAAAGT | 727 |
| mGAP003 | CE | GTCTCGCTCCTGGAAGATGGTTTTTCTCTTGGAAAGAAAGT | 728 |
| mGAP004 | CE | CCCGGCCTTCTCCATGGTTTTTTCTCTTGGAAAGAAAGT | 729 |
| mGAP005 | LE | AACAATCTCCACTTTGCCACTGTTTTTAGGCATAGGACCCGTGTCT | 730 |
| mGAP006 | LE | CATGTAGACCATGTAGTTGAGGTCAATTTTTAGGCATAGGACCCGTGTCT | 731 |
| mGAP007 | LE | GACAAGCTTCCCATTCTCGGTTTTTAGGCATAGGACCCGTGTCT | 732 |
| mGAP008 | LE | TGATGGGCTTCCCGTTGATTTTTTAGGCATAGGACCCGTGTCT | 733 |
| mGAP009 | LE | GACATACTCAGCACCGGCCTTTTTTAGGCATAGGACCCGTGTCT | 734 |
| mGAP010 | BL | TGAAGGGGTCGTTGATGGC | 735 |
| mGAP011 | BL | CCGTGAGTGGAGTCATACTGGAA | 736 |
| mGAP012 | BL | CACCCCATTTGATGTTAGTGGG | 737 |
| mGAP013 | BL | GGTGAAGACACCAGTAGACTCCAC | 738 |

TABLE 13

| SEQ | | | |
|---|---|---|---|
| ID | Sense strand | SEQ | Antisense strand |
| NO | sequence (5′-3′) | ID NO | sequence (5′-3′) |
| 775 | UGCAAACCUCAAUAGGUCG | 776 | CGACCUAUUGAGGUUUGCA |
| 777 | AAACCUCAAUAGGUCGACC | 778 | GGUCGACCUAUUGAGGUUU |
| 779 | AACCUCAAUAGGUCGACCA | 780 | UGGUCGACCUAUUGAGGUU |
| 781 | ACCUCAAUAGGUCGACCAG | 782 | CUGGUCGACCUAUUGAGGU |
| 783 | UUAAUGUCAUUCCACCAAU | 784 | AUUGGUGGAAUGACAUUAA |
| 785 | UGUGAUGGACUUCUAUAAA | 786 | UUUAUAGAAGUCCAUCACA |
| 787 | CCAAGCAGCGAAGACUUUU | 788 | AAAAGUCUUCGCUGCUUGG |
| 789 | UUUCCAAAAGGCUCAGUAA | 790 | UUACUGAGCCUUUUGGAAA |
| 791 | AAGGCUCAGUAAGCAAUGC | 792 | GCAUUGCUUACUGAGCCUU |
| 793 | GGCUCAGUAAGCAAUGCGC | 794 | GCGCAUUGCUUACUGAGCC |
| 795 | CUCAGUAAGCAAUGCGCAG | 796 | CUGCGCAUUGCUUACUGAG |
| 797 | CUCUCAAUGGGACUGUAUA | 798 | UAUACAGUCCCAUUGAGAG |
| 799 | UCUCAAUGGGACUGUAUAU | 800 | AUAUACAGUCCCAUUGAGA |
| 801 | UCAAUGGGACUGUAUAUGG | 802 | CCAUAUACAGUCCCAUUGA |
| 803 | UGGGAAAUGACCUGGGAUU | 804 | AAUCCCAGGUCAUUUCCCA |
| 805 | AGCAUUGCAAACCUCAAUA | 806 | UAUUGAGGUUUGCAAUGCU |
| 807 | UUUGACAUUUUGCAGGAUU | 808 | AAUCCUGCAAAAUGUCAAA |
| 809 | CCCAGGUAAAGAGACGAAU | 810 | AUUCGUCUCUUUACCUGGG |
| 811 | CCAGGUAAAGAGACGAAUG | 812 | CAUUCGUCUCUUUACCUGG |
| 813 | CAGGUAAAGAGACGAAUGA | 814 | UCAUUCGUCUCUUUACCUG |
| 815 | AGACGAAUGAGAGUCCUUG | 816 | CAAGGACUCUCAUUCGUCU |
| 817 | AGAUCAGACCUGUUGAUAG | 818 | CUAUCAACAGGUCUGAUCU |
| 819 | UCAGACCUGUUGAUAGAUG | 820 | CAUCUAUCAACAGGUCUGA |
| 821 | ACGAUUCAUUCCUUUUGGA | 822 | UCCAAAAGGAAUGAAUCGU |
| 823 | AAGCCUCUCAUUUUACCGG | 824 | CCGGUAAAAUGAGAGGCUU |
| 825 | AGCCUCUCAUUUUACCGGA | 826 | UCCGGUAAAAUGAGAGGCU |
| 827 | GCCUCUCAUUUUACCGGAC | 828 | GUCCGGUAAAAUGAGAGGC |
| 829 | CCUCUCAUUUUACCGGACA | 830 | UGUCCGGUAAAAUGAGAGG |
| 831 | UCAUUUUACCGGACACUAA | 832 | UUAGUGUCCGGUAAAAUGA |
| 833 | UUUUACCGGACACUAAACC | 834 | GGUUUAGUGUCCGGUAAAA |
| 835 | UUUACCGGACACUAAACCC | 836 | GGGUUUAGUGUCCGGUAAA |
| 837 | UUACCGGACACUAAACCCA | 838 | UGGGUUUAGUGUCCGGUAA |
| 839 | UACCGGACACUAAACCCAA | 840 | UUGGGUUUAGUGUCCGGUA |
| 841 | AUCUGGUUUUGUCAAGCCC | 842 | GGGCUUGACAAAACCAGAU |
| 843 | AAAAAGAAGAUUUCAUCGA | 844 | UCGAUGAAAUCUUCUUUUU |
| 845 | AGAAGAUUUCAUCGAACUC | 846 | GAGUUCGAUGAAAUCUUCU |
| 847 | AAACUGGGCACAGUUUACU | 848 | AGUAAACUGUGCCCAGUUU |
| 849 | UUCUGUUCAUGGUGUGAGU | 850 | ACUCACACCAUGAACAGAA |
| 851 | GUUCAUGGUGUGAGUACCU | 852 | AGGUACUCACACCAUGAAC |
| 853 | GGAGGACAGAUGUACCACU | 854 | AGUGGUACAUCUGUCCUCC |
| 855 | CAGCAUCCCUUUCUCAACA | 856 | UGUUGAGAAAGGGAUGCUG |
| 857 | AGGAUCAGAAGCCUAUUUU | 858 | AAAAUAGGCUUCUGAUCCU |
| 859 | AUUCCACCAAUUCCCGUUG | 860 | CAACGGGAAUUGGUGGAAU |
| 861 | UUCCACCAAUUCCCGUUGG | 862 | CCAACGGGAAUUGGUGGAA |
| 863 | UCCACCAAUUCCCGUUGGU | 864 | ACCAACGGGAAUUGGUGGA |
| 865 | CCACCAAUUCCCGUUGGUU | 866 | AACCAACGGGAAUUGGUGG |
| 867 | CACCAAUUCCCGUUGGUUC | 868 | GAACCAACGGGAAUUGGUG |
| 869 | CUCUGAACUUCCCUGGUCG | 870 | CGACCAGGGAAGUUCAGAG |
| 871 | ACUUCCCUGGUCGAACAGU | 872 | ACUGUUCGACCAGGGAAGU |
| 873 | UGGUCGAACAGUUUUUUCU | 874 | AGAAAAAACUGUUCGACCA |
| 875 | UUUCUAAUGGCUAUUCAAG | 876 | CUUGAAUAGCCAUUAGAAA |
| 877 | AUGAGACCAGAUGUAAGCU | 878 | AGCUUACAUCUGGUCUCAU |
| 879 | CCAGAUGUAAGCUCUCCUC | 880 | GAGGAGAGCUUACAUCUGG |
| 881 | CUGGUGUGCUCUGAUGAAG | 882 | CUUCAUCAGAGCACACCAG |
| 883 | GUCUUAACUUGUGGAAGCU | 884 | AGCUUCCACAAGUUAAGAC |
| 885 | CAUCAUCGAUAAAAUUCGA | 886 | UCGAAUUUUAUCGAUGAUG |
| 887 | CCCAGCAUGCCGCUAUCGA | 888 | UCGAUAGCGGCAUGCUGGG |
| 889 | CCAGCAUGCCGCUAUCGAA | 890 | UUCGAUAGCGGCAUGCUGG |
| 891 | CAGCAUGCCGCUAUCGAAA | 892 | UUUCGAUAGCGGCAUGCUG |
| 893 | AGCAUGCCGCUAUCGAAAA | 894 | UUUUCGAUAGCGGCAUGCU |
| 895 | AUGCCGCUAUCGAAAAUGU | 896 | ACAUUUUCGAUAGCGGCAU |
| 897 | CCGCUAUCGAAAAUGUCUU | 898 | AAGACAUUUUCGAUAGCGG |
| 899 | CGCUAUCGAAAAUGUCUUC | 900 | GAAGACAUUUUCGAUAGCG |
| 901 | AGGAAUUCAGCAGGCCACU | 902 | AGUGGCCUGCUGAAUUCCU |
| 903 | AUUCAGCAGGCCACUACAG | 904 | CUGUAGUGGCCUGCUGAAU |
| 905 | CUACAGGAGUCUCACAAGA | 906 | UCUUGUGAGACUCCUGUAG |
| 907 | AAAACAAUAGUUCCUGCAA | 908 | UUGCAGGAACUAUUGUUUU |
| 909 | AAACAAUAGUUCCUGCAAC | 910 | GUUGCAGGAACUAUUGUUU |
| 911 | AACAAUAGUUCCUGCAACG | 912 | CGUUGCAGGAACUAUUGUU |
| 913 | ACAAUAGUUCCUGCAACGU | 914 | ACGUUGCAGGAACUAUUGU |
| 915 | AUAGUUCCUGCAACGUUAC | 916 | GUAACGUUGCAGGAACUAU |
| 917 | UAGUUCCUGCAACGUUACC | 918 | GGUAACGUUGCAGGAACUA |
| 919 | CUGCAACGUUACCACAACU | 920 | AGUUGUGGUAACGUUGCAG |
| 921 | UGCAACGUUACCACAACUC | 922 | GAGUUGUGGUAACGUUGCA |
| 923 | UGAACCUGAAGUGUUAUAU | 924 | AUAUAACACUUCAGGUUCA |
| 925 | UGUUAUAUGCAGGAUAUGA | 926 | UCAUAUCCUGCAUAUAACA |
| 927 | GCUCUGUUCCAGACUCAAC | 928 | GUUGAGUCUGGAACAGAGC |
| 929 | GUUCCAGACUCAACUUGGA | 930 | UCCAAGUUGAGUCUGGAAC |
| 931 | CUCAACUUGGAGGAUCAUG | 932 | CAUGAUCCUCCAAGUUGAG |
| 933 | ACGCUCAACAUGUUAGGAG | 934 | CUCCUAACAUGUUGAGCGU |
| 935 | GGGCGGCAAGUGAUUGCAG | 936 | CUGCAAUCACUUGCCGCCC |
| 937 | CAGGUUUCAGGAACUUACA | 938 | UGUAAGUUCCUGAAACCUG |
| 939 | GGUUUCAGGAACUUACACC | 940 | GGUGUAAGUUCCUGAAACC |
| 941 | AACUUACACCUGGAUGACC | 942 | GGUCAUCCAGGUGUAAGUU |
| 943 | ACUUACACCUGGAUGACCA | 944 | UGGUCAUCCAGGUGUAAGU |
| 945 | UGACCAAAUGACCCUACUG | 946 | CAGUAGGGUCAUUUGGUCA |
| 947 | GGGUGGAGAUCAUAUAGAC | 948 | GUCUAUAUGAUCUCCACCC |
| 949 | GGUGGAGAUCAUAUAGACA | 950 | UGUCUAUAUGAUCUCCACC |
| 951 | GAGAUCAUAUAGACAAUCA | 952 | UGAUUGUCUAUAUGAUCUC |
| 953 | CAUAUAGACAAUCAAGUGC | 954 | GCACUUGAUUGUCUAUAUG |
| 955 | CAUGUACGACCAAUGUAAA | 956 | UUUACAUUGGUCGUACAUG |
| 957 | AUGUACGACCAAUGUAAAC | 958 | GUUUACAUUGGUCGUACAU |
| 959 | UGUACGACCAAUGUAAACA | 960 | UGUUUACAUUGGUCGUACA |
| 961 | CAGGCUUCAGGUAUCUUAU | 962 | AUAAGAUACCUGAAGCCUG |
| 963 | UCUGUAUGAAAACCUUACU | 964 | AGUAAGGUUUUCAUACAGA |
| 965 | CUGUAUGAAAACCUUACUG | 966 | CAGUAAGGUUUUCAUACAG |
| 967 | GUAUGAAAACCUUACUGCU | 968 | AGCAGUAAGGUUUUCAUAC |
| 969 | GAAAUUAGAAUGACCUACA | 970 | UGUAGGUCAUUCUAAUUUC |
| 971 | GAACUGGCAGCGGUUUUAU | 972 | AUAAAACCGCUGCCAGUUC |
| 973 | ACUGGCAGCGGUUUUAUCA | 974 | UGAUAAAACCGCUGCCAGU |
| 975 | AACUCUUGGAUUCUAUGCA | 976 | UGCAUAGAAUCCAAGAGUU |
| 977 | CACACAUUAAUCUGAUUUU | 978 | AAAAUCAGAUUAAUGUGUG |
| 979 | UCCCAACAAUCUUGGCGCU | 980 | AGCGCCAAGAUUGUUGGGA |
| 981 | CCCAACAAUCUUGGCGCUC | 982 | GAGCGCCAAGAUUGUUGGG |
| 983 | CCAACAAUCUUGGCGCUCA | 984 | UGAGCGCCAAGAUUGUUGG |
| 985 | AACAAUCUUGGCGCUCAAA | 986 | UUUGAGCGCCAAGAUUGUU |
| 987 | UUGGCGCUCAAAAAAUAGA | 988 | UCUAUUUUUUGAGCGCCAA |
| 989 | UGGCGCUCAAAAAAUAGAA | 990 | UUCUAUUUUUUGAGCGCCA |
| 991 | AGGCUUUUCAUUAAAUGGG | 992 | CCCAUUUAAUGAAAAGCCU |
| 993 | UCCUAUGUAUGUGUUAUCU | 994 | AGAUAACACAUACAUAGGA |
| 995 | CCUAUGUAUGUGUUAUCUG | 996 | CAGAUAACACAUACAUAGG |
| 997 | CAGUGAGAGUUGGUUACUC | 998 | GAGUAACCAACUCUCACUG |
| 999 | AGUGAGAGUUGGUUACUCA | 1000 | UGAGUAACCAACUCUCACU |
| 1001 | GUGAGAGUUGGUUACUCAC | 1002 | GUGAGUAACCAACUCUCAC |
| 1003 | UGAGAGUUGGUUACUCACA | 1004 | UGUGAGUAACCAACUCUCA |
| 1005 | UGGUCCACCCAGGAUUAGU | 1006 | ACUAAUCCUGGGUGGACCA |
| 1007 | GGUCCACCCAGGAUUAGUG | 1008 | CACUAAUCCUGGGUGGACC |
| 1009 | UAGUGACCAGGUUUUCAGG | 1010 | CCUGAAAACCUGGUCACUA |
| 1011 | GGCUGUAUGAAAAUACCCU | 1012 | AGGGUAUUUUCAUACAGCC |
| 1013 | CUGUAUGAAAAUACCCUCC | 1014 | GGAGGGUAUUUUCAUACAG |
| 1015 | AUACCCUCCUCAAAUAACU | 1016 | AGUUAUUUGAGGAGGGUAU |
| 1017 | AAAUAACUUGCUUAACUAC | 1018 | GUAGUUAAGCAAGUUAUUU |
| 1019 | AAUAACUUGCUUAACUACA | 1020 | UGUAGUUAAGCAAGUUAUU |
| 1021 | UUGCUUAACUACAUAUAGA | 1022 | UCUAUAUGUAGUUAAGCAA |
| 1023 | UGCUUAACUACAUAUAGAU | 1024 | AUCUAUAUGUAGUUAAGCA |
| 1025 | UAGUUUUUUAUUCAUGCUG | 1026 | CAGCAUGAAUAAAAAACUA |
| 1027 | CAUGCUGAAUAAUAAUCUG | 1028 | CAGAUUAUUAUUCAGCAUG |
| 1029 | ACUGUAAAACCUUGUGUGG | 1030 | CCACACAAGGUUUUACAGU |
| 1031 | UGCUGUUCUGGUAUUACCA | 1032 | UGGUAAUACCAGAACAGCA |
| 1033 | UGGUCGAACAGUUUUUUCC | 874 | AGAAAAAACUGUUCGACCA |
| 1034 | UGGUCGAACAGUUUUUUCG | 874 | AGAAAAAACUGUUCGACCA |
| 1035 | GUUCCAGACUCAACUUGGC | 930 | UCCAAGUUGAGUCUGGAAC |
| 1036 | GUUCCAGACUCAACUUGGU | 930 | UCCAAGUUGAGUCUGGAAC |

TABLE 14

| | |
|---|---|
| unmodified sequence | modified sequence |

| | | | | | | | |
|---|---|---|---|---|---|---|---|
| SEQ | | SEQ | | SEQ | | SEQ | |
| ID | Sense strand sequence | ID | Antisense strand | ID | Sense strand | ID | Antisense strand sequence |
| NO | (5′-3′) | NO | sequence (5′-3′) | NO | sequence (5′-3′) | NO | (5′-3′) |

| | | | | | | | |
|---|---|---|---|---|---|---|---|
| 955 | CAUGUACGACCAAUG | 956 | UUUACAUUGGUC | 1 | cAuGuAcGAccAAuGuAA | 2 | UfUfUfACfAUfUfGGUfCf |
| | UAAA | | GUACAUG | | AdTsdT | | GUfACfAUfGdTsdT |
| 1021 | UUGCUUAACUACAUA | 1022 | UCUAUAUGUAGU | 3 | uuGcuuAAcuAcAuAuAG | 4 | UfCfUfAUfAUfGUfAGUfU |
| | UAGA | | UAAGCAA | | AdTsdT | | fAAGCfAAdTsdT |
| 1017 | AAAUAACUUGCUUAA | 1018 | GUAGUUAAGCAA | 5 | AAAuAAcuuGcuuAAcuA | 6 | GUfAGUfUfAAGCfAAGUf |
| | CUAC | | GUUAUUU | | cdTsdT | | UfAUfUfUfdTsdT |
| 1023 | UGCUUAACUACAUAU | 1024 | AUCUAUAUGUAG | 7 | uGcuuAAcuAcAuAuAGA | 8 | AUfCfUfAUfAUfGUfAGUf |
| | AGAU | | UUAAGCA | | udTsdT | | UfAAGCfAdTsdT |
| 967 | GUAUGAAAACCUUAC | 968 | AGCAGUAAGGUU | 9 | GuAuGAAAAccuuAcuGc | 10 | AGCfAGUfAAGGUfUfUfU |
| | UGCU | | UUCAUAC | | udTsdT | | fCfAUfACfdTsdT |
| 997 | CAGUGAGAGUUGGUU | 998 | GAGUAACCAACU | 11 | cAGuGAGAGuuGGuuAc | 12 | GAGUfAACfCfAACfUfCf |
| | ACUC | | CUCACUG | | ucdTsdT | | UfCfACfUfGdTsdT |
| 947 | GGGUGGAGAUCAUAU | 948 | GUCUAUAUGAUC | 13 | GGGuGGAGAucAuAuA | 14 | GUCuAuAUGAUCUCcAC |
| | AGAC | | UCCACCC | | GAcdTsdT | | CCdTsdT |
| 947 | GGGUGGAGAUCAUAU | 948 | GUCUAUAUGAUC | 15 | GGGuGGAGAucAuAuA | 16 | GUfCfUfAUfAUfGAUfCfU |
| | AGAC | | UCCACCC | | GAcdTsdT | | fCfCfACfCfCfdTsdT |
| 997 | CAGUGAGAGUUGGUU | 998 | GAGUAACCAACU | 17 | cAGuGAGAGuuGGuuAc | 18 | GAGuAACcAACUCUcAC |
| | ACUC | | CUCACUG | | ucdTsdT | | UGdTsdT |
| 953 | CAUAUAGACAAUCAA | 954 | GCACUUGAUUGU | 19 | cAuAuAGAcAAucAAGu | 20 | GCfACfUfUfGAUfUfGUfC |
| | GUGC | | CUAUAUG | | GcdTsdT | | fUfAUfAUfGdTsdT |
| 995 | CCUAUGUAUGUGUUA | 996 | CAGAUAACACAU | 21 | ccuAuGuAuGuGuuAucuG | 22 | CfAGAUfAACfACfAUfAC |
| | UCUG | | ACAUAGG | | dTsdT | | fAUfAGGdTsdT |
| 783 | UUAAUGUCAUUCCAC | 784 | AUUGGUGGAAU | 23 | uuAAuGucAuuccAccAAu | 24 | AUfUfGGUfGGAAUfGACf |
| | CAAU | | GACAUUAA | | dTsdT | | AUfUfAAdTsdT |
| 1021 | UUGCUUAACUACAUA | 1022 | UCUAUAUGUAGU | 25 | uuGcuuAAcuAcAuAuAG | 26 | UCuAuAUGuAGUuAAGcA |
| | UAGA | | UAAGCAA | | AdTsdT | | AdTsdT |
| 873 | UGGUCGAACAGUUUU | 874 | AGAAAAAACUGU | 27 | uGGucGAAcAGuuuuuucu | 28 | AGAAAAAACfUfGUfUfCf |
| | UUCU | | UCGACCA | | dTsdT | | GACfCfAdTsdT |
| 977 | CACACAUUAAUCUGA | 978 | AAAAUCAGAUUA | 29 | cAcAcAuuAAucuGAuuuu | 30 | AAAAUfCfAGAUfUfAAUf |
| | UUUU | | AUGUGUG | | dTsdT | | GUfGUfGdTsdT |
| 967 | GUAUGAAAACCUUAC | 968 | AGCAGUAAGGUU | 31 | GuAuGAAAAccuuAcuGc | 32 | AGcAGuAAGGUUUUcAu |
| | UGCU | | UUCAUAC | | udTsdT | | ACdTsdT |
| 905 | CUACAGGAGUCUCAC | 906 | UCUUGUGAGACU | 33 | cuAcAGGAGucucAcAAG | 34 | UCUUGUGAGACUCCUGu |
| | AAGA | | CCUGUAG | | AdTsdT | | AGdTsdT |
| 1013 | CUGUAUGAAAAUACC | 1014 | GGAGGGUAUUU | 35 | cuGuAuGAAAAuAcccucc | 36 | GGAGGGuAUUUUcAuAc |
| | CUCC | | UCAUACAG | | dTsdT | | AGdTsdT |
| 993 | UCCUAUGUAUGUGUU | 994 | AGAUAACACAUA | 37 | uccuAuGuAuGuGuuAucu | 38 | AGAuAAcAcAuAcAuAGG |
| | AUCU | | CAUAGGA | | dTsdT | | AdTsdT |
| 949 | GGUGGAGAUCAUAUA | 950 | UGUCUAUAUGAU | 39 | GGuGGAGAucAuAuAG | 40 | UfGUfCfUfAUfAUfGAUfC |
| | GACA | | CUCCACC | | AcAdTsdT | | fUfCfCfACfCfdTsdT |
| 957 | AUGUACGACCAAUGU | 958 | GUUUACAUUGGU | 41 | AuGuAcGAccAAuGuAA | 42 | GUfUfUfACfAUfUfGGUfC |
| | AAAC | | CGUACAU | | AcdTsdT | | fGUfACfAUfdTsdT |
| 973 | ACUGGCAGCGGUUUU | 974 | UGAUAAAACCGC | 43 | AcuGGcAGcGGuuuuAuc | 44 | UfGAUfAAAACfCfGCfUf |
| | AUCA | | UGCCAGU | | AdTsdT | | GCfCfAGUfdTsdT |
| 999 | AGUGAGAGUUGGUUA | 1000 | UGAGUAACCAAC | 45 | AGuGAGAGuuGGuuAcu | 46 | UfGAGUfAACfCfAACfUf |
| | CUCA | | UCUCACU | | cAdTsdT | | CfUfCfACfUfdTsdT |
| 1019 | AAUAACUUGCUUAAC | 1020 | UGUAGUUAAGCA | 47 | AAuAAcuuGcuuAAcuAc | 48 | UfGUfAGUfUfAAGCfAAG |
| | UACA | | AGUUAUU | | AdTsdT | | UfUfAUfUfdTsdT |
| 1001 | GUGAGAGUUGGUUAC | 1002 | GUGAGUAACCAA | 49 | GuGAGAGuuGGuuAcuc | 50 | GUfGAGUfAACfCfAACfU |
| | UCAC | | CUCUCAC | | AcdTsdT | | fCfUfCfACfdTsdT |
| 885 | CAUCAUCGAUAAAAU | 886 | UCGAAUUUUAUC | 51 | cAucAucGAuAAAAuucG | 52 | UfCfGAAUfUfUfUfAUfCf |
| | UCGA | | GAUGAUG | | AdTsdT | | GAUfGAUfGdTsdT |
| 1013 | CUGUAUGAAAAUACC | 1014 | GGAGGGUAUUU | 53 | cuGuAuGAAAAuAcccucc | 54 | GGAGGGUfAUfUfUfUfCf |
| | CUCC | | UCAUACAG | | dTsdT | | AUfACfAGdTsdT |
| 873 | UGGUCGAACAGUUUU | 874 | AGAAAAAACUGU | 55 | uGGucGAAcAGuuuuuucu | 56 | AGAAAAAACUGUUCGA |
| | UUCU | | UCGACCA | | dTsdT | | CcAdTsdT |
| 821 | ACGAUUCAUUCCUUU | 822 | UCCAAAAGGAAU | 57 | AcGAuucAuuccuuuuGGA | 58 | UfCfCfAAAAGGAAUfGA |
| | UGGA | | GAAUCGU | | dTsdT | | AUfCfGUfdTsdT |
| 965 | CUGUAUGAAAACCUU | 966 | CAGUAAGGUUUU | 59 | cuGuAuGAAAAccuuAcu | 60 | cAGuAAGGUUUUcAuAcA |
| | ACUG | | CAUACAG | | GdTsdT | | GdTsdT |
| 1001 | GUGAGAGUUGGUUAC | 1002 | GUGAGUAACCAA | 61 | GuGAGAGuuGGuuAcuc | 62 | GUGAGuAACcAACUCUc |
| | UCAC | | CUCUCAC | | AcdTsdT | | ACdTsdT |
| 959 | UGUACGACCAAUGUA | 960 | UGUUUACAUUGG | 63 | uGuAcGAccAAuGuAAAc | 64 | UfGUfUfUfACfAUfUfGGU |
| | AACA | | UCGUACA | | AdTsdT | | fCfGUfACfAdTsdT |
| 839 | UACCGGACACUAAAC | 840 | UUGGGUUUAGU | 65 | uAccGGAcAcuAAAcccA | 66 | UfUfGGGUfUfUfAGUfGUf |
| | CCAA | | GUCCGGUA | | AdTsdT | | CfCfGGUfAdTsdT |
| 897 | CCGCUAUCGAAAAUG | 898 | AAGACAUUUUCG | 67 | ccGcuAucGAAAAuGucuu | 68 | AAGACfAUfUfUfUfCfGA |
| | UCUU | | AUAGCGG | | dTsdT | | UfAGCfGGdTsdT |
| 817 | AGAUCAGACCUGUUG | 818 | CUAUCAACAGGU | 69 | AGAucAGAccuGuuGAuA | 70 | CfUfAUfCfAACfAGGUfCf |
| | AUAG | | CUGAUCU | | GdTsdT | | UfGAUfCfUfdTsdT |
| 993 | UCCUAUGUAUGUGUU | 994 | AGAUAACACAUA | 71 | uccuAuGuAuGuGuuAucu | 72 | AGAUfAACfACfAUfACfA |
| | AUCU | | CAUAGGA | | dTsdT | | UfAGGAdTsdT |
| 963 | UCUGUAUGAAAACCU | 964 | AGUAAGGUUUUC | 73 | ucuGuAuGAAAAccuuAcu | 74 | AGUfAAGGUfUfUfUfCfA |
| | UACU | | AUACAGA | | dTsdT | | UfACfAGAdTsdT |
| 907 | AAAACAAUAGUUCCU | 908 | UUGCAGGAACUA | 75 | AAAAcAAuAGuuccuGcA | 76 | UfUfGCfAGGAACfUfAUf |
| | GCAA | | UUGUUUU | | AdTsdT | | UfGUfUfUfUfdTsdT |
| 883 | GUCUUAACUUGUGGA | 884 | AGCUUCCACAAG | 77 | GucuuAAcuuGuGGAAGc | 78 | AGCfUfUfCfCfACfAAGUf |
| | AGCU | | UUAAGAC | | udTsdT | | UfAAGACfdTsdT |
| 913 | ACAAUAGUUCCUGCA | 914 | ACGUUGCAGGAA | 79 | AcAAuAGuuccuGcAAcG | 80 | ACfGUfUfGCfAGGAACfU |
| | ACGU | | CUAUUGU | | udTsdT | | fAUfUfGUfdTsdT |
| 991 | AGGCUUUUCAUUAAA | 992 | CCCAUUUAAUGA | 81 | AGGcuuuucAuuAAAuGG | 82 | CfCfCfAUfUfUfAAUfGAA |
| | UGGG | | AAAGCCU | | GdTsdT | | AAGCfCfUfdTsdT |
| 929 | GUUCCAGACUCAACU | 930 | UCCAAGUUGAGU | 83 | GuuccAGAcucAAcuuGG | 84 | UCcAAGUUGAGUCUGG |
| | UGGA | | CUGGAAC | | AdTsdT | | AACdTsdT |
| 957 | AUGUACGACCAAUGU | 958 | GUUUACAUUGGU | 85 | AuGuAcGAccAAuGuAA | 86 | GUUuAcAUUGGUCGuAc |
| | AAAC | | CGUACAU | | AcdTsdT | | AUdTsdT |
| 905 | CUACAGGAGUCUCAC | 906 | UCUUGUGAGACU | 87 | cuAcAGGAGucucAcAAG | 88 | UfCfUfUfGUfGAGACfUfC |
| | AAGA | | CCUGUAG | | AdTsdT | | fCfUfGUfAGdTsdT |
| 959 | UGUACGACCAAUGUA | 960 | UGUUUACAUUGG | 89 | uGuAcGAccAAuGuAAAc | 90 | UGUUuAcAUUGGUCGuA |
| | AACA | | UCGUACA | | AdTsdT | | cAdTsdT |
| 857 | AGGAUCAGAAGCCUA | 858 | AAAAUAGGCUUC | 91 | AGGAucAGAAGccuAuuu | 92 | AAAAUfAGGCfUfUfCfUf |
| | UUUU | | UGAUCCU | | udTsdT | | GAUfCfCfUfdTsdT |
| 969 | GAAAUUAGAAUGACC | 970 | UGUAGGUCAUUC | 93 | GAAAuuAGAAuGAccuA | 94 | UGuAGGUcAUUCuAAUU |
| | UACA | | UAAUUUC | | cAdTsdT | | UCdTsdT |
| 849 | UUCUGUUCAUGGUGU | 850 | ACUCACACCAUG | 95 | uucuGuucAuGGuGuGAG | 96 | ACfUfCfACfACfCfAUfGA |
| | GAGU | | AACAGAA | | udTsdT | | ACfAGAAdTsdT |
| 929 | GUUCCAGACUCAACU | 930 | UCCAAGUUGAGU | 97 | GuuccAGAcucAAcuuGG | 98 | UfCfCfAAGUfUfGAGUfCf |
| | UGGA | | CUGGAAC | | AdTsdT | | UfGGAACfdTsdT |
| 879 | CCAGAUGUAAGCUCU | 880 | GAGGAGAGCUUA | 99 | ccAGAuGuAAGcucuccuc | 100 | GAGGAGAGCUuAcAUCU |
| | CCUC | | CAUCUGG | | dTsdT | | GGdTsdT |
| 875 | UUUCUAAUGGCUAUU | 876 | CUUGAAUAGCCA | 101 | uuucuAAuGGcuAuucAA | 102 | CfUfUfGAAUfAGCfCfAUf |
| | CAAG | | UUAGAAA | | GdTsdT | | UfAGAAAdTsdT |
| 895 | AUGCCGCUAUCGAAA | 896 | ACAUUUUCGAUA | 103 | AuGccGcuAucGAAAAuG | 104 | ACfAUfUfUfUfCfGAUfAG |
| | AUGU | | GCGGCAU | | udTsdT | | CfGGCfAUfdTsdT |
| 889 | CCAGCAUGCCGCUAU | 890 | UUCGAUAGCGGC | 105 | ccAGcAuGccGcuAucGA | 106 | UfUfCfGAUfAGCfGGCfA |
| | CGAA | | AUGCUGG | | AdTsdT | | UfGCfUfGGdTsdT |
| 987 | UUGGCGCUCAAAAAA | 988 | UCUAUUUUUUGA | 107 | uuGGcGcucAAAAAAuA | 108 | UCuAUUUUUUGAGCGCc |
| | UAGA | | GCGCCAA | | GAdTsdT | | AAdTsdT |
| 863 | UCCACCAAUUCCCGU | 864 | ACCAACGGGAAU | 109 | uccAccAAuucccGuuGGud | 110 | ACfCfAACfGGGAAUfUfG |
| | UGGU | | UGGUGGA | | TsdT | | GUfGGAdTsdT |
| 909 | AAACAAUAGUUCCUG | 910 | GUUGCAGGAACU | 111 | AAAcAAuAGuuccuGcAA | 112 | GUfUfGCfAGGAACfUfAU |
| | CAAC | | AUUGUUU | | cdTsdT | | fUfGUfUfUfdTsdT |
| 849 | UUCUGUUCAUGGUGU | 850 | ACUCACACCAUG | 113 | uucuGuucAuGGuGuGAG | 114 | ACUcAcACcAUGAAcAGA |
| | GAGU | | AACAGAA | | udTsdT | | AdTsdT |
| 805 | AGCAUUGCAAACCUC | 806 | UAUUGAGGUUU | 115 | AGcAuuGcAAAccucAAu | 116 | uAUUGAGGUUUGcAAUG |
| | AAUA | | GCAAUGCU | | AdTsdT | | CUdTsdT |
| 827 | GCCUCUCAUUUUACC | 828 | GUCCGGUAAAAU | 117 | GccucucAuuuuAccGGAcd | 118 | GUfCfCfGGUfAAAAUfGA |
| | GGAC | | GAGAGGC | | TsdT | | GAGGCfdTsdT |
| 855 | CAGCAUCCCUUUCUC | 856 | UGUUGAGAAAG | 119 | cAGcAucccuuucucAAcAd | 120 | UGUUGAGAAAGGGAUG |
| | AACA | | GGAUGCUG | | TsdT | | CUGdTsdT |
| 951 | GAGAUCAUAUAGACA | 952 | UGAUUGUCUAUA | 121 | GAGAucAuAuAGAcAAu | 122 | UGAUUGUCuAuAUGAUC |
| | AUCA | | UGAUCUC | | cAdTsdT | | UCdTsdT |
| 1011 | GGCUGUAUGAAAAUA | 1012 | AGGGUAUUUUCA | 123 | GGcuGuAuGAAAAuAccc | 124 | AGGGuAUUUUcAuAcAG |
| | CCCU | | UACAGCC | | udTsdT | | CCdTsdT |
| 821 | ACGAUUCAUUCCUUU | 822 | UCCAAAAGGAAU | 125 | AcGAuucAuuccuuuuGGA | 126 | UCcAAAAGGAAUGAAU |
| | UGGA | | GAAUCGU | | dTsdT | | CGUdTsdT |
| 803 | UGGGAAAUGACCUGG | 804 | AAUCCCAGGUCA | 127 | uGGGAAAuGAccuGGGA | 128 | AAUCCcAGGUcAUUUCC |
| | GAUU | | UUUCCCA | | uudTsdT | | cAdTsdT |
| 809 | CCCAGGUAAAGAGAC | 810 | AUUCGUCUCUUU | 129 | cccAGGuAAAGAGAcGA | 130 | AUfUfCfGUfCfUfCfUfUfUf |
| | GAAU | | ACCUGGG | | AudTsdT | | ACfCfUfGGGdTsdT |
| 855 | CAGCAUCCCUUUCUC | 856 | UGUUGAGAAAG | 131 | cAGcAucccuuucucAAcAd | 132 | UfGUfUfGAGAAAGGGAU |
| | AACA | | GGAUGCUG | | TsdT | | fGCfUfGdTsdT |
| 813 | CAGGUAAAGAGACGA | 814 | UCAUUCGUCUCU | 133 | cAGGuAAAGAGAcGAA | 134 | UfCfAUfUfCfGUfCfUfCfUf |
| | AUGA | | UUACCUG | | uGAdTsdT | | UfUfACfCfUfGdTsdT |
| 1019 | AAUAACUUGCUUAAC | 1020 | UGUAGUUAAGCA | 135 | AAuAAcuuGcuuAAcuAc | 136 | UGuAGUuAAGcAAGUuA |
| | UACA | | AGUUAUU | | AdTsdT | | UUdTsdT |
| 965 | CUGUAUGAAAACCUU | 966 | CAGUAAGGUUUU | 137 | cuGuAuGAAAAccuuAcu | 138 | CfAGUfAAGGUfUfUfUfCf |
| | ACUG | | CAUACAG | | GdTsdT | | AUfACfAGdTsdT |
| 927 | GCUCUGUUCCAGACU | 928 | GUUGAGUCUGGA | 139 | GcucuGuuccAGAcucAAc | 140 | GUfUfGAGUfCfUfGGAAC |
| | CAAC | | ACAGAGC | | dTsdT | | fAGAGCfdTsdT |
| 793 | GGCUCAGUAAGCAAU | 794 | GCGCAUUGCUUA | 141 | GGcucAGuAAGcAAuGc | 142 | GCfGCfAUfUfGCfUfUfACf |
| | GCGC | | CUGAGCC | | GcdTsdT | | UfGAGCfCfdTsdT |
| 951 | GAGAUCAUAUAGACA | 952 | UGAUUGUCUAUA | 143 | GAGAucAuAuAGAcAAu | 144 | UfGAUfUfGUfCfUfAUfAU |
| | AUCA | | UGAUCUC | | cAdTsdT | | fGAUfCfUfCfdTsdT |
| 857 | AGGAUCAGAAGCCUA | 858 | AAAAUAGGCUUC | 145 | AGGAucAGAAGccuAuuu | 146 | AAAAuAGGCUUCUGAU |
| | UUUU | | UGAUCCU | | udTsdT | | CCUdTsdT |
| 891 | CAGCAUGCCGCUAUC | 892 | UUUCGAUAGCGG | 147 | cAGcAuGccGcuAucGAA | 148 | UUUCGAuAGCGGcAUGC |
| | GAAA | | CAUGCUG | | AdTsdT | | UGdTsdT |
| 925 | UGUUAUAUGCAGGAU | 926 | UCAUAUCCUGCA | 149 | uGuuAuAuGcAGGAuAu | 150 | UfCfAUfAUfCfCfUfGCfAU |
| | AUGA | | UAUAACA | | GAdTsdT | | fAUfAACfAdTsdT |
| 899 | CGCUAUCGAAAAUGU | 900 | GAAGACAUUUUC | 151 | cGcuAucGAAAAuGucuuc | 152 | GAAGACfAUfUfUfUfCfG |
| | CUUC | | GAUAGCG | | dTsdT | | AUfAGCfGdTsdT |
| 949 | GGUGGAGAUCAUAUA | 950 | UGUCUAUAUGAU | 153 | GGuGGAGAucAuAuAG | 154 | UGUCuAuAUGAUCUCcA |
| | GACA | | CUCCACC | | AcAdTsdT | | CCdTsdT |
| 987 | UUGGCGCUCAAAAAA | 988 | UCUAUUUUUUGA | 155 | uuGGcGcucAAAAAAuA | 156 | UfCfUfAUfUfUfUfUfUfGA |
| | UAGA | | GCGCCAA | | GAdTsdT | | GCfGCfCfAAdTsdT |
| 831 | UCAUUUUACCGGACA | 832 | UUAGUGUCCGGU | 157 | ucAuuuuAccGGAcAcuAA | 158 | UuAGUGUCCGGuAAAAU |
| | CUAA | | AAAAUGA | | dTsdT | | GAdTsdT |
| 885 | CAUCAUCGAUAAAAU | 886 | UCGAAUUUUAUC | 159 | cAucAucGAuAAAAuucG | 160 | UCGAAUUUuAUCGAUG |
| | UCGA | | GAUGAUG | | AdTsdT | | AUGdTsdT |
| 811 | CCAGGUAAAGAGACG | 812 | CAUUCGUCUCUU | 161 | ccAGGuAAAGAGAcGA | 162 | cAUUCGUCUCUUuACCU |
| | AAUG | | UACCUGG | | AuGdTsdT | | GGdTsdT |
| 961 | CAGGCUUCAGGUAUC | 962 | AUAAGAUACCUG | 163 | cAGGcuucAGGuAucuuAu | 164 | AuAAGAuACCUGAAGCC |
| | UUAU | | AAGCCUG | | dTsdT | | UGdTsdT |
| 789 | UUUCCAAAAGGCUCA | 790 | UUACUGAGCCUU | 165 | uuuccAAAAGGcucAGuA | 166 | UuACUGAGCCUUUUGG |
| | GUAA | | UUGGAAA | | AdTsdT | | AAAdTsdT |
| 977 | CACACAUUAAUCUGA | 978 | AAAAUCAGAUUA | 167 | cAcAcAuuAAucuGAuuuu | 168 | AAAAUcAGAUuAAUGUG |
| | UUUU | | AUGUGUG | | dTsdT | | UGdTsdT |
| 1011 | GGCUGUAUGAAAAUA | 1012 | AGGGUAUUUUCA | 169 | GGcuGuAuGAAAAuAccc | 170 | AGGGUfAUfUfUfUfCfAUf |
| | CCCU | | UACAGCC | | udTsdT | | ACfAGCfCfdTsdT |
| 937 | CAGGUUUCAGGAACU | 938 | UGUAAGUUCCUG | 171 | cAGGuuucAGGAAcuuAc | 172 | UGuAAGUUCCUGAAACC |
| | UACA | | AAACCUG | | AdTsdT | | UGdTsdT |
| 969 | GAAAUUAGAAUGACC | 970 | UGUAGGUCAUUC | 173 | GAAAuuAGAAuGAccuA | 174 | UfGUfAGGUfCfAUfUfCfU |
| | UACA | | UAAUUUC | | cAdTsdT | | fAAUfUfUfCfdTsdT |
| 787 | CCAAGCAGCGAAGAC | 788 | AAAAGUCUUCGC | 175 | ccAAGcAGcGAAGAcuuu | 176 | AAAAGUfCfUfUfCfGCfUf |
| | UUUU | | UGCUUGG | | udTsdT | | GCfUfUfGGdTsdT |
| 863 | UCCACCAAUUCCCGU | 864 | ACCAACGGGAAU | 177 | uccAccAAuucccGuuGGud | 178 | ACcAACGGGAAUUGGU |
| | UGGU | | UGGUGGA | | TsdT | | GGAdTsdT |
| 983 | CCAACAAUCUUGGCG | 984 | UGAGCGCCAAGA | 179 | ccAAcAAucuuGGcGcucA | 180 | UGAGCGCcAAGAUUGU |
| | CUCA | | UUGUUGG | | dTsdT | | UGGdTsdT |
| 795 | CUCAGUAAGCAAUGC | 796 | CUGCGCAUUGCU | 181 | cucAGuAAGcAAuGcGcA | 182 | CUGCGcAUUGCUuACUG |
| | GCAG | | UACUGAG | | GdTsdT | | AGdTsdT |
| 799 | UCUCAAUGGGACUGU | 800 | AUAUACAGUCCC | 183 | ucucAAuGGGAcuGuAuA | 184 | AUfAUfACfAGUfCfCfCfA |
| | AUAU | | AUUGAGA | | udTsdT | | UfUfGAGAdTsdT |
| 843 | AAAAAGAAGAUUUCA | 844 | UCGAUGAAAUCU | 185 | AAAAAGAAGAuuucAuc | 186 | UfCfGAUfGAAAUfCfUfUf |
| | UCGA | | UCUUUUU | | GAdTsdT | | CfUfUfUfUfUfdTsdT |
| 971 | GAACUGGCAGCGGUU | 972 | AUAAAACCGCUG | 187 | GAAcuGGcAGcGGuuuuA | 188 | AUfAAAACfCfGCfUfGCfC |
| | UUAU | | CCAGUUC | | udTsdT | | fAGUfUfCfdTsdT |
| 927 | GCUCUGUUCCAGACU | 928 | GUUGAGUCUGGA | 189 | GcucuGuuccAGAcucAAc | 190 | GUUGAGUCUGGAAcAG |
| | CAAC | | ACAGAGC | | dTsdT | | AGCdTsdT |
| 867 | CACCAAUUCCCGUUG | 868 | GAACCAACGGGA | 191 | cAccAAuucccGuuGGuucd | 192 | GAACfCfAACfGGGAAUf |
| | GUUC | | AUUGGUG | | TsdT | | UfGGUfGdTsdT |
| 899 | CGCUAUCGAAAAUGU | 900 | GAAGACAUUUUC | 193 | cGcuAucGAAAAuGucuuc | 194 | GAAGAcAUUUUCGAuAG |
| | CUUC | | GAUAGCG | | dTsdT | | CGdTsdT |
| 893 | AGCAUGCCGCUAUCG | 894 | UUUUCGAUAGCG | 195 | AGcAuGccGcuAucGAAA | 196 | UfUfUfUfCfGAUfAGCfGG |
| | AAAA | | GCAUGCU | | AdTsdT | | CfAUfGCfUfdTsdT |
| 931 | CUCAACUUGGAGGAU | 932 | CAUGAUCCUCCA | 197 | cucAAcuuGGAGGAucAu | 198 | cAUGAUCCUCcAAGUUG |
| | CAUG | | AGUUGAG | | GdTsdT | | AGdTsdT |
| 879 | CCAGAUGUAAGCUCU | 880 | GAGGAGAGCUUA | 199 | ccAGAuGuAAGcucuccuc | 200 | GAGGAGAGCfUfUfACfA |
| | CCUC | | CAUCUGG | | dTsdT | | UfCfUfGGdTsdT |
| 999 | AGUGAGAGUUGGUUA | 1000 | UGAGUAACCAAC | 201 | AGuGAGAGuuGGuuAcu | 202 | UGAGuAACcAACUCUcA |
| | CUCA | | UCUCACU | | cAdTsdT | | CUdTsdT |
| 935 | GGGCGGCAAGUGAUU | 936 | CUGCAAUCACUU | 203 | GGGcGGcAAGuGAuuGc | 204 | CUGcAAUcACUUGCCGC |
| | GCAG | | GCCGCCC | | AGdTsdT | | CCdTsdT |
| 785 | UGUGAUGGACUUCUA | 786 | UUUAUAGAAGUC | 205 | uGuGAuGGAcuucuAuAA | 206 | UfUfUfAUfAGAAGUfCfCf |
| | UAAA | | CAUCACA | | AdTsdT | | AUfCfACfAdTsdT |
| 787 | CCAAGCAGCGAAGAC | 788 | AAAAGUCUUCGC | 207 | ccAAGcAGcGAAGAcuuu | 208 | AAAAGUCUUCGCUGCU |
| | UUUU | | UGCUUGG | | udTsdT | | UGGdTsdT |
| 907 | AAAACAAUAGUUCCU | 908 | UUGCAGGAACUA | 209 | AAAAcAAuAGuuccuGcA | 210 | UUGcAGGAACuAUUGUU |
| | GCAA | | UUGUUUU | | AdTsdT | | UUdTsdT |
| 897 | CCGCUAUCGAAAAUG | 898 | AAGACAUUUUCG | 211 | ccGcuAucGAAAAuGucuu | 212 | AAGAcAUUUUCGAuAGC |
| | UCUU | | AUAGCGG | | dTsdT | | GGdTsdT |
| 891 | CAGCAUGCCGCUAUC | 892 | UUUCGAUAGCGG | 213 | cAGcAuGccGcuAucGAA | 214 | UfUfUfCfGAUfAGCfGGCf |
| | GAAA | | CAUGCUG | | AdTsdT | | AUfGCfUfGdTsdT |
| 881 | CUGGUGUGCUCUGAU | 882 | CUUCAUCAGAGC | 215 | cuGGuGuGcucuGAuGAA | 216 | CfUfUfCfAUfCfAGAGCfA |
| | GAAG | | ACACCAG | | GdTsdT | | CfACfCfAGdTsdT |
| 933 | ACGCUCAACAUGUUA | 934 | CUCCUAACAUGU | 217 | AcGcucAAcAuGuuAGGA | 218 | CUCCuAAcAUGUUGAGC |
| | GGAG | | UGAGCGU | | GdTsdT | | GUdTsdT |
| 979 | UCCCAACAAUCUUGG | 980 | AGCGCCAAGAUU | 219 | ucccAAcAAucuuGGcGcu | 220 | AGCfGCfCfAAGAUfUfGU |
| | CGCU | | GUUGGGA | | dTsdT | | fUfGGGAdTsdT |
| 815 | AGACGAAUGAGAGUC | 816 | CAAGGACUCUCA | 221 | AGAcGAAuGAGAGuccu | 222 | CfAAGGACfUfCfUfCfAUf |
| | CUUG | | UUCGUCU | | uGdTsdT | | UfCfGUfCfUfdTsdT |
| 839 | UACCGGACACUAAAC | 840 | UUGGGUUUAGU | 223 | uAccGGAcAcuAAAcccA | 224 | UUGGGUUuAGUGUCCG |
| | CCAA | | GUCCGGUA | | AdTsdT | | GuAdTsdT |
| 919 | CUGCAACGUUACCAC | 920 | AGUUGUGGUAAC | 225 | cuGcAAcGuuAccAcAAcu | 226 | AGUUGUGGuAACGUUGc |
| | AACU | | GUUGCAG | | dTsdT | | AGdTsdT |
| 889 | CCAGCAUGCCGCUAU | 890 | UUCGAUAGCGGC | 227 | ccAGcAuGccGcuAucGA | 228 | UUCGAuAGCGGcAUGCU |
| | CGAA | | AUGCUGG | | AdTsdT | | GGdTsdT |
| 805 | AGCAUUGCAAACCUC | 806 | UAUUGAGGUUU | 229 | AGcAuuGcAAAccucAAu | 230 | UfAUfUfGAGGUfUfUfGCf |
| | AAUA | | GCAAUGCU | | AdTsdT | | AAUfGCfUfdTsdT |
| 979 | UCCCAACAAUCUUGG | 980 | AGCGCCAAGAUU | 231 | ucccAAcAAucuuGGcGcu | 232 | AGCGCcAAGAUUGUUG |
| | CGCU | | GUUGGGA | | dTsdT | | GGAdTsdT |
| 865 | CCACCAAUUCCCGUU | 866 | AACCAACGGGAA | 233 | ccAccAAuucccGuuGGuud | 234 | AACcAACGGGAAUUGG |
| | GGUU | | UUGGUGG | | TsdT | | UGGdTsdT |
| 819 | UCAGACCUGUUGAUA | 820 | CAUCUAUCAACA | 235 | ucAGAccuGuuGAuAGAu | 236 | CfAUfCfUfAUfCfAACfAG |
| | GAUG | | GGUCUGA | | GdTsdT | | GUfCfUfGAdTsdT |
| 837 | UUACCGGACACUAAA | 838 | UGGGUUUAGUG | 237 | uuAccGGAcAcuAAAccc | 238 | UGGGUUuAGUGUCCGGu |
| | CCCA | | UCCGGUAA | | AdTsdT | | AAdTsdT |
| 981 | CCCAACAAUCUUGGC | 982 | GAGCGCCAAGAU | 239 | cccAAcAAucuuGGcGcuc | 240 | GAGCfGCfCfAAGAUfUfG |
| | GCUC | | UGUUGGG | | dTsdT | | UfUfGGGdTsdT |
| 875 | UUUCUAAUGGCUAUU | 876 | CUUGAAUAGCCA | 241 | uuucuAAuGGcuAuucAA | 242 | CUUGAAuAGCcAUuAGA |
| | CAAG | | UUAGAAA | | GdTsdT | | AAdTsdT |
| 783 | UUAAUGUCAUUCCAC | 784 | AUUGGUGGAAU | 243 | uuAAuGucAuuccAccAAu | 244 | AUUGGUGGAAUGAcAUu |
| | CAAU | | GACAUUAA | | dTsdT | | AAdTsdT |
| 793 | GGCUCAGUAAGCAAU | 794 | GCGCAUUGCUUA | 245 | GGcucAGuAAGcAAuGc | 246 | GCGcAUUGCUuACUGAG |
| | GCGC | | CUGAGCC | | GcdTsdT | | CCdTsdT |
| 883 | GUCUUAACUUGUGGA | 884 | AGCUUCCACAAG | 247 | GucuuAAcuuGuGGAAGc | 248 | AGCUUCcAcAAGUuAAG |
| | AGCU | | UUAAGAC | | udTsdT | | ACdTsdT |
| 831 | UCAUUUUACCGGACA | 832 | UUAGUGUCCGGU | 249 | ucAuuuuAccGGAcAcuAA | 250 | UfUfAGUfGUfCfCfGGUfA |
| | CUAA | | AAAAUGA | | dTsdT | | AAAUfGAdTsdT |
| 815 | AGACGAAUGAGAGUC | 816 | CAAGGACUCUCA | 251 | AGAcGAAuGAGAGuccu | 252 | cAAGGACUCUcAUUCGU |
| | CUUG | | UUCGUCU | | uGdTsdT | | CUdTsdT |
| 1029 | ACUGUAAAACCUUGU | 1030 | CCACACAAGGUU | 253 | AcuGuAAAAccuuGuGuG | 254 | CfCfACfACfAAGGUfUfUf |
| | GUGG | | UUACAGU | | GdTsdT | | UfACfAGUfdTsdT |
| 779 | AACCUCAAUAGGUCG | 780 | UGGUCGACCUAU | 255 | AAccucAAuAGGucGAcc | 256 | UGGUCGACCuAUUGAG |
| | ACCA | | UGAGGUU | | AdTsdT | | GUUdTsdT |
| 1027 | CAUGCUGAAUAAUAA | 1028 | CAGAUUAUUAUU | 257 | cAuGcuGAAuAAuAAucu | 258 | CfAGAUfUfAUfUfAUfUfC |
| | UCUG | | CAGCAUG | | GdTsdT | | fAGCfAUfGdTsdT |
| 775 | UGCAAACCUCAAUAG | 776 | CGACCUAUUGAG | 259 | uGcAAAccucAAuAGGuc | 260 | CGACCuAUUGAGGUUU |
| | GUCG | | GUUUGCA | | GdTsdT | | GcAdTsdT |
| 983 | CCAACAAUCUUGGCG | 984 | UGAGCGCCAAGA | 261 | ccAAcAAucuuGGcGcucA | 262 | UfGAGCfGCfCfAAGAUfU |
| | CUCA | | UUGUUGG | | dTsdT | | fGUfUfGGdTsdT |
| 939 | GGUUUCAGGAACUUA | 940 | GGUGUAAGUUCC | 263 | GGuuucAGGAAcuuAcAc | 264 | GGUGuAAGUUCCUGAA |
| | CACC | | UGAAACC | | cdTsdT | | ACCdTsdT |
| 939 | GGUUUCAGGAACUUA | 940 | GGUGUAAGUUCC | 265 | GGuuucAGGAAcuuAcAc | 266 | GGUfGUfAAGUfUfCfCfUf |
| | CACC | | UGAAACC | | cdTsdT | | GAAACfCfdTsdT |
| 1009 | UAGUGACCAGGUUUU | 1010 | CCUGAAAACCUG | 267 | uAGuGAccAGGuuuucAG | 268 | CCUGAAAACCUGGUcAC |
| | CAGG | | GUCACUA | | GdTsdT | | uAdTsdT |
| 919 | CUGCAACGUUACCAC | 920 | AGUUGUGGUAAC | 269 | cuGcAAcGuuAccAcAAcu | 270 | AGUfUfGUfGGUfAACfGU |
| | AACU | | GUUGCAG | | dTsdT | | fUfGCfAGdTsdT |
| 893 | AGCAUGCCGCUAUCG | 894 | UUUUCGAUAGCG | 271 | AGcAuGccGcuAucGAAA | 272 | UUUUCGAuAGCGGcAUG |
| | AAAA | | GCAUGCU | | AdTsdT | | CUdTsdT |
| 921 | UGCAACGUUACCACA | 922 | GAGUUGUGGUA | 273 | uGcAAcGuuAccAcAAcuc | 274 | GAGUUGUGGuAACGUU |
| | ACUC | | ACGUUGCA | | dTsdT | | GcAdTsdT |
| 923 | UGAACCUGAAGUGUU | 924 | AUAUAACACUUC | 275 | uGAAccuGAAGuGuuAu | 276 | AUfAUfAACfACfUfUfCfA |
| | AUAU | | AGGUUCA | | AudTsdT | | GGUfUfCfAdTsdT |
| 867 | CACCAAUUCCCGUUG | 868 | GAACCAACGGGA | 277 | cAccAAuucccGuuGGuucd | 278 | GAACcAACGGGAAUUG |
| | GUUC | | AUUGGUG | | TsdT | | GUGdTsdT |
| 811 | CCAGGUAAAGAGACG | 812 | CAUUCGUCUCUU | 279 | ccAGGuAAAGAGAcGA | 280 | CfAUfUfCfGUfCfUfCfUfUf |
| | AAUG | | UACCUGG | | AuGdTsdT | | UfACfCfUfGGdTsdT |
| 797 | CUCUCAAUGGGACUG | 798 | UAUACAGUCCCA | 281 | cucucAAuGGGAcuGuAu | 282 | UfAUfACfAGUfCfCfCfAUf |
| | UAUA | | UUGAGAG | | AdTsdT | | UfGAGAGdTsdT |
| 989 | UGGCGCUCAAAAAAU | 990 | UUCUAUUUUUUG | 283 | uGGcGcucAAAAAAuAG | 284 | UfUfCfUfAUfUfUfUfUfUf |
| | AGAA | | AGCGCCA | | AAdTsdT | | GAGCfGCfCfAdTsdT |
| 1015 | AUACCCUCCUCAAAU | 1016 | AGUUAUUUGAG | 285 | AuAcccuccucAAAuAAcu | 286 | AGUfUfAUfUfUfGAGGAG |
| | AACU | | GAGGGUAU | | dTsdT | | GGUfAUfdTsdT |
| 935 | GGGCGGCAAGUGAUU | 936 | CUGCAAUCACUU | 287 | GGGcGGcAAGuGAuuGc | 288 | CfUfGCfAAUfCfACfUfUfG |
| | GCAG | | GCCGCCC | | AGdTsdT | | CfCfGCfCfCfdTsdT |
| 1023 | UGCUUAACUACAUAU | 1024 | AUCUAUAUGUAG | 289 | uGcuuAAcuAcAuAuAGA | 290 | AUCuAuAUGuAGUuAAGc |
| | AGAU | | UUAAGCA | | udTsdT | | AdTsdT |
| 859 | AUUCCACCAAUUCCC | 860 | CAACGGGAAUUG | 291 | AuuccAccAAuucccGuuGd | 292 | CfAACfGGGAAUfUfGGUf |
| | GUUG | | GUGGAAU | | TsdT | | GGAAUfdTsdT |
| 781 | ACCUCAAUAGGUCGA | 782 | CUGGUCGACCUA | 293 | AccucAAuAGGucGAccA | 294 | CUGGUCGACCuAUUGAG |
| | CCAG | | UUGAGGU | | GdTsdT | | GUdTsdT |
| 851 | GUUCAUGGUGUGAGU | 852 | AGGUACUCACAC | 295 | GuucAuGGuGuGAGuAcc | 296 | AGGUfACfUfCfACfACfCf |
| | ACCU | | CAUGAAC | | udTsdT | | AUfGAACfdTsdT |
| 829 | CCUCUCAUUUUACCG | 830 | UGUCCGGUAAAA | 297 | ccucucAuuuuAccGGAcAd | 298 | UGUCCGGuAAAAUGAG |
| | GACA | | UGAGAGG | | TsdT | | AGGdTsdT |
| 825 | AGCCUCUCAUUUUAC | 826 | UCCGGUAAAAUG | 299 | AGccucucAuuuuAccGGA | 300 | UfCfCfGGUfAAAAUfGAG |
| | CGGA | | AGAGGCU | | dTsdT | | AGGCfUfdTsdT |
| 801 | UCAAUGGGACUGUAU | 802 | CCAUAUACAGUC | 301 | ucAAuGGGAcuGuAuAu | 302 | CfCfAUfAUfACfAGUfCfCf |
| | AUGG | | CCAUUGA | | GGdTsdT | | CfAUfUfGAdTsdT |
| 961 | CAGGCUUCAGGUAUC | 962 | AUAAGAUACCUG | 303 | cAGGcuucAGGuAucuuAu | 304 | AUfAAGAUfACfCfUfGAA |
| | UUAU | | AAGCCUG | | dTsdT | | GCfCfUfGdTsdT |
| 903 | AUUCAGCAGGCCACU | 904 | CUGUAGUGGCCU | 305 | AuucAGcAGGccAcuAcA | 306 | CfUfGUfAGUfGGCfCfUfG |
| | ACAG | | GCUGAAU | | GdTsdT | | CfUfGAAUfdTsdT |
| 981 | CCCAACAAUCUUGGC | 982 | GAGCGCCAAGAU | 307 | cccAAcAAucuuGGcGcuc | 308 | GAGCGCcAAGAUUGUU |
| | GCUC | | UGUUGGG | | dTsdT | | GGGdTsdT |
| 877 | AUGAGACCAGAUGUA | 878 | AGCUUACAUCUG | 309 | AuGAGAccAGAuGuAAG | 310 | AGCUuAcAUCUGGUCUc |
| | AGCU | | GUCUCAU | | cudTsdT | | AUdTsdT |
| 1027 | CAUGCUGAAUAAUAA | 1028 | CAGAUUAUUAUU | 311 | cAuGcuGAAuAAuAAucu | 312 | cAGAUuAUuAUUcAGcAU |
| | UCUG | | CAGCAUG | | GdTsdT | | GdTsdT |
| 973 | ACUGGCAGCGGUUUU | 974 | UGAUAAAACCGC | 313 | AcuGGcAGcGGuuuuAuc | 314 | UGAuAAAACCGCUGCcA |
| | AUCA | | UGCCAGU | | AdTsdT | | GUdTsdT |
| 841 | AUCUGGUUUUGUCAA | 842 | GGGCUUGACAAA | 315 | AucuGGuuuuGucAAGccc | 316 | GGGCfUfUfGACfAAAACf |
| | GCCC | | ACCAGAU | | dTsdT | | CfAGAUfdTsdT |
| 1003 | UGAGAGUUGGUUACU | 1004 | UGUGAGUAACCA | 317 | uGAGAGuuGGuuAcucAc | 318 | UGUGAGuAACcAACUCU |
| | CACA | | ACUCUCA | | AdTsdT | | cAdTsdT |
| 865 | CCACCAAUUCCCGUU | 866 | AACCAACGGGAA | 319 | ccAccAAuucccGuuGGuud | 320 | AACfCfAACfGGGAAUfUf |
| | GGUU | | UUGGUGG | | TsdT | | GGUfGGdTsdT |
| 847 | AAACUGGGCACAGUU | 848 | AGUAAACUGUGC | 321 | AAAcuGGGcAcAGuuuAc | 322 | AGUfAAACfUfGUfGCfCf |
| | UACU | | CCAGUUU | | udTsdT | | CfAGUfUfUfdTsdT |
| 851 | GUUCAUGGUGUGAGU | 852 | AGGUACUCACAC | 323 | GuucAuGGuGuGAGuAcc | 324 | AGGuACUcAcACcAUGAA |
| | ACCU | | CAUGAAC | | udTsdT | | CdTsdT |
| 1015 | AUACCCUCCUCAAAU | 1016 | AGUUAUUUGAG | 325 | AuAcccuccucAAAuAAcu | 326 | AGUuAUUUGAGGAGGGu |
| | AACU | | GAGGGUAU | | dTsdT | | AUdTsdT |
| 837 | UUACCGGACACUAAA | 838 | UGGGUUUAGUG | 327 | uuAccGGAcAcuAAAccc | 328 | UfGGGUfUfUfAGUfGUfCf |
| | CCCA | | UCCGGUAA | | AdTsdT | | CfGGUfAAdTsdT |
| 943 | ACUUACACCUGGAUG | 944 | UGGUCAUCCAGG | 329 | AcuuAcAccuGGAuGAcc | 330 | UfGGUfCfAUfCfCfAGGUf |
| | ACCA | | UGUAAGU | | AdTsdT | | GUfAAGUfdTsdT |
| 795 | CUCAGUAAGCAAUGC | 796 | CUGCGCAUUGCU | 331 | cucAGuAAGcAAuGcGcA | 332 | CfUfGCfGCfAUfUfGCfUfU |
| | GCAG | | UACUGAG | | GdTsdT | | fACfUfGAGdTsdT |
| 807 | UUUGACAUUUUGCAG | 808 | AAUCCUGCAAAA | 333 | uuuGAcAuuuuGcAGGAu | 334 | AAUfCfCfUfGCfAAAAUf |
| | GAUU | | UGUCAAA | | udTsdT | | GUfCfAAAdTsdT |
| 819 | UCAGACCUGUUGAUA | 820 | CAUCUAUCAACA | 335 | ucAGAccuGuuGAuAGAu | 336 | cAUCuAUcAAcAGGUCUG |
| | GAUG | | GGUCUGA | | GdTsdT | | AdTsdT |
| 903 | AUUCAGCAGGCCACU | 904 | CUGUAGUGGCCU | 337 | AuucAGcAGGccAcuAcA | 338 | CUGuAGUGGCCUGCUGA |
| | ACAG | | GCUGAAU | | GdTsdT | | AUdTsdT |
| 915 | AUAGUUCCUGCAACG | 916 | GUAACGUUGCAG | 339 | AuAGuuccuGcAAcGuuAc | 340 | GUfAACfGUfUfGCfAGGA |
| | UUAC | | GAACUAU | | dTsdT | | ACfUfAUfdTsdT |
| 921 | UGCAACGUUACCACA | 922 | GAGUUGUGGUA | 341 | uGcAAcGuuAccAcAAcuc | 342 | GAGUfUfGUfGGUfAACfG |
| | ACUC | | ACGUUGCA | | dTsdT | | UfUfGCfAdTsdT |
| 1025 | UAGUUUUUUAUUCAU | 1026 | CAGCAUGAAUAA | 343 | uAGuuuuuuAuucAuGcuG | 344 | CfAGCfAUfGAAUfAAAA |
| | GCUG | | AAAACUA | | dTsdT | | AACfUfAdTsdT |
| 803 | UGGGAAAUGACCUGG | 804 | AAUCCCAGGUCA | 345 | uGGGAAAuGAccuGGGA | 346 | AAUfCfCfCfAGGUfCfAUf |
| | GAUU | | UUUCCCA | | uudTsdT | | UfUfCfCfCfAdTsdT |
| 807 | UUUGACAUUUUGCAG | 808 | AAUCCUGCAAAA | 347 | uuuGAcAuuuuGcAGGAu | 348 | AAUCCUGcAAAAUGUcA |
| | GAUU | | UGUCAAA | | udTsdT | | AAdTsdT |
| 933 | ACGCUCAACAUGUUA | 934 | CUCCUAACAUGU | 349 | AcGcucAAcAuGuuAGGA | 350 | CfUfCfCfUfAACfAUfGUfU |
| | GGAG | | UGAGCGU | | GdTsdT | | fGAGCfGUfdTsdT |
| 1031 | UGCUGUUCUGGUAUU | 1032 | UGGUAAUACCAG | 351 | uGcuGuucuGGuAuuAccA | 352 | UfGGUfAAUfACfCfAGAA |
| | ACCA | | AACAGCA | | dTsdT | | CfAGCfAdTsdT |
| 809 | CCCAGGUAAAGAGAC | 810 | AUUCGUCUCUUU | 353 | cccAGGuAAAGAGAcGA | 354 | AUUCGUCUCUUuACCUG |
| | GAAU | | ACCUGGG | | AudTsdT | | GGdTsdT |
| 775 | UGCAAACCUCAAUAG | 776 | CGACCUAUUGAG | 355 | uGcAAAccucAAuAGGuc | 356 | CfGACfCfUfAUfUfGAGG |
| | GUCG | | GUUUGCA | | GdTsdT | | UfUfUfGCfAdTsdT |
| 827 | GCCUCUCAUUUUACC | 828 | GUCCGGUAAAAU | 357 | GccucucAuuuuAccGGAcd | 358 | GUCCGGuAAAAUGAGA |
| | GGAC | | GAGAGGC | | TsdT | | GGCdTsdT |
| 1031 | UGCUGUUCUGGUAUU | 1032 | UGGUAAUACCAG | 359 | uGcuGuucuGGuAuuAccA | 360 | UGGuAAuACcAGAAcAGc |
| | ACCA | | AACAGCA | | dTsdT | | AdTsdT |
| 971 | GAACUGGCAGCGGUU | 972 | AUAAAACCGCUG | 361 | GAAcuGGcAGcGGuuuuA | 362 | AuAAAACCGCUGCcAGU |
| | UUAU | | CCAGUUC | | udTsdT | | UCdTsdT |
| 995 | CCUAUGUAUGUGUUA | 996 | CAGAUAACACAU | 363 | ccuAuGuAuGuGuuAucuG | 364 | cAGAuAAcAcAuAcAuAG |
| | UCUG | | ACAUAGG | | dTsdT | | GdTsdT |
| 845 | AGAAGAUUUCAUCGA | 846 | GAGUUCGAUGAA | 365 | AGAAGAuuucAucGAAcu | 366 | GAGUfUfCfGAUfGAAAUf |
| | ACUC | | AUCUUCU | | cdTsdT | | CfUfUfCfUfdTsdT |
| 869 | CUCUGAACUUCCCUG | 870 | CGACCAGGGAAG | 367 | cucuGAAcuucccuGGucGd | 368 | CfGACfCfAGGGAAGUfUf |
| | GUCG | | UUCAGAG | | TsdT | | CfAGAGdTsdT |
| 881 | CUGGUGUGCUCUGAU | 882 | CUUCAUCAGAGC | 369 | cuGGuGuGcucuGAuGAA | 370 | CUUcAUcAGAGcAcACcA |
| | GAAG | | ACACCAG | | GdTsdT | | GdTsdT |
| 931 | CUCAACUUGGAGGAU | 932 | CAUGAUCCUCCA | 371 | cucAAcuuGGAGGAucAu | 372 | CfAUfGAUfCfCfUfCfCfAA |
| | CAUG | | AGUUGAG | | GdTsdT | | GUfUfGAGdTsdT |
| 825 | AGCCUCUCAUUUUAC | 826 | UCCGGUAAAAUG | 373 | AGccucucAuuuuAccGGA | 374 | UCCGGuAAAAUGAGAG |
| | CGGA | | AGAGGCU | | dTsdT | | GCUdTsdT |
| 915 | AUAGUUCCUGCAACG | 916 | GUAACGUUGCAG | 375 | AuAGuuccuGcAAcGuuAc | 376 | GuAACGUUGcAGGAACu |
| | UUAC | | GAACUAU | | dTsdT | | AUdTsdT |
| 911 | AACAAUAGUUCCUGC | 912 | CGUUGCAGGAAC | 377 | AAcAAuAGuuccuGcAAc | 378 | CfGUfUfGCfAGGAACfUf |
| | AACG | | UAUUGUU | | GdTsdT | | AUfUfGUfUfdTsdT |
| 841 | AUCUGGUUUUGUCAA | 842 | GGGCUUGACAAA | 379 | AucuGGuuuuGucAAGccc | 380 | GGGCUUGAcAAAACcAG |
| | GCCC | | ACCAGAU | | dTsdT | | AUdTsdT |
| 1029 | ACUGUAAAACCUUGU | 1030 | CCACACAAGGUU | 381 | AcuGuAAAAccuuGuGuG | 382 | CcAcAcAAGGUUUuAcAG |
| | GUGG | | UUACAGU | | GdTsdT | | UdTsdT |
| 975 | AACUCUUGGAUUCUA | 976 | UGCAUAGAAUCC | 383 | AAcucuuGGAuucuAuGcA | 384 | UGcAuAGAAUCcAAGAG |
| | UGCA | | AAGAGUU | | dTsdT | | UUdTsdT |
| 1009 | UAGUGACCAGGUUUU | 1010 | CCUGAAAACCUG | 385 | uAGuGAccAGGuuuucAG | 386 | CfCfUfGAAAACfCfUfGG |
| | CAGG | | GUCACUA | | GdTsdT | | UfCfACfUfAdTsdT |
| 779 | AACCUCAAUAGGUCG | 780 | UGGUCGACCUAU | 387 | AAccucAAuAGGucGAcc | 388 | UfGGUfCfGACfCfUfAUfU |
| | ACCA | | UGAGGUU | | AdTsdT | | fGAGGUfUfdTsdT |
| 829 | CCUCUCAUUUUACCG | 830 | UGUCCGGUAAAA | 389 | ccucucAuuuuAccGGAcAd | 390 | UfGUfCfCfGGUfAAAAUf |
| | GACA | | UGAGAGG | | TsdT | | GAGAGGdTsdT |
| 945 | UGACCAAAUGACCCU | 946 | CAGUAGGGUCAU | 391 | uGAccAAAuGAcccuAcu | 392 | CfAGUfAGGGUfCfAUfUf |
| | ACUG | | UUGGUCA | | GdTsdT | | UfGGUfCfAdTsdT |
| 817 | AGAUCAGACCUGUUG | 818 | CUAUCAACAGGU | 393 | AGAucAGAccuGuuGAuA | 394 | CuAUcAAcAGGUCUGAU |
| | AUAG | | CUGAUCU | | GdTsdT | | CUdTsdT |
| 937 | CAGGUUUCAGGAACU | 938 | UGUAAGUUCCUG | 395 | cAGGuuucAGGAAcuuAc | 396 | UfGUfAAGUfUfCfCfUfGA |
| | UACA | | AAACCUG | | AdTsdT | | AACfCfUfGdTsdT |
| 1025 | UAGUUUUUUAUUCAU | 1026 | CAGCAUGAAUAA | 397 | uAGuuuuuuAuucAuGcuG | 398 | cAGcAUGAAuAAAAAAC |
| | GCUG | | AAAACUA | | dTsdT | | uAdTsdT |
| 785 | UGUGAUGGACUUCUA | 786 | UUUAUAGAAGUC | 399 | uGuGAuGGAcuucuAuAA | 400 | UUuAuAGAAGUCcAUcAc |
| | UAAA | | CAUCACA | | AdTsdT | | AdTsdT |
| 989 | UGGCGCUCAAAAAAU | 990 | UUCUAUUUUUUG | 401 | uGGcGcucAAAAAAuAG | 402 | UUCuAUUUUUUGAGCG |
| | AGAA | | AGCGCCA | | AAdTsdT | | CcAdTsdT |
| 911 | AACAAUAGUUCCUGC | 912 | CGUUGCAGGAAC | 403 | AAcAAuAGuuccuGcAAc | 404 | CGUUGcAGGAACuAUUG |
| | AACG | | UAUUGUU | | GdTsdT | | UUdTsdT |
| 923 | UGAACCUGAAGUGUU | 924 | AUAUAACACUUC | 405 | uGAAccuGAAGuGuuAu | 406 | AuAuAAcACUUcAGGUUc |
| | AUAU | | AGGUUCA | | AudTsdT | | AdTsdT |
| 797 | CUCUCAAUGGGACUG | 798 | UAUACAGUCCCA | 407 | cucucAAuGGGAcuGuAu | 408 | uAuAcAGUCCcAUUGAG |
| | UAUA | | UUGAGAG | | AdTsdT | | AGdTsdT |
| 963 | UCUGUAUGAAAACCU | 964 | AGUAAGGUUUUC | 409 | ucuGuAuGAAAAccuuAcu | 410 | AGuAAGGUUUUcAuAcA |
| | UACU | | AUACAGA | | dTsdT | | GAdTsdT |
| 895 | AUGCCGCUAUCGAAA | 896 | ACAUUUUCGAUA | 411 | AuGccGcuAucGAAAAuG | 412 | AcAUUUUCGAuAGCGGc |
| | AUGU | | GCGGCAU | | udTsdT | | AUdTsdT |
| 917 | UAGUUCCUGCAACGU | 918 | GGUAACGUUGCA | 413 | uAGuuccuGcAAcGuuAcc | 414 | GGuAACGUUGcAGGAAC |
| | UACC | | GGAACUA | | dTsdT | | uAdTsdT |
| 985 | AACAAUCUUGGCGCU | 986 | UUUGAGCGCCAA | 415 | AAcAAucuuGGcGcucAA | 416 | UfUfUfGAGCfGCfCfAAG |
| | CAAA | | GAUUGUU | | AdTsdT | | AUfUfGUfUfdTsdT |
| 777 | AAACCUCAAUAGGUC | 778 | GGUCGACCUAUU | 417 | AAAccucAAuAGGucGAc | 418 | GGUfCfGACfCfUfAUfUfG |
| | GACC | | GAGGUUU | | cdTsdT | | AGGUfUfUfdTsdT |
| 789 | UUUCCAAAAGGCUCA | 790 | UUACUGAGCCUU | 419 | uuuccAAAAGGcucAGuA | 420 | UfUfACfUfGAGCfCfUfUf |
| | GUAA | | UUGGAAA | | AdTsdT | | UfUfGGAAAdTsdT |
| 799 | UCUCAAUGGGACUGU | 800 | AUAUACAGUCCC | 421 | ucucAAuGGGAcuGuAuA | 422 | AuAuAcAGUCCcAUUGA |
| | AUAU | | AUUGAGA | | udTsdT | | GAdTsdT |
| 985 | AACAAUCUUGGCGCU | 986 | UUUGAGCGCCAA | 423 | AAcAAucuuGGcGcucAA | 424 | UUUGAGCGCcAAGAUU |
| | CAAA | | GAUUGUU | | AdTsdT | | GUUdTsdT |
| 975 | AACUCUUGGAUUCUA | 976 | UGCAUAGAAUCC | 425 | AAcucuuGGAuucuAuGcA | 426 | UfGCfAUfAGAAUfCfCfA |
| | UGCA | | AAGAGUU | | dTsdT | | AGAGUfUfdTsdT |
| 843 | AAAAAGAAGAUUUCA | 844 | UCGAUGAAAUCU | 427 | AAAAAGAAGAuuucAuc | 428 | UCGAUGAAAUCUUCUU |
| | UCGA | | UCUUUUU | | GAdTsdT | | UUUdTsdT |
| 953 | CAUAUAGACAAUCAA | 954 | GCACUUGAUUGU | 429 | cAuAuAGAcAAucAAGu | 430 | GcACUUGAUUGUCuAuA |
| | GUGC | | CUAUAUG | | GcdTsdT | | UGdTsdT |
| 943 | ACUUACACCUGGAUG | 944 | UGGUCAUCCAGG | 431 | AcuuAcAccuGGAuGAcc | 432 | UGGUcAUCcAGGUGuAA |
| | ACCA | | UGUAAGU | | AdTsdT | | GUdTsdT |
| 835 | UUUACCGGACACUAA | 836 | GGGUUUAGUGUC | 433 | uuuAccGGAcAcuAAAccc | 434 | GGGUfUfUfAGUfGUfCfCf |
| | ACCC | | CGGUAAA | | dTsdT | | GGUfAAAdTsdT |
| 813 | CAGGUAAAGAGACGA | 814 | UCAUUCGUCUCU | 435 | cAGGuAAAGAGAcGAA | 436 | UcAUUCGUCUCUUuACC |
| | AUGA | | UUACCUG | | uGAdTsdT | | UGdTsdT |
| 887 | CCCAGCAUGCCGCUA | 888 | UCGAUAGCGGCA | 437 | cccAGcAuGccGcuAucGA | 438 | UfCfGAUfAGCfGGCfAUf |
| | UCGA | | UGCUGGG | | dTsdT | | GCfUfGGGdTsdT |
| 887 | CCCAGCAUGCCGCUA | 888 | UCGAUAGCGGCA | 439 | cccAGcAuGccGcuAucGA | 440 | UCGAuAGCGGcAUGCUG |
| | UCGA | | UGCUGGG | | dTsdT | | GGdTsdT |
| 853 | GGAGGACAGAUGUAC | 854 | AGUGGUACAUCU | 441 | GGAGGAcAGAuGuAccA | 442 | AGUfGGUfACfAUfCfUfG |
| | CACU | | GUCCUCC | | cudTsdT | | UfCfCfUfCfCfdTsdT |
| 955 | CAUGUACGACCAAUG | 956 | UUUACAUUGGUC | 443 | cAuGuAcGAccAAuGuAA | 444 | UUuAcAUUGGUCGuAcA |
| | UAAA | | GUACAUG | | AdTsdT | | UGdTsdT |
| 917 | UAGUUCCUGCAACGU | 918 | GGUAACGUUGCA | 445 | uAGuuccuGcAAcGuuAcc | 446 | GGUfAACfGUfUfGCfAGG |
| | UACC | | GGAACUA | | dTsdT | | AACfUfAdTsdT |
| 941 | AACUUACACCUGGAU | 942 | GGUCAUCCAGGU | 447 | AAcuuAcAccuGGAuGAc | 448 | GGUfCfAUfCfCfAGGUfG |
| | GACC | | GUAAGUU | | cdTsdT | | UfAAGUfUfdTsdT |
| 909 | AAACAAUAGUUCCUG | 910 | GUUGCAGGAACU | 449 | AAAcAAuAGuuccuGcAA | 450 | GUUGcAGGAACuAUUGU |
| | CAAC | | AUUGUUU | | cdTsdT | | UUdTsdT |
| 833 | UUUUACCGGACACUA | 834 | GGUUUAGUGUCC | 451 | uuuuAccGGAcAcuAAAcc | 452 | GGUfUfUfAGUfGUfCfCfG |
| | AACC | | GGUAAAA | | dTsdT | | GUfAAAAdTsdT |
| 1003 | UGAGAGUUGGUUACU | 1004 | UGUGAGUAACCA | 453 | uGAGAGuuGGuuAcucAc | 454 | UfGUfGAGUfAACfCfAAC |
| | CACA | | ACUCUCA | | AdTsdT | | fUfCfUfCfAdTsdT |
| 913 | ACAAUAGUUCCUGCA | 914 | ACGUUGCAGGAA | 455 | AcAAuAGuuccuGcAAcG | 456 | ACGUUGcAGGAACuAUU |
| | ACGU | | CUAUUGU | | udTsdT | | GUdTsdT |
| 1007 | GGUCCACCCAGGAUU | 1008 | CACUAAUCCUGG | 457 | GGuccAcccAGGAuuAGu | 458 | CfACfUfAAUfCfCfUfGGG |
| | AGUG | | GUGGACC | | GdTsdT | | UfGGACfCfdTsdT |
| 925 | UGUUAUAUGCAGGAU | 926 | UCAUAUCCUGCA | 459 | uGuuAuAuGcAGGAuAu | 460 | UcAuAUCCUGcAuAuAAc |
| | AUGA | | UAUAACA | | GAdTsdT | | AdTsdT |
| 877 | AUGAGACCAGAUGUA | 878 | AGCUUACAUCUG | 461 | AuGAGAccAGAuGuAAG | 462 | AGCfUfUfACfAUfCfUfGG |
| | AGCU | | GUCUCAU | | cudTsdT | | UfCfUfCfAUfdTsdT |
| 781 | ACCUCAAUAGGUCGA | 782 | CUGGUCGACCUA | 463 | AccucAAuAGGucGAccA | 464 | CfUfGGUfCfGACfCfUfAUf |
| | CCAG | | UUGAGGU | | GdTsdT | | UfGAGGUfdTsdT |
| 845 | AGAAGAUUUCAUCGA | 846 | GAGUUCGAUGAA | 465 | AGAAGAuuucAucGAAcu | 466 | GAGUUCGAUGAAAUCU |
| | ACUC | | AUCUUCU | | cdTsdT | | UCUdTsdT |
| 777 | AAACCUCAAUAGGUC | 778 | GGUCGACCUAUU | 467 | AAAccucAAuAGGucGAc | 468 | GGUCGACCuAUUGAGG |
| | GACC | | GAGGUUU | | cdTsdT | | UUUdTsdT |
| 861 | UUCCACCAAUUCCCG | 862 | CCAACGGGAAUU | 469 | uuccAccAAuucccGuuGGd | 470 | CfCfAACfGGGAAUfUfGG |
| | UUGG | | GGUGGAA | | TsdT | | UfGGAAdTsdT |
| 945 | UGACCAAAUGACCCU | 946 | CAGUAGGGUCAU | 471 | uGAccAAAuGAcccuAcu | 472 | cAGuAGGGUcAUUUGGU |
| | ACUG | | UUGGUCA | | GdTsdT | | cAdTsdT |
| 859 | AUUCCACCAAUUCCC | 860 | CAACGGGAAUUG | 473 | AuuccAccAAuucccGuuGd | 474 | cAACGGGAAUUGGUGG |
| | GUUG | | GUGGAAU | | TsdT | | AAUdTsdT |
| 1005 | UGGUCCACCCAGGAU | 1006 | ACUAAUCCUGGG | 475 | uGGuccAcccAGGAuuAG | 476 | ACfUfAAUfCfCfUfGGGUf |
| | UAGU | | UGGACCA | | udTsdT | | GGACfCfAdTsdT |
| 901 | AGGAAUUCAGCAGGC | 902 | AGUGGCCUGCUG | 477 | AGGAAuucAGcAGGccA | 478 | AGUGGCCUGCUGAAUU |
| | CACU | | AAUUCCU | | cudTsdT | | CCUdTsdT |
| 871 | ACUUCCCUGGUCGAA | 872 | ACUGUUCGACCA | 479 | AcuucccuGGucGAAcAGu | 480 | ACfUfGUfUfCfGACfCfAG |
| | CAGU | | GGGAAGU | | dTsdT | | GGAAGUfdTsdT |
| 833 | UUUUACCGGACACUA | 834 | GGUUUAGUGUCC | 481 | uuuuAccGGAcAcuAAAcc | 482 | GGUUuAGUGUCCGGuAA |
| | AACC | | GGUAAAA | | dTsdT | | AAdTsdT |
| 1017 | AAAUAACUUGCUUAA | 1018 | GUAGUUAAGCAA | 483 | AAAuAAcuuGcuuAAcuA | 484 | GuAGUuAAGcAAGUuAU |
| | CUAC | | GUUAUUU | | cdTsdT | | UUdTsdT |
| 791 | AAGGCUCAGUAAGCA | 792 | GCAUUGCUUACU | 485 | AAGGcucAGuAAGcAAu | 486 | GCfAUfUfGCfUfUfACfUf |
| | AUGC | | GAGCCUU | | GcdTsdT | | GAGCfCfUfUfdTsdT |
| 901 | AGGAAUUCAGCAGGC | 902 | AGUGGCCUGCUG | 487 | AGGAAuucAGcAGGccA | 488 | AGUfGGCfCfUfGCfUfGA |
| | CACU | | AAUUCCU | | cudTsdT | | AUfUfCfCfUfdTsdT |
| 869 | CUCUGAACUUCCCUG | 870 | CGACCAGGGAAG | 489 | cucuGAAcuucccuGGucGd | 490 | CGACcAGGGAAGUUcAG |
| | GUCG | | UUCAGAG | | TsdT | | AGdTsdT |
| 801 | UCAAUGGGACUGUAU | 802 | CCAUAUACAGUC | 491 | ucAAuGGGAcuGuAuAu | 492 | CcAuAuAcAGUCCcAUUG |
| | AUGG | | CCAUUGA | | GGdTsdT | | AdTsdT |
| 847 | AAACUGGGCACAGUU | 848 | AGUAAACUGUGC | 493 | AAAcuGGGcAcAGuuuAc | 494 | AGuAAACUGUGCCcAGU |
| | UACU | | CCAGUUU | | udTsdT | | UUdTsdT |
| 823 | AAGCCUCUCAUUUUA | 824 | CCGGUAAAAUGA | 495 | AAGccucucAuuuuAccGG | 496 | CfCfGGUfAAAAUfGAGA |
| | CCGG | | GAGGCUU | | dTsdT | | GGCfUfUfdTsdT |
| 871 | ACUUCCCUGGUCGAA | 872 | ACUGUUCGACCA | 497 | AcuucccuGGucGAAcAGu | 498 | ACUGUUCGACcAGGGAA |
| | CAGU | | GGGAAGU | | dTsdT | | GUdTsdT |
| 941 | AACUUACACCUGGAU | 942 | GGUCAUCCAGGU | 499 | AAcuuAcAccuGGAuGAc | 500 | GGUcAUCcAGGUGuAAG |
| | GACC | | GUAAGUU | | cdTsdT | | UUdTsdT |
| 853 | GGAGGACAGAUGUAC | 854 | AGUGGUACAUCU | 501 | GGAGGAcAGAuGuAccA | 502 | AGUGGuAcAUCUGUCCU |
| | CACU | | GUCCUCC | | cudTsdT | | CCdTsdT |
| 1007 | GGUCCACCCAGGAUU | 1008 | CACUAAUCCUGG | 503 | GGuccAcccAGGAuuAGu | 504 | cACuAAUCCUGGGUGGA |
| | AGUG | | GUGGACC | | GdTsdT | | CCdTsdT |
| 791 | AAGGCUCAGUAAGCA | 792 | GCAUUGCUUACU | 505 | AAGGcucAGuAAGcAAu | 506 | GcAUUGCUuACUGAGCC |
| | AUGC | | GAGCCUU | | GcdTsdT | | UUdTsdT |
| 861 | UUCCACCAAUUCCCG | 862 | CCAACGGGAAUU | 507 | uuccAccAAuucccGuuGGd | 508 | CcAACGGGAAUUGGUG |
| | UUGG | | GGUGGAA | | TsdT | | GAAdTsdT |
| 835 | UUUACCGGACACUAA | 836 | GGGUUUAGUGUC | 509 | uuuAccGGAcAcuAAAccc | 510 | GGGUUuAGUGUCCGGuA |
| | ACCC | | CGGUAAA | | dTsdT | | AAdTsdT |
| 1005 | UGGUCCACCCAGGAU | 1006 | ACUAAUCCUGGG | 511 | uGGuccAcccAGGAuuAG | 512 | ACuAAUCCUGGGUGGAC |
| | UAGU | | UGGACCA | | udTsdT | | cAdTsdT |
| 823 | AAGCCUCUCAUUUUA | 824 | CCGGUAAAAUGA | 513 | AAGccucucAuuuuAccGG | 514 | CCGGuAAAAUGAGAGG |
| | CCGG | | GAGGCUU | | dTsdT | | CUUdTsdT |
| 991 | AGGCUUUUCAUUAAA | 992 | CCCAUUUAAUGA | 515 | AGGcuuuucAuuAAAuGG | 516 | CCcAUUuAAUGAAAAGC |
| | UGGG | | AAAGCCU | | GdTsdT | | CUdTsdT |
| 873 | UGGUCGAACAGUUUU | 874 | AGAAAAAACUGU | 55 | uGGucGAAcAGuuuuuucu | 744 | pAGAAAAAACUGUUCG |
| | UUCU | | UCGACCA | | dTsdT | | ACcAdTsdT |
| 873 | UGGUCGAACAGUUUU | 874 | AGAAAAAACUGU | 739 | ugGucGAAcAGuuuuuucu | 744 | pAGAAAAAACUGUUCG |
| | UUCU | | UCGACCA | | dTsdT | | ACcAdTsdT |
| 873 | UGGUCGAACAGUUUU | 874 | AGAAAAAACUGU | 739 | ugGucGAAcAGuuuuuucu | 56 | AGAAAAAACUGUUCGA |
| | UUCU | | UCGACCA | | dTsdT | | CcAdTsdT |
| 1033 | UGGUCGAACAGUUUU | 874 | AGAAAAAACUGU | 740 | uGGucGAAcAGuuuuuucc | 744 | pAGAAAAAACUGUUCG |
| | UUCC | | UCGACCA | | dTsdT | | ACcAdTsdT |
| 1033 | UGGUCGAACAGUUUU | 874 | AGAAAAAACUGU | 741 | ugGucGAAcAGuuuuuucc | 744 | pAGAAAAAACUGUUCG |
| | UUCC | | UCGACCA | | dTsdT | | ACcAdTsdT |
| 1033 | UGGUCGAACAGUUUU | 874 | AGAAAAAACUGU | 740 | uGGucGAAcAGuuuuuucc | 56 | AGAAAAAACUGUUCGA |
| | UUCC | | UCGACCA | | dTsdT | | CcAdTsdT |
| 1033 | UGGUCGAACAGUUUU | 874 | AGAAAAAACUGU | 741 | ugGucGAAcAGuuuuuucc | 56 | AGAAAAAACUGUUCGA |
| | UUCC | | UCGACCA | | dTsdT | | CcAdTsdT |
| 1034 | UGGUCGAACAGUUUU | 874 | AGAAAAAACUGU | 742 | uGGucGAAcAGuuuuuuc | 745 | pAGAAAAAACUGUUCG |
| | UUCG | | UCGACCA | | GdTsdT | | ACcAdTsdT |
| 1034 | UGGUCGAACAGUUUU | 874 | AGAAAAAACUGU | 743 | ugGucGAAcAGuuuuuucG | 745 | pAGAAAAAACUGUUCG |
| | UUCG | | UCGACCA | | dTsdT | | ACcAdTsdT |
| 1034 | UGGUCGAACAGUUUU | 874 | AGAAAAAACUGU | 742 | uGGucGAAcAGuuuuuuc | 56 | AGAAAAAACUGUUCGA |
| | UUCG | | UCGACCA | | GdTsdT | | CcAdTsdT |
| 1034 | UGGUCGAACAGUUUU | 874 | AGAAAAAACUGU | 743 | ugGucGAAcAGuuuuuucG | 56 | AGAAAAAACUGUUCGA |
| | UUCG | | UCGACCA | | dTsdT | | CcAdTsdT |
| 873 | UGGUCGAACAGUUUU | 874 | AGAAAAAACUGU | 746 | uGGucGAAcAGuuuuuucu | 752 | pAGAAAAAACUGUUCG |
| | UUCU | | UCGACCA | | dT(invdT) | | ACcAdT(invdT) |
| 873 | UGGUCGAACAGUUUU | 874 | AGAAAAAACUGU | 747 | ugGucGAAcAGuuuuuucu | 752 | pAGAAAAAACUGUUCG |
| | UUCU | | UCGACCA | | dT(invdT) | | ACcAdT(invdT) |
| 873 | UGGUCGAACAGUUUU | 874 | AGAAAAAACUGU | 746 | uGGucGAAcAGuuuuuucu | 753 | AGAAAAAACUGUUCGA |
| | UUCU | | UCGACCA | | dT(invdT) | | CcAdT(invdT) |
| 873 | UGGUCGAACAGUUUU | 874 | AGAAAAAACUGU | 747 | ugGucGAAcAGuuuuuucu | 753 | AGAAAAAACUGUUCGA |
| | UUCU | | UCGACCA | | dT(invdT) | | CcAdT(invdT) |
| 1033 | UGGUCGAACAGUUUU | 874 | AGAAAAAACUGU | 748 | uGGucGAAcAGuuuuuucc | 752 | pAGAAAAAACUGUUCG |
| | UUCC | | UCGACCA | | dT(invdT) | | ACcAdT(invdT) |
| 1033 | UGGUCGAACAGUUUU | 874 | AGAAAAAACUGU | 749 | ugGucGAAcAGuuuuuucc | 752 | pAGAAAAAACUGUUCG |
| | UUCC | | UCGACCA | | dT(invdT) | | ACcAdT(invdT) |
| 1033 | UGGUCGAACAGUUUU | 874 | AGAAAAAACUGU | 748 | uGGucGAAcAGuuuuuucc | 753 | AGAAAAAACUGUUCGA |
| | UUCC | | UCGACCA | | dT(invdT) | | CcAdT(invdT) |
| 1033 | UGGUCGAACAGUUUU | 874 | AGAAAAAACUGU | 749 | ugGucGAAcAGuuuuuucc | 753 | AGAAAAAACUGUUCGA |
| | UUCC | | UCGACCA | | dT(invdT) | | CcAdT(invdT) |
| 1034 | UGGUCGAACAGUUUU | 874 | AGAAAAAACUGU | 750 | uGGucGAAcAGuuuuuuc | 752 | pAGAAAAAACUGUUCG |
| | UUCG | | UCGACCA | | GdT(invdT) | | ACcAdT(invdT) |
| 1034 | UGGUCGAACAGUUUU | 874 | AGAAAAAACUGU | 751 | ugGucGAAcAGuuuuuucG | 752 | pAGAAAAAACUGUUCG |
| | UUCG | | UCGACCA | | dT(invdT) | | ACcAdT(invdT) |
| 1034 | UGGUCGAACAGUUUU | 874 | AGAAAAAACUGU | 750 | uGGucGAAcAGuuuuuuc | 753 | AGAAAAAACUGUUCGA |
| | UUCG | | UCGACCA | | GdT(invdT) | | CcAdT(invdT) |
| 1034 | UGGUCGAACAGUUUU | 874 | AGAAAAAACUGU | 751 | ugGucGAAcAGuuuuuucG | 753 | AGAAAAAACUGUUCGA |
| | UUCG | | UCGACCA | | dT(invdT) | | CcAdT(invdT) |
| 873 | UGGUCGAACAGUUUU | 874 | AGAAAAAACUGU | 754 | uGGucGAAcAGuuuuuucu | 760 | pAGAAAAAACUGUUCG |
| | UUCU | | UCGACCA | | dT(abasic) | | ACcAdT(abasic) |
| 873 | UGGUCGAACAGUUUU | 874 | AGAAAAAACUGU | 755 | ugGucGAAcAGuuuuuucu | 760 | pAGAAAAAACUGUUCG |
| | UUCU | | UCGACCA | | dT(abasic) | | ACcAdT(abasic) |
| 873 | UGGUCGAACAGUUUU | 874 | AGAAAAAACUGU | 754 | uGGucGAAcAGuuuuuucu | 761 | AGAAAAAACUGUUCGA |
| | UUCU | | UCGACCA | | dT(abasic) | | CcAdT(abasic) |
| 873 | UGGUCGAACAGUUUU | 874 | AGAAAAAACUGU | 755 | ugGucGAAcAGuuuuuucu | 761 | AGAAAAAACUGUUCGA |
| | UUCU | | UCGACCA | | dT(abasic) | | CcAdT(abasic) |
| 1033 | UGGUCGAACAGUUUU | 874 | AGAAAAAACUGU | 756 | uGGucGAAcAGuuuuuucc | 760 | pAGAAAAAACUGUUCG |
| | UUCC | | UCGACCA | | dT(abasic) | | ACcAdT(abasic) |
| 1033 | UGGUCGAACAGUUUU | 874 | AGAAAAAACUGU | 757 | ugGucGAAcAGuuuuuucc | 760 | pAGAAAAAACUGUUCG |
| | UUCC | | UCGACCA | | dT(abasic) | | ACcAdT(abasic) |
| 1033 | UGGUCGAACAGUUUU | 874 | AGAAAAAACUGU | 756 | uGGucGAAcAGuuuuuucc | 761 | AGAAAAAACUGUUCGA |
| | UUCC | | UCGACCA | | dT(abasic) | | CcAdT(abasic) |
| 1033 | UGGUCGAACAGUUUU | 874 | AGAAAAAACUGU | 757 | ugGucGAAcAGuuuuuucc | 761 | AGAAAAAACUGUUCGA |
| | UUCC | | UCGACCA | | dT(abasic) | | CcAdT(abasic) |
| 1034 | UGGUCGAACAGUUUU | 874 | AGAAAAAACUGU | 758 | ugGucGAAcAGuuuuuucG | 760 | pAGAAAAAACUGUUCG |
| | UUCG | | UCGACCA | | dT(abasic) | | ACcAdT(abasic) |
| 1034 | UGGUCGAACAGUUUU | 874 | AGAAAAAACUGU | 759 | uGGucGAAcAGuuuuuuc | 761 | AGAAAAAACUGUUCGA |
| | UUCG | | UCGACCA | | GdT(abasic) | | CcAdT(abasic) |
| 1034 | UGGUCGAACAGUUUU | 874 | AGAAAAAACUGU | 758 | ugGucGAAcAGuuuuuucG | 761 | AGAAAAAACUGUUCGA |
| | UUCG | | UCGACCA | | dT(abasic) | | CcAdT(abasic) |
| 929 | GUUCCAGACUCAACU | 930 | UCCAAGUUGAGU | 83 | GuuccAGAcucAAcuuGG | 770 | pUCcAAGUUGAGUCUGG |
| | UGGA | | CUGGAAC | | AdTsdT | | AACdTsdT |
| 1035 | GUUCCAGACUCAACU | 930 | UCCAAGUUGAGU | 762 | GuuccAGAcucAAcuuGGc | 770 | pUCcAAGUUGAGUCUGG |
| | UGGC | | CUGGAAC | | dTsdT | | AACdTsdT |
| 1035 | GUUCCAGACUCAACU | 930 | UCCAAGUUGAGU | 762 | GuuccAGAcucAAcuuGGc | 84 | UCcAAGUUGAGUCUGG |
| | UGGC | | CUGGAAC | | dTsdT | | AACdTsdT |
| 1036 | GUUCCAGACUCAACU | 930 | UCCAAGUUGAGU | 763 | GuuccAGAcucAAcuuGGu | 770 | pUCcAAGUUGAGUCUGG |
| | UGGU | | CUGGAAC | | dTsdT | | AACdTsdT |
| 1036 | GUUCCAGACUCAACU | 930 | UCCAAGUUGAGU | 763 | GuuccAGAcucAAcuuGGu | 84 | UCcAAGUUGAGUCUGG |
| | UGGU | | CUGGAAC | | dTsdT | | AACdTsdT |
| 929 | GUUCCAGACUCAACU | 930 | UCCAAGUUGAGU | 764 | GuuccAGAcucAAcuuGG | 771 | pUCcAAGUUGAGUCUGG |
| | UGGA | | CUGGAAC | | AdT(invdT) | | AACdT(invdT) |
| 929 | GUUCCAGACUCAACU | 930 | UCCAAGUUGAGU | 764 | GuuccAGAcucAAcuuGG | 772 | UCcAAGUUGAGUCUGG |
| | UGGA | | CUGGAAC | | AdT(invdT) | | AACdT(invdT) |
| 1035 | GUUCCAGACUCAACU | 930 | UCCAAGUUGAGU | 765 | GuuccAGAcucAAcuuGGc | 771 | pUCcAAGUUGAGUCUGG |
| | UGGC | | CUGGAAC | | dT(invdT) | | AACdT(invdT) |
| 1035 | GUUCCAGACUCAACU | 930 | UCCAAGUUGAGU | 765 | GuuccAGAcucAAcuuGGc | 772 | UCcAAGUUGAGUCUGG |
| | UGGC | | CUGGAAC | | dT(invdT) | | AACdT(invdT) |
| 1036 | GUUCCAGACUCAACU | 930 | UCCAAGUUGAGU | 766 | GuuccAGAcucAAcuuGGu | 771 | pUCcAAGUUGAGUCUGG |
| | UGGU | | CUGGAAC | | dT(invdT) | | AACdT(invdT) |
| 1036 | GUUCCAGACUCAACU | 930 | UCCAAGUUGAGU | 766 | GuuccAGAcucAAcuuGGu | 772 | UCcAAGUUGAGUCUGG |
| | UGGU | | CUGGAAC | | dT(invdT) | | AACdT(invdT) |
| 929 | GUUCCAGACUCAACU | 930 | UCCAAGUUGAGU | 767 | GuuccAGAcucAAcuuGG | 773 | pUCcAAGUUGAGUCUGG |
| | UGGA | | CUGGAAC | | AdT(abasic) | | AACdT(abasic) |
| 929 | GUUCCAGACUCAACU | 930 | UCCAAGUUGAGU | 767 | GuuccAGAcucAAcuuGG | 774 | UCcAAGUUGAGUCUGG |
| | UGGA | | CUGGAAC | | AdT(abasic) | | AACdT(abasic) |
| 1035 | GUUCCAGACUCAACU | 930 | UCCAAGUUGAGU | 768 | GuuccAGAcucAAcuuGGc | 773 | pUCcAAGUUGAGUCUGG |
| | UGGC | | CUGGAAC | | dT(abasic) | | AACdT(abasic) |
| 1035 | GUUCCAGACUCAACU | 930 | UCCAAGUUGAGU | 768 | GuuccAGAcucAAcuuGGc | 774 | UCcAAGUUGAGUCUGG |
| | UGGC | | CUGGAAC | | dT(abasic) | | AACdT(abasic) |
| 1036 | GUUCCAGACUCAACU | 930 | UCCAAGUUGAGU | 769 | GuuccAGAcucAAcuuGGu | 773 | pUCcAAGUUGAGUCUGG |
| | UGGU | | CUGGAAC | | dT(abasic) | | AACdT(abasic) |
| 1036 | GUUCCAGACUCAACU | 930 | UCCAAGUUGAGU | 769 | GuuccAGAcucAAcuuGGu | 774 | UCcAAGUUGAGUCUGG |
| | UGGU | | CUGGAAC | | dT(abasic) | | AACdT(abasic) |

1. A double-stranded ribonucleic acid molecule capable of inhibiting the expression of Glucocorticoid Receptor (GCR) gene in vitro by at least 70%, preferably by at least 80% and most preferably by at least 90%.

2. The double-stranded ribonucleic acid molecule of claim 1, wherein said double-stranded ribonucleic acid molecule comprises a sense strand and an antisense strand, the antisense strand being at least partially complementary to the sense strand, whereby the sense strand comprises a sequence which has an identity of at least 90% to at least a portion of an mRNA encoding GCR, wherein said sequence is (i) located in the region of complementarity of said sense strand to said antisense strand; and (ii) wherein said sequence is less than 30 nucleotides in length.

3. The double-stranded ribonucleic acid molecule of claim 1 or 2, wherein said sense strand comprises a nucleotide acid sequence depicted in SEQ ID Nos: 873, 929, 1021, 1023, 967 and 905, and said antisense strand comprises a nucleic acid sequence depicted in SEQ ID Nos: 874, 930, 1022, 1024, 968 and 906, wherein said double-stranded ribonucleic acid molecule comprises a sequence pair selected from the group consisting of SEQ ID NOs: 873/874, 929/930, 1021/1022, 1023/1024, 967/968 and 905/906.

4. The double-stranded ribonucleic acid molecule of claim 3, wherein the antisense strand further comprises a 3′ overhang of 1-5 nucleotides in length, preferably of 1-2 nucleotides in length.

5. The double-stranded ribonucleic acid molecule of claim 4, wherein the overhang of the antisense strand comprises uracil or nucleotides which are complementary to the mRNA encoding GCR.

6. The double-stranded ribonucleic acid molecule of any one of claims 3 to 5, wherein the sense strand further comprises a 3′ overhang of 1-5 nucleotides in length, preferably of 1-2 nucleotides in length.

7. The double-stranded ribonucleic acid molecule of claim 6 wherein the overhang of the sense strand comprises uracil or nucleotides which are identical to the mRNA encoding GCR.

8. The double-stranded ribonucleic acid molecule of any one of claims 1 to 7, wherein said double-stranded ribonucleic acid molecule comprises at least one modified nucleotide.

9. The double-stranded ribonucleic acid molecule of claim 8, wherein said modified nucleotide is selected from the group consisting of a 2′-O-methyl modified nucleotide, a nucleotide comprising a 5′-phosphorothioate group, and a terminal nucleotide linked to a cholesteryl derivative or a dodecanoic acid bisdecylamide group, a 2′-deoxy-2′-fluoro modified nucleotide, an inverted deoxythymidine, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

10. The double-stranded ribonucleic acid molecule of claim 8 or 9, wherein said modified nucleotide is a 2′-O-methyl modified nucleotide, a nucleotide comprising a 5′-phosphorothioate group, an inverted deoxythymidine or a deoxythymidine.

11. The double-stranded ribonucleic acid molecule of any one of claims 3 to 10, wherein said sense strand and/or said antisense strand comprises an overhang of 1-2 deoxythymidines and/or inverted deoxythymidine.

12. The double-stranded ribonucleic acid molecule of any one of claims 1 to 11, wherein said sense strand is selected from the group consisting of the nucleic acid sequences depicted in SEQ ID Nos: 3, 7, 55, 25, 83, 31, 33, 747 and 764 and said antisense strand is selected from the group consisting of the nucleic acid sequences depicted in SEQ ID Nos: 4, 8, 56, 26, 84, 32, 34, 753 and 772 wherein said double-stranded ribonucleic acid molecule comprises the sequence pairs selected from the group consisting of SEQ ID NOs: 3/4, 7/8, 55/56, 25/26, 83/84, 31/32, 33/34, 747/753 and 764/772.

13. A nucleic acid molecule encoding a sense strand and/or an antisense strand comprised in the double-stranded ribonucleic acid molecule as defined in any one of claims 1 to 12.

14. A vector comprising a regulatory sequence operably linked to a nucleotide sequence that encodes at least one of a sense strand or an antisense strand comprised in the double-stranded ribonucleic acid molecule as defined in any one of claims 1 to 12 or comprising the nucleic acid molecule of claim 13.

15. A cell, tissue or non-human organism comprising the double-stranded ribonucleic acid molecule as defined in any one of claims 1 to 12, the nucleic acid molecule of claim 13 or the vector of claim 14.

16. A pharmaceutical composition comprising the double-stranded ribonucleic acid molecule as defined in any one of claims 1 to 12, the nucleic acid molecule of claim 13, the vector of claim 14 or the cell or tissue of claim 15.

17. The pharmaceutical composition of claim 16, further comprising a pharmaceutically acceptable carrier, stabilizer and/or diluent.

18. A method for inhibiting the expression of GCR gene in a cell, a tissue or an organism comprising the following steps:
(a) introducing into the cell, tissue or organism the double-stranded ribonucleic acid molecule as defined in any one of claims 1 to 12, the nucleic acid molecule of claim 13, the vector of claim 14; and
(b) maintaining the cell, tissue or organism produced in step (a) for a time sufficient to obtain degradation of the mRNA transcript of a GCR gene, thereby inhibiting expression of a GCR gene in the cell.

19. A method of treating, preventing or managing a pathological condition or disease caused by the expression of the GCR gene comprising administering to a subject in need of such treatment, prevention or management a therapeutically or prophylactically effective amount of the double-stranded ribonucleic acid molecule of any one of claims 1 to 12, the nucleic acid molecule of claim 13, the vector of claim 14 and/or the pharmaceutical composition of claim 16 or 17.

20. The method of claim 19, wherein said subject is a human.

21. The method of claim 18 or 19, wherein said disease caused by the expression of the GCR gene is selected from the group consisting of type 2 diabetes, obesity, dislipidemia, diabetic atherosclerosis, hypertension and depression.

22. A method for treating type 2 diabetes, obesity, dislipidemia, diabetic atherosclerosis, hypertension or depression comprising administering to a subject in need of such treatment, a therapeutically or prophylactically effective amount of the double-stranded ribonucleic acid molecule of any one of claims 1 to 12, the nucleic acid molecule of claim 13, the vector of claim 14 and/or the pharmaceutical composition of claim 16 or 17.


SEQUENCE LISTING (1036 sequences)

SEQ ID NO: 1 - DNA, 21 bp, Artificial Sequence
  Description of Artificial Sequence Synthetic oligonucleotide
cauguacgaccaauguaaatt

SEQ ID NO: 2 - DNA, 21 bp, Artificial Sequence
  Description of Artificial Sequence Synthetic oligonucleotide
uuuacauuggucguacaugtt

SEQ ID NO: 3 - DNA, 21 bp, Artificial Sequence
  Description of Artificial Sequence Synthetic oligonucleotide
uugcuuaacuacauauagatt

SEQ ID NO: 4 - DNA, 21 bp, Artificial Sequence
  Description of Artificial Sequence Synthetic oligonucleotide
ucuauauguaguuaagcaatt

SEQ ID NO: 5 - DNA, 21 bp, Artificial Sequence
  Description of Artificial Sequence Synthetic oligonucleotide
aaauaacuugcuuaacuactt

SEQ ID NO: 6 - DNA, 21 bp, Artificial Sequence
  Description of Artificial Sequence Synthetic oligonucleotide
guaguuaagcaaguuauuutt

SEQ ID NO: 7 - DNA, 21 bp, Artificial Sequence
  Description of Artificial Sequence Synthetic oligonucleotide
ugcuuaacuacauauagautt

SEQ ID NO: 8 - DNA, 21 bp, Artificial Sequence
  Description of Artificial Sequence Synthetic oligonucleotide
aucuauauguaguuaagcatt

SEQ ID NO: 9 - DNA, 21 bp, Artificial Sequence
  Description of Artificial Sequence Synthetic oligonucleotide
guaugaaaaccuuacugcutt

SEQ ID NO: 10 - DNA, 21 bp, Artificial Sequence
  Description of Artificial Sequence Synthetic oligonucleotide
agcaguaagguuuucauactt

SEQ ID NO: 11 - DNA, 21 bp, Artificial Sequence
  Description of Artificial Sequence Synthetic oligonucleotide
cagugagaguugguuacuctt

SEQ ID NO: 12 - DNA, 21 bp, Artificial Sequence
  Description of Artificial Sequence Synthetic oligonucleotide
gaguaaccaacucucacugtt

SEQ ID NO: 13 - DNA, 21 bp, Artificial Sequence
  Description of Artificial Sequence Synthetic oligonucleotide
ggguggagaucauauagactt

SEQ ID NO: 14 - DNA, 21 bp, Artificial Sequence
  Description of Artificial Sequence Synthetic oligonucleotide
gucuauaugaucuccaccctt

SEQ ID NO: 15 - DNA, 21 bp, Artificial Sequence
  Description of Artificial Sequence Synthetic oligonucleotide
ggguggagaucauauagactt

SEQ ID NO: 16 - DNA, 21 bp, Artificial Sequence
  Description of Artificial Sequence Synthetic oligonucleotide
gucuauaugaucuccaccctt

SEQ ID NO: 17 - DNA, 21 bp, Artificial Sequence
  Description of Artificial Sequence Synthetic oligonucleotide
cagugagaguugguuacuctt

SEQ ID NO: 18 - DNA, 21 bp, Artificial Sequence
  Description of Artificial Sequence Synthetic oligonucleotide
gaguaaccaacucucacugtt

SEQ ID NO: 19 - DNA, 21 bp, Artificial Sequence
  Description of Artificial Sequence Synthetic oligonucleotide
cauauagacaaucaagugctt

SEQ ID NO: 20 - DNA, 21 bp, Artificial Sequence
  Description of Artificial Sequence Synthetic oligonucleotide
gcacuugauugucuauaugtt

SEQ ID NO: 21 - DNA, 21 bp, Artificial Sequence
  Description of Artificial Sequence Synthetic oligonucleotide
ccuauguauguguuaucugtt

SEQ ID NO: 22 - DNA, 21 bp, Artificial Sequence
  Description of Artificial Sequence Synthetic oligonucleotide
cagauaacacauacauaggtt

SEQ ID NO: 23 - DNA, 21 bp, Artificial Sequence
  Description of Artificial Sequence Synthetic oligonucleotide
uuaaugucauuccaccaautt

SEQ ID NO: 24 - DNA, 21 bp, Artificial Sequence
  Description of Artificial Sequence Synthetic oligonucleotide
auugguggaaugacauuaatt

SEQ ID NO: 25 - DNA, 21 bp, Artificial Sequence
  Description of Artificial Sequence Synthetic oligonucleotide
uugcuuaacuacauauagatt

SEQ ID NO: 26 - DNA, 21 bp, Artificial Sequence
  Description of Artificial Sequence Synthetic oligonucleotide
ucuauauguaguuaagcaatt

SEQ ID NO: 27 - DNA, 21 bp, Artificial Sequence
  Description of Artificial Sequence Synthetic oligonucleotide
uggucgaacaguuuuuucutt

SEQ ID NO: 28 - DNA, 21 bp, Artificial Sequence
  Description of Artificial Sequence Synthetic oligonucleotide
agaaaaaacuguucgaccatt

SEQ ID NO: 29 - DNA, 21 bp, Artificial Sequence
  Description of Artificial Sequence Synthetic oligonucleotide
cacacauuaaucugauuuutt

SEQ ID NO: 30 - DNA, 21 bp, Artificial Sequence
  Description of Artificial Sequence Synthetic oligonucleotide
aaaaucagauuaaugugugtt

SEQ ID NO: 31 - DNA, 21 bp, Artificial Sequence
  Description of Artificial Sequence Synthetic oligonucleotide
guaugaaaaccuuacugcutt

SEQ ID NO: 32 - DNA, 21 bp, Artificial Sequence
  Description of Artificial Sequence Synthetic oligonucleotide
agcaguaagguuuucauactt

SEQ ID NO: 33 - DNA, 21 bp, Artificial Sequence
  Description of Artificial Sequence Synthetic oligonucleotide
cuacaggagucucacaagatt

SEQ ID NO: 34 - DNA, 21 bp, Artificial Sequence
  Description of Artificial Sequence Synthetic oligonucleotide
ucuugugagacuccuguagtt

SEQ ID NO: 35 - DNA, 21 bp, Artificial Sequence
  Description of Artificial Sequence Synthetic oligonucleotide
cuguaugaaaauacccucctt

SEQ ID NO: 36 - DNA, 21 bp, Artificial Sequence
  Description of Artificial Sequence Synthetic oligonucleotide
ggaggguauuuucauacagtt

SEQ ID NO: 37 - DNA, 21 bp, Artificial Sequence
  Description of Artificial Sequence Synthetic oligonucleotide
uccuauguauguguuaucutt

SEQ ID NO: 38 - DNA, 21 bp, Artificial Sequence
  Description of Artificial Sequence Synthetic oligonucleotide
agauaacacauacauaggatt

SEQ ID NO: 39 - DNA, 21 bp, Artificial Sequence
  Description of Artificial Sequence Synthetic oligonucleotide
gguggagaucauauagacatt

SEQ ID NO: 40 - DNA, 21 bp, Artificial Sequence
  Description of Artificial Sequence Synthetic oligonucleotide
ugucuauaugaucuccacctt

SEQ ID NO: 41 - DNA, 21 bp, Artificial Sequence
  Description of Artificial Sequence Synthetic oligonucleotide
auguacgaccaauguaaactt

SEQ ID NO: 42 - DNA, 21 bp, Artificial Sequence
  Description of Artificial Sequence Synthetic oligonucleotide
guuuacauuggucguacautt

SEQ ID NO: 43 - DNA, 21 bp, Artificial Sequence
  Description of Artificial Sequence Synthetic oligonucleotide
acuggcagcgguuuuaucatt

SEQ ID NO: 44 - DNA, 21 bp, Artificial Sequence
  Description of Artificial Sequence Synthetic oligonucleotide
ugauaaaaccgcugccagutt

SEQ ID NO: 45 - DNA, 21 bp, Artificial Sequence
  Description of Artificial Sequence Synthetic oligonucleotide
agugagaguugguuacucatt

SEQ ID NO: 46 - DNA, 21 bp, Artificial Sequence
  Description of Artificial Sequence Synthetic oligonucleotide
ugaguaaccaacucucacutt

SEQ ID NO: 47 - DNA, 21 bp, Artificial Sequence
  Description of Artificial Sequence Synthetic oligonucleotide
aauaacuugcuuaacuacatt

SEQ ID NO: 48 - DNA, 21 bp, Artificial Sequence
  Description of Artificial Sequence Synthetic oligonucleotide
uguaguuaagcaaguuauutt

SEQ ID NO: 49 - DNA, 21 bp, Artificial Sequence
  Description of Artificial Sequence Synthetic oligonucleotide
gugagaguugguuacucactt

SEQ ID NO: 50 - DNA, 21 bp, Artificial Sequence
  Description of Artificial Sequence Synthetic oligonucleotide
gugaguaaccaacucucactt

SEQ ID NO: 51 - DNA, 21 bp, Artificial Sequence
  Description of Artificial Sequence Synthetic oligonucleotide
caucaucgauaaaauucgatt

SEQ ID NO: 52 - DNA, 21 bp, Artificial Sequence
  Description of Artificial Sequence Synthetic oligonucleotide
ucgaauuuuaucgaugaugtt

SEQ ID NO: 53 - DNA, 21 bp, Artificial Sequence
  Description of Artificial Sequence Synthetic oligonucleotide
cuguaugaaaauacccucctt

SEQ ID NO: 54 - DNA, 21 bp, Artificial Sequence
  Description of Artificial Sequence Synthetic oligonucleotide
ggaggguauuuucauacagtt

SEQ ID NO: 55 - DNA, 21 bp, Artificial Sequence
  Description of Artificial Sequence Synthetic oligonucleotide
uggucgaacaguuuuuucutt

SEQ ID NO: 56 - DNA, 21 bp, Artificial Sequence
  Description of Artificial Sequence Synthetic oligonucleotide
agaaaaaacuguucgaccatt

SEQ ID NO: 57 - DNA, 21 bp, Artificial Sequence
  Description of Artificial Sequence Synthetic oligonucleotide
acgauucauuccuuuuggatt

SEQ ID NO: 58 - DNA, 21 bp, Artificial Sequence
  Description of Artificial Sequence Synthetic oligonucleotide
uccaaaaggaaugaaucgutt

SEQ ID NO: 59 - DNA, 21 bp, Artificial Sequence
  Description of Artificial Sequence Synthetic oligonucleotide
cuguaugaaaaccuuacugtt

SEQ ID NO: 60 - DNA, 21 bp, Artificial Sequence
  Description of Artificial Sequence Synthetic oligonucleotide
caguaagguuuucauacagtt

SEQ ID NO: 61 - DNA, 21 bp, Artificial Sequence
  Description of Artificial Sequence Synthetic oligonucleotide
gugagaguugguuacucactt

SEQ ID NO: 62 - DNA, 21 bp, Artificial Sequence
  Description of Artificial Sequence Synthetic oligonucleotide
gugaguaaccaacucucactt

SEQ ID NO: 63 - DNA, 21 bp, Artificial Sequence
  Description of Artificial Sequence Synthetic oligonucleotide
uguacgaccaauguaaacatt

SEQ ID NO: 64 - DNA, 21 bp, Artificial Sequence
  Description of Artificial Sequence Synthetic oligonucleotide
uguuuacauuggucguacatt

SEQ ID NO: 65 - DNA, 21 bp, Artificial Sequence
  Description of Artificial Sequence Synthetic oligonucleotide
uaccggacacuaaacccaatt

SEQ ID NO: 66 - DNA, 21 bp, Artificial Sequence
  Description of Artificial Sequence Synthetic oligonucleotide
uuggguuuaguguccgguatt

SEQ ID NO: 67 - DNA, 21 bp, Artificial Sequence
  Description of Artificial Sequence Synthetic oligonucleotide
ccgcuaucgaaaaugucuutt

SEQ ID NO: 68 - DNA, 21 bp, Artificial Sequence
  Description of Artificial Sequence Synthetic oligonucleotide
aagacauuuucgauagcggtt

SEQ ID NO: 69 - DNA, 21 bp, Artificial Sequence
  Description of Artificial Sequence Synthetic oligonucleotide
agaucagaccuguugauagtt

SEQ ID NO: 70 - DNA, 21 bp, Artificial Sequence
  Description of Artificial Sequence Synthetic oligonucleotide
cuaucaacaggucugaucutt

SEQ ID NO: 71 - DNA, 21 bp, Artificial Sequence
  Description of Artificial Sequence Synthetic oligonucleotide
uccuauguauguguuaucutt

SEQ ID NO: 72 - DNA, 21 bp, Artificial Sequence
  Description of Artificial Sequence Synthetic oligonucleotide
agauaacacauacauaggatt

SEQ ID NO: 73 - DNA, 21 bp, Artificial Sequence
  Description of Artificial Sequence Synthetic oligonucleotide
ucuguaugaaaaccuuacutt

SEQ ID NO: 74 - DNA, 21 bp, Artificial Sequence
  Description of Artificial Sequence Synthetic oligonucleotide
aguaagguuuucauacagatt

SEQ ID NO: 75 - DNA, 21 bp, Artificial Sequence
  Description of Artificial Sequence Synthetic oligonucleotide
aaaacaauaguuccugcaatt

SEQ ID NO: 76 - DNA, 21 bp, Artificial Sequence
  Description of Artificial Sequence Synthetic oligonucleotide
uugcaggaacuauuguuuutt

SEQ ID NO: 77 - DNA, 21 bp, Artificial Sequence
  Description of Artificial Sequence Synthetic oligonucleotide
gucuuaacuuguggaagcutt

SEQ ID NO: 78 - DNA, 21 bp, Artificial Sequence
  Description of Artificial Sequence Synthetic oligonucleotide
agcuuccacaaguuaagactt

SEQ ID NO: 79 - DNA, 21 bp, Artificial Sequence
  Description of Artificial Sequence Synthetic oligonucleotide
acaauaguuccugcaacgutt

SEQ ID NO: 80 - DNA, 21 bp, Artificial Sequence
  Description of Artificial Sequence Synthetic oligonucleotide
acguugcaggaacuauugutt

SEQ ID NO: 81 - DNA, 21 bp, Artificial Sequence
  Description of Artificial Sequence Synthetic oligonucleotide
aggcuuuucauuaaaugggtt

SEQ ID NO: 82 - DNA, 21 bp, Artificial Sequence
  Description of Artificial Sequence Synthetic oligonucleotide
cccauuuaaugaaaagccutt

SEQ ID NO: 83 - DNA, 21 bp, Artificial Sequence
  Description of Artificial Sequence Synthetic oligonucleotide
guuccagacucaacuuggatt

SEQ ID NO: 84 - DNA, 21 bp, Artificial Sequence
  Description of Artificial Sequence Synthetic oligonucleotide
uccaaguugagucuggaactt

SEQ ID NO: 85 - DNA, 21 bp, Artificial Sequence
  Description of Artificial Sequence Synthetic oligonucleotide
auguacgaccaauguaaactt

SEQ ID NO: 86 - DNA, 21 bp, Artificial Sequence
  Description of Artificial Sequence Synthetic oligonucleotide
guuuacauuggucguacautt

SEQ ID NO: 87 - DNA, 21 bp, Artificial Sequence
  Description of Artificial Sequence Synthetic oligonucleotide
cuacaggagucucacaagatt

SEQ ID NO: 88 - DNA, 21 bp, Artificial Sequence
  Description of Artificial Sequence Synthetic oligonucleotide
ucuugugagacuccuguagtt

SEQ ID NO: 89 - DNA, 21 bp, Artificial Sequence
  Description of Artificial Sequence Synthetic oligonucleotide
uguacgaccaauguaaacatt

SEQ ID NO: 90 - DNA, 21 bp, Artificial Sequence
  Description of Artificial Sequence Synthetic oligonucleotide
uguuuacauuggucguacatt

SEQ ID NO: 91 - DNA, 21 bp, Artificial Sequence
  Description of Artificial Sequence Synthetic oligonucleotide
aggaucagaagccuauuuutt

SEQ ID NO: 92 - DNA, 21 bp, Artificial Sequence
  Description of Artificial Sequence Synthetic oligonucleotide
aaaauaggcuucugauccutt

SEQ ID NO: 93 - DNA, 21 bp, Artificial Sequence
  Description of Artificial Sequence Synthetic oligonucleotide
gaaauuagaaugaccuacatt

SEQ ID NO: 94 - DNA, 21 bp, Artificial Sequence
  Description of Artificial Sequence Synthetic oligonucleotide
uguaggucauucuaauuuctt

SEQ ID NO: 95 - DNA, 21 bp, Artificial Sequence
  Description of Artificial Sequence Synthetic oligonucleotide
uucuguucauggugugagutt

SEQ ID NO: 96 - DNA, 21 bp, Artificial Sequence
  Description of Artificial Sequence Synthetic oligonucleotide
acucacaccaugaacagaatt

SEQ ID NO: 97 - DNA, 21 bp, Artificial Sequence
  Description of Artificial Sequence Synthetic oligonucleotide
guuccagacucaacuuggatt

SEQ ID NO: 98 - DNA, 21 bp, Artificial Sequence
  Description of Artificial Sequence Synthetic oligonucleotide
uccaaguugagucuggaactt

SEQ ID NO: 99 - DNA, 21 bp, Artificial Sequence
  Description of Artificial Sequence Synthetic oligonucleotide
ccagauguaagcucuccuctt

SEQ ID NO: 100 - DNA, 21 bp, Artificial Sequence
  Description of Artificial Sequence Synthetic oligonucleotide
gaggagagcuuacaucuggtt

SEQ ID NO: 101 - DNA, 21 bp, Artificial Sequence
  Description of Artificial Sequence Synthetic oligonucleotide
uuucuaauggcuauucaagtt

SEQ ID NO: 102 - DNA, 21 bp, Artificial Sequence
  Description of Artificial Sequence Synthetic oligonucleotide
cuugaauagccauuagaaatt

SEQ ID NO: 103 - DNA, 21 bp, Artificial Sequence
  Description of Artificial Sequence Synthetic oligonucleotide
augccgcuaucgaaaaugutt

SEQ ID NO: 104 - DNA, 21 bp, Artificial Sequence
  Description of Artificial Sequence Synthetic oligonucleotide
acauuuucgauagcggcautt

SEQ ID NO: 105 - DNA, 21 bp, Artificial Sequence
  Description of Artificial Sequence Synthetic oligonucleotide
ccagcaugccgcuaucgaatt

SEQ ID NO: 106 - DNA, 21 bp, Artificial Sequence
  Description of Artificial Sequence Synthetic oligonucleotide
uucgauagcggcaugcuggtt

SEQ ID NO: 107 - DNA, 21 bp, Artificial Sequence
  Description of Artificial Sequence Synthetic oligonucleotide
uuggcgcucaaaaaauagatt

SEQ ID NO: 108 - DNA, 21 bp, Artificial Sequence
  Description of Artificial Sequence Synthetic oligonucleotide
ucuauuuuuugagcgccaatt

SEQ ID NO: 109 - DNA, 21 bp, Artificial Sequence
  Description of Artificial Sequence Synthetic oligonucleotide
uccaccaauucccguuggutt

SEQ ID NO: 110 - DNA, 21 bp, Artificial Sequence
  Description of Artificial Sequence Synthetic oligonucleotide
accaacgggaauugguggatt

SEQ ID NO: 111 - DNA, 21 bp, Artificial Sequence
  Description of Artificial Sequence Synthetic oligonucleotide
aaacaauaguuccugcaactt

SEQ ID NO: 112 - DNA, 21 bp, Artificial Sequence
  Description of Artificial Sequence Synthetic oligonucleotide
guugcaggaacuauuguuutt

SEQ ID NO: 113 - DNA, 21 bp, Artificial Sequence
  Description of Artificial Sequence Synthetic oligonucleotide
uucuguucauggugugagutt

SEQ ID NO: 114 - DNA, 21 bp, Artificial Sequence
  Description of Artificial Sequence Synthetic oligonucleotide
acucacaccaugaacagaatt

SEQ ID NO: 115 - DNA, 21 bp, Artificial Sequence
  Description of Artificial Sequence Synthetic oligonucleotide
agcauugcaaaccucaauatt

SEQ ID NO: 116 - DNA, 21 bp, Artificial Sequence
  Description of Artificial Sequence Synthetic oligonucleotide
uauugagguuugcaaugcutt

SEQ ID NO: 117 - DNA, 21 bp, Artificial Sequence
  Description of Artificial Sequence Synthetic oligonucleotide
gccucucauuuuaccggactt

SEQ ID NO: 118 - DNA, 21 bp, Artificial Sequence
  Description of Artificial Sequence Synthetic oligonucleotide
guccgguaaaaugagaggctt

SEQ ID NO: 119 - DNA, 21 bp, Artificial Sequence
  Description of Artificial Sequence Synthetic oligonucleotide
cagcaucccuuucucaacatt

SEQ ID NO: 120 - DNA, 21 bp, Artificial Sequence
  Description of Artificial Sequence Synthetic oligonucleotide
uguugagaaagggaugcugtt

SEQ ID NO: 121 - DNA, 21 bp, Artificial Sequence
  Description of Artificial Sequence Synthetic oligonucleotide
gagaucauauagacaaucatt

SEQ ID NO: 122 - DNA, 21 bp, Artificial Sequence
  Description of Artificial Sequence Synthetic oligonucleotide
ugauugucuauaugaucuctt

SEQ ID NO: 123 - DNA, 21 bp, Artificial Sequence
  Description of Artificial Sequence Synthetic oligonucleotide
ggcuguaugaaaauacccutt

SEQ ID NO: 124 - DNA, 21 bp, Artificial Sequence
  Description of Artificial Sequence Synthetic oligonucleotide
aggguauuuucauacagcctt

SEQ ID NO: 125 - DNA, 21 bp, Artificial Sequence
  Description of Artificial Sequence Synthetic oligonucleotide
acgauucauuccuuuuggatt

SEQ ID NO: 126 - DNA, 21 bp, Artificial Sequence
  Description of Artificial Sequence Synthetic oligonucleotide
uccaaaaggaaugaaucgutt

SEQ ID NO: 127 - DNA, 21 bp, Artificial Sequence
  Description of Artificial Sequence Synthetic oligonucleotide
ugggaaaugaccugggauutt

SEQ ID NO: 128 - DNA, 21 bp, Artificial Sequence
  Description of Artificial Sequence Synthetic oligonucleotide
aaucccaggucauuucccatt

SEQ ID NO: 129 - DNA, 21 bp, Artificial Sequence
  Description of Artificial Sequence Synthetic oligonucleotide
cccagguaaagagacgaautt

SEQ ID NO: 130 - DNA, 21 bp, Artificial Sequence
  Description of Artificial Sequence Synthetic oligonucleotide
auucgucucuuuaccugggtt

SEQ ID NO: 131 - DNA, 21 bp, Artificial Sequence
  Description of Artificial Sequence Synthetic oligonucleotide
cagcaucccuuucucaacatt

SEQ ID NO: 132 - DNA, 21 bp, Artificial Sequence
  Description of Artificial Sequence Synthetic oligonucleotide
uguugagaaagggaugcugtt

SEQ ID NO: 133 - DNA, 21 bp, Artificial Sequence
  Description of Artificial Sequence Synthetic oligonucleotide
cagguaaagagacgaaugatt

SEQ ID NO: 134 - DNA, 21 bp, Artificial Sequence
  Description of Artificial Sequence Synthetic oligonucleotide
ucauucgucucuuuaccugtt

SEQ ID NO: 135 - DNA, 21 bp, Artificial Sequence
  Description of Artificial Sequence Synthetic oligonucleotide
aauaacuugcuuaacuacatt

SEQ ID NO: 136 - DNA, 21 bp, Artificial Sequence
  Description of Artificial Sequence Synthetic oligonucleotide
uguaguuaagcaaguuauutt

SEQ ID NO: 137 - DNA, 21 bp, Artificial Sequence
  Description of Artificial Sequence Synthetic oligonucleotide
cuguaugaaaaccuuacugtt

SEQ ID NO: 138 - DNA, 21 bp, Artificial Sequence
  Description of Artificial Sequence Synthetic oligonucleotide
caguaagguuuucauacagtt

SEQ ID NO: 139 - DNA, 21 bp, Artificial Sequence
  Description of Artificial Sequence Synthetic oligonucleotide
gcucuguuccagacucaactt

SEQ ID NO: 140 - DNA, 21 bp, Artificial Sequence
  Description of Artificial Sequence Synthetic oligonucleotide
guugagucuggaacagagctt

SEQ ID NO: 141 - DNA, 21 bp, Artificial Sequence
  Description of Artificial Sequence Synthetic oligonucleotide
ggcucaguaagcaaugcgctt

SEQ ID NO: 142 - DNA, 21 bp, Artificial Sequence
  Description of Artificial Sequence Synthetic oligonucleotide
gcgcauugcuuacugagcctt

SEQ ID NO: 143 - DNA, 21 bp, Artificial Sequence
  Description of Artificial Sequence Synthetic oligonucleotide
gagaucauauagacaaucatt

SEQ ID NO: 144 - DNA, 21 bp, Artificial Sequence
  Description of Artificial Sequence Synthetic oligonucleotide
ugauugucuauaugaucuctt

SEQ ID NO: 145 - DNA, 21 bp, Artificial Sequence
  Description of Artificial Sequence Synthetic oligonucleotide
aggaucagaagccuauuuutt

SEQ ID NO: 146 - DNA, 21 bp, Artificial Sequence
  Description of Artificial Sequence Synthetic oligonucleotide
aaaauaggcuucugauccutt

SEQ ID NO: 147 - DNA, 21 bp, Artificial Sequence
  Description of Artificial Sequence Synthetic oligonucleotide
cagcaugccgcuaucgaaatt

SEQ ID NO: 148 - DNA, 21 bp, Artificial Sequence
  Description of Artificial Sequence Synthetic oligonucleotide
uuucgauagcggcaugcugtt

SEQ ID NO: 149 - DNA, 21 bp, Artificial Sequence
  Description of Artificial Sequence Synthetic oligonucleotide
uguuauaugcaggauaugatt

SEQ ID NO: 150 - DNA, 21 bp, Artificial Sequence
  Description of Artificial Sequence Synthetic oligonucleotide
ucauauccugcauauaacatt

SEQ ID NO: 151 - DNA, 21 bp, Artificial Sequence
  Description of Artificial Sequence Synthetic oligonucleotide
cgcuaucgaaaaugucuuctt

SEQ ID NO: 152 - DNA, 21 bp, Artificial Sequence
  Description of Artificial Sequence Synthetic oligonucleotide
gaagacauuuucgauagcgtt

SEQ ID NO: 153 - DNA, 21 bp, Artificial Sequence
  Description of Artificial Sequence Synthetic oligonucleotide
gguggagaucauauagacatt

SEQ ID NO: 154 - DNA, 21 bp, Artificial Sequence
  Description of Artificial Sequence Synthetic oligonucleotide
ugucuauaugaucuccacctt

SEQ ID NO: 155 - DNA, 21 bp, Artificial Sequence
  Description of Artificial Sequence Synthetic oligonucleotide
uuggcgcucaaaaaauagatt

SEQ ID NO: 156 - DNA, 21 bp, Artificial Sequence
  Description of Artificial Sequence Synthetic oligonucleotide
ucuauuuuuugagcgccaatt

SEQ ID NO: 157 - DNA, 21 bp, Artificial Sequence
  Description of Artificial Sequence Synthetic oligonucleotide
ucauuuuaccggacacuaatt

SEQ ID NO: 158 - DNA, 21 bp, Artificial Sequence
  Description of Artificial Sequence Synthetic oligonucleotide
uuaguguccgguaaaaugatt

SEQ ID NO: 159 - DNA, 21 bp, Artificial Sequence
  Description of Artificial Sequence Synthetic oligonucleotide
caucaucgauaaaauucgatt

SEQ ID NO: 160 - DNA, 21 bp, Artificial Sequence
  Description of Artificial Sequence Synthetic oligonucleotide
ucgaauuuuaucgaugaugtt

SEQ ID NO: 161 - DNA, 21 bp, Artificial Sequence
  Description of Artificial Sequence Synthetic oligonucleotide
ccagguaaagagacgaaugtt

SEQ ID NO: 162 - DNA, 21 bp, Artificial Sequence
  Description of Artificial Sequence Synthetic oligonucleotide
cauucgucucuuuaccuggtt

SEQ ID NO: 163 - DNA, 21 bp, Artificial Sequence
  Description of Artificial Sequence Synthetic oligonucleotide
caggcuucagguaucuuautt

SEQ ID NO: 164 - DNA, 21 bp, Artificial Sequence
  Description of Artificial Sequence Synthetic oligonucleotide
auaagauaccugaagccugtt

SEQ ID NO: 165 - DNA, 21 bp, Artificial Sequence
  Description of Artificial Sequence Synthetic oligonucleotide
uuuccaaaaggcucaguaatt

SEQ ID NO: 166 - DNA, 21 bp, Artificial Sequence
  Description of Artificial Sequence Synthetic oligonucleotide
uuacugagccuuuuggaaatt

SEQ ID NO: 167 - DNA, 21 bp, Artificial Sequence
  Description of Artificial Sequence Synthetic oligonucleotide
cacacauuaaucugauuuutt

SEQ ID NO: 168 - DNA, 21 bp, Artificial Sequence
  Description of Artificial Sequence Synthetic oligonucleotide
aaaaucagauuaaugugugtt

SEQ ID NO: 169 - DNA, 21 bp, Artificial Sequence
  Description of Artificial Sequence Synthetic oligonucleotide
ggcuguaugaaaauacccutt

SEQ ID NO: 170 - DNA, 21 bp, Artificial Sequence
  Description of Artificial Sequence Synthetic oligonucleotide
aggguauuuucauacagcctt

SEQ ID NO: 171 - DNA, 21 bp, Artificial Sequence
  Description of Artificial Sequence Synthetic oligonucleotide
cagguuucaggaacuuacatt

SEQ ID NO: 172 - DNA, 21 bp, Artificial Sequence
  Description of Artificial Sequence Synthetic oligonucleotide
uguaaguuccugaaaccugtt

SEQ ID NO: 173 - DNA, 21 bp, Artificial Sequence
  Description of Artificial Sequence Synthetic oligonucleotide
gaaauuagaaugaccuacatt

SEQ ID NO: 174 - DNA, 21 bp, Artificial Sequence
  Description of Artificial Sequence Synthetic oligonucleotide
uguaggucauucuaauuuctt

SEQ ID NO: 175 - DNA, 21 bp, Artificial Sequence
  Description of Artificial Sequence Synthetic oligonucleotide
ccaagcagcgaagacuuuutt

SEQ ID NO: 176 - DNA, 21 bp, Artificial Sequence
  Description of Artificial Sequence Synthetic oligonucleotide
aaaagucuucgcugcuuggtt

SEQ ID NO: 177 - DNA, 21 bp, Artificial Sequence
  Description of Artificial Sequence Synthetic oligonucleotide
uccaccaauucccguuggutt

SEQ ID NO: 178 - DNA, 21 bp, Artificial Sequence
  Description of Artificial Sequence Synthetic oligonucleotide
accaacgggaauugguggatt

SEQ ID NO: 179 - DNA, 21 bp, Artificial Sequence
  Description of Artificial Sequence Synthetic oligonucleotide
ccaacaaucuuggcgcucatt

SEQ ID NO: 180 - DNA, 21 bp, Artificial Sequence
  Description of Artificial Sequence Synthetic oligonucleotide
ugagcgccaagauuguuggtt

SEQ ID NO: 181 - DNA, 21 bp, Artificial Sequence
  Description of Artificial Sequence Synthetic oligonucleotide
cucaguaagcaaugcgcagtt

SEQ ID NO: 182 - DNA, 21 bp, Artificial Sequence
  Description of Artificial Sequence Synthetic oligonucleotide
cugcgcauugcuuacugagtt

SEQ ID NO: 183 - DNA, 21 bp, Artificial Sequence
  Description of Artificial Sequence Synthetic oligonucleotide
ucucaaugggacuguauautt

SEQ ID NO: 184 - DNA, 21 bp, Artificial Sequence
  Description of Artificial Sequence Synthetic oligonucleotide
auauacagucccauugagatt

SEQ ID NO: 185 - DNA, 21 bp, Artificial Sequence
  Description of Artificial Sequence Synthetic oligonucleotide
aaaaagaagauuucaucgatt

SEQ ID NO: 186 - DNA, 21 bp, Artificial Sequence
  Description of Artificial Sequence Synthetic oligonucleotide
ucgaugaaaucuucuuuuutt

SEQ ID NO: 187 - DNA, 21 bp, Artificial Sequence
  Description of Artificial Sequence Synthetic oligonucleotide
gaacuggcagcgguuuuautt

SEQ ID NO: 188 - DNA, 21 bp, Artificial Sequence
  Description of Artificial Sequence Synthetic oligonucleotide
auaaaaccgcugccaguuctt

SEQ ID NO: 189 - DNA, 21 bp, Artificial Sequence
  Description of Artificial Sequence Synthetic oligonucleotide
gcucuguuccagacucaactt

SEQ ID NO: 190 - DNA, 21 bp, Artificial Sequence
  Description of Artificial Sequence Synthetic oligonucleotide
guugagucuggaacagagctt

SEQ ID NO: 191 - DNA, 21 bp, Artificial Sequence
  Description of Artificial Sequence Synthetic oligonucleotide
caccaauucccguugguuctt

SEQ ID NO: 192 - DNA, 21 bp, Artificial Sequence
  Description of Artificial Sequence Synthetic oligonucleotide
gaaccaacgggaauuggugtt

SEQ ID NO: 193 - DNA, 21 bp, Artificial Sequence
  Description of Artificial Sequence Synthetic oligonucleotide
cgcuaucgaaaaugucuuctt

SEQ ID NO: 194 - DNA, 21 bp, Artificial Sequence
  Description of Artificial Sequence Synthetic oligonucleotide
gaagacauuuucgauagcgtt

SEQ ID NO: 195 - DNA, 21 bp, Artificial Sequence
  Description of Artificial Sequence Synthetic oligonucleotide
agcaugccgcuaucgaaaatt

SEQ ID NO: 196 - DNA, 21 bp, Artificial Sequence
  Description of Artificial Sequence Synthetic oligonucleotide
uuuucgauagcggcaugcutt

SEQ ID NO: 197 - DNA, 21 bp, Artificial Sequence
  Description of Artificial Sequence Synthetic oligonucleotide
cucaacuuggaggaucaugtt

SEQ ID NO: 198 - DNA, 21 bp, Artificial Sequence
  Description of Artificial Sequence Synthetic oligonucleotide
caugauccuccaaguugagtt

SEQ ID NO: 199 - DNA, 21 bp, Artificial Sequence
  Description of Artificial Sequence Synthetic oligonucleotide
ccagauguaagcucuccuctt

SEQ ID NO: 200 - DNA, 21 bp, Artificial Sequence
  Description of Artificial Sequence Synthetic oligonucleotide
gaggagagcuuacaucuggtt

SEQ ID NO: 201 - DNA, 21 bp, Artificial Sequence
  Description of Artificial Sequence Synthetic oligonucleotide
agugagaguugguuacucatt

SEQ ID NO: 202 - DNA, 21 bp, Artificial Sequence
  Description of Artificial Sequence Synthetic oligonucleotide
ugaguaaccaacucucacutt

SEQ ID NO: 203 - DNA, 21 bp, Artificial Sequence
  Description of Artificial Sequence Synthetic oligonucleotide
gggcggcaagugauugcagtt

SEQ ID NO: 204 - DNA, 21 bp, Artificial Sequence
  Description of Artificial Sequence Synthetic oligonucleotide
cugcaaucacuugccgccctt

SEQ ID NO: 205 - DNA, 21 bp, Artificial Sequence
  Description of Artificial Sequence Synthetic oligonucleotide
ugugauggacuucuauaaatt

SEQ ID NO: 206 - DNA, 21 bp, Artificial Sequence
  Description of Artificial Sequence Synthetic oligonucleotide
uuuauagaaguccaucacatt

SEQ ID NO: 207 - DNA, 21 bp, Artificial Sequence
  Description of Artificial Sequence Synthetic oligonucleotide
ccaagcagcgaagacuuuutt

SEQ ID NO: 208 - DNA, 21 bp, Artificial Sequence
  Description of Artificial Sequence Synthetic oligonucleotide
aaaagucuucgcugcuuggtt

SEQ ID NO: 209 - DNA, 21 bp, Artificial Sequence
  Description of Artificial Sequence Synthetic oligonucleotide
aaaacaauaguuccugcaatt

SEQ ID NO: 210 - DNA, 21 bp, Artificial Sequence
  Description of Artificial Sequence Synthetic oligonucleotide
uugcaggaacuauuguuuutt

SEQ ID NO: 211 - DNA, 21 bp, Artificial Sequence
  Description of Artificial Sequence Synthetic oligonucleotide
ccgcuaucgaaaaugucuutt

SEQ ID NO: 212 - DNA, 21 bp, Artificial Sequence
  Description of Artificial Sequence Synthetic oligonucleotide
aagacauuuucgauagcggtt

SEQ ID NO: 213 - DNA, 21 bp, Artificial Sequence
  Description of Artificial Sequence Synthetic oligonucleotide
cagcaugccgcuaucgaaatt

SEQ ID NO: 214 - DNA, 21 bp, Artificial Sequence
  Description of Artificial Sequence Synthetic oligonucleotide
uuucgauagcggcaugcugtt

SEQ ID NO: 215 - DNA, 21 bp, Artificial Sequence
  Description of Artificial Sequence Synthetic oligonucleotide
cuggugugcucugaugaagtt

SEQ ID NO: 216 - DNA, 21 bp, Artificial Sequence
  Description of Artificial Sequence Synthetic oligonucleotide
cuucaucagagcacaccagtt

SEQ ID NO: 217 - DNA, 21 bp, Artificial Sequence
  Description of Artificial Sequence Synthetic oligonucleotide
acgcucaacauguuaggagtt

SEQ ID NO: 218 - DNA, 21 bp, Artificial Sequence
  Description of Artificial Sequence Synthetic oligonucleotide
cuccuaacauguugagcgutt

SEQ ID NO: 219 - DNA, 21 bp, Artificial Sequence
  Description of Artificial Sequence Synthetic oligonucleotide
ucccaacaaucuuggcgcutt

SEQ ID NO: 220 - DNA, 21 bp, Artificial Sequence
  Description of Artificial Sequence Synthetic oligonucleotide
agcgccaagauuguugggatt

SEQ ID NO: 221 - DNA, 21 bp, Artificial Sequence
  Description of Artificial Sequence Synthetic oligonucleotide
agacgaaugagaguccuugtt

SEQ ID NO: 222 - DNA, 21 bp, Artificial Sequence
  Description of Artificial Sequence Synthetic oligonucleotide
caaggacucucauucgucutt

SEQ ID NO: 223 - DNA, 21 bp, Artificial Sequence
  Description of Artificial Sequence Synthetic oligonucleotide
uaccggacacuaaacccaatt

SEQ ID NO: 224 - DNA, 21 bp, Artificial Sequence
  Description of Artificial Sequence Synthetic oligonucleotide
uuggguuuaguguccgguatt

SEQ ID NO: 225 - DNA, 21 bp, Artificial Sequence
  Description of Artificial Sequence Synthetic oligonucleotide
cugcaacguuaccacaacutt

SEQ ID NO: 226 - DNA, 21 bp, Artificial Sequence
  Description of Artificial Sequence Synthetic oligonucleotide
aguugugguaacguugcagtt

SEQ ID NO: 227 - DNA, 21 bp, Artificial Sequence
  Description of Artificial Sequence Synthetic oligonucleotide
ccagcaugccgcuaucgaatt

SEQ ID NO: 228 - DNA, 21 bp, Artificial Sequence
  Description of Artificial Sequence Synthetic oligonucleotide
uucgauagcggcaugcuggtt

SEQ ID NO: 229 - DNA, 21 bp, Artificial Sequence
  Description of Artificial Sequence Synthetic oligonucleotide
agcauugcaaaccucaauatt

SEQ ID NO: 230 - DNA, 21 bp, Artificial Sequence
  Description of Artificial Sequence Synthetic oligonucleotide
uauugagguuugcaaugcutt

SEQ ID NO: 231 - DNA, 21 bp, Artificial Sequence
  Description of Artificial Sequence Synthetic oligonucleotide
ucccaacaaucuuggcgcutt

SEQ ID NO: 232 - DNA, 21 bp, Artificial Sequence
  Description of Artificial Sequence Synthetic oligonucleotide
agcgccaagauuguugggatt

SEQ ID NO: 233 - DNA, 21 bp, Artificial Sequence
  Description of Artificial Sequence Synthetic oligonucleotide
ccaccaauucccguugguutt

SEQ ID NO: 234 - DNA, 21 bp, Artificial Sequence
  Description of Artificial Sequence Synthetic oligonucleotide
aaccaacgggaauugguggtt

SEQ ID NO: 235 - DNA, 21 bp, Artificial Sequence
  Description of Artificial Sequence Synthetic oligonucleotide
ucagaccuguugauagaugtt

SEQ ID NO: 236 - DNA, 21 bp, Artificial Sequence
  Description of Artificial Sequence Synthetic oligonucleotide
caucuaucaacaggucugatt

SEQ ID NO: 237 - DNA, 21 bp, Artificial Sequence
  Description of Artificial Sequence Synthetic oligonucleotide
uuaccggacacuaaacccatt

SEQ ID NO: 238 - DNA, 21 bp, Artificial Sequence
  Description of Artificial Sequence Synthetic oligonucleotide
uggguuuaguguccgguaatt

SEQ ID NO: 239 - DNA, 21 bp, Artificial Sequence
  Description of Artificial Sequence Synthetic oligonucleotide
cccaacaaucuuggcgcuctt

SEQ ID NO: 240 - DNA, 21 bp, Artificial Sequence
  Description of Artificial Sequence Synthetic oligonucleotide
gagcgccaagauuguugggtt

SEQ ID NO: 241 - DNA, 21 bp, Artificial Sequence
  Description of Artificial Sequence Synthetic oligonucleotide
uuucuaauggcuauucaagtt

SEQ ID NO: 242 - DNA, 21 bp, Artificial Sequence
  Description of Artificial Sequence Synthetic oligonucleotide
cuugaauagccauuagaaatt

SEQ ID NO: 243 - DNA, 21 bp, Artificial Sequence
  Description of Artificial Sequence Synthetic oligonucleotide
uuaaugucauuccaccaautt

SEQ ID NO: 244 - DNA, 21 bp, Artificial Sequence
  Description of Artificial Sequence Synthetic oligonucleotide
auugguggaaugacauuaatt

SEQ ID NO: 245 - DNA, 21 bp, Artificial Sequence
  Description of Artificial Sequence Synthetic oligonucleotide
ggcucaguaagcaaugcgctt

SEQ ID NO: 246 - DNA, 21 bp, Artificial Sequence
  Description of Artificial Sequence Synthetic oligonucleotide
gcgcauugcuuacugagcctt

SEQ ID NO: 247 - DNA, 21 bp, Artificial Sequence
  Description of Artificial Sequence Synthetic oligonucleotide
gucuuaacuuguggaagcutt

SEQ ID NO: 248 - DNA, 21 bp, Artificial Sequence
  Description of Artificial Sequence Synthetic oligonucleotide
agcuuccacaaguuaagactt

SEQ ID NO: 249 - DNA, 21 bp, Artificial Sequence
  Description of Artificial Sequence Synthetic oligonucleotide
ucauuuuaccggacacuaatt

SEQ ID NO: 250 - DNA, 21 bp, Artificial Sequence
  Description of Artificial Sequence Synthetic oligonucleotide
uuaguguccgguaaaaugatt

SEQ ID NO: 251 - DNA, 21 bp, Artificial Sequence
  Description of Artificial Sequence Synthetic oligonucleotide
agacgaaugagaguccuugtt

SEQ ID NO: 252 - DNA, 21 bp, Artificial Sequence
  Description of Artificial Sequence Synthetic oligonucleotide
caaggacucucauucgucutt

SEQ ID NO: 253 - DNA, 21 bp, Artificial Sequence
  Description of Artificial Sequence Synthetic oligonucleotide
acuguaaaaccuuguguggtt

SEQ ID NO: 254 - DNA, 21 bp, Artificial Sequence
  Description of Artificial Sequence Synthetic oligonucleotide
ccacacaagguuuuacagutt

SEQ ID NO: 255 - DNA, 21 bp, Artificial Sequence
  Description of Artificial Sequence Synthetic oligonucleotide
aaccucaauaggucgaccatt

SEQ ID NO: 256 - DNA, 21 bp, Artificial Sequence
  Description of Artificial Sequence Synthetic oligonucleotide
uggucgaccuauugagguutt

SEQ ID NO: 257 - DNA, 21 bp, Artificial Sequence
  Description of Artificial Sequence Synthetic oligonucleotide
caugcugaauaauaaucugtt

SEQ ID NO: 258 - DNA, 21 bp, Artificial Sequence
  Description of Artificial Sequence Synthetic oligonucleotide
cagauuauuauucagcaugtt

SEQ ID NO: 259 - DNA, 21 bp, Artificial Sequence
  Description of Artificial Sequence Synthetic oligonucleotide
ugcaaaccucaauaggucgtt

SEQ ID NO: 260 - DNA, 21 bp, Artificial Sequence
  Description of Artificial Sequence Synthetic oligonucleotide
cgaccuauugagguuugcatt

SEQ ID NO: 261 - DNA, 21 bp, Artificial Sequence
  Description of Artificial Sequence Synthetic oligonucleotide
ccaacaaucuuggcgcucatt

SEQ ID NO: 262 - DNA, 21 bp, Artificial Sequence
  Description of Artificial Sequence Synthetic oligonucleotide
ugagcgccaagauuguuggtt

SEQ ID NO: 263 - DNA, 21 bp, Artificial Sequence
  Description of Artificial Sequence Synthetic oligonucleotide
gguuucaggaacuuacacctt

SEQ ID NO: 264 - DNA, 21 bp, Artificial Sequence
  Description of Artificial Sequence Synthetic oligonucleotide
gguguaaguuccugaaacctt

SEQ ID NO: 265 - DNA, 21 bp, Artificial Sequence
  Description of Artificial Sequence Synthetic oligonucleotide
gguuucaggaacuuacacctt

SEQ ID NO: 266 - DNA, 21 bp, Artificial Sequence
  Description of Artificial Sequence Synthetic oligonucleotide
gguguaaguuccugaaacctt

SEQ ID NO: 267 - DNA, 21 bp, Artificial Sequence
  Description of Artificial Sequence Synthetic oligonucleotide
uagugaccagguuuucaggtt

SEQ ID NO: 268 - DNA, 21 bp, Artificial Sequence
  Description of Artificial Sequence Synthetic oligonucleotide
ccugaaaaccuggucacuatt

SEQ ID NO: 269 - DNA, 21 bp, Artificial Sequence
  Description of Artificial Sequence Synthetic oligonucleotide
cugcaacguuaccacaacutt

SEQ ID NO: 270 - DNA, 21 bp, Artificial Sequence
  Description of Artificial Sequence Synthetic oligonucleotide
aguugugguaacguugcagtt

SEQ ID NO: 271 - DNA, 21 bp, Artificial Sequence
  Description of Artificial Sequence Synthetic oligonucleotide
agcaugccgcuaucgaaaatt

SEQ ID NO: 272 - DNA, 21 bp, Artificial Sequence
  Description of Artificial Sequence Synthetic oligonucleotide
uuuucgauagcggcaugcutt

SEQ ID NO: 273 - DNA, 21 bp, Artificial Sequence
  Description of Artificial Sequence Synthetic oligonucleotide
ugcaacguuaccacaacuctt

SEQ ID NO: 274 - DNA, 21 bp, Artificial Sequence
  Description of Artificial Sequence Synthetic oligonucleotide
gaguugugguaacguugcatt

SEQ ID NO: 275 - DNA, 21 bp, Artificial Sequence
  Description of Artificial Sequence Synthetic oligonucleotide
ugaaccugaaguguuauautt

SEQ ID NO: 276 - DNA, 21 bp, Artificial Sequence
  Description of Artificial Sequence Synthetic oligonucleotide
auauaacacuucagguucatt

SEQ ID NO: 277 - DNA, 21 bp, Artificial Sequence
  Description of Artificial Sequence Synthetic oligonucleotide
caccaauucccguugguuctt

SEQ ID NO: 278 - DNA, 21 bp, Artificial Sequence
  Description of Artificial Sequence Synthetic oligonucleotide
gaaccaacgggaauuggugtt

SEQ ID NO: 279 - DNA, 21 bp, Artificial Sequence
  Description of Artificial Sequence Synthetic oligonucleotide
ccagguaaagagacgaaugtt

SEQ ID NO: 280 - DNA, 21 bp, Artificial Sequence
  Description of Artificial Sequence Synthetic oligonucleotide
cauucgucucuuuaccuggtt

SEQ ID NO: 281 - DNA, 21 bp, Artificial Sequence
  Description of Artificial Sequence Synthetic oligonucleotide
cucucaaugggacuguauatt

SEQ ID NO: 282 - DNA, 21 bp, Artificial Sequence
  Description of Artificial Sequence Synthetic oligonucleotide
uauacagucccauugagagtt

SEQ ID NO: 283 - DNA, 21 bp, Artificial Sequence
  Description of Artificial Sequence Synthetic oligonucleotide
uggcgcucaaaaaauagaatt

SEQ ID NO: 284 - DNA, 21 bp, Artificial Sequence
  Description of Artificial Sequence Synthetic oligonucleotide
uucuauuuuuugagcgccatt

SEQ ID NO: 285 - DNA, 21 bp, Artificial Sequence
  Description of Artificial Sequence Synthetic oligonucleotide
auacccuccucaaauaacutt

SEQ ID NO: 286 - DNA, 21 bp, Artificial Sequence
  Description of Artificial Sequence Synthetic oligonucleotide
aguuauuugaggaggguautt

SEQ ID NO: 287 - DNA, 21 bp, Artificial Sequence
  Description of Artificial Sequence Synthetic oligonucleotide
gggcggcaagugauugcagtt

SEQ ID NO: 288 - DNA, 21 bp, Artificial Sequence
  Description of Artificial Sequence Synthetic oligonucleotide
cugcaaucacuugccgccctt

SEQ ID NO: 289 - DNA, 21 bp, Artificial Sequence
  Description of Artificial Sequence Synthetic oligonucleotide
ugcuuaacuacauauagautt

SEQ ID NO: 290 - DNA, 21 bp, Artificial Sequence
  Description of Artificial Sequence Synthetic oligonucleotide
aucuauauguaguuaagcatt

SEQ ID NO: 291 - DNA, 21 bp, Artificial Sequence
  Description of Artificial Sequence Synthetic oligonucleotide
auuccaccaauucccguugtt

SEQ ID NO: 292 - DNA, 21 bp, Artificial Sequence
  Description of Artificial Sequence Synthetic oligonucleotide
caacgggaauugguggaautt

SEQ ID NO: 293 - DNA, 21 bp, Artificial Sequence
  Description of Artificial Sequence Synthetic oligonucleotide
accucaauaggucgaccagtt

SEQ ID NO: 294 - DNA, 21 bp, Artificial Sequence
  Description of Artificial Sequence Synthetic oligonucleotide
cuggucgaccuauugaggutt

SEQ ID NO: 295 - DNA, 21 bp, Artificial Sequence
  Description of Artificial Sequence Synthetic oligonucleotide
guucauggugugaguaccutt

SEQ ID NO: 296 - DNA, 21 bp, Artificial Sequence
  Description of Artificial Sequence Synthetic oligonucleotide
agguacucacaccaugaactt

SEQ ID NO: 297 - DNA, 21 bp, Artificial Sequence
  Description of Artificial Sequence Synthetic oligonucleotide
ccucucauuuuaccggacatt

SEQ ID NO: 298 - DNA, 21 bp, Artificial Sequence
  Description of Artificial Sequence Synthetic oligonucleotide
uguccgguaaaaugagaggtt

SEQ ID NO: 299 - DNA, 21 bp, Artificial Sequence
  Description of Artificial Sequence Synthetic oligonucleotide
agccucucauuuuaccggatt

SEQ ID NO: 300 - DNA, 21 bp, Artificial Sequence
  Description of Artificial Sequence Synthetic oligonucleotide
uccgguaaaaugagaggcutt

SEQ ID NO: 301 - DNA, 21 bp, Artificial Sequence
  Description of Artificial Sequence Synthetic oligonucleotide
ucaaugggacuguauauggtt

SEQ ID NO: 302 - DNA, 21 bp, Artificial Sequence
  Description of Artificial Sequence Synthetic oligonucleotide
ccauauacagucccauugatt

SEQ ID NO: 303 - DNA, 21 bp, Artificial Sequence
  Description of Artificial Sequence Synthetic oligonucleotide
caggcuucagguaucuuautt

SEQ ID NO: 304 - DNA, 21 bp, Artificial Sequence
  Description of Artificial Sequence Synthetic oligonucleotide
auaagauaccugaagccugtt

SEQ ID NO: 305 - DNA, 21 bp, Artificial Sequence
  Description of Artificial Sequence Synthetic oligonucleotide
auucagcaggccacuacagtt

SEQ ID NO: 306 - DNA, 21 bp, Artificial Sequence
  Description of Artificial Sequence Synthetic oligonucleotide
cuguaguggccugcugaautt

SEQ ID NO: 307 - DNA, 21 bp, Artificial Sequence
  Description of Artificial Sequence Synthetic oligonucleotide
cccaacaaucuuggcgcuctt

SEQ ID NO: 308 - DNA, 21 bp, Artificial Sequence
  Description of Artificial Sequence Synthetic oligonucleotide
gagcgccaagauuguugggtt

SEQ ID NO: 309 - DNA, 21 bp, Artificial Sequence
  Description of Artificial Sequence Synthetic oligonucleotide
augagaccagauguaagcutt

SEQ ID NO: 310 - DNA, 21 bp, Artificial Sequence
  Description of Artificial Sequence Synthetic oligonucleotide
agcuuacaucuggucucautt

SEQ ID NO: 311 - DNA, 21 bp, Artificial Sequence
  Description of Artificial Sequence Synthetic oligonucleotide
caugcugaauaauaaucugtt

SEQ ID NO: 312 - DNA, 21 bp, Artificial Sequence
  Description of Artificial Sequence Synthetic oligonucleotide
cagauuauuauucagcaugtt

SEQ ID NO: 313 - DNA, 21 bp, Artificial Sequence
  Description of Artificial Sequence Synthetic oligonucleotide
acuggcagcgguuuuaucatt

SEQ ID NO: 314 - DNA, 21 bp, Artificial Sequence
  Description of Artificial Sequence Synthetic oligonucleotide
ugauaaaaccgcugccagutt

SEQ ID NO: 315 - DNA, 21 bp, Artificial Sequence
  Description of Artificial Sequence Synthetic oligonucleotide
aucugguuuugucaagccctt

SEQ ID NO: 316 - DNA, 21 bp, Artificial Sequence
  Description of Artificial Sequence Synthetic oligonucleotide
gggcuugacaaaaccagautt

SEQ ID NO: 317 - DNA, 21 bp, Artificial Sequence
  Description of Artificial Sequence Synthetic oligonucleotide
ugagaguugguuacucacatt

SEQ ID NO: 318 - DNA, 21 bp, Artificial Sequence
  Description of Artificial Sequence Synthetic oligonucleotide
ugugaguaaccaacucucatt

SEQ ID NO: 319 - DNA, 21 bp, Artificial Sequence
  Description of Artificial Sequence Synthetic oligonucleotide
ccaccaauucccguugguutt

SEQ ID NO: 320 - DNA, 21 bp, Artificial Sequence
  Description of Artificial Sequence Synthetic oligonucleotide
aaccaacgggaauugguggtt

SEQ ID NO: 321 - DNA, 21 bp, Artificial Sequence
  Description of Artificial Sequence Synthetic oligonucleotide
aaacugggcacaguuuacutt

SEQ ID NO: 322 - DNA, 21 bp, Artificial Sequence
  Description of Artificial Sequence Synthetic oligonucleotide
aguaaacugugcccaguuutt

SEQ ID NO: 323 - DNA, 21 bp, Artificial Sequence
  Description of Artificial Sequence Synthetic oligonucleotide
guucauggugugaguaccutt

SEQ ID NO: 324 - DNA, 21 bp, Artificial Sequence
  Description of Artificial Sequence Synthetic oligonucleotide
agguacucacaccaugaactt

SEQ ID NO: 325 - DNA, 21 bp, Artificial Sequence
  Description of Artificial Sequence Synthetic oligonucleotide
auacccuccucaaauaacutt

SEQ ID NO: 326 - DNA, 21 bp, Artificial Sequence
  Description of Artificial Sequence Synthetic oligonucleotide
aguuauuugaggaggguautt

SEQ ID NO: 327 - DNA, 21 bp, Artificial Sequence
  Description of Artificial Sequence Synthetic oligonucleotide
uuaccggacacuaaacccatt

SEQ ID NO: 328 - DNA, 21 bp, Artificial Sequence
  Description of Artificial Sequence Synthetic oligonucleotide
uggguuuaguguccgguaatt

SEQ ID NO: 329 - DNA, 21 bp, Artificial Sequence
  Description of Artificial Sequence Synthetic oligonucleotide
acuuacaccuggaugaccatt

SEQ ID NO: 330 - DNA, 21 bp, Artificial Sequence
  Description of Artificial Sequence Synthetic oligonucleotide
uggucauccagguguaagutt

SEQ ID NO: 331 - DNA, 21 bp, Artificial Sequence
  Description of Artificial Sequence Synthetic oligonucleotide
cucaguaagcaaugcgcagtt

SEQ ID NO: 332 - DNA, 21 bp, Artificial Sequence
  Description of Artificial Sequence Synthetic oligonucleotide
cugcgcauugcuuacugagtt

SEQ ID NO: 333 - DNA, 21 bp, Artificial Sequence
  Description of Artificial Sequence Synthetic oligonucleotide
uuugacauuuugcaggauutt

SEQ ID NO: 334 - DNA, 21 bp, Artificial Sequence
  Description of Artificial Sequence Synthetic oligonucleotide
aauccugcaaaaugucaaatt

SEQ ID NO: 335 - DNA, 21 bp, Artificial Sequence
  Description of Artificial Sequence Synthetic oligonucleotide
ucagaccuguugauagaugtt

SEQ ID NO: 336 - DNA, 21 bp, Artificial Sequence
  Description of Artificial Sequence Synthetic oligonucleotide
caucuaucaacaggucugatt

SEQ ID NO: 337 - DNA, 21 bp, Artificial Sequence
  Description of Artificial Sequence Synthetic oligonucleotide
auucagcaggccacuacagtt

SEQ ID NO: 338 - DNA, 21 bp, Artificial Sequence
  Description of Artificial Sequence Synthetic oligonucleotide
cuguaguggccugcugaautt

SEQ ID NO: 339 - DNA, 21 bp, Artificial Sequence
  Description of Artificial Sequence Synthetic oligonucleotide
auaguuccugcaacguuactt

SEQ ID NO: 340 - DNA, 21 bp, Artificial Sequence
  Description of Artificial Sequence Synthetic oligonucleotide
guaacguugcaggaacuautt

SEQ ID NO: 341 - DNA, 21 bp, Artificial Sequence
  Description of Artificial Sequence Synthetic oligonucleotide
ugcaacguuaccacaacuctt

SEQ ID NO: 342 - DNA, 21 bp, Artificial Sequence
  Description of Artificial Sequence Synthetic oligonucleotide
gaguugugguaacguugcatt

SEQ ID NO: 343 - DNA, 21 bp, Artificial Sequence
  Description of Artificial Sequence Synthetic oligonucleotide
uaguuuuuuauucaugcugtt

SEQ ID NO: 344 - DNA, 21 bp, Artificial Sequence
  Description of Artificial Sequence Synthetic oligonucleotide
cagcaugaauaaaaaacuatt

SEQ ID NO: 345 - DNA, 21 bp, Artificial Sequence
  Description of Artificial Sequence Synthetic oligonucleotide
ugggaaaugaccugggauutt

SEQ ID NO: 346 - DNA, 21 bp, Artificial Sequence
  Description of Artificial Sequence Synthetic oligonucleotide
aaucccaggucauuucccatt

SEQ ID NO: 347 - DNA, 21 bp, Artificial Sequence
  Description of Artificial Sequence Synthetic oligonucleotide
uuugacauuuugcaggauutt

SEQ ID NO: 348 - DNA, 21 bp, Artificial Sequence
  Description of Artificial Sequence Synthetic oligonucleotide
aauccugcaaaaugucaaatt

SEQ ID NO: 349 - DNA, 21 bp, Artificial Sequence
  Description of Artificial Sequence Synthetic oligonucleotide
acgcucaacauguuaggagtt

SEQ ID NO: 350 - DNA, 21 bp, Artificial Sequence
  Description of Artificial Sequence Synthetic oligonucleotide
cuccuaacauguugagcgutt

SEQ ID NO: 351 - DNA, 21 bp, Artificial Sequence
  Description of Artificial Sequence Synthetic oligonucleotide
ugcuguucugguauuaccatt

SEQ ID NO: 352 - DNA, 21 bp, Artificial Sequence
  Description of Artificial Sequence Synthetic oligonucleotide
ugguaauaccagaacagcatt

SEQ ID NO: 353 - DNA, 21 bp, Artificial Sequence
  Description of Artificial Sequence Synthetic oligonucleotide
cccagguaaagagacgaautt

SEQ ID NO: 354 - DNA, 21 bp, Artificial Sequence
  Description of Artificial Sequence Synthetic oligonucleotide
auucgucucuuuaccugggtt

SEQ ID NO: 355 - DNA, 21 bp, Artificial Sequence
  Description of Artificial Sequence Synthetic oligonucleotide
ugcaaaccucaauaggucgtt

SEQ ID NO: 356 - DNA, 21 bp, Artificial Sequence
  Description of Artificial Sequence Synthetic oligonucleotide
cgaccuauugagguuugcatt

SEQ ID NO: 357 - DNA, 21 bp, Artificial Sequence
  Description of Artificial Sequence Synthetic oligonucleotide
gccucucauuuuaccggactt

SEQ ID NO: 358 - DNA, 21 bp, Artificial Sequence
  Description of Artificial Sequence Synthetic oligonucleotide
guccgguaaaaugagaggctt

SEQ ID NO: 359 - DNA, 21 bp, Artificial Sequence
  Description of Artificial Sequence Synthetic oligonucleotide
ugcuguucugguauuaccatt

SEQ ID NO: 360 - DNA, 21 bp, Artificial Sequence
  Description of Artificial Sequence Synthetic oligonucleotide
ugguaauaccagaacagcatt

SEQ ID NO: 361 - DNA, 21 bp, Artificial Sequence
  Description of Artificial Sequence Synthetic oligonucleotide
gaacuggcagcgguuuuautt

SEQ ID NO: 362 - DNA, 21 bp, Artificial Sequence
  Description of Artificial Sequence Synthetic oligonucleotide
auaaaaccgcugccaguuctt

SEQ ID NO: 363 - DNA, 21 bp, Artificial Sequence
  Description of Artificial Sequence Synthetic oligonucleotide
ccuauguauguguuaucugtt

SEQ ID NO: 364 - DNA, 21 bp, Artificial Sequence
  Description of Artificial Sequence Synthetic oligonucleotide
cagauaacacauacauaggtt

SEQ ID NO: 365 - DNA, 21 bp, Artificial Sequence
  Description of Artificial Sequence Synthetic oligonucleotide
agaagauuucaucgaacuctt

SEQ ID NO: 366 - DNA, 21 bp, Artificial Sequence
  Description of Artificial Sequence Synthetic oligonucleotide
gaguucgaugaaaucuucutt

SEQ ID NO: 367 - DNA, 21 bp, Artificial Sequence
  Description of Artificial Sequence Synthetic oligonucleotide
cucugaacuucccuggucgtt

SEQ ID NO: 368 - DNA, 21 bp, Artificial Sequence
  Description of Artificial Sequence Synthetic oligonucleotide
cgaccagggaaguucagagtt

SEQ ID NO: 369 - DNA, 21 bp, Artificial Sequence
  Description of Artificial Sequence Synthetic oligonucleotide
cuggugugcucugaugaagtt

SEQ ID NO: 370 - DNA, 21 bp, Artificial Sequence
  Description of Artificial Sequence Synthetic oligonucleotide
cuucaucagagcacaccagtt

SEQ ID NO: 371 - DNA, 21 bp, Artificial Sequence
  Description of Artificial Sequence Synthetic oligonucleotide
cucaacuuggaggaucaugtt

SEQ ID NO: 372 - DNA, 21 bp, Artificial Sequence
  Description of Artificial Sequence Synthetic oligonucleotide
caugauccuccaaguugagtt

SEQ ID NO: 373 - DNA, 21 bp, Artificial Sequence
  Description of Artificial Sequence Synthetic oligonucleotide
agccucucauuuuaccggatt

SEQ ID NO: 374 - DNA, 21 bp, Artificial Sequence
  Description of Artificial Sequence Synthetic oligonucleotide
uccgguaaaaugagaggcutt

SEQ ID NO: 375 - DNA, 21 bp, Artificial Sequence
  Description of Artificial Sequence Synthetic oligonucleotide
auaguuccugcaacguuactt

SEQ ID NO: 376 - DNA, 21 bp, Artificial Sequence
  Description of Artificial Sequence Synthetic oligonucleotide
guaacguugcaggaacuautt

SEQ ID NO: 377 - DNA, 21 bp, Artificial Sequence
  Description of Artificial Sequence Synthetic oligonucleotide
aacaauaguuccugcaacgtt

SEQ ID NO: 378 - DNA, 21 bp, Artificial Sequence
  Description of Artificial Sequence Synthetic oligonucleotide
cguugcaggaacuauuguutt

SEQ ID NO: 379 - DNA, 21 bp, Artificial Sequence
  Description of Artificial Sequence Synthetic oligonucleotide
aucugguuuugucaagccctt

SEQ ID NO: 380 - DNA, 21 bp, Artificial Sequence
  Description of Artificial Sequence Synthetic oligonucleotide
gggcuugacaaaaccagautt

SEQ ID NO: 381 - DNA, 21 bp, Artificial Sequence
  Description of Artificial Sequence Synthetic oligonucleotide
acuguaaaaccuuguguggtt

SEQ ID NO: 382 - DNA, 21 bp, Artificial Sequence
  Description of Artificial Sequence Synthetic oligonucleotide
ccacacaagguuuuacagutt

SEQ ID NO: 383 - DNA, 21 bp, Artificial Sequence
  Description of Artificial Sequence Synthetic oligonucleotide
aacucuuggauucuaugcatt

SEQ ID NO: 384 - DNA, 21 bp, Artificial Sequence
  Description of Artificial Sequence Synthetic oligonucleotide
ugcauagaauccaagaguutt

SEQ ID NO: 385 - DNA, 21 bp, Artificial Sequence
  Description of Artificial Sequence Synthetic oligonucleotide
uagugaccagguuuucaggtt

SEQ ID NO: 386 - DNA, 21 bp, Artificial Sequence
  Description of Artificial Sequence Synthetic oligonucleotide
ccugaaaaccuggucacuatt

SEQ ID NO: 387 - DNA, 21 bp, Artificial Sequence
  Description of Artificial Sequence Synthetic oligonucleotide
aaccucaauaggucgaccatt

SEQ ID NO: 388 - DNA, 21 bp, Artificial Sequence
  Description of Artificial Sequence Synthetic oligonucleotide
uggucgaccuauugagguutt

SEQ ID NO: 389 - DNA, 21 bp, Artificial Sequence
  Description of Artificial Sequence Synthetic oligonucleotide
ccucucauuuuaccggacatt

SEQ ID NO: 390 - DNA, 21 bp, Artificial Sequence
  Description of Artificial Sequence Synthetic oligonucleotide
uguccgguaaaaugagaggtt

SEQ ID NO: 391 - DNA, 21 bp, Artificial Sequence
  Description of Artificial Sequence Synthetic oligonucleotide
ugaccaaaugacccuacugtt

SEQ ID NO: 392 - DNA, 21 bp, Artificial Sequence
  Description of Artificial Sequence Synthetic oligonucleotide
caguagggucauuuggucatt

SEQ ID NO: 393 - DNA, 21 bp, Artificial Sequence
  Description of Artificial Sequence Synthetic oligonucleotide
agaucagaccuguugauagtt

SEQ ID NO: 394 - DNA, 21 bp, Artificial Sequence
  Description of Artificial Sequence Synthetic oligonucleotide
cuaucaacaggucugaucutt

SEQ ID NO: 395 - DNA, 21 bp, Artificial Sequence
  Description of Artificial Sequence Synthetic oligonucleotide
cagguuucaggaacuuacatt

SEQ ID NO: 396 - DNA, 21 bp, Artificial Sequence
  Description of Artificial Sequence Synthetic oligonucleotide
uguaaguuccugaaaccugtt

SEQ ID NO: 397 - DNA, 21 bp, Artificial Sequence
  Description of Artificial Sequence Synthetic oligonucleotide
uaguuuuuuauucaugcugtt

SEQ ID NO: 398 - DNA, 21 bp, Artificial Sequence
  Description of Artificial Sequence Synthetic oligonucleotide
cagcaugaauaaaaaacuatt

SEQ ID NO: 399 - DNA, 21 bp, Artificial Sequence
  Description of Artificial Sequence Synthetic oligonucleotide
ugugauggacuucuauaaatt

SEQ ID NO: 400 - DNA, 21 bp, Artificial Sequence
  Description of Artificial Sequence Synthetic oligonucleotide
uuuauagaaguccaucacatt

SEQ ID NO: 401 - DNA, 21 bp, Artificial Sequence
  Description of Artificial Sequence Synthetic oligonucleotide
uggcgcucaaaaaauagaatt

SEQ ID NO: 402 - DNA, 21 bp, Artificial Sequence
  Description of Artificial Sequence Synthetic oligonucleotide
uucuauuuuuugagcgccatt

SEQ ID NO: 403 - DNA, 21 bp, Artificial Sequence
  Description of Artificial Sequence Synthetic oligonucleotide
aacaauaguuccugcaacgtt

SEQ ID NO: 404 - DNA, 21 bp, Artificial Sequence
  Description of Artificial Sequence Synthetic oligonucleotide
cguugcaggaacuauuguutt

SEQ ID NO: 405 - DNA, 21 bp, Artificial Sequence
  Description of Artificial Sequence Synthetic oligonucleotide
ugaaccugaaguguuauautt

SEQ ID NO: 406 - DNA, 21 bp, Artificial Sequence
  Description of Artificial Sequence Synthetic oligonucleotide
auauaacacuucagguucatt

SEQ ID NO: 407 - DNA, 21 bp, Artificial Sequence
  Description of Artificial Sequence Synthetic oligonucleotide
cucucaaugggacuguauatt

SEQ ID NO: 408 - DNA, 21 bp, Artificial Sequence
  Description of Artificial Sequence Synthetic oligonucleotide
uauacagucccauugagagtt

SEQ ID NO: 409 - DNA, 21 bp, Artificial Sequence
  Description of Artificial Sequence Synthetic oligonucleotide
ucuguaugaaaaccuuacutt

SEQ ID NO: 410 - DNA, 21 bp, Artificial Sequence
  Description of Artificial Sequence Synthetic oligonucleotide
aguaagguuuucauacagatt

SEQ ID NO: 411 - DNA, 21 bp, Artificial Sequence
  Description of Artificial Sequence Synthetic oligonucleotide
augccgcuaucgaaaaugutt

SEQ ID NO: 412 - DNA, 21 bp, Artificial Sequence
  Description of Artificial Sequence Synthetic oligonucleotide
acauuuucgauagcggcautt

SEQ ID NO: 413 - DNA, 21 bp, Artificial Sequence
  Description of Artificial Sequence Synthetic oligonucleotide
uaguuccugcaacguuacctt

SEQ ID NO: 414 - DNA, 21 bp, Artificial Sequence
  Description of Artificial Sequence Synthetic oligonucleotide
gguaacguugcaggaacuatt

SEQ ID NO: 415 - DNA, 21 bp, Artificial Sequence
  Description of Artificial Sequence Synthetic oligonucleotide
aacaaucuuggcgcucaaatt

SEQ ID NO: 416 - DNA, 21 bp, Artificial Sequence
  Description of Artificial Sequence Synthetic oligonucleotide
uuugagcgccaagauuguutt

SEQ ID NO: 417 - DNA, 21 bp, Artificial Sequence
  Description of Artificial Sequence Synthetic oligonucleotide
aaaccucaauaggucgacctt

SEQ ID NO: 418 - DNA, 21 bp, Artificial Sequence
  Description of Artificial Sequence Synthetic oligonucleotide
ggucgaccuauugagguuutt

SEQ ID NO: 419 - DNA, 21 bp, Artificial Sequence
  Description of Artificial Sequence Synthetic oligonucleotide
uuuccaaaaggcucaguaatt

SEQ ID NO: 420 - DNA, 21 bp, Artificial Sequence
  Description of Artificial Sequence Synthetic oligonucleotide
uuacugagccuuuuggaaatt

SEQ ID NO: 421 - DNA, 21 bp, Artificial Sequence
  Description of Artificial Sequence Synthetic oligonucleotide
ucucaaugggacuguauautt

SEQ ID NO: 422 - DNA, 21 bp, Artificial Sequence
  Description of Artificial Sequence Synthetic oligonucleotide
auauacagucccauugagatt

SEQ ID NO: 423 - DNA, 21 bp, Artificial Sequence
  Description of Artificial Sequence Synthetic oligonucleotide
aacaaucuuggcgcucaaatt

SEQ ID NO: 424 - DNA, 21 bp, Artificial Sequence
  Description of Artificial Sequence Synthetic oligonucleotide
uuugagcgccaagauuguutt

SEQ ID NO: 425 - DNA, 21 bp, Artificial Sequence
  Description of Artificial Sequence Synthetic oligonucleotide
aacucuuggauucuaugcatt

SEQ ID NO: 426 - DNA, 21 bp, Artificial Sequence
  Description of Artificial Sequence Synthetic oligonucleotide
ugcauagaauccaagaguutt

SEQ ID NO: 427 - DNA, 21 bp, Artificial Sequence
  Description of Artificial Sequence Synthetic oligonucleotide
aaaaagaagauuucaucgatt

SEQ ID NO: 428 - DNA, 21 bp, Artificial Sequence
  Description of Artificial Sequence Synthetic oligonucleotide
ucgaugaaaucuucuuuuutt

SEQ ID NO: 429 - DNA, 21 bp, Artificial Sequence
  Description of Artificial Sequence Synthetic oligonucleotide
cauauagacaaucaagugctt

SEQ ID NO: 430 - DNA, 21 bp, Artificial Sequence
  Description of Artificial Sequence Synthetic oligonucleotide
gcacuugauugucuauaugtt

SEQ ID NO: 431 - DNA, 21 bp, Artificial Sequence
  Description of Artificial Sequence Synthetic oligonucleotide
acuuacaccuggaugaccatt

SEQ ID NO: 432 - DNA, 21 bp, Artificial Sequence
  Description of Artificial Sequence Synthetic oligonucleotide
uggucauccagguguaagutt

SEQ ID NO: 433 - DNA, 21 bp, Artificial Sequence
  Description of Artificial Sequence Synthetic oligonucleotide
uuuaccggacacuaaaccctt

SEQ ID NO: 434 - DNA, 21 bp, Artificial Sequence
  Description of Artificial Sequence Synthetic oligonucleotide
ggguuuaguguccgguaaatt

SEQ ID NO: 435 - DNA, 21 bp, Artificial Sequence
  Description of Artificial Sequence Synthetic oligonucleotide
cagguaaagagacgaaugatt

SEQ ID NO: 436 - DNA, 21 bp, Artificial Sequence
  Description of Artificial Sequence Synthetic oligonucleotide
ucauucgucucuuuaccugtt

SEQ ID NO: 437 - DNA, 21 bp, Artificial Sequence
  Description of Artificial Sequence Synthetic oligonucleotide
cccagcaugccgcuaucgatt

SEQ ID NO: 438 - DNA, 21 bp, Artificial Sequence
  Description of Artificial Sequence Synthetic oligonucleotide
ucgauagcggcaugcugggtt

SEQ ID NO: 439 - DNA, 21 bp, Artificial Sequence
  Description of Artificial Sequence Synthetic oligonucleotide
cccagcaugccgcuaucgatt

SEQ ID NO: 440 - DNA, 21 bp, Artificial Sequence
  Description of Artificial Sequence Synthetic oligonucleotide
ucgauagcggcaugcugggtt

SEQ ID NO: 441 - DNA, 21 bp, Artificial Sequence
  Description of Artificial Sequence Synthetic oligonucleotide
ggaggacagauguaccacutt

SEQ ID NO: 442 - DNA, 21 bp, Artificial Sequence
  Description of Artificial Sequence Synthetic oligonucleotide
agugguacaucuguccucctt

SEQ ID NO: 443 - DNA, 21 bp, Artificial Sequence
  Description of Artificial Sequence Synthetic oligonucleotide
cauguacgaccaauguaaatt

SEQ ID NO: 444 - DNA, 21 bp, Artificial Sequence
  Description of Artificial Sequence Synthetic oligonucleotide
uuuacauuggucguacaugtt

SEQ ID NO: 445 - DNA, 21 bp, Artificial Sequence
  Description of Artificial Sequence Synthetic oligonucleotide
uaguuccugcaacguuacctt

SEQ ID NO: 446 - DNA, 21 bp, Artificial Sequence
  Description of Artificial Sequence Synthetic oligonucleotide
gguaacguugcaggaacuatt

SEQ ID NO: 447 - DNA, 21 bp, Artificial Sequence
  Description of Artificial Sequence Synthetic oligonucleotide
aacuuacaccuggaugacctt

SEQ ID NO: 448 - DNA, 21 bp, Artificial Sequence
  Description of Artificial Sequence Synthetic oligonucleotide
ggucauccagguguaaguutt

SEQ ID NO: 449 - DNA, 21 bp, Artificial Sequence
  Description of Artificial Sequence Synthetic oligonucleotide
aaacaauaguuccugcaactt

SEQ ID NO: 450 - DNA, 21 bp, Artificial Sequence
  Description of Artificial Sequence Synthetic oligonucleotide
guugcaggaacuauuguuutt

SEQ ID NO: 451 - DNA, 21 bp, Artificial Sequence
  Description of Artificial Sequence Synthetic oligonucleotide
uuuuaccggacacuaaacctt

SEQ ID NO: 452 - DNA, 21 bp, Artificial Sequence
  Description of Artificial Sequence Synthetic oligonucleotide
gguuuaguguccgguaaaatt

SEQ ID NO: 453 - DNA, 21 bp, Artificial Sequence
  Description of Artificial Sequence Synthetic oligonucleotide
ugagaguugguuacucacatt

SEQ ID NO: 454 - DNA, 21 bp, Artificial Sequence
  Description of Artificial Sequence Synthetic oligonucleotide
ugugaguaaccaacucucatt

SEQ ID NO: 455 - DNA, 21 bp, Artificial Sequence
  Description of Artificial Sequence Synthetic oligonucleotide
acaauaguuccugcaacgutt

SEQ ID NO: 456 - DNA, 21 bp, Artificial Sequence
  Description of Artificial Sequence Synthetic oligonucleotide
acguugcaggaacuauugutt

SEQ ID NO: 457 - DNA, 21 bp, Artificial Sequence
  Description of Artificial Sequence Synthetic oligonucleotide
gguccacccaggauuagugtt

SEQ ID NO: 458 - DNA, 21 bp, Artificial Sequence
  Description of Artificial Sequence Synthetic oligonucleotide
cacuaauccuggguggacctt

SEQ ID NO: 459 - DNA, 21 bp, Artificial Sequence
  Description of Artificial Sequence Synthetic oligonucleotide
uguuauaugcaggauaugatt

SEQ ID NO: 460 - DNA, 21 bp, Artificial Sequence
  Description of Artificial Sequence Synthetic oligonucleotide
ucauauccugcauauaacatt

SEQ ID NO: 461 - DNA, 21 bp, Artificial Sequence
  Description of Artificial Sequence Synthetic oligonucleotide
augagaccagauguaagcutt

SEQ ID NO: 462 - DNA, 21 bp, Artificial Sequence
  Description of Artificial Sequence Synthetic oligonucleotide
agcuuacaucuggucucautt

SEQ ID NO: 463 - DNA, 21 bp, Artificial Sequence
  Description of Artificial Sequence Synthetic oligonucleotide
accucaauaggucgaccagtt

SEQ ID NO: 464 - DNA, 21 bp, Artificial Sequence
  Description of Artificial Sequence Synthetic oligonucleotide
cuggucgaccuauugaggutt

SEQ ID NO: 465 - DNA, 21 bp, Artificial Sequence
  Description of Artificial Sequence Synthetic oligonucleotide
agaagauuucaucgaacuctt

SEQ ID NO: 466 - DNA, 21 bp, Artificial Sequence
  Description of Artificial Sequence Synthetic oligonucleotide
gaguucgaugaaaucuucutt

SEQ ID NO: 467 - DNA, 21 bp, Artificial Sequence
  Description of Artificial Sequence Synthetic oligonucleotide
aaaccucaauaggucgacctt

SEQ ID NO: 468 - DNA, 21 bp, Artificial Sequence
  Description of Artificial Sequence Synthetic oligonucleotide
ggucgaccuauugagguuutt

SEQ ID NO: 469 - DNA, 21 bp, Artificial Sequence
  Description of Artificial Sequence Synthetic oligonucleotide
uuccaccaauucccguuggtt

SEQ ID NO: 470 - DNA, 21 bp, Artificial Sequence
  Description of Artificial Sequence Synthetic oligonucleotide
ccaacgggaauugguggaatt

SEQ ID NO: 471 - DNA, 21 bp, Artificial Sequence
  Description of Artificial Sequence Synthetic oligonucleotide
ugaccaaaugacccuacugtt

SEQ ID NO: 472 - DNA, 21 bp, Artificial Sequence
  Description of Artificial Sequence Synthetic oligonucleotide
caguagggucauuuggucatt

SEQ ID NO: 473 - DNA, 21 bp, Artificial Sequence
  Description of Artificial Sequence Synthetic oligonucleotide
auuccaccaauucccguugtt

SEQ ID NO: 474 - DNA, 21 bp, Artificial Sequence
  Description of Artificial Sequence Synthetic oligonucleotide
caacgggaauugguggaautt

SEQ ID NO: 475 - DNA, 21 bp, Artificial Sequence
  Description of Artificial Sequence Synthetic oligonucleotide
ugguccacccaggauuagutt

SEQ ID NO: 476 - DNA, 21 bp, Artificial Sequence
  Description of Artificial Sequence Synthetic oligonucleotide
acuaauccuggguggaccatt

SEQ ID NO: 477 - DNA, 21 bp, Artificial Sequence
  Description of Artificial Sequence Synthetic oligonucleotide
aggaauucagcaggccacutt

SEQ ID NO: 478 - DNA, 21 bp, Artificial Sequence
  Description of Artificial Sequence Synthetic oligonucleotide
aguggccugcugaauuccutt

SEQ ID NO: 479 - DNA, 21 bp, Artificial Sequence
  Description of Artificial Sequence Synthetic oligonucleotide
acuucccuggucgaacagutt

SEQ ID NO: 480 - DNA, 21 bp, Artificial Sequence
  Description of Artificial Sequence Synthetic oligonucleotide
acuguucgaccagggaagutt

SEQ ID NO: 481 - DNA, 21 bp, Artificial Sequence
  Description of Artificial Sequence Synthetic oligonucleotide
uuuuaccggacacuaaacctt

SEQ ID NO: 482 - DNA, 21 bp, Artificial Sequence
  Description of Artificial Sequence Synthetic oligonucleotide
gguuuaguguccgguaaaatt

SEQ ID NO: 483 - DNA, 21 bp, Artificial Sequence
  Description of Artificial Sequence Synthetic oligonucleotide
aaauaacuugcuuaacuactt

SEQ ID NO: 484 - DNA, 21 bp, Artificial Sequence
  Description of Artificial Sequence Synthetic oligonucleotide
guaguuaagcaaguuauuutt

SEQ ID NO: 485 - DNA, 21 bp, Artificial Sequence
  Description of Artificial Sequence Synthetic oligonucleotide
aaggcucaguaagcaaugctt

SEQ ID NO: 486 - DNA, 21 bp, Artificial Sequence
  Description of Artificial Sequence Synthetic oligonucleotide
gcauugcuuacugagccuutt

SEQ ID NO: 487 - DNA, 21 bp, Artificial Sequence
  Description of Artificial Sequence Synthetic oligonucleotide
aggaauucagcaggccacutt

SEQ ID NO: 488 - DNA, 21 bp, Artificial Sequence
  Description of Artificial Sequence Synthetic oligonucleotide
aguggccugcugaauuccutt

SEQ ID NO: 489 - DNA, 21 bp, Artificial Sequence
  Description of Artificial Sequence Synthetic oligonucleotide
cucugaacuucccuggucgtt

SEQ ID NO: 490 - DNA, 21 bp, Artificial Sequence
  Description of Artificial Sequence Synthetic oligonucleotide
cgaccagggaaguucagagtt

SEQ ID NO: 491 - DNA, 21 bp, Artificial Sequence
  Description of Artificial Sequence Synthetic oligonucleotide
ucaaugggacuguauauggtt

SEQ ID NO: 492 - DNA, 21 bp, Artificial Sequence
  Description of Artificial Sequence Synthetic oligonucleotide
ccauauacagucccauugatt

SEQ ID NO: 493 - DNA, 21 bp, Artificial Sequence
  Description of Artificial Sequence Synthetic oligonucleotide
aaacugggcacaguuuacutt

SEQ ID NO: 494 - DNA, 21 bp, Artificial Sequence
  Description of Artificial Sequence Synthetic oligonucleotide
aguaaacugugcccaguuutt

SEQ ID NO: 495 - DNA, 21 bp, Artificial Sequence
  Description of Artificial Sequence Synthetic oligonucleotide
aagccucucauuuuaccggtt

SEQ ID NO: 496 - DNA, 21 bp, Artificial Sequence
  Description of Artificial Sequence Synthetic oligonucleotide
ccgguaaaaugagaggcuutt

SEQ ID NO: 497 - DNA, 21 bp, Artificial Sequence
  Description of Artificial Sequence Synthetic oligonucleotide
acuucccuggucgaacagutt

SEQ ID NO: 498 - DNA, 21 bp, Artificial Sequence
  Description of Artificial Sequence Synthetic oligonucleotide
acuguucgaccagggaagutt

SEQ ID NO: 499 - DNA, 21 bp, Artificial Sequence
  Description of Artificial Sequence Synthetic oligonucleotide
aacuuacaccuggaugacctt

SEQ ID NO: 500 - DNA, 21 bp, Artificial Sequence
  Description of Artificial Sequence Synthetic oligonucleotide
ggucauccagguguaaguutt

SEQ ID NO: 501 - DNA, 21 bp, Artificial Sequence
  Description of Artificial Sequence Synthetic oligonucleotide
ggaggacagauguaccacutt

SEQ ID NO: 502 - DNA, 21 bp, Artificial Sequence
  Description of Artificial Sequence Synthetic oligonucleotide
agugguacaucuguccucctt

SEQ ID NO: 503 - DNA, 21 bp, Artificial Sequence
  Description of Artificial Sequence Synthetic oligonucleotide
gguccacccaggauuagugtt

SEQ ID NO: 504 - DNA, 21 bp, Artificial Sequence
  Description of Artificial Sequence Synthetic oligonucleotide
cacuaauccuggguggacctt

SEQ ID NO: 505 - DNA, 21 bp, Artificial Sequence
  Description of Artificial Sequence Synthetic oligonucleotide
aaggcucaguaagcaaugctt

SEQ ID NO: 506 - DNA, 21 bp, Artificial Sequence
  Description of Artificial Sequence Synthetic oligonucleotide
gcauugcuuacugagccuutt

SEQ ID NO: 507 - DNA, 21 bp, Artificial Sequence
  Description of Artificial Sequence Synthetic oligonucleotide
uuccaccaauucccguuggtt

SEQ ID NO: 508 - DNA, 21 bp, Artificial Sequence
  Description of Artificial Sequence Synthetic oligonucleotide
ccaacgggaauugguggaatt

SEQ ID NO: 509 - DNA, 21 bp, Artificial Sequence
  Description of Artificial Sequence Synthetic oligonucleotide
uuuaccggacacuaaaccctt

SEQ ID NO: 510 - DNA, 21 bp, Artificial Sequence
  Description of Artificial Sequence Synthetic oligonucleotide
ggguuuaguguccgguaaatt

SEQ ID NO: 511 - DNA, 21 bp, Artificial Sequence
  Description of Artificial Sequence Synthetic oligonucleotide
ugguccacccaggauuagutt

SEQ ID NO: 512 - DNA, 21 bp, Artificial Sequence
  Description of Artificial Sequence Synthetic oligonucleotide
acuaauccuggguggaccatt

SEQ ID NO: 513 - DNA, 21 bp, Artificial Sequence
  Description of Artificial Sequence Synthetic oligonucleotide
aagccucucauuuuaccggtt

SEQ ID NO: 514 - DNA, 21 bp, Artificial Sequence
  Description of Artificial Sequence Synthetic oligonucleotide
ccgguaaaaugagaggcuutt

SEQ ID NO: 515 - DNA, 21 bp, Artificial Sequence
  Description of Artificial Sequence Synthetic oligonucleotide
aggcuuuucauuaaaugggtt

SEQ ID NO: 516 - DNA, 21 bp, Artificial Sequence
  Description of Artificial Sequence Synthetic oligonucleotide
cccauuuaaugaaaagccutt

SEQ ID NO: 517 - DNA, 21 bp, Artificial Sequence
  Description of Artificial Sequence Synthetic oligonucleotide
ugaacuaugcuugcucguutt

SEQ ID NO: 518 - DNA, 21 bp, Artificial Sequence
  Description of Artificial Sequence Synthetic oligonucleotide
aacgagcaagcauaguucatt

SEQ ID NO: 519 - DNA, 21 bp, Artificial Sequence
  Description of Artificial Sequence Synthetic oligonucleotide
augaauacagcaucccuuutt

SEQ ID NO: 520 - DNA, 21 bp, Artificial Sequence
  Description of Artificial Sequence Synthetic oligonucleotide
aaagggaugcuguauucautt

SEQ ID NO: 521 - DNA, 21 bp, Artificial Sequence
  Description of Artificial Sequence Synthetic oligonucleotide
uucucaggcagauuccaagtt

SEQ ID NO: 522 - DNA, 21 bp, Artificial Sequence
  Description of Artificial Sequence Synthetic oligonucleotide
cuuggaaucugccugagaatt

SEQ ID NO: 523 - DNA, 21 bp, Artificial Sequence
  Description of Artificial Sequence Synthetic oligonucleotide
aacauuaauuuccgugugatt

SEQ ID NO: 524 - DNA, 21 bp, Artificial Sequence
  Description of Artificial Sequence Synthetic oligonucleotide
ucacacggaaauuaauguutt

SEQ ID NO: 525 - DNA, 21 bp, Artificial Sequence
  Description of Artificial Sequence Synthetic oligonucleotide
gaacuaugcuugcucguuutt

SEQ ID NO: 526 - DNA, 21 bp, Artificial Sequence
  Description of Artificial Sequence Synthetic oligonucleotide
aaacgagcaagcauaguuctt

SEQ ID NO: 527 - DNA, 21 bp, Artificial Sequence
  Description of Artificial Sequence Synthetic oligonucleotide
uccuagacgcuaacauuaatt

SEQ ID NO: 528 - DNA, 21 bp, Artificial Sequence
  Description of Artificial Sequence Synthetic oligonucleotide
uuaauguuagcgucuaggatt

SEQ ID NO: 529 - DNA, 21 bp, Artificial Sequence
  Description of Artificial Sequence Synthetic oligonucleotide
uaaugucauuccaccaauutt

SEQ ID NO: 530 - DNA, 21 bp, Artificial Sequence
  Description of Artificial Sequence Synthetic oligonucleotide
aauugguggaaugacauuatt

SEQ ID NO: 531 - DNA, 21 bp, Artificial Sequence
  Description of Artificial Sequence Synthetic oligonucleotide
uuauuuuaccggacacuaatt

SEQ ID NO: 532 - DNA, 21 bp, Artificial Sequence
  Description of Artificial Sequence Synthetic oligonucleotide
uuaguguccgguaaaauaatt

SEQ ID NO: 533 - DNA, 21 bp, Artificial Sequence
  Description of Artificial Sequence Synthetic oligonucleotide
aacauuaauuuccgugugatt

SEQ ID NO: 534 - DNA, 21 bp, Artificial Sequence
  Description of Artificial Sequence Synthetic oligonucleotide
ucacacggaaauuaauguutt

SEQ ID NO: 535 - DNA, 21 bp, Artificial Sequence
  Description of Artificial Sequence Synthetic oligonucleotide
auaucaaagagcuaggaaatt

SEQ ID NO: 536 - DNA, 21 bp, Artificial Sequence
  Description of Artificial Sequence Synthetic oligonucleotide
uuuccuagcucuuugauautt

SEQ ID NO: 537 - DNA, 21 bp, Artificial Sequence
  Description of Artificial Sequence Synthetic oligonucleotide
gacgcuaacauuaauuucctt

SEQ ID NO: 538 - DNA, 21 bp, Artificial Sequence
  Description of Artificial Sequence Synthetic oligonucleotide
ggaaauuaauguuagcguctt

SEQ ID NO: 539 - DNA, 21 bp, Artificial Sequence
  Description of Artificial Sequence Synthetic oligonucleotide
uuccgugugaaaauggguctt

SEQ ID NO: 540 - DNA, 21 bp, Artificial Sequence
  Description of Artificial Sequence Synthetic oligonucleotide
gacccauuuucacacggaatt

SEQ ID NO: 541 - DNA, 21 bp, Artificial Sequence
  Description of Artificial Sequence Synthetic oligonucleotide
gugaacuaugcuugcucgutt

SEQ ID NO: 542 - DNA, 21 bp, Artificial Sequence
  Description of Artificial Sequence Synthetic oligonucleotide
acgagcaagcauaguucactt

SEQ ID NO: 543 - DNA, 21 bp, Artificial Sequence
  Description of Artificial Sequence Synthetic oligonucleotide
auaucaaagagcuaggaaatt

SEQ ID NO: 544 - DNA, 21 bp, Artificial Sequence
  Description of Artificial Sequence Synthetic oligonucleotide
uuuccuagcucuuugauautt

SEQ ID NO: 545 - DNA, 21 bp, Artificial Sequence
  Description of Artificial Sequence Synthetic oligonucleotide
uccuagacgcuaacauuaatt

SEQ ID NO: 546 - DNA, 21 bp, Artificial Sequence
  Description of Artificial Sequence Synthetic oligonucleotide
uuaauguuagcgucuaggatt

SEQ ID NO: 547 - DNA, 21 bp, Artificial Sequence
  Description of Artificial Sequence Synthetic oligonucleotide
ugcauguaugaccaauguatt

SEQ ID NO: 548 - DNA, 21 bp, Artificial Sequence
  Description of Artificial Sequence Synthetic oligonucleotide
uacauuggucauacaugcatt

SEQ ID NO: 549 - DNA, 21 bp, Artificial Sequence
  Description of Artificial Sequence Synthetic oligonucleotide
cccccugguagagacgaagtt

SEQ ID NO: 550 - DNA, 21 bp, Artificial Sequence
  Description of Artificial Sequence Synthetic oligonucleotide
cuuggaaucugccugagaatt

SEQ ID NO: 551 - DNA, 21 bp, Artificial Sequence
  Description of Artificial Sequence Synthetic oligonucleotide
uuuaucaugacauguuauatt

SEQ ID NO: 552 - DNA, 21 bp, Artificial Sequence
  Description of Artificial Sequence Synthetic oligonucleotide
uauaacaugucaugauaaatt

SEQ ID NO: 553 - DNA, 21 bp, Artificial Sequence
  Description of Artificial Sequence Synthetic oligonucleotide
aaccucaauaggucgaccatt

SEQ ID NO: 554 - DNA, 21 bp, Artificial Sequence
  Description of Artificial Sequence Synthetic oligonucleotide
uggucgaccuauugagguutt

SEQ ID NO: 555 - DNA, 21 bp, Artificial Sequence
  Description of Artificial Sequence Synthetic oligonucleotide
uuauccaaagccguuucactt

SEQ ID NO: 556 - DNA, 21 bp, Artificial Sequence
  Description of Artificial Sequence Synthetic oligonucleotide
gugaaacggcuuuggauaatt

SEQ ID NO: 557 - DNA, 21 bp, Artificial Sequence
  Description of Artificial Sequence Synthetic oligonucleotide
uuccgugugaaaauggguctt

SEQ ID NO: 558 - DNA, 21 bp, Artificial Sequence
  Description of Artificial Sequence Synthetic oligonucleotide
gacccauuuucacacggaatt

SEQ ID NO: 559 - DNA, 21 bp, Artificial Sequence
  Description of Artificial Sequence Synthetic oligonucleotide
accucaauaggucgaccagtt

SEQ ID NO: 560 - DNA, 21 bp, Artificial Sequence
  Description of Artificial Sequence Synthetic oligonucleotide
cuggucgaccuauugaggutt

SEQ ID NO: 561 - DNA, 21 bp, Artificial Sequence
  Description of Artificial Sequence Synthetic oligonucleotide
gaacuaugcuugcucguuutt

SEQ ID NO: 562 - DNA, 21 bp, Artificial Sequence
  Description of Artificial Sequence Synthetic oligonucleotide
aaacgagcaagcauaguuctt

SEQ ID NO: 563 - DNA, 21 bp, Artificial Sequence
  Description of Artificial Sequence Synthetic oligonucleotide
agacgcuaacauuaauuuctt

SEQ ID NO: 564 - DNA, 21 bp, Artificial Sequence
  Description of Artificial Sequence Synthetic oligonucleotide
gaaauuaauguuagcgucutt

SEQ ID NO: 565 - DNA, 21 bp, Artificial Sequence
  Description of Artificial Sequence Synthetic oligonucleotide
uuuaucaugacauguuauatt

SEQ ID NO: 566 - DNA, 21 bp, Artificial Sequence
  Description of Artificial Sequence Synthetic oligonucleotide
uauaacaugucaugauaaatt

SEQ ID NO: 567 - DNA, 21 bp, Artificial Sequence
  Description of Artificial Sequence Synthetic oligonucleotide
gugaacuaugcuugcucgutt

SEQ ID NO: 568 - DNA, 21 bp, Artificial Sequence
  Description of Artificial Sequence Synthetic oligonucleotide
acgagcaagcauaguucactt

SEQ ID NO: 569 - DNA, 21 bp, Artificial Sequence
  Description of Artificial Sequence Synthetic oligonucleotide
agacgcuaacauuaauuuctt

SEQ ID NO: 570 - DNA, 21 bp, Artificial Sequence
  Description of Artificial Sequence Synthetic oligonucleotide
gaaauuaauguuagcgucutt

SEQ ID NO: 571 - DNA, 21 bp, Artificial Sequence
  Description of Artificial Sequence Synthetic oligonucleotide
ccggacacuaaaccuaaaatt

SEQ ID NO: 572 - DNA, 21 bp, Artificial Sequence
  Description of Artificial Sequence Synthetic oligonucleotide
uuuuagguuuaguguccggtt

SEQ ID NO: 573 - DNA, 21 bp, Artificial Sequence
  Description of Artificial Sequence Synthetic oligonucleotide
ugcaaaccucaauaggucgtt

SEQ ID NO: 574 - DNA, 21 bp, Artificial Sequence
  Description of Artificial Sequence Synthetic oligonucleotide
cgaccuauugagguuugcatt

SEQ ID NO: 575 - DNA, 21 bp, Artificial Sequence
  Description of Artificial Sequence Synthetic oligonucleotide
cugaaaacuggaauaggugtt

SEQ ID NO: 576 - DNA, 21 bp, Artificial Sequence
  Description of Artificial Sequence Synthetic oligonucleotide
caccuauuccaguuuucagtt

SEQ ID NO: 577 - DNA, 21 bp, Artificial Sequence
  Description of Artificial Sequence Synthetic oligonucleotide
uguuauaugguuaaacccatt

SEQ ID NO: 578 - DNA, 21 bp, Artificial Sequence
  Description of Artificial Sequence Synthetic oligonucleotide
uggguuuaaccauauaacatt

SEQ ID NO: 579 - DNA, 21 bp, Artificial Sequence
  Description of Artificial Sequence Synthetic oligonucleotide
uguuauaugguuaaacccatt

SEQ ID NO: 580 - DNA, 21 bp, Artificial Sequence
  Description of Artificial Sequence Synthetic oligonucleotide
uggguuuaaccauauaacatt

SEQ ID NO: 581 - DNA, 21 bp, Artificial Sequence
  Description of Artificial Sequence Synthetic oligonucleotide
ugguuuaaauuggucucaatt

SEQ ID NO: 582 - DNA, 21 bp, Artificial Sequence
  Description of Artificial Sequence Synthetic oligonucleotide
uugagaccaauuuaaaccatt

SEQ ID NO: 583 - DNA, 21 bp, Artificial Sequence
  Description of Artificial Sequence Synthetic oligonucleotide
ccggacacuaaaccuaaaatt

SEQ ID NO: 584 - DNA, 21 bp, Artificial Sequence
  Description of Artificial Sequence Synthetic oligonucleotide
uuuuagguuuaguguccggtt

SEQ ID NO: 585 - DNA, 21 bp, Artificial Sequence
  Description of Artificial Sequence Synthetic oligonucleotide
uuaaugucauuccaccaautt

SEQ ID NO: 586 - DNA, 21 bp, Artificial Sequence
  Description of Artificial Sequence Synthetic oligonucleotide
auugguggaaugacauuaatt

SEQ ID NO: 587 - DNA, 21 bp, Artificial Sequence
  Description of Artificial Sequence Synthetic oligonucleotide
uguaaugguuuaaauuggutt

SEQ ID NO: 588 - DNA, 21 bp, Artificial Sequence
  Description of Artificial Sequence Synthetic oligonucleotide
accaauuuaaaccauuacatt

SEQ ID NO: 589 - DNA, 21 bp, Artificial Sequence
  Description of Artificial Sequence Synthetic oligonucleotide
ugguuuaaauuggucucaatt

SEQ ID NO: 590 - DNA, 21 bp, Artificial Sequence
  Description of Artificial Sequence Synthetic oligonucleotide
uugagaccaauuuaaaccatt

SEQ ID NO: 591 - DNA, 21 bp, Artificial Sequence
  Description of Artificial Sequence Synthetic oligonucleotide
uuuaauuacugguaggacatt

SEQ ID NO: 592 - DNA, 21 bp, Artificial Sequence
  Description of Artificial Sequence Synthetic oligonucleotide
uguccuaccaguaauuaaatt

SEQ ID NO: 593 - DNA, 21 bp, Artificial Sequence
  Description of Artificial Sequence Synthetic oligonucleotide
gacgcuaacauuaauuucctt

SEQ ID NO: 594 - DNA, 21 bp, Artificial Sequence
  Description of Artificial Sequence Synthetic oligonucleotide
ggaaauuaauguuagcguctt

SEQ ID NO: 595 - DNA, 21 bp, Artificial Sequence
  Description of Artificial Sequence Synthetic oligonucleotide
uuauuuuaccggacacuaatt

SEQ ID NO: 596 - DNA, 21 bp, Artificial Sequence
  Description of Artificial Sequence Synthetic oligonucleotide
uuaguguccgguaaaauaatt

SEQ ID NO: 597 - DNA, 21 bp, Artificial Sequence
  Description of Artificial Sequence Synthetic oligonucleotide
uuauccaaagccguuucactt

SEQ ID NO: 598 - DNA, 21 bp, Artificial Sequence
  Description of Artificial Sequence Synthetic oligonucleotide
gugaaacggcuuuggauaatt

SEQ ID NO: 599 - DNA, 21 bp, Artificial Sequence
  Description of Artificial Sequence Synthetic oligonucleotide
uuuaccggacacuaaaccutt

SEQ ID NO: 600 - DNA, 21 bp, Artificial Sequence
  Description of Artificial Sequence Synthetic oligonucleotide
agguuuaguguccgguaaatt

SEQ ID NO: 601 - DNA, 21 bp, Artificial Sequence
  Description of Artificial Sequence Synthetic oligonucleotide
uuuaauuacugguaggacatt

SEQ ID NO: 602 - DNA, 21 bp, Artificial Sequence
  Description of Artificial Sequence Synthetic oligonucleotide
uguccuaccaguaauuaaatt

SEQ ID NO: 603 - DNA, 21 bp, Artificial Sequence
  Description of Artificial Sequence Synthetic oligonucleotide
ugaacuaugcuugcucguutt

SEQ ID NO: 604 - DNA, 21 bp, Artificial Sequence
  Description of Artificial Sequence Synthetic oligonucleotide
aacgagcaagcauaguucatt

SEQ ID NO: 605 - DNA, 21 bp, Artificial Sequence
  Description of Artificial Sequence Synthetic oligonucleotide
gguuuaaauuggucucaaatt

SEQ ID NO: 606 - DNA, 21 bp, Artificial Sequence
  Description of Artificial Sequence Synthetic oligonucleotide
uuugagaccaauuuaaacctt

SEQ ID NO: 607 - DNA, 21 bp, Artificial Sequence
  Description of Artificial Sequence Synthetic oligonucleotide
gguuuaaauuggucucaaatt

SEQ ID NO: 608 - DNA, 21 bp, Artificial Sequence
  Description of Artificial Sequence Synthetic oligonucleotide
uuugagaccaauuuaaacctt

SEQ ID NO: 609 - DNA, 21 bp, Artificial Sequence
  Description of Artificial Sequence Synthetic oligonucleotide
ugcugaauaaccuguaguutt

SEQ ID NO: 610 - DNA, 21 bp, Artificial Sequence
  Description of Artificial Sequence Synthetic oligonucleotide
aacuacagguuauucagcatt

SEQ ID NO: 611 - DNA, 21 bp, Artificial Sequence
  Description of Artificial Sequence Synthetic oligonucleotide
aaaugggcaaaggcgauactt

SEQ ID NO: 612 - DNA, 21 bp, Artificial Sequence
  Description of Artificial Sequence Synthetic oligonucleotide
guaucgccuuugcccauuutt

SEQ ID NO: 613 - DNA, 21 bp, Artificial Sequence
  Description of Artificial Sequence Synthetic oligonucleotide
uguaaugguuuaaauuggutt

SEQ ID NO: 614 - DNA, 21 bp, Artificial Sequence
  Description of Artificial Sequence Synthetic oligonucleotide
accaauuuaaaccauuacatt

SEQ ID NO: 615 - DNA, 21 bp, Artificial Sequence
  Description of Artificial Sequence Synthetic oligonucleotide
augaauacagcaucccuuutt

SEQ ID NO: 616 - DNA, 21 bp, Artificial Sequence
  Description of Artificial Sequence Synthetic oligonucleotide
aaagggaugcuguauucautt

SEQ ID NO: 617 - DNA, 21 bp, Artificial Sequence
  Description of Artificial Sequence Synthetic oligonucleotide
uguuagucagccauuuacatt

SEQ ID NO: 618 - DNA, 21 bp, Artificial Sequence
  Description of Artificial Sequence Synthetic oligonucleotide
uguaaauggcugacuaacatt

SEQ ID NO: 619 - DNA, 21 bp, Artificial Sequence
  Description of Artificial Sequence Synthetic oligonucleotide
uuaaugucauuccaccaautt

SEQ ID NO: 620 - DNA, 21 bp, Artificial Sequence
  Description of Artificial Sequence Synthetic oligonucleotide
auugguggaaugacauuaatt

SEQ ID NO: 621 - DNA, 21 bp, Artificial Sequence
  Description of Artificial Sequence Synthetic oligonucleotide
guguggcuucauaccguuctt

SEQ ID NO: 622 - DNA, 21 bp, Artificial Sequence
  Description of Artificial Sequence Synthetic oligonucleotide
gaacgguaugaagccacactt

SEQ ID NO: 623 - DNA, 21 bp, Artificial Sequence
  Description of Artificial Sequence Synthetic oligonucleotide
guguggcuucauaccguuctt

SEQ ID NO: 624 - DNA, 21 bp, Artificial Sequence
  Description of Artificial Sequence Synthetic oligonucleotide
gaacgguaugaagccacactt

SEQ ID NO: 625 - DNA, 21 bp, Artificial Sequence
  Description of Artificial Sequence Synthetic oligonucleotide
uguuagucagccauuuacatt

SEQ ID NO: 626 - DNA, 21 bp, Artificial Sequence
  Description of Artificial Sequence Synthetic oligonucleotide
uguaaauggcugacuaacatt

SEQ ID NO: 627 - DNA, 21 bp, Artificial Sequence
  Description of Artificial Sequence Synthetic oligonucleotide
uguggcuucauaccguucctt

SEQ ID NO: 628 - DNA, 21 bp, Artificial Sequence
  Description of Artificial Sequence Synthetic oligonucleotide
ggaacgguaugaagccacatt

SEQ ID NO: 629 - DNA, 21 bp, Artificial Sequence
  Description of Artificial Sequence Synthetic oligonucleotide
ugcugaauaaccuguaguutt

SEQ ID NO: 630 - DNA, 21 bp, Artificial Sequence
  Description of Artificial Sequence Synthetic oligonucleotide
aacuacagguuauucagcatt

SEQ ID NO: 631 - DNA, 21 bp, Artificial Sequence
  Description of Artificial Sequence Synthetic oligonucleotide
uuuaccggacacuaaaccutt

SEQ ID NO: 632 - DNA, 21 bp, Artificial Sequence
  Description of Artificial Sequence Synthetic oligonucleotide
agguuuaguguccgguaaatt

SEQ ID NO: 633 - DNA, 21 bp, Artificial Sequence
  Description of Artificial Sequence Synthetic oligonucleotide
cugaaaacuggaauaggugtt

SEQ ID NO: 634 - DNA, 21 bp, Artificial Sequence
  Description of Artificial Sequence Synthetic oligonucleotide
caccuauuccaguuuucagtt

SEQ ID NO: 635 - DNA, 21 bp, Artificial Sequence
  Description of Artificial Sequence Synthetic oligonucleotide
aaaccucaauaggucgacctt

SEQ ID NO: 636 - DNA, 21 bp, Artificial Sequence
  Description of Artificial Sequence Synthetic oligonucleotide
ggucgaccuauugagguuutt

SEQ ID NO: 637 - DNA, 21 bp, Artificial Sequence
  Description of Artificial Sequence Synthetic oligonucleotide
aaccucaauaggucgaccatt

SEQ ID NO: 638 - DNA, 21 bp, Artificial Sequence
  Description of Artificial Sequence Synthetic oligonucleotide
uggucgaccuauugagguutt

SEQ ID NO: 639 - DNA, 21 bp, Artificial Sequence
  Description of Artificial Sequence Synthetic oligonucleotide
aguaaauguuagucagccatt

SEQ ID NO: 640 - DNA, 21 bp, Artificial Sequence
  Description of Artificial Sequence Synthetic oligonucleotide
uggcugacuaacauuuacutt

SEQ ID NO: 641 - DNA, 21 bp, Artificial Sequence
  Description of Artificial Sequence Synthetic oligonucleotide
ugcauguaugaccaauguatt

SEQ ID NO: 642 - DNA, 21 bp, Artificial Sequence
  Description of Artificial Sequence Synthetic oligonucleotide
uacauuggucauacaugcatt

SEQ ID NO: 643 - DNA, 21 bp, Artificial Sequence
  Description of Artificial Sequence Synthetic oligonucleotide
ugcaaaccucaauaggucgtt

SEQ ID NO: 644 - DNA, 21 bp, Artificial Sequence
  Description of Artificial Sequence Synthetic oligonucleotide
cgaccuauugagguuugcatt

SEQ ID NO: 645 - DNA, 21 bp, Artificial Sequence
  Description of Artificial Sequence Synthetic oligonucleotide
aaaccucaauaggucgacctt

SEQ ID NO: 646 - DNA, 21 bp, Artificial Sequence
  Description of Artificial Sequence Synthetic oligonucleotide
ggucgaccuauugagguuutt

SEQ ID NO: 647 - DNA, 21 bp, Artificial Sequence
  Description of Artificial Sequence Synthetic oligonucleotide
aguaaauguuagucagccatt

SEQ ID NO: 648 - DNA, 21 bp, Artificial Sequence
  Description of Artificial Sequence Synthetic oligonucleotide
uggcugacuaacauuuacutt

SEQ ID NO: 649 - DNA, 21 bp, Artificial Sequence
  Description of Artificial Sequence Synthetic oligonucleotide
ucuuauuuuaccggacacutt

SEQ ID NO: 650 - DNA, 21 bp, Artificial Sequence
  Description of Artificial Sequence Synthetic oligonucleotide
aguguccgguaaaauaagatt

SEQ ID NO: 651 - DNA, 21 bp, Artificial Sequence
  Description of Artificial Sequence Synthetic oligonucleotide
accucaauaggucgaccagtt

SEQ ID NO: 652 - DNA, 21 bp, Artificial Sequence
  Description of Artificial Sequence Synthetic oligonucleotide
cuggucgaccuauugaggutt

SEQ ID NO: 653 - DNA, 21 bp, Artificial Sequence
  Description of Artificial Sequence Synthetic oligonucleotide
aaaugggcaaaggcgauactt

SEQ ID NO: 654 - DNA, 21 bp, Artificial Sequence
  Description of Artificial Sequence Synthetic oligonucleotide
guaucgccuuugcccauuutt

SEQ ID NO: 655 - DNA, 21 bp, Artificial Sequence
  Description of Artificial Sequence Synthetic oligonucleotide
uguggcuucauaccguucctt

SEQ ID NO: 656 - DNA, 21 bp, Artificial Sequence
  Description of Artificial Sequence Synthetic oligonucleotide
ggaacgguaugaagccacatt

SEQ ID NO: 657 - DNA, 21 bp, Artificial Sequence
  Description of Artificial Sequence Synthetic oligonucleotide
ucuuauuuuaccggacacutt

SEQ ID NO: 658 - DNA, 21 bp, Artificial Sequence
  Description of Artificial Sequence Synthetic oligonucleotide
aguguccgguaaaauaagatt

SEQ ID NO: 659 - DNA, 6784 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 660 - DNA, 6614 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaaggtcagaaatctttcaagccctgcaggaccgtaaaatgcgcatgtgtccaacggaagcactggggcatgagtggggaaggaatagaaacagaaagaggttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 661 - DNA, 6517 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 662 - DNA, 6410 bp, Homo sapiens
cttctctcccagtgcgagagcgcggcggcggcagctgaagacccggccgcccagatgatgcggtggtgggggacctgccggcacgcgactccccccgggcccaaattgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 663 - DNA, 7286 bp, Homo sapiens
aggttatgtaagggtttgctttcaccccattcaaaaggtacctcttcctcttctcttgctccctctcgccctcattcttgtgcctatgcagacatttgagtagaggcgaatcactttcacttctgctggggaaattgcaacacgcttctttaaatggcagagagaaggagaaaacttagatcttctgataccaaatcactggaccttagaaggtcagaaatctttcaagccctgcaggaccgtaaaatgcgcatgtgtccaacggaagcactggggcatgagtggggaaggaatagaaacagaaagagggtaagagaagaaaaaagggaaagtggtgaaggcagggaggaaaattgcttagtgtgaatatgcacgcattcatttagttttcaaatccttgttgagcatgataaaattcccagcatcagacctcacatgttggtttccattaggatctgcctgggggaatatctgctgaatcagtggctctgagctgaactaggaaattcaccataattaggagagtcactgtatttctctccaaaaaaaaaaaagttatacccgagagacaggatcttctgatctgaaattttcttcacttctgaaattctctggtttgtgctcatcgttggtagctatttgttcatcaagagttgtgtagctggcttcttctgaaaaaaggaatctgcgtcatatctaagtcagatttcattctggtgctctcagagcagttagcccaggaaaggggccagcttctgtgacgactgctgcagaggcaggtgcagtttgtgtgccacagatattaactttgataagcacttaatgagtgccttctctgtgcgagaatggggaggaacaaaatgcagctcctaccctcctcgggctttagttgtaccttaataacaggaattttcatctgcctggctcctttcctcaaagaacaaagaagactttgcttcattaaagtgtctgagaaggaagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 664 - DNA, 4154 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaaaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 665 - DNA, 6787 bp, Homo sapiens
ggcgccgcctccacccgctccccgctcggtcccgctcgctcgcccaggccgggctgccctttcgcgtgtccgcgctctcttccctccgccgccgcctcctccattttgcgagctcgtgtctgtgacgggagcccgagtcaccgcctgcccgtcggggacggattctgtgggtggaaggagacgccgcagccggagcggccgaagcagctgggaccgggacggggcacgcgcgcccggaacctcgacccgcggagcccggcgcggggcggagggctggcttgtcagctgggcaatgggagactttcttaaataggggctctccccccacccatggagaaaggggcggctgtttacttcctttttttagaaaaaaaaaatatatttccctcctgctccttctgcgttcacaagctaagttgtttatctcggctgcggcgggaactgcggacggtggcgggcgagcggctcctctgccagagttgatattcactgatggactccaaagaatcattaactcctggtagagaagaaaaccccagcagtgtgcttgctcaggagaggggagatgtgatggacttctataaaaccctaagaggaggagctactgtgaaggtttctgcgtcttcaccctcactggctgtcgcttctcaatcagactccaagcagcgaagacttttggttgattttccaaaaggctcagtaagcaatgcgcagcagccagatctgtccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacagagaaggagtttccaaaaactcactctgatgtatcttcagaacagcaacatttgaagggccagactggcaccaacggtggcaatgtgaaattgtataccacagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaactcgaatgaggactgcaagcctctcattttaccggacactaaacccaaaattaaggataatggagatctggttttgtcaagccccagtaatgtaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttccgaaaattggaataggtgccaaggatctggagatgacaacttgacttctctggggactctgaacttccctggtcgaacagttttttctaatggctattcaagccccagcatgagaccagatgtaagctctcctccatccagctcctcaacagcaacaacaggaccacctcccaaactctgcctggtgtgctctgatgaagcttcaggatgtcattatggagtcttaacttgtggaagctgtaaagttttcttcaaaagagcagtggaaggtagacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctggtaacaaaacaatagttcctgcaacgttaccacaactcacccctaccctggtgtcactgttggaggttattgaacctgaagtgttatatgcaggatatgatagctctgttccagactcaacttggaggatcatgactacgctcaacatgttaggagggcggcaagtgattgcagcagtgaaatgggcaaaggcaataccaggtttcaggaacttacacctggatgaccaaatgaccctactgcagtactcctggatgtttcttatggcatttgctctggggtggagatcatatagacaatcaagtgcaaacctgctgtgttttgctcctgatctgattattaatgagcagagaatgactctaccctgcatgtacgaccaatgtaaacacatgctgtatgtttcctctgagttacacaggcttcaggtatcttatgaagagtatctctgtatgaaaaccttactgcttctctcttcagttcctaaggacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatctccttaactattgcttccaaacatttttggataagaccatgagtattgaattccccgagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactatcagtttgtcctgtagaggttttgttgttttattttttattgttttcatctgttgttttgttttaaatacgcactacatgtggtttatagagggccaagacttggcaacagaagcagttgagtcgtcatcacttttcagtgatgggagagtagatggtgaaatttattagttaatatatcccagaaattagaaaccttaatatgtggacgtaatctccacagtcaaagaaggatggcacctaaaccaccagtgcccaaagtctgtgtgatgaactttctcttcatactttttttcacagttggctggatgaaattttctagactttctgttggtgtatcccccccctgtatagttaggatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcaatcatctgtgtgaaaatgggttggtgcttctaacctgatggcacttagctatcagaagaccacaaaaattgactcaaatctccagtattcttgtcaaaaaaaaaaaaaaaaaagctcatattttgtatatatctgcttcagtggagaattatataggttgtgcaaattaacagtcctaactggtatagagcacctagtccagtgacctgctgggtaaactgtggatgatggttgcaaaagactaatttaaaaaataactaccaagaggccctgtctgtacctaacgccctatttttgcaatggctatatggcaagaaagctggtaaactatttgtctttcaggaccttttgaagtagtttgtataacttcttaaaagttgtgattccagataaccagctgtaacacagctgagagacttttaatcagacaaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgatggtctcagaaaccaaacagtttgctctaggggaagagggagatggagactggtcctgtgtgcagtgaaggttgctgaggctctgacccagtgagattacagaggaagttatcctctgcctcccattctgaccacccttctcattccaacagtgagtctgtcagcgcaggtttagtttactcaatctccccttgcactaaagtatgtaaagtatgtaaacaggagacaggaaggtggtgcttacatccttaaaggcaccatctaatagcgggttactttcacatacagccctcccccagcagttgaatgacaacagaagcttcagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaaacaatcatttgctttttgaatgctctctaaaagggaatgtaatattttaagatggtgtgtaacccggctggataaatttttggtgcctaagaaaactgcttgaatattcttatcaatgacagtgttaagtttcaaaaagagcttctaaaacgtagattatcattcctttatagaatgttatgtggttaaaaccagaaagcacatctcacacattaatctgattttcatcccaacaatcttggcgctcaaaaaatagaactcaatgagaaaaagaagattatgtgcacttcgttgtcaataataagtcaactgatgctcatcgacaactataggaggcttttcattaaatgggaaaagaagctgtgcccttttaggatacgtgggggaaaagaaagtcatcttaattatgtttaattgtggatttaagtgctatatggtggtgctgtttgaaagcagatttatttcctatgtatgtgttatctggccatcccaacccaaactgttgaagtttgtagtaacttcagtgagagttggttactcacaacaaatcctgaaaagtatttttagtgtttgtaggtattctgtgggatactatacaagcagaactgaggcacttaggacataacacttttggggtatatatatccaaatgcctaaaactatgggaggaaaccttggccaccccaaaaggaaaactaacatgatttgtgtctatgaagtgctggataattagcatgggatgagctctgggcatgccatgaaggaaagccacgctcccttcagaattcagaggcagggagcaattccagtttcacctaagtctcataattttagttcccttttaaaaaccctgaaaactacatcaccatggaatgaaaaatattgttatacaatacattgatctgtcaaacttccagaaccatggtagccttcagtgagatttccatcttggctggtcactccctgactgtagctgtaggtgaatgtgtttttgtgtgtgtgtgtctggttttagtgtcagaagggaaataaaagtgtaaggaggacactttaaaccctttgggtggagtttcgtaatttcccagactattttcaagcaacctggtccacccaggattagtgaccaggttttcaggaaaggatttgcttctctctagaaaatgtctgaaaggattttattttctgatgaaaggctgtatgaaaataccctcctcaaataacttgcttaactacatatagattcaagtgtgtcaatattctattttgtatattaaatgctatataatggggacaaatctatattatactgtgtatggcattattaagaagctttttcattattttttatcacagtaattttaaaatgtgtaaaaattaaaaccagtgactcctgtttaaaaataaaagttgtagttttttattcatgctgaataataatctgtagttaaaaaaaaagtgtctttttacctacgcagtgaaatgtcagactgtaaaaccttgtgtggaaatgtttaacttttattttttcatttaaatttgctgttctggtattaccaaaccacacatttgtaccgaattggcagtaaatgttagccatttacagcaatgccaaatatggagaaacatcataataaaaaaatctgctttttcatta

SEQ ID NO: 666 - DNA, 5288 bp, Macaca mulatta
tgcgagcgcgcggcggcggcagctgaagacccggccgcccagacgatgcggtggtgggggacctgccggcacgcgactgcccccgggcccaaattgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttttcttttttttctgcttgcttttccttttagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 667 - DNA, 5258 bp, Macaca mulatta
tgcgagcgcgcggcggcggcagctgaagacccggccgcccagacgatgcggtggtgggggacctgccggcacgcgactgcccccgggcccaaattgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgagactctaccctgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 668 - DNA, 5223 bp, Macaca mulatta
aatccagctcgctggaggttttgcgtttggcgtgcaacttccttcgagtttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 669 - DNA, 5236 bp, Macaca mulatta
gtagtgagaagagaaactggagaaactcggtggccctcctaacgccgccccagatagaccagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 670 - DNA, 5272 bp, Macaca mulatta
cgtgcaggcgccgtcggggccggggtggcggggcccgcgcgtagggcgtgggggcagggaccgcgggcgcccctgcagttgccaagcgtcgccaacagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 671 - DNA, 5315 bp, Macaca mulatta
gtacttaaaggtttggatgtgtgagtagctggtaggagggaaatttggaagtaattagggattgaggaattctagcacagtatttatcaaatgttatatgtattgattctcagaaaagcaaacagccttgattgaaaagagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 672 - DNA, 5383 bp, Macaca mulatta
ttctactcgctcgaatatttgcactccaccccggcgcgcccgagcgcgagcccgggctctggggaggccccgtcgcgcctggcttggggagggcgtgcagggcgcgtgagagtacacacgcggggggctgacagcttgctacttggagactccggcaggggctagcgttatctggtggaagtgggcgtgtcggagagagaactcaacagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 673 - DNA, 5227 bp, Macaca mulatta
ccattttgcgagctcgtgtctgtgacgggagcccgacggctcctctgtcagagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 674 - DNA, 5375 bp, Macaca mulatta
tgcagtttgtgtcccacagatattaacttcaataagcacttaatgagggccttccctgtgcgagaatggggaggaacaaaatgcagctcctgccctcctggggctttagttgtaccttagtaagaggaattttcatctgcctggctcctttcctcaaagaacaaagaagactttgcttcattaaagtgtctgagaaggaagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaatgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggttctggagacgacaacttgacttccttggggactctgaacttccctggtcgaacagttttttctaatggctattcaagccccagcatgagaccagatgtaagctctcctccatccagctcctcaacagcaacaacaggaccacctccgaaactctgcctggtgtgctctgatgaagcatcaggatgtcattatggagtcttaacttgtggaagctgtaaagttttcttcaaaagagcagtggaaggacagcacaattacctatgtgctggaaggaatgattgcatcatcgataaaattcgaagaaaaaactgcccagcatgccgctatcgaaaatgtcttcaggctggaatgaacctggaagctcgaaaaacaaagaaaaaaataaaaggaattcagcaggccactacaggagtctcacaagaaacctctgaaaatcctgctaacaaaacaatagttcctgcaacgttaccacaactcacccctaccctggtgtcactgttggaggttattgaacctgaagtgttatatgcaggatatgatagctctgttccagactcaacttggaggatcatgaccacgctcaacatgttaggagggcggcaagtgattgcagcagtgaaatgggcaaaagcgataccaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgaatacacagcagagaagtcacgcatgtacgaccaatgtaaacacatgctgtatgtttcctctgagttacacaggcttcaggtatcttatgaagaatatctctgtatgaaaaccttactgcttctctcttcagttcctaaagacggtctgaagagccaagagctatttgatgaaattagaatgacctacatcaaagagctaggaaaagccattgtcaagagggaaggaaactccagccagaactggcagcggttttatcaactgacaaaactcttggattctatgcatgaagtggttgaaaatcttcttaactattgcttccaaacatttttggataagaccatgagtattgaattcccagagatgttagctgaaatcatcaccaatcagataccaaaatattcaaatggaaatatcaaaaaacttctgtttcatcaaaagtgactgccttaataagaatggttgccttaaagaaagtcgaattaatagcttttattgtataaactctcagtttgtcctgtagaggttttgttgttttattttttattgttttcgtctgttgttttgttttaaatacgcactacatgtggtttatagagggccaagacttggcaacagaagcaattgagtcatcacttttcagtgatgggagagtagacggtgaaatttcattaagttagtatatcccagaaattagaaaccttaatatgtggacgtaatctccatagtcaaagaaggatggcacctaaaccaccagtgcccaaagtctgtgtgatgaactttctgctcatactttttcacagttggctggatgaaattttctagactttctgttggtgtatccccccctgtatagttaagatagcatttttgatttatgcatggaaacctgaaaaaagtttacaagtgtatatcagaaaagggaagttgtgccttttatagctattactgtctggttttaacaatttcctttatatttagtgaactacgcttgctcattttttcttacataattttttattcaagttattgtacagctgtttaagatgggcagctagttcgtagctttcccaaataaactctaaacattaatcttctgtgtgaaaatgggttggtgcttctaacctgatggcacttagctatcagaagaccacaaaattgactcaaatctccagtattcttgtcaaaaaaaagctcacattttgtatatatctgcttcagtggagaattatataggttgtgcaaattcaccatcctaactggtatgagcacctagtccagggacctgctgggtaaactgtggatgatggttgcaaaagactgatttaaaaatcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcagagcaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctgtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaaatgattgtaaaatagcttgtatagtgtaaaataagaatgatttttagatgagattgttttatcatgacatgttatatattttttgtaggggtcaaagaaatgctgatggataacctatatgatttatagtttgtacatgcattcatacaggcagcgttggtctcagaacccaaacaatttgctctaggggaagagggagatggagactggtcctgtgtgcagtgaaggttgctgaggctctgacccaatgagattacagaggaagttaccctctgcctcccattctgaccacccttctcattccaacagtgagtctgtcagtgcaggtttagtttactcaatctccccttgcactaaagtatgtaaacaggagacaggaaagtggtgcttacatacttaaaggcaccatctaatagtgggttactttcacatacaggcctcccccagcagttgaatgacaacagaagtttggcaatagtttgcatagaggtaccagcaatatgtaaatagtgcagaatctcataggttgccaataatacactaattcctttctatcctacaacaagagtttatttccaaataaaatgaggacatgtttttgttttctttgaatgctttttgaatgttatttgttattttcagtattttggagaaattatttaataaaaaacaatcatttgctttttgaatgctctctaaaagggaatgtaatattttaagatggtttgtaacccagctggataaatttttggtgcctaagaaaactgcttgaatatttttatcaatgacagtgttaagtttcaaaaagagcttctacaatgtagattatcattcatttatagaacgttatgtggttaaaaccagaaagcacatctcacacattaatctgattttcgtcccaacaatcttggcgctcaaaaaatagaactcaatgaaaaaaagattatgtgtactttgctgtcaataataagtcaactgatattcatcaacaactataggaggcttttcattaaatgggaaaagaagctgtgcccttttagaatacatgggggaaaagaaagtcatcttaattatgtttaactagggacttaagtgctatagggtggtgctgtttgaaagcagctttatttcctatgtatgtgttatctggttatcccaacccaaactattgaagtttgtagtaacttcagtgagagttggttactcacaacaaatcctgaaaagtatttttaa

SEQ ID NO: 675 - DNA, 5315 bp, Macaca mulatta; identical to SEQ ID NO: 671

SEQ ID NO: 676 - DNA, 618 bp, Macaca fascicularis
agttaggcgcgttttcttttttagtttctcctatttggcattgctgtaaatggctaactaacatttactgccaatttggtacaaatgtgtggtttggtaataccagaacagcaaatttaaatgaaaaaataaaagttagacatttccacacaaggttttacagtctgacatttcactgcgtaggtaaaaagacattttttttttaactacagattattattcagcatgaataaaaaactacaacttttatttttaaacaggagtcactggttttaatttttacacattttaaaattactgtgataaaaaataatgaaaaagctttttaataatgccatacacagtataatatagatttgtccccattatatagcatttaatatacaaaatagaatattgacacacttgaatctatatgtagttaagcaagttatttgaggagggtattttcatacagcctttcatcaaaaaataaaatcctttcaaacattttctaaagagaagcaaatcctttcctgaaaacctggtcactaatcctgggtggaccaggttgcttgaaaatagtctgggatattatgaaactccacccaaagggcttaaagtgtcctccttacactt

SEQ ID NO: 677 - DNA, 20 bp, Artificial Sequence
  Description of Artificial Sequence Synthetic primer
tgtccgcaactacaacgcct

SEQ ID NO: 678 - DNA, 6123 bp, Macaca fascicularis
  modified_base (257)..(258): a, c, t, g, unknown or other
aggttatgtaagggtttgctttcaccccattcaaaagatacctcttcctcttctcttgctccctcttgccctcattcttgtgcctgtgcagacatttgagtagaggcgaatcactttcacttctgctggggaaattgcaacacgcttctttaaatggcagagagaaggagaaaacttagatcttctgataccaaatcactgaaccttggaaggtcagaaatctttcaagccctgcaggaccgtaaaatgcccatgtnnccaacagaagcactggggcatgagtggggaaggaatagaaacagagtcagaaaggggataagagaagaataaaagggaaagtggtgaaggcagggaggcaaattgcttagtgtgaatatgcacgcgttcatttagttttcaaatccttgttgagcatgataaagttcccagcatcaatcctcacgtgttggtttccgttaggatctgcctgggggaatatctgctgaatcagtgactctgagctgaaccaggaaattcaccatgattaggagagtagctgtgttagtcagggtctctaccnnaaaaaaagttatacccaagagacaggatcttctcatccaaaattttcttcacttctgaaattctctggtttgtgctcatcattggcagctatttgttcatcaagagttgtgtagttggcttcttctggaaaaaggaatctgcgtcatatctaagtcagatttcattctggtgctctcagagcagttagcccaggaagggggccggcttctgtggctactggtgcagaggcagatgcagtttgtgtcccacagatattaacttcaataagcacttaatgagggccttccctgtgcgagaatggggaggaacaaaatgcagctcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacttctctcccagtgcgagagcgcggcggcggcagctgaagacccggccgcccagacgatgcggtggtgggggacctgccggcacgcgactccccccgggcccaaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccttctgcgttcacacgctaagttgtttatctctgctgcggcaggagctgcggacggtggcgggcgagcggctcctctgtcagagttgatattcactgatggactccaaagaatcattaactcccagtagagaagaaaaccccagcagtgtgcttgctcaggagaggggaaatgtgatggacttctataaaaccctaaggggaggagctactgtgaaggtttctgcatcttcaccctcactggctgtcgcttctcagtcagactccaagcagcgaagacttttggttgattttccaaaaggctcagtaagcaatgcgcagcagccagatctctccaaagcagtttcactctcaatgggactgtatatgggagagacagaaacaaaagtgatgggaaatgacctgggattcccacagcagggccaaatcagcctttcctcgggggaaacagacttaaagcttttggaagaaagcattgcaaacctcaataggtcgaccagtgttccagagaaccccaagagttcagcatccactgctgtgtctgctgcccccacaaagaaggagtttccaaaaactcactctgatggatcttcagaacagcaaaatttgaagggccatactggcaccaacggcggcaatgtgaaattgtataccgcagaccaaagcacctttgacattttgcaggatttggagttttcttctgggtccccaggtaaagagacgaatgagagtccttggagatcagacctgttgatagatgaaaactgtttgctttctcctctggcgggagaagacgattcattccttttggaaggaaattcgaacgaggactgtaagcctctcattttaccggacactaaacccaaaattaaggataatggagatctggttttgtcaagccccaataatgcaacactgccccaagtgaaaacagaaaaagaagatttcatcgaactctgcacccctggggtaattaagcaagagaaactgggcacagtttactgtcaggcaagctttcctggagcaaatataattggtaataaaatgtctgccatttctgttcatggtgtgagtacctctggaggacagatgtaccactatgacatgaatacagcatccctttctcaacagcaggatcagaagcctatttttaatgtcattccaccaattcccgttggttctgaaaattggaataggtgccaaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcatcaggatgtcattatggagtcttaacttgtggaagctgtaaagttttcttcaaaagagcagtggaaggtagacagcacaattacctatgtgctggaaggaatgattgcatcatcgataaaattcgaagaaaaaactgcccagcatgccgctatcgaaaatgtcttcaggctggaatgaacctggaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaccacaactcacccctaccctggtgtcactgttggaggttattgaacctgaagtgttatatgcaggatatgatagctctgttccagactcaacttggaggatcatgaccacgctcaacatgttaggagggcggcaagtgattgnnnnnnnnnnnnnnnnnnnnnnnnnnncaggtttcaggaacttacacctggatgaccaaatgaccctactgcaatactcctggatgtttcttatggcatttgccctggggtggagatcatatagacaatcaagtgcaaacctgctgtgttttgctcctgatctgattattaatgagtanagtntgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntctntgcangnngtggttgaaaatcttcttaactattgcttccaaacatttttggataagaccatgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttttgttttaaatacgcactacatgtggtttatagagggccaagacttggcaacagaagcaattgagtcnnnatcacttttcagtgatgggagagtagacggtgaaatttcattagttagtatatcccagaaattagaaaccttaatatgtggacgtaatctccatagtcaaagaaggatggcacctaaaccaccagtgcccaaagtctgtgtgaagaactttctgctncatacntttttncacagttggctggatgaaattttctagactttctgttggtgtatnccccccctgtatagttaagatagcatttttgatttatgcatggaaacctgaaaaaaagtttacaagtgtatatcagaaaagggaagttgtgccttttatagctattactgtctggttttaacaatttcctttatatttagtgaactacgcttgctcattttttcttacataatttttttattcaagttattgtannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaattacaccgtcctaactggtatngagcacctagtccagggacctgctgggtaaactgtggatgatggttgcaaaagactgatttaaaaantcactaccaagaggccctgtctgtacctaatgccctatttttgcaaaggctatatggcaagaaagctggtaaactatttgtctttcaggaccttttgaagtagtttgtataacttcttaaaagttgtgattccagacaaccagctgtaacacagctgagagaattttaatcggagcnaagtaattcctctcactaaactttacccaaaaactaaatctctaatatggcaaaaatggctagacacccattttcacattcccatctgtcaccaattggttaatctttcctgatggtacaggaaagctcagctactgatttttgtgatttagaactgtatgtcagacatccatgtttgtaaaactacacatccctaatgtgtgccatagagtttaacacaagtcctgtgaatttcttcactgttgaaaattattttaaacaaaatagaagctgtagtagccctttctgtgtgcaccttaccaactttctagtaaactcaaaacttaacatatttactaagccacaagaaatttgatttctattcaaggtggccaaattatttgtgtaatagaaaactgaaaatctaatattaaaaatatggaacttctaatatatttttatatttagttatagtttcagatatatatcatattggtattcactaatctgggaagggaagggctactgcagctttacatgcaatttattaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttccaacagtgagtctgtcagtgcaggtttagtttactcaatttccccttgcactaaagtatgtaaannnnnnnnncaggagacaggaaagtggtgcttacatacttaaaggcaccatctaatagtgggttactttcaacatacaggcctcccccagcagttgaatgnnancnnaannnncagaagtttggcaatagtttgcatagaggtaccagcaatatgtaaatagtgcagaatctcataggttgccaataatacactaattcctttctatc

SEQ ID NO: 679 - DNA, 6345 bp, Mus musculus
ttaatatttgccaatggactccaaagaatccttagctccccctggtagagacgaagtccccagcagtttgcttggccgggggaggggaagcgtgatggacttgtataaaaccctgaggggtggagctacagtcaaggtttctgcgtcttcaccctcagtggctgctgcttctcaggcagattccaagcagcagaggattctccttgatttttcaaaaggctcagcaagcaatgcgcagcagcagcagcagcagcagcagcagcagcagcagcagcagcagcagcagccgcagccagatttatccaaagccgtttcactgtccatgggactgtatatgggagagaccgaaacaaaagtgatggggaatgacttgggctacccacagcagggccagcttggcctctcctctggggaaacagactttcggcttctggaagaaagcattgcaaacctcaataggtcgaccagccgtccagagaaccccaagagttcaacacctgcagctgggtgtgctaccccgacagagaaggagtttccccagactcactctgatccatcttcagaacagcaaaatagaaaaagccagcctggcaccaacggtggcagtgtgaaattgtataccacagaccaaagcacctttgacatcttgcaggatttggagttttctgccgggtccccaggtaaagagacaaacgagagtccttggaggtcagacctgttgatagatgaaaacttgctttctcctttggcgggagaagatgatccattccttctggaaggggacgtgaatgaggattgcaagcctcttattttaccggacactaaacctaaaattcaggatactggagatacaatcttatcaagccccagcagtgtggcactgccccaagtgaaaacagagaaagatgatttcattgagctttgcacccctggggtaattaagcaagagaaactgggcccggtttattgccaggcaagcttttctgggacaaatataattgggaataaaatgtctgccatttctgttcatggcgtgagtacctctggaggacagatgtaccactatgacatgaatacagcatccctttctcagcagcaggatcagaagcctgtttttaatgtcattccaccaattcctgttggttctgaaaactggaataggtgccaagggtctggagaggacaacctgacttccttgggggctatgaacttcgcaggccgctcagtgttttctaatggatattcaagccctggaatgagaccagatgtgagttctcctccgtccagctcctccacagcaacgggaccacctcccaaactctgcctggtgtgctccgatgaagcttcgggatgccattatggggtgctgacgtgtggaagctgtaaagtcttctttaaaagagcagtggaaggacagcacaattacctttgtgctggaagaaatgattgcatcattgataaaattcgaagaaaaaactgtccagcatgccgctatcgaaaatgtcttcaagctggaatgaacctggaagctcgaaaaacgaagaaaaaaattaaaggaattcagcaagccactgcaggagtctcacaagacacttctgaaaacgctaacaaaacaatagttcctgccgcgctgccacagcttacccctaccctggtgtcactgctggaggtgatcgagcctgaggtgttatatgcaggatatgacagctctgttccagactcagcatggagaattatgaccacgctcaacatgttaggtgggcgccaagtgattgccgcagtgaaatgggcaaaggcgataccaggattcagaaacttacacctggatgaccaaatgacccttctacagtactcatggatgtttctcatggcatttgccctgggttggagatcatacagacaagcaagtggaaacctgctatgctttgctcctgatctgattattaatgagcagagaatgactctaccctgcatgtatgaccaatgtaaacacatgctgtttatctccactgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccattgtcaaaagggaaggaaactccagtcagaattggcagcggttttatcaactgacaaaacttttggactccatgcatgatgtggttgaaaatctccttagctactgcttccaaacatttttggataagtccatgagtattgaattcccagagatgttagctgaaatcatcactaatcagataccaaaatactcaaatggaaatatcaaaaagcttctgtttcatcagaaatgactgccttactaagaaaggctgccttaaagaaagttgaatttatagcttttactgtacaaacttatcaacttgtcttgtagatgttttgtcgttctttttgtttgtcttgtttgttttctatacgcactacatgtggtctctagagggccaagacttggcaacagaagcagatgagccatcacttttcagtgacaggaaagcagacagtgatgtgcattggctggtgtatcacagaaactagaacagttagtggagacatgtccactatcagagaaggaccgcacctgaaccaccagtgcccaaagtccatgtgatcaactttctgctcaactttcagttggctggataacactttctagacttttctgttggtgtatttttcccatgtatagttaggatagcattttgatttatgcatggaaacctgaaaaaagtttacacgtgtatatcagaaaagggaagttgtgccttttatagctattactgtctggttttaacaatttcctttatattcagtgaactatgcttgctcgtttttcttaaataatttttgtattctagttattgtatagctgtttaagatgggcagctgcctcacagctctcctagacgctaacattaatttccgtgtgaaaatgggtcggtgctcctaccctgatggcactcagctatcagaagaccacagaaattgactcagatctccagtattcttgtcaaaagctcttactctgtatatatctgcttccatggggaattatataggttgtgcagattaaccgtcctaactggtatagagcacctagtccagtgacctgctgggtaaactgtggatgatggttacaaaagactaattgtaaaacagtgcccaccaacaggccccgtttgcacccaatgcaccatctcttcagtggtgcgatagcaacaaagtttgtaactcagctctttcaggaccttcgggagtagtttgtgtaacattttaaaatgtattattccagataaccagctgtgataaagccgagagattgttttaatcagaccaagtaacttctctcattaaacgttaccctcaactaagtctctaatatggcaagaatggctagacacccattttcacatcccacctgtcaccaattggtctagctttcctggtggtacaggaaaatcagctactgattttttgttatttagaactgaatgtcaggcatccatgtttgtccaactatacatccctacatgtgccatagaatctaacacaagtcttgtgaacttcttcacactgagagttatcattttaaacaaaacagaagctgtagtagccctttctgtgtgcaccttaccaactttctgtgactcaaagcttaacacacttactaagccacaagaaatctgatttctacttaaggtggccaaattatttgtgtaatagaaaactgaaaatctaatattaaaaatatgaaacttctaatatatttttatatttagttctagtttcagatatatatcatattggtattcactaatctgggaagggaagggctactgcagctgtacatgcaatttattaacatgattgtaaaatagctgtatagtgtaaaataagaatgatttttagatgagattgttttatcatgacatgttatatattttttgtaggggtcaaagaaatgttgatggatatcctataagatttatagtatataagagcatccatacaggcctcagtggtcttggaaattaaaacaggtttgctctaagctagggagagggagctgggactggccctgtgtgcagtgcaggtcctgagggtttgacccgatcagatcacaggggaactaattccctcccatctaaccatcctcatccgaccatggccctgtcagtgcaggctggctttattaaatccaggacagaaaggtggcgcttatgtacttagaggcaccgtccagtaacagggttgttcccacatgcagcctccgcacgggttaacagaaacagaggctttagaagtttggcaataatgtgcatagaggttccagcaatatgtaaatactaaagaatcgcataggaagccaataatacactaatcctctccatcctacaagagtccatttccaagtaagatgaggacatgtttatgttttctttgaatgctttttgaatgttgttattttcagtattttgcagaaattatttaataaaaaaaagtataatcatttgctttttgaattctctctaaaagggaatgttcagtttgtaatggtttaaattggtctcaaagtactttaaaataattgtaacccagctggatgtgaaatttatggtgcctaagaaataccacttgaagattatcaatgacagtgttaagtttcaaaatgagcttctcaaaaatagattattgtacatttatggaatgttatatggttaaacccaaaaagcacatcacacataaatctgctttcagttccaaccagcttggctttcaaaaatagagctccaaaaaaaaaaaaggaaaaaaaagatatatatgctttgttattaacagaaggcagcagacattcataaaactactatcggaagttttccattagatgtataaagagctatcctttggtatgtgggaaagaagaaagctgtcataattctgattgagtataagtgagagagatacggtactgtttgagagcagctccttttctgcgtgtggcttcataccgttccaaactatgtagattttataatagcttcagtgagaattggtaacatgcctgtatgactcacaacagatcttgaaaactatctttaattactggtaggacaaaaagggacattctggttattttaggcactggcttggaacactgtatatgcagaagaaagaagacaggcaatctggggaaaggaaggggacctgggaagcactgccttctttaaggaaagacacaccaatagatgagatcatcccaaaggcacagggaccacagagtgtgagtccttagtgacgagtcaggtgagctctggtgagcttggagaagccagccccaccagcagagcaggcacggcagggatgggacaagcagggacgacaattccagctggacactggtcccagtattttgctccctcttatataccgtgaggcagtatcaccgtgggatgaaccatggtagcacgttttgatctgtcagcactcaaggatcatggtagccttcgggagctttaggttttggttggtcaccccaacgatcagctgtagttgaatgtgtttcttatgtgcctggtttcagtgttagaaggtgaaatagagtgtgcaaaggacactgcaaaccacttcggatggaagttttctcattttccagactattttcggtcagcctggtctatcaagatcggtaaccaggtcttcaggaaagggttggcttctatctaggacatgcctgaaaggattttattttctgataaatggctgtatgaaaataccctcctaaataccctgcttaactacatatagatttcagtgtgtcaatattctattttgtatattaaacaaatgctatataatggggacaaatctatattatactgtgtatggcattattaagaagctttttcattattttttatcacagtaatttttaaatgtgtaaaaattaaaaaccagtgactcctgtttaaaaataaaagttgtagttttttattcatgctgaataacctgtagtttaaaaacctgtctttctactacacagtgagatgtcagactgtaaagttttgtgtggaaatgtttaacttttatttttcatttcaatttgctgttctggtattaccaaaccacacatttgtaatgaattggcagtaaatgttagtcagccatttacagcaatgccaaatatggataaacatcataataaaagtatctgctttttcattatgtgactcccaaaaaaaaaaaaaaaaaa

SEQ ID NO: 680 - DNA, 6285 bp, Rattus norvegicus
gacgctgcgggggtgggggacctcggcggcacggagtccccccccgggctcacattaatatttgccaatggactccaaagaatccttagctccccctggtagagacgaagtccctggcagtttgcttggccaagggagggggagcgtaatggacttttataaaagcctgaggggaggagctacagtcaaggtttctgcatcttcgccctcagtggctgctgcttctcaggcagattccaagcagcagaggattctccttgatttctcgaaaggctccacaagcaatgtgcagcagcgacagcagcagcagcagcagcagcagcagcagcagcagcagcagcagcagcagcagcagccaggcttatccaaagccgtttcactgtccatggggctgtatatgggagagacagaaacaaaagtgatggggaatgacttgggctacccacagcagggccaacttggcctttcctctggggaaacagactttcggcttctggaagaaagcattgcaaacctcaataggtcgaccagcgttccagagaaccccaagagttcaacgtctgcaactgggtgtgctaccccgacagagaaggagtttcccaaaactcactcggatgcatcttcagaacagcaaaatcgaaaaagccagaccggcaccaacggaggcagtgtgaaattgtatcccacagaccaaagcacctttgacctcttgaaggatttggagttttccgctgggtccccaagtaaagacacaaacgagagtccctggagatcagatctgttgatagatgaaaacttgctttctcctttggcgggagaagatgatccattccttctcgaagggaacacgaatgaggattgtaagcctcttattttaccggacactaaacctaaaattaaggatactggagatacaatcttatcaagtcccagcagtgtggcactaccccaagtgaaaacagaaaaagatgatttcattgaactttgcacccccggggtaattaagcaagagaaactgggcccagtttattgtcaggcaagcttttctgggacaaatataattggtaataaaatgtctgccatttctgttcatggtgtgagtacctctggaggacagatgtaccactatgacatgaatacagcatccctttctcagcagcaggatcagaagcctgtttttaatgtcattccaccaattcctgttggttctgaaaactggaataggtgccaaggctccggagaggacagcctgacttccttgggggctctgaacttcccaggccggtcagtgttttctaatgggtactcaagccctggaatgagaccagatgtaagctctcctccatccagctcgtcagcagccacgggaccacctcccaagctctgcctggtgtgctccgatgaagcttcaggatgtcattacggggtgctgacatgtggaagctgcaaagtattctttaaaagagcagtggaaggacagcacaattacctttgtgctggaagaaacgattgcatcattgataaaattcgaaggaaaaactgcccagcatgccgctatcggaaatgtcttcaggctggaatgaaccttgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgatactaggcttgagaaacttacacctcgatgaccaaatgaccctgctacagtactcatggatgtttctcatggcatttgccttgggttggagatcatacagacaatcaagcggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatgactgccttactaagaaaggttgccttaaagaaagttgaatttatagcttttactgtacaaacttatcaatttgtcttgtagatgttttgttgttctttttgtttctgtcttgttttgttttaaacacgcagtacatgtggtttatagagggccaagacttggcgacagaagcagttgagtcaacactctgaagtgatgacacagcacacagtgaagtgtattgttggtgtatcacagaaactaacagttacgtggaggcatggccactgtcagagagggaccgcacctaaaccaccgtgcccaagtccatgtggttcaactttctgactcagaactttacagttggctgggtaaaactttctagactttctgttggtgtatttttcccatgtatagttaggatggtattttgatttatgcatgcaaacctgaaaaaagtttacaagtgtatatcagaaaagggaagttgtgccttttatagctattactgtctggttttaacaatttcctttatattcagtgaactatgcttgctcgtttctcttcaataatttttgtattccagttattgtacagctgtttaagatgggcagctgcttcacagctttcctagacgctaacattaatttccgtgtgaaaatgggtcggtgcttctaccctgttggcaccagctatcagaagaccacagaaattgactcagatctccagtattcttgttaaaaagctcttactctgtatatatctgcttccatggagaattacataggctgagcagattacataggctgagcagattaaccgtcctaactggtgtagagcacctagtccagtgaccttctgggtaaaccgtggatgatggttacagaagactggtgggaaaacagtaactaccaaaaggcccctttccatctaatgcaccatctcttcaatggggagatagcaaccaagcccgtaaatcagctctttcaggaccttctggagtggtttgcataacattttaaaatgtattattccagatagccagctctgataaagccgagagattgtttaatcagaccaagtaacttctctcattaaacttacccccaactaaatcgctaatacagcaagaatggctagacacccattttcacatctcacccgcaccgattggtctagctctcatggtggtcaggagaatcagctactgatttttgttacttagaatttcaggactcgcattttccctacacatccctacatgtgccatagaatttaacacaagtcctgtgaacttcttcacattgagaattatcattttaaacaaaacagaagcagtagtagccctttcttgtgcaccttaccctttcttgactcaaagcttaatatgcttactaagccacaagaaatcgatttcacttaaaggcgccaaattatttgtgtaatagaaaaactgaaaatctaatattaaaaatatgaaacttctaatatatttttatatttagttatagtttcgatatatatcatatcggtattcactgatcttgggaaagggaaagggctactgcagctttacatgcaatttattaactgactgtaaaatagctgtatagtaataagaatgacttttagtgagattgctttatcatgacatgttatatatttttcgtaggggtcaaagaaatattgatggatatgatagcctatatgatttaatgtatataaaagcatcaaacaggccttaacgcgtcttggaaaaaaatacctttgttctaagctagggaagggagcggagaggccccgtgtgtatggaggttccgaggctcggataagagatcaaggggatctaattcctacctccatctaattacctcaccacccatgatcctgtcagtgaggggttattaaatcccccgttatactaatataaataggaagaagggtggcgctcacgtctgttccaggcgccgcagtagcagggttattttccatgcagcctcccgacaaggttagcagagggaggctttggcaagtttggcgtggcgtgcatagaggcaccagcaacatgtaaacctaaagagcccataggaagccaagaatacactaatcctccccacccttcaatagtccatttccaagtaagatgaggacatgcttatgttttctttgaatgcttttagaatgttgttattttcagtattttgcagaaattatttaataaaaaagtataatttgaattctctctaaaagggattgttcagtttgtaatggtttaaattggtctcaaagtactttaagataattgtaacccagctggatgtgaaatttatggtgcctaagaaataccacttgaatattatcaagacagtgttaagttttaaaatgagcttctcaaaaatagattattgtacatttatggaatgttatatggttaaacccaaaaaagcacatcacacataaatctgctttcagcttggctttcaaaaatagagctccaaaaacgaaaaaggagaagaaaaagtatatatatgcgttgttattaacagaaggcaacagacattcataaaactactaccgaagctttccttgaagcgtataaagagccatgctcctttagtatgtggggaagaagagagccgtcatagtttcgagtacagagagaagatgcggtactgtctccgtgtgtggcttcataccgttcctaactatttaggtttataataacttcagtgagactcggtgacatgcctgtatgactcatgaccgatcttgaaagatatctttaattactggtaggacaaaagggacactctggttattttaggccttggcttgggatactgtatatccagaagaaaggagacaggaaacttggggaagggaagggaacctaggaagcactgccttctgtaggaaagaacacaccaataagtgagagtacccaaagggacaaggccacacagtgtggggtctaaggatgagtcagggtgagctctggtgggcatggagaagccagcaactccagtgctacagagcagggcagggcagggatgggacaagatggatgcggatcccagtcccagtagtttgctccctcttatttaccatgggatgaaccatggagtattgatctgtcagcactcaaggatcatggagcttgagattccggttggtcaccccaacggtaagctgagattgaatgtgtttcttatgtgccggtttcagtgttagaaggcgaaacagagtgtacagaagacactgcaaaccggtcagatgaaagtcttctcattcccaaactattttcagtcagcctgctctatcaggactggtgaccagctgctaggacagggtcggcgcttctgtctagaatatgcctgaaaggattttattttctgataaatggctgtatgaaaataccctcctcaataacctgcttaactacatagagatttcagtgtgtcaatattctattttgtatattaaacaaaggctatataatggggacaaatctatattatactgtgtatggcattattaagaagcttttaattttttatcacagtaatttttaaatgtgtaaaaaattaaaaattagtgatccgtttaaaaataaaagttgtagttttttattcatgctgaataacctgtagtttaaaaatccgtctttctacctacaagtgaaatgtcagacgtaaaattttgtgtggaaatgtttaacttttatttttctttaaatttgctgtcttggtattaccaaaccacacattgtactgaattggcagtaaatgttagtcagccatttacagcaatgccaaatatggataaacatcataataaaatatctgctttttc

SEQ ID NO: 681 - DNA, 21 bp, Artificial Sequence
  Description of Artificial Sequence Synthetic oligonucleotide
cuuacgcugaguacuucgatt

SEQ ID NO: 682 - DNA, 21 bp, Artificial Sequence
  Description of Artificial Sequence Synthetic oligonucleotide
ucgaaguacucagcguaagtt

SEQ ID NO: 683 - DNA, 41 bp, Artificial Sequence
  Description of Artificial Sequence Synthetic probe
gaatttgccatgggtggaattttttctcttggaaagaaagt

SEQ ID NO: 684 - DNA, 41 bp, Artificial Sequence
  Description of Artificial Sequence Synthetic probe
ggagggatctcgctcctggatttttctcttggaaagaaagt

SEQ ID NO: 685 - DNA, 40 bp, Artificial Sequence
  Description of Artificial Sequence Synthetic probe
ccccagccttctccatggttttttctcttggaaagaaagt

SEQ ID NO: 686 - DNA, 40 bp, Artificial Sequence
  Description of Artificial Sequence Synthetic probe
gctcccccctgcaaatgagtttttctcttggaaagaaagt

SEQ ID NO: 687 - DNA, 42 bp, Artificial Sequence
  Description of Artificial Sequence Synthetic probe
agccttgacggtgccatgtttttaggcataggacccgtgtct

SEQ ID NO: 688 - DNA, 45 bp, Artificial Sequence
  Description of Artificial Sequence Synthetic probe
gatgacaagcttcccgttctctttttaggcataggacccgtgtct

SEQ ID NO: 689 - DNA, 46 bp, Artificial Sequence
  Description of Artificial Sequence Synthetic probe
agatggtgatgggatttccatttttttaggcataggacccgtgtct

SEQ ID NO: 690 - DNA, 44 bp, Artificial Sequence
  Description of Artificial Sequence Synthetic probe
gcatcgccccacttgatttttttttaggcataggacccgtgtct

SEQ ID NO: 691 - DNA, 43 bp, Artificial Sequence
  Description of Artificial Sequence Synthetic probe
cacgacgtactcagcgccatttttaggcataggacccgtgtct

SEQ ID NO: 692 - DNA, 46 bp, Artificial Sequence
  Description of Artificial Sequence Synthetic probe
ggcagagatgatgacccttttgtttttaggcataggacccgtgtct

SEQ ID NO: 693 - DNA, 21 bp, Artificial Sequence
  Description of Artificial Sequence Synthetic probe
ggtgaagacgccagtggactc

SEQ ID NO: 694 - DNA, 43 bp, Artificial Sequence
  Description of Artificial Sequence Synthetic probe
tcccatgctaattatccagcactttttctcttggaaagaaagt

SEQ ID NO: 695 - DNA, 39 bp, Artificial Sequence
  Description of Artificial Sequence Synthetic probe
tggcatgcccagagctcatttttctcttggaaagaaagt

SEQ ID NO: 696 - DNA, 40 bp, Artificial Sequence
  Description of Artificial Sequence Synthetic probe
ggagcgtggctttccttcatttttctcttggaaagaaagt

SEQ ID NO: 697 - DNA, 42 bp, Artificial Sequence
  Description of Artificial Sequence Synthetic probe
ccctgcctctgaattctgaagtttttctcttggaaagaaagt

SEQ ID NO: 698 - DNA, 46 bp, Artificial Sequence
  Description of Artificial Sequence Synthetic probe
cctccttacacttttatttcccttctttttctcttggaaagaaagt

SEQ ID NO: 699 - DNA, 46 bp, Artificial Sequence
  Description of Artificial Sequence Synthetic probe
ttttctagagagaagcaaatccttttttttctcttggaaagaaagt

SEQ ID NO: 700 - DNA, 45 bp, Artificial Sequence
  Description of Artificial Sequence Synthetic probe
gagggtattttcatacagcctttctttttctcttggaaagaaagt

SEQ ID NO: 701 - DNA, 52 bp, Artificial Sequence
  Description of Artificial Sequence Synthetic probe
ttcatagacacaaatcatgttagttttctttttaggcataggacccgtgtct

SEQ ID NO: 702 - DNA, 47 bp, Artificial Sequence
  Description of Artificial Sequence Synthetic probe
tccatggtgatgtagttttcaggtttttaggcataggacccgtgtct

SEQ ID NO: 703 - DNA, 50 bp, Artificial Sequence
  Description of Artificial Sequence Synthetic probe
acaaaaacacattcacctacagctactttttaggcataggacccgtgtct

SEQ ID NO: 704 - DNA, 49 bp, Artificial Sequence
  Description of Artificial Sequence Synthetic probe
tgacactaaaaccagacacacacactttttaggcataggacccgtgtct

SEQ ID NO: 705 - DNA, 55 bp, Artificial Sequence
  Description of Artificial Sequence Synthetic probe
aatctatatgtagttaagcaagttatttgagtttttaggcataggacccgtgtct

SEQ ID NO: 706 - DNA, 24 bp, Artificial Sequence
  Description of Artificial Sequence Synthetic probe
gacttaggtgaaactggaattgct

SEQ ID NO: 707 - DNA, 27 bp, Artificial Sequence
  Description of Artificial Sequence Synthetic probe
gtttttaaaagggaactaaaattatga

SEQ ID NO: 708 - DNA, 31 bp, Artificial Sequence
  Description of Artificial Sequence Synthetic probe
gatcaatgtattgtataacaatatttttcat

SEQ ID NO: 709 - DNA, 42 bp, Artificial Sequence
  Description of Artificial Sequence Synthetic probe
atctggtctcattccagggcttttttctcttggaaagaaagt

SEQ ID NO: 710 - DNA, 42 bp, Artificial Sequence
  Description of Artificial Sequence Synthetic probe
caggcagagtttgggaggtggtttttctcttggaaagaaagt

SEQ ID NO: 711 - DNA, 42 bp, Artificial Sequence
  Description of Artificial Sequence Synthetic probe
ttccaggttcattccagcttgtttttctcttggaaagaaagt

SEQ ID NO: 712 - DNA, 43 bp, Artificial Sequence
  Description of Artificial Sequence Synthetic probe
tttttttcttcgtttttcgagctttttctcttggaaagaaagt

SEQ ID NO: 713 - DNA, 44 bp, Artificial Sequence
  Description of Artificial Sequence Synthetic probe
agtggcttgctgaattcctttaatttttctcttggaaagaaagt

SEQ ID NO: 714 - DNA, 47 bp, Artificial Sequence
  Description of Artificial Sequence Synthetic probe
ggaactattgttttgttagcgttttctttttctcttggaaagaaagt

SEQ ID NO: 715 - DNA, 42 bp, Artificial Sequence
  Description of Artificial Sequence Synthetic probe
tcccgttgctgtggaggatttttaggcataggacccgtgtct

SEQ ID NO: 716 - DNA, 46 bp, Artificial Sequence
  Description of Artificial Sequence Synthetic probe
ccgaagcttcatcggagcacactttttaggcataggacccgtgtct

SEQ ID NO: 717 - DNA, 44 bp, Artificial Sequence
  Description of Artificial Sequence Synthetic probe
cagcaccccataatggcatctttttaggcataggacccgtgtct

SEQ ID NO: 718 - DNA, 46 bp, Artificial Sequence
  Description of Artificial Sequence Synthetic probe
tccagcacaaaggtaattgtgctttttaggcataggacccgtgtct

SEQ ID NO: 719 - DNA, 49 bp, Artificial Sequence
  Description of Artificial Sequence Synthetic probe
ttttatcaatgatgcaatcatttcttttttaggcataggacccgtgtct

SEQ ID NO: 720 - DNA, 45 bp, Artificial Sequence
  Description of Artificial Sequence Synthetic probe
aagacattttcgatagcggcatttttaggcataggacccgtgtct

SEQ ID NO: 721 - DNA, 21 bp, Artificial Sequence
  Description of Artificial Sequence Synthetic probe
gctggacggaggagaactcac

SEQ ID NO: 722 - DNA, 24 bp, Artificial Sequence
  Description of Artificial Sequence Synthetic probe
gaagactttacagcttccacacgt

SEQ ID NO: 723 - DNA, 23 bp, Artificial Sequence
  Description of Artificial Sequence Synthetic probe
tgtccttccactgctcttttaaa

SEQ ID NO: 724 - DNA, 23 bp, Artificial Sequence
  Description of Artificial Sequence Synthetic probe
tgctggacagttttttcttcgaa

SEQ ID NO: 725 - DNA, 24 bp, Artificial Sequence
  Description of Artificial Sequence Synthetic probe
agaagtgtcttgtgagactcctgc

SEQ ID NO: 726 - DNA, 40 bp, Artificial Sequence
  Description of Artificial Sequence Synthetic probe
caaatggcagccctggtgatttttctcttggaaagaaagt

SEQ ID NO: 727 - DNA, 43 bp, Artificial Sequence
  Description of Artificial Sequence Synthetic probe
ccttgactgtgccgttgaattttttttctcttggaaagaaagt

SEQ ID NO: 728 - DNA, 41 bp, Artificial Sequence
  Description of Artificial Sequence Synthetic probe
gtctcgctcctggaagatggtttttctcttggaaagaaagt

SEQ ID NO: 729 - DNA, 39 bp, Artificial Sequence
  Description of Artificial Sequence Synthetic probe
cccggccttctccatggttttttctcttggaaagaaagt

SEQ ID NO: 730 - DNA, 46 bp, Artificial Sequence
  Description of Artificial Sequence Synthetic probe
aacaatctccactttgccactgtttttaggcataggacccgtgtct

SEQ ID NO: 731 - DNA, 50 bp, Artificial Sequence
  Description of Artificial Sequence Synthetic probe
catgtagaccatgtagttgaggtcaatttttaggcataggacccgtgtct

SEQ ID NO: 732 - DNA, 44 bp, Artificial Sequence
  Description of Artificial Sequence Synthetic probe
gacaagcttcccattctcggtttttaggcataggacccgtgtct

SEQ ID NO: 733 - DNA, 43 bp, Artificial Sequence
  Description of Artificial Sequence Synthetic probe
tgatgggcttcccgttgattttttaggcataggacccgtgtct

SEQ ID NO: 734 - DNA, 44 bp, Artificial Sequence
  Description of Artificial Sequence Synthetic probe
gacatactcagcaccggccttttttaggcataggacccgtgtct

SEQ ID NO: 735 - DNA, 19 bp, Artificial Sequence
  Description of Artificial Sequence Synthetic probe
tgaaggggtcgttgatggc

SEQ ID NO: 736 - DNA, 23 bp, Artificial Sequence
  Description of Artificial Sequence Synthetic probe
ccgtgagtggagtcatactggaa

SEQ ID NO: 737 - DNA, 22 bp, Artificial Sequence
  Description of Artificial Sequence Synthetic probe
caccccatttgatgttagtggg

SEQ ID NO: 738 - DNA, 24 bp, Artificial Sequence
  Description of Artificial Sequence Synthetic probe
ggtgaagacaccagtagactccac

SEQ ID NO: 739 - DNA, 21 bp, Artificial Sequence
  Description of Artificial Sequence Synthetic oligonucleotide
uggucgaacaguuuuuucutt

SEQ ID NO: 740 - DNA, 21 bp, Artificial Sequence
  Description of Artificial Sequence Synthetic oligonucleotide
uggucgaacaguuuuuucctt

SEQ ID NO: 741 - DNA, 21 bp, Artificial Sequence
  Description of Artificial Sequence Synthetic oligonucleotide
uggucgaacaguuuuuucctt

SEQ ID NO: 742 - DNA, 21 bp, Artificial Sequence
  Description of Artificial Sequence Synthetic oligonucleotide
uggucgaacaguuuuuucgtt

SEQ ID NO: 743 - DNA, 21 bp, Artificial Sequence
  Description of Artificial Sequence Synthetic oligonucleotide
uggucgaacaguuuuuucgtt

SEQ ID NO: 744 - DNA, 21 bp, Artificial Sequence
  Description of Artificial Sequence Synthetic oligonucleotide
agaaaaaacuguucgaccatt

SEQ ID NO: 745 - DNA, 21 bp, Artificial Sequence
  Description of Artificial Sequence Synthetic oligonucleotide
agaaaaaacuguucgaccatt

SEQ ID NO: 746 - DNA, 20 bp, Artificial Sequence
  Description of Artificial Sequence Synthetic oligonucleotide
uggucgaacaguuuuuucut

SEQ ID NO: 747 - DNA, 20 bp, Artificial Sequence
  Description of Artificial Sequence Synthetic oligonucleotide
uggucgaacaguuuuuucut

SEQ ID NO: 748 - DNA, 20 bp, Artificial Sequence
  Description of Artificial Sequence Synthetic oligonucleotide
uggucgaacaguuuuuucct

SEQ ID NO: 749 - DNA, 20 bp, Artificial Sequence
  Description of Artificial Sequence Synthetic oligonucleotide
uggucgaacaguuuuuucct

SEQ ID NO: 750 - DNA, 20 bp, Artificial Sequence
  Description of Artificial Sequence Synthetic oligonucleotide
uggucgaacaguuuuuucgt

SEQ ID NO: 751 - DNA, 20 bp, Artificial Sequence
  Description of Artificial Sequence Synthetic oligonucleotide
uggucgaacaguuuuuucgt

SEQ ID NO: 752 - DNA, 20 bp, Artificial Sequence
  Description of Artificial Sequence Synthetic oligonucleotide
agaaaaaacuguucgaccat

SEQ ID NO: 753 - DNA, 20 bp, Artificial Sequence
  Description of Artificial Sequence Synthetic oligonucleotide
agaaaaaacuguucgaccat

SEQ ID NO: 754 - DNA, 20 bp, Artificial Sequence
  Description of Artificial Sequence Synthetic oligonucleotide
uggucgaacaguuuuuucut

SEQ ID NO: 755 - DNA, 20 bp, Artificial Sequence
  Description of Artificial Sequence Synthetic oligonucleotide
uggucgaacaguuuuuucut

SEQ ID NO: 756 - DNA, 20 bp, Artificial Sequence
  Description of Artificial Sequence Synthetic oligonucleotide
uggucgaacaguuuuuucct

SEQ ID NO: 757 - DNA, 20 bp, Artificial Sequence
  Description of Artificial Sequence Synthetic oligonucleotide
uggucgaacaguuuuuucct

SEQ ID NO: 758 - DNA, 20 bp, Artificial Sequence
  Description of Artificial Sequence Synthetic oligonucleotide
uggucgaacaguuuuuucgt

SEQ ID NO: 759 - DNA, 20 bp, Artificial Sequence
  Description of Artificial Sequence Synthetic oligonucleotide
uggucgaacaguuuuuucgt

SEQ ID NO: 760 - DNA, 20 bp, Artificial Sequence
  Description of Artificial Sequence Synthetic oligonucleotide
agaaaaaacuguucgaccat

SEQ ID NO: 761 - DNA, 20 bp, Artificial Sequence
  Description of Artificial Sequence Synthetic oligonucleotide
agaaaaaacuguucgaccat

SEQ ID NO: 762 - DNA, 21 bp, Artificial Sequence
  Description of Artificial Sequence Synthetic oligonucleotide
guuccagacucaacuuggctt

SEQ ID NO: 763 - DNA, 21 bp, Artificial Sequence
  Description of Artificial Sequence Synthetic oligonucleotide
guuccagacucaacuuggutt

SEQ ID NO: 764 - DNA, 20 bp, Artificial Sequence
  Description of Artificial Sequence Synthetic oligonucleotide
guuccagacucaacuuggat

SEQ ID NO: 765 - DNA, 20 bp, Artificial Sequence
  Description of Artificial Sequence Synthetic oligonucleotide
guuccagacucaacuuggct

SEQ ID NO: 766 - DNA, 20 bp, Artificial Sequence
  Description of Artificial Sequence Synthetic oligonucleotide
guuccagacucaacuuggut

SEQ ID NO: 767 - DNA, 20 bp, Artificial Sequence
  Description of Artificial Sequence Synthetic oligonucleotide
guuccagacucaacuuggat

SEQ ID NO: 768 - DNA, 20 bp, Artificial Sequence
  Description of Artificial Sequence Synthetic oligonucleotide
guuccagacucaacuuggct

SEQ ID NO: 769 - DNA, 20 bp, Artificial Sequence
  Description of Artificial Sequence Synthetic oligonucleotide
guuccagacucaacuuggut

SEQ ID NO: 770 - DNA, 21 bp, Artificial Sequence
  Description of Artificial Sequence Synthetic oligonucleotide
uccaaguugagucuggaactt

SEQ ID NO: 771 - DNA, 20 bp, Artificial Sequence
  Description of Artificial Sequence Synthetic oligonucleotide
uccaaguugagucuggaact

SEQ ID NO: 772 - DNA, 20 bp, Artificial Sequence
  Description of Artificial Sequence Synthetic oligonucleotide
uccaaguugagucuggaact

SEQ ID NO: 773 - DNA, 20 bp, Artificial Sequence
  Description of Artificial Sequence Synthetic oligonucleotide
uccaaguugagucuggaact

SEQ ID NO: 774 - DNA, 20 bp, Artificial Sequence
  Description of Artificial Sequence Synthetic oligonucleotide
uccaaguugagucuggaact

SEQ ID NO: 775 - RNA, 19 nt, Artificial Sequence
  Description of Artificial Sequence Synthetic oligonucleotide
ugcaaaccucaauaggucg

SEQ ID NO: 776 - RNA, 19 nt, Artificial Sequence
  Description of Artificial Sequence Synthetic oligonucleotide
cgaccuauugagguuugca

SEQ ID NO: 777 - RNA, 19 nt, Artificial Sequence
  Description of Artificial Sequence Synthetic oligonucleotide
aaaccucaauaggucgacc

SEQ ID NO: 778 - RNA, 19 nt, Artificial Sequence
  Description of Artificial Sequence Synthetic oligonucleotide
ggucgaccuauugagguuu

SEQ ID NO: 779 - RNA, 19 nt, Artificial Sequence
  Description of Artificial Sequence Synthetic oligonucleotide
aaccucaauaggucgacca

SEQ ID NO: 780 - RNA, 19 nt, Artificial Sequence
  Description of Artificial Sequence Synthetic oligonucleotide
uggucgaccuauugagguu

SEQ ID NO: 781 - RNA, 19 nt, Artificial Sequence
  Description of Artificial Sequence Synthetic oligonucleotide
accucaauaggucgaccag

SEQ ID NO: 782 - RNA, 19 nt, Artificial Sequence
  Description of Artificial Sequence Synthetic oligonucleotide
cuggucgaccuauugaggu

SEQ ID NO: 783 - RNA, 19 nt, Artificial Sequence
  Description of Artificial Sequence Synthetic oligonucleotide
uuaaugucauuccaccaau

SEQ ID NO: 784 - RNA, 19 nt, Artificial Sequence
  Description of Artificial Sequence Synthetic oligonucleotide
auugguggaaugacauuaa

SEQ ID NO: 785 - RNA, 19 nt, Artificial Sequence
  Description of Artificial Sequence Synthetic oligonucleotide
ugugauggacuucuauaaa

SEQ ID NO: 786 - RNA, 19 nt, Artificial Sequence
  Description of Artificial Sequence Synthetic oligonucleotide
uuuauagaaguccaucaca

SEQ ID NO: 787 - RNA, 19 nt, Artificial Sequence
  Description of Artificial Sequence Synthetic oligonucleotide
ccaagcagcgaagacuuuu

SEQ ID NO: 788 - RNA, 19 nt, Artificial Sequence
  Description of Artificial Sequence Synthetic oligonucleotide
aaaagucuucgcugcuugg

SEQ ID NO: 789 - RNA, 19 nt, Artificial Sequence
  Description of Artificial Sequence Synthetic oligonucleotide
uuuccaaaaggcucaguaa

SEQ ID NO: 790 - RNA, 19 nt, Artificial Sequence
  Description of Artificial Sequence Synthetic oligonucleotide
uuacugagccuuuuggaaa

SEQ ID NO: 791 - RNA, 19 nt, Artificial Sequence
  Description of Artificial Sequence Synthetic oligonucleotide
aaggcucaguaagcaaugc

SEQ ID NO: 792 - RNA, 19 nt, Artificial Sequence
  Description of Artificial Sequence Synthetic oligonucleotide
gcauugcuuacugagccuu

SEQ ID NO: 793 - RNA, 19 nt, Artificial Sequence
  Description of Artificial Sequence Synthetic oligonucleotide
ggcucaguaagcaaugcgc

SEQ ID NO: 794 - RNA, 19 nt, Artificial Sequence
  Description of Artificial Sequence Synthetic oligonucleotide
gcgcauugcuuacugagcc

SEQ ID NO: 795 - RNA, 19 nt, Artificial Sequence
  Description of Artificial Sequence Synthetic oligonucleotide
cucaguaagcaaugcgcag

SEQ ID NO: 796 - RNA, 19 nt, Artificial Sequence
  Description of Artificial Sequence Synthetic oligonucleotide
cugcgcauugcuuacugag

SEQ ID NO: 797 - RNA, 19 nt, Artificial Sequence
  Description of Artificial Sequence Synthetic oligonucleotide
cucucaaugggacuguaua

SEQ ID NO: 798 - RNA, 19 nt, Artificial Sequence
  Description of Artificial Sequence Synthetic oligonucleotide
uauacagucccauugagag

SEQ ID NO: 799 - RNA, 19 nt, Artificial Sequence
  Description of Artificial Sequence Synthetic oligonucleotide
ucucaaugggacuguauau

SEQ ID NO: 800 - RNA, 19 nt, Artificial Sequence
  Description of Artificial Sequence Synthetic oligonucleotide
auauacagucccauugaga

SEQ ID NO: 801 - RNA, 19 nt, Artificial Sequence
  Description of Artificial Sequence Synthetic oligonucleotide
ucaaugggacuguauaugg

SEQ ID NO: 802 - RNA, 19 nt, Artificial Sequence
  Description of Artificial Sequence Synthetic oligonucleotide
ccauauacagucccauuga

SEQ ID NO: 803 - RNA, 19 nt, Artificial Sequence
  Description of Artificial Sequence Synthetic oligonucleotide
ugggaaaugaccugggauu

SEQ ID NO: 804 - RNA, 19 nt, Artificial Sequence
  Description of Artificial Sequence Synthetic oligonucleotide
aaucccaggucauuuccca

SEQ ID NO: 805 - RNA, 19 nt, Artificial Sequence
  Description of Artificial Sequence Synthetic oligonucleotide
agcauugcaaaccucaaua

SEQ ID NO: 806 - RNA, 19 nt, Artificial Sequence
  Description of Artificial Sequence Synthetic oligonucleotide
uauugagguuugcaaugcu

SEQ ID NO: 807 - RNA, 19 nt, Artificial Sequence
  Description of Artificial Sequence Synthetic oligonucleotide
uuugacauuuugcaggauu

SEQ ID NO: 808 - RNA, 19 nt, Artificial Sequence
  Description of Artificial Sequence Synthetic oligonucleotide
aauccugcaaaaugucaaa

SEQ ID NO: 809 - RNA, 19 nt, Artificial Sequence
  Description of Artificial Sequence Synthetic oligonucleotide
cccagguaaagagacgaau

SEQ ID NO: 810 - RNA, 19 nt, Artificial Sequence
  Description of Artificial Sequence Synthetic oligonucleotide
auucgucucuuuaccuggg

SEQ ID NO: 811 - RNA, 19 nt, Artificial Sequence
  Description of Artificial Sequence Synthetic oligonucleotide
ccagguaaagagacgaaug

SEQ ID NO: 812 - RNA, 19 nt, Artificial Sequence
  Description of Artificial Sequence Synthetic oligonucleotide
cauucgucucuuuaccugg

SEQ ID NO: 813 - RNA, 19 nt, Artificial Sequence
  Description of Artificial Sequence Synthetic oligonucleotide
cagguaaagagacgaauga

SEQ ID NO: 814 - RNA, 19 nt, Artificial Sequence
  Description of Artificial Sequence Synthetic oligonucleotide
ucauucgucucuuuaccug

SEQ ID NO: 815 - RNA, 19 nt, Artificial Sequence
  Description of Artificial Sequence Synthetic oligonucleotide
agacgaaugagaguccuug

SEQ ID NO: 816 - RNA, 19 nt, Artificial Sequence
  Description of Artificial Sequence Synthetic oligonucleotide
caaggacucucauucgucu

SEQ ID NO: 817 - RNA, 19 nt, Artificial Sequence
  Description of Artificial Sequence Synthetic oligonucleotide
agaucagaccuguugauag

SEQ ID NO: 818 - RNA, 19 nt, Artificial Sequence
  Description of Artificial Sequence Synthetic oligonucleotide
cuaucaacaggucugaucu

SEQ ID NO: 819 - RNA, 19 nt, Artificial Sequence
  Description of Artificial Sequence Synthetic oligonucleotide
ucagaccuguugauagaug

SEQ ID NO: 820 - RNA, 19 nt, Artificial Sequence
  Description of Artificial Sequence Synthetic oligonucleotide
caucuaucaacaggucuga

SEQ ID NO: 821 - RNA, 19 nt, Artificial Sequence
  Description of Artificial Sequence Synthetic oligonucleotide
acgauucauuccuuuugga

SEQ ID NO: 822 - RNA, 19 nt, Artificial Sequence
  Description of Artificial Sequence Synthetic oligonucleotide
uccaaaaggaaugaaucgu

SEQ ID NO: 823 - RNA, 19 nt, Artificial Sequence
  Description of Artificial Sequence Synthetic oligonucleotide
aagccucucauuuuaccgg

SEQ ID NO: 824 - RNA, 19 nt, Artificial Sequence
  Description of Artificial Sequence Synthetic oligonucleotide
ccgguaaaaugagaggcuu

SEQ ID NO: 825 - RNA, 19 nt, Artificial Sequence
  Description of Artificial Sequence Synthetic oligonucleotide
agccucucauuuuaccgga

SEQ ID NO: 826 - RNA, 19 nt, Artificial Sequence
  Description of Artificial Sequence Synthetic oligonucleotide
uccgguaaaaugagaggcu

SEQ ID NO: 827 - RNA, 19 nt, Artificial Sequence
  Description of Artificial Sequence Synthetic oligonucleotide
gccucucauuuuaccggac

SEQ ID NO: 828 - RNA, 19 nt, Artificial Sequence
  Description of Artificial Sequence Synthetic oligonucleotide
guccgguaaaaugagaggc

SEQ ID NO: 829 - RNA, 19 nt, Artificial Sequence
  Description of Artificial Sequence Synthetic oligonucleotide
ccucucauuuuaccggaca

SEQ ID NO: 830 - RNA, 19 nt, Artificial Sequence
  Description of Artificial Sequence Synthetic oligonucleotide
uguccgguaaaaugagagg

SEQ ID NO: 831 - RNA, 19 nt, Artificial Sequence
  Description of Artificial Sequence Synthetic oligonucleotide
ucauuuuaccggacacuaa

SEQ ID NO: 832 - RNA, 19 nt, Artificial Sequence
  Description of Artificial Sequence Synthetic oligonucleotide
uuaguguccgguaaaauga

SEQ ID NO: 833 - RNA, 19 nt, Artificial Sequence
  Description of Artificial Sequence Synthetic oligonucleotide
uuuuaccggacacuaaacc

SEQ ID NO: 834 - RNA, 19 nt, Artificial Sequence
  Description of Artificial Sequence Synthetic oligonucleotide
gguuuaguguccgguaaaa

SEQ ID NO: 835 - RNA, 19 nt, Artificial Sequence
  Description of Artificial Sequence Synthetic oligonucleotide
uuuaccggacacuaaaccc

SEQ ID NO: 836 - RNA, 19 nt, Artificial Sequence
  Description of Artificial Sequence Synthetic oligonucleotide
ggguuuaguguccgguaaa

SEQ ID NO: 837 - RNA, 19 nt, Artificial Sequence
  Description of Artificial Sequence Synthetic oligonucleotide
uuaccggacacuaaaccca

SEQ ID NO: 838 - RNA, 19 nt, Artificial Sequence
  Description of Artificial Sequence Synthetic oligonucleotide
uggguuuaguguccgguaa

SEQ ID NO: 839 - RNA, 19 nt, Artificial Sequence
  Description of Artificial Sequence Synthetic oligonucleotide
uaccggacacuaaacccaa

SEQ ID NO: 840 - RNA, 19 nt, Artificial Sequence
  Description of Artificial Sequence Synthetic oligonucleotide
uuggguuuaguguccggua

SEQ ID NO: 841 - RNA, 19 nt, Artificial Sequence
  Description of Artificial Sequence Synthetic oligonucleotide
aucugguuuugucaagccc

SEQ ID NO: 842 - RNA, 19 nt, Artificial Sequence
  Description of Artificial Sequence Synthetic oligonucleotide
gggcuugacaaaaccagau

SEQ ID NO: 843 - RNA, 19 nt, Artificial Sequence
  Description of Artificial Sequence Synthetic oligonucleotide
aaaaagaagauuucaucga

SEQ ID NO: 844 - RNA, 19 nt, Artificial Sequence
  Description of Artificial Sequence Synthetic oligonucleotide
ucgaugaaaucuucuuuuu

SEQ ID NO: 845 - RNA, 19 nt, Artificial Sequence
  Description of Artificial Sequence Synthetic oligonucleotide
agaagauuucaucgaacuc

SEQ ID NO: 846 - RNA, 19 nt, Artificial Sequence
  Description of Artificial Sequence Synthetic oligonucleotide
gaguucgaugaaaucuucu

SEQ ID NO: 847 - RNA, 19 nt, Artificial Sequence
  Description of Artificial Sequence Synthetic oligonucleotide
aaacugggcacaguuuacu

SEQ ID NO: 848 - RNA, 19 nt, Artificial Sequence
  Description of Artificial Sequence Synthetic oligonucleotide
aguaaacugugcccaguuu

SEQ ID NO: 849 - RNA, 19 nt, Artificial Sequence
  Description of Artificial Sequence Synthetic oligonucleotide
uucuguucauggugugagu

SEQ ID NO: 850 - RNA, 19 nt, Artificial Sequence
  Description of Artificial Sequence Synthetic oligonucleotide
acucacaccaugaacagaa

SEQ ID NO: 851 - RNA, 19 nt, Artificial Sequence
  Description of Artificial Sequence Synthetic oligonucleotide
guucauggugugaguaccu

SEQ ID NO: 852 - RNA, 19 nt, Artificial Sequence
  Description of Artificial Sequence Synthetic oligonucleotide
agguacucacaccaugaac

SEQ ID NO: 853 - RNA, 19 nt, Artificial Sequence
  Description of Artificial Sequence Synthetic oligonucleotide
ggaggacagauguaccacu

SEQ ID NO: 854 - RNA, 19 nt, Artificial Sequence
  Description of Artificial Sequence Synthetic oligonucleotide
agugguacaucuguccucc

SEQ ID NO: 855 - RNA, 19 nt, Artificial Sequence
  Description of Artificial Sequence Synthetic oligonucleotide
cagcaucccuuucucaaca

SEQ ID NO: 856 - RNA, 19 nt, Artificial Sequence
  Description of Artificial Sequence Synthetic oligonucleotide
uguugagaaagggaugcug

SEQ ID NO: 857 - RNA, 19 nt, Artificial Sequence
  Description of Artificial Sequence Synthetic oligonucleotide
aggaucagaagccuauuuu

SEQ ID NO: 858 - RNA, 19 nt, Artificial Sequence
  Description of Artificial Sequence Synthetic oligonucleotide
aaaauaggcuucugauccu

SEQ ID NO: 859 - RNA, 19 nt, Artificial Sequence
  Description of Artificial Sequence Synthetic oligonucleotide
auuccaccaauucccguug

SEQ ID NO: 860 - RNA, 19 nt, Artificial Sequence
  Description of Artificial Sequence Synthetic oligonucleotide
caacgggaauugguggaau

SEQ ID NO: 861 - RNA, 19 nt, Artificial Sequence
  Description of Artificial Sequence Synthetic oligonucleotide
uuccaccaauucccguugg

SEQ ID NO: 862 - RNA, 19 nt, Artificial Sequence
  Description of Artificial Sequence Synthetic oligonucleotide
ccaacgggaauugguggaa

SEQ ID NO: 863 - RNA, 19 nt, Artificial Sequence
  Description of Artificial Sequence Synthetic oligonucleotide
uccaccaauucccguuggu

SEQ ID NO: 864 - RNA, 19 nt, Artificial Sequence
  Description of Artificial Sequence Synthetic oligonucleotide
accaacgggaauuggugga

SEQ ID NO: 865 - RNA, 19 nt, Artificial Sequence
  Description of Artificial Sequence Synthetic oligonucleotide
ccaccaauucccguugguu

SEQ ID NO: 866 - RNA, 19 nt, Artificial Sequence
  Description of Artificial Sequence Synthetic oligonucleotide
aaccaacgggaauuggugg

SEQ ID NO: 867 - RNA, 19 nt, Artificial Sequence
  Description of Artificial Sequence Synthetic oligonucleotide
caccaauucccguugguuc

SEQ ID NO: 868 - RNA, 19 nt, Artificial Sequence
  Description of Artificial Sequence Synthetic oligonucleotide
gaaccaacgggaauuggug

SEQ ID NO: 869 - RNA, 19 nt, Artificial Sequence
  Description of Artificial Sequence Synthetic oligonucleotide
cucugaacuucccuggucg

SEQ ID NO: 870 - RNA, 19 nt, Artificial Sequence
  Description of Artificial Sequence Synthetic oligonucleotide
cgaccagggaaguucagag

SEQ ID NO: 871 - RNA, 19 nt, Artificial Sequence
  Description of Artificial Sequence Synthetic oligonucleotide
acuucccuggucgaacagu

SEQ ID NO: 872 - RNA, 19 nt, Artificial Sequence
  Description of Artificial Sequence Synthetic oligonucleotide
acuguucgaccagggaagu

SEQ ID NO: 873 - RNA, 19 nt, Artificial Sequence
  Description of Artificial Sequence Synthetic oligonucleotide
uggucgaacaguuuuuucu

SEQ ID NO: 874 - RNA, 19 nt, Artificial Sequence
  Description of Artificial Sequence Synthetic oligonucleotide
agaaaaaacuguucgacca

SEQ ID NO: 875 - RNA, 19 nt, Artificial Sequence
  Description of Artificial Sequence Synthetic oligonucleotide
uuucuaauggcuauucaag

SEQ ID NO: 876 - RNA, 19 nt, Artificial Sequence
  Description of Artificial Sequence Synthetic oligonucleotide
cuugaauagccauuagaaa

SEQ ID NO: 877 - RNA, 19 nt, Artificial Sequence
  Description of Artificial Sequence Synthetic oligonucleotide
augagaccagauguaagcu

SEQ ID NO: 878 - RNA, 19 nt, Artificial Sequence
  Description of Artificial Sequence Synthetic oligonucleotide
agcuuacaucuggucucau

SEQ ID NO: 879 - RNA, 19 nt, Artificial Sequence
  Description of Artificial Sequence Synthetic oligonucleotide
ccagauguaagcucuccuc

SEQ ID NO: 880 - RNA, 19 nt, Artificial Sequence
  Description of Artificial Sequence Synthetic oligonucleotide
gaggagagcuuacaucugg

SEQ ID NO: 881 - RNA, 19 nt, Artificial Sequence
  Description of Artificial Sequence Synthetic oligonucleotide
cuggugugcucugaugaag

SEQ ID NO: 882 - RNA, 19 nt, Artificial Sequence
  Description of Artificial Sequence Synthetic oligonucleotide
cuucaucagagcacaccag

SEQ ID NO: 883 - RNA, 19 nt, Artificial Sequence
  Description of Artificial Sequence Synthetic oligonucleotide
gucuuaacuuguggaagcu

SEQ ID NO: 884 - RNA, 19 nt, Artificial Sequence
  Description of Artificial Sequence Synthetic oligonucleotide
agcuuccacaaguuaagac

SEQ ID NO: 885 - RNA, 19 nt, Artificial Sequence
  Description of Artificial Sequence Synthetic oligonucleotide
caucaucgauaaaauucga

SEQ ID NO: 886 - RNA, 19 nt, Artificial Sequence
  Description of Artificial Sequence Synthetic oligonucleotide
ucgaauuuuaucgaugaug

SEQ ID NO: 887 - RNA, 19 nt, Artificial Sequence
  Description of Artificial Sequence Synthetic oligonucleotide
cccagcaugccgcuaucga

SEQ ID NO: 888 - RNA, 19 nt, Artificial Sequence
  Description of Artificial Sequence Synthetic oligonucleotide
ucgauagcggcaugcuggg

SEQ ID NO: 889 - RNA, 19 nt, Artificial Sequence
  Description of Artificial Sequence Synthetic oligonucleotide
ccagcaugccgcuaucgaa

SEQ ID NO: 890 - RNA, 19 nt, Artificial Sequence
  Description of Artificial Sequence Synthetic oligonucleotide
uucgauagcggcaugcugg

SEQ ID NO: 891 - RNA, 19 nt, Artificial Sequence
  Description of Artificial Sequence Synthetic oligonucleotide
cagcaugccgcuaucgaaa

SEQ ID NO: 892 - RNA, 19 nt, Artificial Sequence
  Description of Artificial Sequence Synthetic oligonucleotide
uuucgauagcggcaugcug

SEQ ID NO: 893 - RNA, 19 nt, Artificial Sequence
  Description of Artificial Sequence Synthetic oligonucleotide
agcaugccgcuaucgaaaa

SEQ ID NO: 894 - RNA, 19 nt, Artificial Sequence
  Description of Artificial Sequence Synthetic oligonucleotide
uuuucgauagcggcaugcu

SEQ ID NO: 895 - RNA, 19 nt, Artificial Sequence
  Description of Artificial Sequence Synthetic oligonucleotide
augccgcuaucgaaaaugu

SEQ ID NO: 896 - RNA, 19 nt, Artificial Sequence
  Description of Artificial Sequence Synthetic oligonucleotide
acauuuucgauagcggcau

SEQ ID NO: 897 - RNA, 19 nt, Artificial Sequence
  Description of Artificial Sequence Synthetic oligonucleotide
ccgcuaucgaaaaugucuu

SEQ ID NO: 898 - RNA, 19 nt, Artificial Sequence
  Description of Artificial Sequence Synthetic oligonucleotide
aagacauuuucgauagcgg

SEQ ID NO: 899 - RNA, 19 nt, Artificial Sequence
  Description of Artificial Sequence Synthetic oligonucleotide
cgcuaucgaaaaugucuuc

SEQ ID NO: 900 - RNA, 19 nt, Artificial Sequence
  Description of Artificial Sequence Synthetic oligonucleotide
gaagacauuuucgauagcg

SEQ ID NO: 901 - RNA, 19 nt, Artificial Sequence
  Description of Artificial Sequence Synthetic oligonucleotide
aggaauucagcaggccacu

SEQ ID NO: 902 - RNA, 19 nt, Artificial Sequence
  Description of Artificial Sequence Synthetic oligonucleotide
aguggccugcugaauuccu

SEQ ID NO: 903 - RNA, 19 nt, Artificial Sequence
  Description of Artificial Sequence Synthetic oligonucleotide
auucagcaggccacuacag

SEQ ID NO: 904 - RNA, 19 nt, Artificial Sequence
  Description of Artificial Sequence Synthetic oligonucleotide
cuguaguggccugcugaau

SEQ ID NO: 905 - RNA, 19 nt, Artificial Sequence
  Description of Artificial Sequence Synthetic oligonucleotide
cuacaggagucucacaaga

SEQ ID NO: 906 - RNA, 19 nt, Artificial Sequence
  Description of Artificial Sequence Synthetic oligonucleotide
ucuugugagacuccuguag

SEQ ID NO: 907 - RNA, 19 nt, Artificial Sequence
  Description of Artificial Sequence Synthetic oligonucleotide
aaaacaauaguuccugcaa

SEQ ID NO: 908 - RNA, 19 nt, Artificial Sequence
  Description of Artificial Sequence Synthetic oligonucleotide
uugcaggaacuauuguuuu

SEQ ID NO: 909 - RNA, 19 nt, Artificial Sequence
  Description of Artificial Sequence Synthetic oligonucleotide
aaacaauaguuccugcaac

SEQ ID NO: 910 - RNA, 19 nt, Artificial Sequence
  Description of Artificial Sequence Synthetic oligonucleotide
guugcaggaacuauuguuu

SEQ ID NO: 911 - RNA, 19 nt, Artificial Sequence
  Description of Artificial Sequence Synthetic oligonucleotide
aacaauaguuccugcaacg

SEQ ID NO: 912 - RNA, 19 nt, Artificial Sequence
  Description of Artificial Sequence Synthetic oligonucleotide
cguugcaggaacuauuguu

SEQ ID NO: 913 - RNA, 19 nt, Artificial Sequence
  Description of Artificial Sequence Synthetic oligonucleotide
acaauaguuccugcaacgu

SEQ ID NO: 914 - RNA, 19 nt, Artificial Sequence
  Description of Artificial Sequence Synthetic oligonucleotide
acguugcaggaacuauugu

SEQ ID NO: 915 - RNA, 19 nt, Artificial Sequence
  Description of Artificial Sequence Synthetic oligonucleotide
auaguuccugcaacguuac

SEQ ID NO: 916 - RNA, 19 nt, Artificial Sequence
  Description of Artificial Sequence Synthetic oligonucleotide
guaacguugcaggaacuau

SEQ ID NO: 917 - RNA, 19 nt, Artificial Sequence
  Description of Artificial Sequence Synthetic oligonucleotide
uaguuccugcaacguuacc

SEQ ID NO: 918 - RNA, 19 nt, Artificial Sequence
  Description of Artificial Sequence Synthetic oligonucleotide
gguaacguugcaggaacua

SEQ ID NO: 919 - RNA, 19 nt, Artificial Sequence
  Description of Artificial Sequence Synthetic oligonucleotide
cugcaacguuaccacaacu

SEQ ID NO: 920 - RNA, 19 nt, Artificial Sequence
  Description of Artificial Sequence Synthetic oligonucleotide
aguugugguaacguugcag

SEQ ID NO: 921 - RNA, 19 nt, Artificial Sequence
  Description of Artificial Sequence Synthetic oligonucleotide
ugcaacguuaccacaacuc

SEQ ID NO: 922 - RNA, 19 nt, Artificial Sequence
  Description of Artificial Sequence Synthetic oligonucleotide
gaguugugguaacguugca

SEQ ID NO: 923 - RNA, 19 nt, Artificial Sequence
  Description of Artificial Sequence Synthetic oligonucleotide
ugaaccugaaguguuauau

SEQ ID NO: 924 - RNA, 19 nt, Artificial Sequence
  Description of Artificial Sequence Synthetic oligonucleotide
auauaacacuucagguuca

SEQ ID NO: 925 - RNA, 19 nt, Artificial Sequence
  Description of Artificial Sequence Synthetic oligonucleotide
uguuauaugcaggauauga

SEQ ID NO: 926 - RNA, 19 nt, Artificial Sequence
  Description of Artificial Sequence Synthetic oligonucleotide
ucauauccugcauauaaca

SEQ ID NO: 927 - RNA, 19 nt, Artificial Sequence
  Description of Artificial Sequence Synthetic oligonucleotide
gcucuguuccagacucaac

SEQ ID NO: 928 - RNA, 19 nt, Artificial Sequence
  Description of Artificial Sequence Synthetic oligonucleotide
guugagucuggaacagagc

SEQ ID NO: 929 - RNA, 19 nt, Artificial Sequence
  Description of Artificial Sequence Synthetic oligonucleotide
guuccagacucaacuugga

SEQ ID NO: 930 - RNA, 19 nt, Artificial Sequence
  Description of Artificial Sequence Synthetic oligonucleotide
uccaaguugagucuggaac

SEQ ID NO: 931 - RNA, 19 nt, Artificial Sequence
  Description of Artificial Sequence Synthetic oligonucleotide
cucaacuuggaggaucaug

SEQ ID NO: 932 - RNA, 19 nt, Artificial Sequence
  Description of Artificial Sequence Synthetic oligonucleotide
caugauccuccaaguugag

SEQ ID NO: 933 - RNA, 19 nt, Artificial Sequence
  Description of Artificial Sequence Synthetic oligonucleotide
acgcucaacauguuaggag

SEQ ID NO: 934 - RNA, 19 nt, Artificial Sequence
  Description of Artificial Sequence Synthetic oligonucleotide
cuccuaacauguugagcgu

SEQ ID NO: 935 - RNA, 19 nt, Artificial Sequence
  Description of Artificial Sequence Synthetic oligonucleotide
gggcggcaagugauugcag

SEQ ID NO: 936 - RNA, 19 nt, Artificial Sequence
  Description of Artificial Sequence Synthetic oligonucleotide
cugcaaucacuugccgccc

SEQ ID NO: 937 - RNA, 19 nt, Artificial Sequence
  Description of Artificial Sequence Synthetic oligonucleotide
cagguuucaggaacuuaca

SEQ ID NO: 938 - RNA, 19 nt, Artificial Sequence
  Description of Artificial Sequence Synthetic oligonucleotide
uguaaguuccugaaaccug

SEQ ID NO: 939 - RNA, 19 nt, Artificial Sequence
  Description of Artificial Sequence Synthetic oligonucleotide
gguuucaggaacuuacacc

SEQ ID NO: 940 - RNA, 19 nt, Artificial Sequence
  Description of Artificial Sequence Synthetic oligonucleotide
gguguaaguuccugaaacc

SEQ ID NO: 941 - RNA, 19 nt, Artificial Sequence
  Description of Artificial Sequence Synthetic oligonucleotide
aacuuacaccuggaugacc

SEQ ID NO: 942 - RNA, 19 nt, Artificial Sequence
  Description of Artificial Sequence Synthetic oligonucleotide
ggucauccagguguaaguu

SEQ ID NO: 943 - RNA, 19 nt, Artificial Sequence
  Description of Artificial Sequence Synthetic oligonucleotide
acuuacaccuggaugacca

SEQ ID NO: 944 - RNA, 19 nt, Artificial Sequence
  Description of Artificial Sequence Synthetic oligonucleotide
uggucauccagguguaagu

SEQ ID NO: 945 - RNA, 19 nt, Artificial Sequence
  Description of Artificial Sequence Synthetic oligonucleotide
ugaccaaaugacccuacug

SEQ ID NO: 946 - RNA, 19 nt, Artificial Sequence
  Description of Artificial Sequence Synthetic oligonucleotide
caguagggucauuugguca

SEQ ID NO: 947 - RNA, 19 nt, Artificial Sequence
  Description of Artificial Sequence Synthetic oligonucleotide
ggguggagaucauauagac

SEQ ID NO: 948 - RNA, 19 nt, Artificial Sequence
  Description of Artificial Sequence Synthetic oligonucleotide
gucuauaugaucuccaccc

SEQ ID NO: 949 - RNA, 19 nt, Artificial Sequence
  Description of Artificial Sequence Synthetic oligonucleotide
gguggagaucauauagaca

SEQ ID NO: 950 - RNA, 19 nt, Artificial Sequence
  Description of Artificial Sequence Synthetic oligonucleotide
ugucuauaugaucuccacc

SEQ ID NO: 951 - RNA, 19 nt, Artificial Sequence
  Description of Artificial Sequence Synthetic oligonucleotide
gagaucauauagacaauca

SEQ ID NO: 952 - RNA, 19 nt, Artificial Sequence
  Description of Artificial Sequence Synthetic oligonucleotide
ugauugucuauaugaucuc

SEQ ID NO: 953 - RNA, 19 nt, Artificial Sequence
  Description of Artificial Sequence Synthetic oligonucleotide
cauauagacaaucaagugc

SEQ ID NO: 954 - RNA, 19 nt, Artificial Sequence
  Description of Artificial Sequence Synthetic oligonucleotide
gcacuugauugucuauaug

SEQ ID NO: 955 - RNA, 19 nt, Artificial Sequence
  Description of Artificial Sequence Synthetic oligonucleotide
cauguacgaccaauguaaa

SEQ ID NO: 956 - RNA, 19 nt, Artificial Sequence
  Description of Artificial Sequence Synthetic oligonucleotide
uuuacauuggucguacaug

SEQ ID NO: 957 - RNA, 19 nt, Artificial Sequence
  Description of Artificial Sequence Synthetic oligonucleotide
auguacgaccaauguaaac

SEQ ID NO: 958 - RNA, 19 nt, Artificial Sequence
  Description of Artificial Sequence Synthetic oligonucleotide
guuuacauuggucguacau

SEQ ID NO: 959 - RNA, 19 nt, Artificial Sequence
  Description of Artificial Sequence Synthetic oligonucleotide
uguacgaccaauguaaaca

SEQ ID NO: 960 - RNA, 19 nt, Artificial Sequence
  Description of Artificial Sequence Synthetic oligonucleotide
uguuuacauuggucguaca

SEQ ID NO: 961 - RNA, 19 nt, Artificial Sequence
  Description of Artificial Sequence Synthetic oligonucleotide
caggcuucagguaucuuau

SEQ ID NO: 962 - RNA, 19 nt, Artificial Sequence
  Description of Artificial Sequence Synthetic oligonucleotide
auaagauaccugaagccug

SEQ ID NO: 963 - RNA, 19 nt, Artificial Sequence
  Description of Artificial Sequence Synthetic oligonucleotide
ucuguaugaaaaccuuacu

SEQ ID NO: 964 - RNA, 19 nt, Artificial Sequence
  Description of Artificial Sequence Synthetic oligonucleotide
aguaagguuuucauacaga

SEQ ID NO: 965 - RNA, 19 nt, Artificial Sequence
  Description of Artificial Sequence Synthetic oligonucleotide
cuguaugaaaaccuuacug

SEQ ID NO: 966 - RNA, 19 nt, Artificial Sequence
  Description of Artificial Sequence Synthetic oligonucleotide
caguaagguuuucauacag

SEQ ID NO: 967 - RNA, 19 nt, Artificial Sequence
  Description of Artificial Sequence Synthetic oligonucleotide
guaugaaaaccuuacugcu

SEQ ID NO: 968 - RNA, 19 nt, Artificial Sequence
  Description of Artificial Sequence Synthetic oligonucleotide
agcaguaagguuuucauac

SEQ ID NO: 969 - RNA, 19 nt, Artificial Sequence
  Description of Artificial Sequence Synthetic oligonucleotide
gaaauuagaaugaccuaca

SEQ ID NO: 970 - RNA, 19 nt, Artificial Sequence
  Description of Artificial Sequence Synthetic oligonucleotide
uguaggucauucuaauuuc

SEQ ID NO: 971 - RNA, 19 nt, Artificial Sequence
  Description of Artificial Sequence Synthetic oligonucleotide
gaacuggcagcgguuuuau

SEQ ID NO: 972 - RNA, 19 nt, Artificial Sequence
  Description of Artificial Sequence Synthetic oligonucleotide
auaaaaccgcugccaguuc

SEQ ID NO: 973 - RNA, 19 nt, Artificial Sequence
  Description of Artificial Sequence Synthetic oligonucleotide
acuggcagcgguuuuauca

SEQ ID NO: 974 - RNA, 19 nt, Artificial Sequence
  Description of Artificial Sequence Synthetic oligonucleotide
ugauaaaaccgcugccagu

SEQ ID NO: 975 - RNA, 19 nt, Artificial Sequence
  Description of Artificial Sequence Synthetic oligonucleotide
aacucuuggauucuaugca

SEQ ID NO: 976 - RNA, 19 nt, Artificial Sequence
  Description of Artificial Sequence Synthetic oligonucleotide
ugcauagaauccaagaguu

SEQ ID NO: 977 - RNA, 19 nt, Artificial Sequence
  Description of Artificial Sequence Synthetic oligonucleotide
cacacauuaaucugauuuu

SEQ ID NO: 978 - RNA, 19 nt, Artificial Sequence
  Description of Artificial Sequence Synthetic oligonucleotide
aaaaucagauuaaugugug

SEQ ID NO: 979 - RNA, 19 nt, Artificial Sequence
  Description of Artificial Sequence Synthetic oligonucleotide
ucccaacaaucuuggcgcu

SEQ ID NO: 980 - RNA, 19 nt, Artificial Sequence
  Description of Artificial Sequence Synthetic oligonucleotide
agcgccaagauuguuggga

SEQ ID NO: 981 - RNA, 19 nt, Artificial Sequence
  Description of Artificial Sequence Synthetic oligonucleotide
cccaacaaucuuggcgcuc

SEQ ID NO: 982 - RNA, 19 nt, Artificial Sequence
  Description of Artificial Sequence Synthetic oligonucleotide
gagcgccaagauuguuggg

SEQ ID NO: 983 - RNA, 19 nt, Artificial Sequence
  Description of Artificial Sequence Synthetic oligonucleotide
ccaacaaucuuggcgcuca

SEQ ID NO: 984 - RNA, 19 nt, Artificial Sequence
  Description of Artificial Sequence Synthetic oligonucleotide
ugagcgccaagauuguugg

SEQ ID NO: 985 - RNA, 19 nt, Artificial Sequence
  Description of Artificial Sequence Synthetic oligonucleotide
aacaaucuuggcgcucaaa

SEQ ID NO: 986 - RNA, 19 nt, Artificial Sequence
  Description of Artificial Sequence Synthetic oligonucleotide
uuugagcgccaagauuguu

SEQ ID NO: 987 - RNA, 19 nt, Artificial Sequence
  Description of Artificial Sequence Synthetic oligonucleotide
uuggcgcucaaaaaauaga

SEQ ID NO: 988 - RNA, 19 nt, Artificial Sequence
  Description of Artificial Sequence Synthetic oligonucleotide
ucuauuuuuugagcgccaa

SEQ ID NO: 989 - RNA, 19 nt, Artificial Sequence
  Description of Artificial Sequence Synthetic oligonucleotide
uggcgcucaaaaaauagaa

SEQ ID NO: 990 - RNA, 19 nt, Artificial Sequence
  Description of Artificial Sequence Synthetic oligonucleotide
uucuauuuuuugagcgcca

SEQ ID NO: 991 - RNA, 19 nt, Artificial Sequence
  Description of Artificial Sequence Synthetic oligonucleotide
aggcuuuucauuaaauggg

SEQ ID NO: 992 - RNA, 19 nt, Artificial Sequence
  Description of Artificial Sequence Synthetic oligonucleotide
cccauuuaaugaaaagccu

SEQ ID NO: 993 - RNA, 19 nt, Artificial Sequence
  Description of Artificial Sequence Synthetic oligonucleotide
uccuauguauguguuaucu

SEQ ID NO: 994 - RNA, 19 nt, Artificial Sequence
  Description of Artificial Sequence Synthetic oligonucleotide
agauaacacauacauagga

SEQ ID NO: 995 - RNA, 19 nt, Artificial Sequence
  Description of Artificial Sequence Synthetic oligonucleotide
ccuauguauguguuaucug

SEQ ID NO: 996 - RNA, 19 nt, Artificial Sequence
  Description of Artificial Sequence Synthetic oligonucleotide
cagauaacacauacauagg

SEQ ID NO: 997 - RNA, 19 nt, Artificial Sequence
  Description of Artificial Sequence Synthetic oligonucleotide
cagugagaguugguuacuc

SEQ ID NO: 998 - RNA, 19 nt, Artificial Sequence
  Description of Artificial Sequence Synthetic oligonucleotide
gaguaaccaacucucacug

SEQ ID NO: 999 - RNA, 19 nt, Artificial Sequence
  Description of Artificial Sequence Synthetic oligonucleotide
agugagaguugguuacuca

SEQ ID NO: 1000 - RNA, 19 nt, Artificial Sequence
  Description of Artificial Sequence Synthetic oligonucleotide
ugaguaaccaacucucacu

SEQ ID NO: 1001 - RNA, 19 nt, Artificial Sequence
  Description of Artificial Sequence Synthetic oligonucleotide
gugagaguugguuacucac

SEQ ID NO: 1002 - RNA, 19 nt, Artificial Sequence
  Description of Artificial Sequence Synthetic oligonucleotide
gugaguaaccaacucucac

SEQ ID NO: 1003 - RNA, 19 nt, Artificial Sequence
  Description of Artificial Sequence Synthetic oligonucleotide
ugagaguugguuacucaca

SEQ ID NO: 1004 - RNA, 19 nt, Artificial Sequence
  Description of Artificial Sequence Synthetic oligonucleotide
ugugaguaaccaacucuca

SEQ ID NO: 1005 - RNA, 19 nt, Artificial Sequence
  Description of Artificial Sequence Synthetic oligonucleotide
ugguccacccaggauuagu

SEQ ID NO: 1006 - RNA, 19 nt, Artificial Sequence
  Description of Artificial Sequence Synthetic oligonucleotide
acuaauccuggguggacca

SEQ ID NO: 1007 - RNA, 19 nt, Artificial Sequence
  Description of Artificial Sequence Synthetic oligonucleotide
gguccacccaggauuagug

SEQ ID NO: 1008 - RNA, 19 nt, Artificial Sequence
  Description of Artificial Sequence Synthetic oligonucleotide
cacuaauccuggguggacc

SEQ ID NO: 1009 - RNA, 19 nt, Artificial Sequence
  Description of Artificial Sequence Synthetic oligonucleotide
uagugaccagguuuucagg

SEQ ID NO: 1010 - RNA, 19 nt, Artificial Sequence
  Description of Artificial Sequence Synthetic oligonucleotide
ccugaaaaccuggucacua

SEQ ID NO: 1011 - RNA, 19 nt, Artificial Sequence
  Description of Artificial Sequence Synthetic oligonucleotide
ggcuguaugaaaauacccu

SEQ ID NO: 1012 - RNA, 19 nt, Artificial Sequence
  Description of Artificial Sequence Synthetic oligonucleotide
aggguauuuucauacagcc

SEQ ID NO: 1013 - RNA, 19 nt, Artificial Sequence
  Description of Artificial Sequence Synthetic oligonucleotide
cuguaugaaaauacccucc

SEQ ID NO: 1014 - RNA, 19 nt, Artificial Sequence
  Description of Artificial Sequence Synthetic oligonucleotide
ggaggguauuuucauacag

SEQ ID NO: 1015 - RNA, 19 nt, Artificial Sequence
  Description of Artificial Sequence Synthetic oligonucleotide
auacccuccucaaauaacu

SEQ ID NO: 1016 - RNA, 19 nt, Artificial Sequence
  Description of Artificial Sequence Synthetic oligonucleotide
aguuauuugaggaggguau

SEQ ID NO: 1017 - RNA, 19 nt, Artificial Sequence
  Description of Artificial Sequence Synthetic oligonucleotide
aaauaacuugcuuaacuac

SEQ ID NO: 1018 - RNA, 19 nt, Artificial Sequence
  Description of Artificial Sequence Synthetic oligonucleotide
guaguuaagcaaguuauuu

SEQ ID NO: 1019 - RNA, 19 nt, Artificial Sequence
  Description of Artificial Sequence Synthetic oligonucleotide
aauaacuugcuuaacuaca

SEQ ID NO: 1020 - RNA, 19 nt, Artificial Sequence
  Description of Artificial Sequence Synthetic oligonucleotide
uguaguuaagcaaguuauu

SEQ ID NO: 1021 - RNA, 19 nt, Artificial Sequence
  Description of Artificial Sequence Synthetic oligonucleotide
uugcuuaacuacauauaga

SEQ ID NO: 1022 - RNA, 19 nt, Artificial Sequence
  Description of Artificial Sequence Synthetic oligonucleotide
ucuauauguaguuaagcaa

SEQ ID NO: 1023 - RNA, 19 nt, Artificial Sequence
  Description of Artificial Sequence Synthetic oligonucleotide
ugcuuaacuacauauagau

SEQ ID NO: 1024 - RNA, 19 nt, Artificial Sequence
  Description of Artificial Sequence Synthetic oligonucleotide
aucuauauguaguuaagca

SEQ ID NO: 1025 - RNA, 19 nt, Artificial Sequence
  Description of Artificial Sequence Synthetic oligonucleotide
uaguuuuuuauucaugcug

SEQ ID NO: 1026 - RNA, 19 nt, Artificial Sequence
  Description of Artificial Sequence Synthetic oligonucleotide
cagcaugaauaaaaaacua

SEQ ID NO: 1027 - RNA, 19 nt, Artificial Sequence
  Description of Artificial Sequence Synthetic oligonucleotide
caugcugaauaauaaucug

SEQ ID NO: 1028 - RNA, 19 nt, Artificial Sequence
  Description of Artificial Sequence Synthetic oligonucleotide
cagauuauuauucagcaug

SEQ ID NO: 1029 - RNA, 19 nt, Artificial Sequence
  Description of Artificial Sequence Synthetic oligonucleotide
acuguaaaaccuugugugg

SEQ ID NO: 1030 - RNA, 19 nt, Artificial Sequence
  Description of Artificial Sequence Synthetic oligonucleotide
ccacacaagguuuuacagu

SEQ ID NO: 1031 - RNA, 19 nt, Artificial Sequence
  Description of Artificial Sequence Synthetic oligonucleotide
ugcuguucugguauuacca

SEQ ID NO: 1032 - RNA, 19 nt, Artificial Sequence
  Description of Artificial Sequence Synthetic oligonucleotide
ugguaauaccagaacagca

SEQ ID NO: 1033 - RNA, 19 nt, Artificial Sequence
  Description of Artificial Sequence Synthetic oligonucleotide
uggucgaacaguuuuuucc

SEQ ID NO: 1034 - RNA, 19 nt, Artificial Sequence
  Description of Artificial Sequence Synthetic oligonucleotide
uggucgaacaguuuuuucg

SEQ ID NO: 1035 - RNA, 19 nt, Artificial Sequence
  Description of Artificial Sequence Synthetic oligonucleotide
guuccagacucaacuuggc

SEQ ID NO: 1036 - RNA, 19 nt, Artificial Sequence
  Description of Artificial Sequence Synthetic oligonucleotide
guuccagacucaacuuggu